(19) United States
(12) Patent Application Publication
(10) Pub. No.: US 2026/0021190 A1
(43) Pub. Date: Jan. 22, 2026

(54) GLUCOSE-RESPONSIVE INSULIN CONJUGATES COMPRISING A PENTA-VALENT SUGAR CLUSTER FOR TREATMENT OF DIABETES

(71) Applicant: Merck Sharp & Dohme LLC, Rahway, NJ (US)

(72) Inventors: David N. Hunter, Hatfield, PA (US); Pei Huo, Millburn, NJ (US); Songnian Lin, Holmdel, NJ (US); Christopher R. Moyes, Westfield, NJ (US); Dmitri A. Pissarnitski, Scotch Plains, NJ (US); Lin Yan, East Brunswick, NJ (US); Yuping Zhu, Basking Ridge, NJ (US)

(73) Assignee: Merck Sharp & Dohme LLC, Rahway, NJ (US)

(21) Appl. No.: 19/125,537

(22) PCT Filed: Nov. 6, 2023
(86) PCT No.: PCT/US2023/078774

§ 371 Date: Apr. 29, 2025

Related U.S. Application Data
(60) Provisional application No. 63/423,850, filed on Nov. 9, 2022.

(51) Int. Cl. A61K 47/61; A61P 3/10

(52) U.S. Cl. CPC A61K 47/61; A61P 3/10

(57) ABSTRACT

An insulin conjugate comprising or consisting of a penta-valent sugar cluster is described. In particular aspects, the insulin conjugate displays a pharmacokinetic (PK) and/or pharmacodynamic (PD) profile that is responsive to the systemic concentrations of a saccharide, such as glucose or alpha-methylmannose.

### FIELD OF THE INVENTION

The present disclosure relates to an insulin conjugate comprising or consisting of a penta-valent sugar cluster. In particular aspects, the insulin conjugate that displays a pharmacokinetic (“PK”) and/or pharmacodynamic (“PD”) profile that is responsive to the systemic concentrations of a saccharide such as glucose or alpha-methylmannose.

### BACKGROUND OF THE INVENTION

The majority of “controlled-release” drug delivery systems known in the prior art (e.g., U.S. Pat. No. 4,145,410 to Sears, which describes drug release from capsules that are enzymatically labile) are incapable of providing drugs to a patient at intervals and concentrations which are in direct proportion to the amount of a molecular indicator (e.g., a metabolite) present in the human body. The drugs in these prior art systems are thus not literally “controlled,” but simply provided in a slow release format that is independent of external or internal factors. The treatment of diabetes mellitus with injectable insulin is a well-known and studied example where uncontrolled, slow release of insulin is undesirable. In fact, it is apparent that the simple replacement of the hormone is not sufficient to prevent the pathological sequelae associated with this disease. The development of these sequelae is believed to reflect an inability to provide exogenous insulin proportional to varying blood glucose concentrations experienced by the patient. To solve this problem several biological and bioengineering approaches to develop a more physiological insulin delivery system have been suggested (e.g., see U.S. Pat. No. 4,348,387 to Brownlee et al. U.S. Pat. Nos. 5,830,506, 5,902,603, and 6,410,053 to Taylor et al. and U.S. Patent Application Publication No. 2004-0202719 to Zion et al.).

Insulin replacement therapy for glycemic control in diabetic patients, however, is often insufficient due to the inability of exogenous insulins to function in response to the varying glucose concentration. Among approaches to develop glucose responsive insulins, conjugation of a cluster of sugars, e.g., D-mannose and L-fucose, to insulin has been reported in patent literature that potentially offer such glucose responsive insulins. See Neils C. Kaarsholm et al., Engineering Glucose Responsiveness into Insulin, 67 Diabetes 299-308 (February 2018). The cluster of sugar moieties, acting as substrate of endogenous mannose receptor, potentially affect the pharmacokinetic properties of their corresponding insulin conjugates in a way that is sensitive to the endogenous glucose concentration, rendering these insulin conjugates low risk of hypoglycemia. However, there remains a need for additional insulin replacement therapies for glycemic control in diabetic patients.

### SUMMARY OF THE INVENTION

The present disclosure provides insulin conjugates comprising a cluster of penta-valent sugar moieties onto one, two, or three amino groups of Gly^{A1}, Lys^{εB29}, or Phe^{B1} of insulin offers a balanced binding profile against both insulin receptor and mannose receptor. Such penta-valent sugar cluster conjugates may provide glucose lowering in the presence of alpha-methylmannose, a surrogate for glucose, and may allow for improved glycemic controls in the treatment of diabetes with lower risk of hypoglycemia.

Other embodiments, aspects and features of the present invention are either further described in or will be apparent from the ensuing description, examples and appended claims.

### BRIEF DESCRIPTION OF THE DRAWINGS

FIG. 1 shows blood glucose depression curves in non-diabetic male Yucatan minipigs equipped with dual vascular access ports (n=3 per study) following intravenous (“i.v.” or “IV”) injection of conjugate IOC-1 at 0.69 nmol/kg under conditions of phosphate buffered saline (“PBS”) infusion or i.v. alpha-methylmannose (“αMM”) infusion.

FIG. 2 shows blood glucose depression curves in non-diabetic male Yucatan minipigs equipped with dual vascular access ports (n=3 per study) following i.v. injection of conjugate IOC-9 at 0.69 nmol/kg under conditions of PBS infusion or i.v. αMM infusion.

FIG. 3 shows blood glucose depression curves in non-diabetic male Yucatan minipigs equipped with dual vascular access ports (n=3 per study) following i.v. injection of conjugate IOC-14 at 0.69 nmol/kg under conditions of PBS infusion or i.v. αMM infusion.

FIG. 4 shows blood glucose depression curves in non-diabetic male Yucatan minipigs equipped with dual vascular access ports (n=3 per study) following i.v. injection of conjugate IOC-26 at 0.69 nmol/kg under conditions of PBS infusion or i.v. αMM infusion.

FIG. 5 shows blood glucose depression curves in non-diabetic male Yucatan minipigs equipped with dual vascular access ports (n=3 per study) following i.v. injection of conjugate IOC-32 at 0.69 nmol/kg under conditions of PBS infusion or i.v. αMM infusion.

FIG. 6 shows blood glucose depression curves in non-diabetic male Yucatan minipigs equipped with dual vascular access ports (n=3 per study) following i.v. injection of conjugate IOC-33 at 0.69 nmol/kg under conditions of PBS infusion or i.v. αMM infusion.

FIG. 7 shows blood glucose depression curves in non-diabetic male Yucatan minipigs equipped with dual vascular access ports (n=3 per study) following i.v. injection of conjugate IOC-36 at 0.69 nmol/kg under conditions of PBS infusion or i.v. αMM infusion.

FIG. 8 shows blood glucose depression curves in non-diabetic male Yucatan minipigs equipped with dual vascular access ports (n=3 per study) following i.v. injection of conjugate IOC-47 at 0.69 nmol/kg under conditions of PBS infusion or i.v. αMM infusion.

FIG. 9 shows blood glucose depression curves in non-diabetic male Yucatan minipigs equipped with dual vascular access ports (n=3 per study) following i.v. injection of conjugate IOC-49 at 0.69 nmol/kg under conditions of PBS infusion or i.v. αMM infusion.

FIG. 10 shows blood glucose depression curves in non-diabetic male Yucatan minipigs equipped with dual vascular access ports (n=3 per study) following i.v. injection of conjugate IOC-51 at 0.69 nmol/kg under conditions of PBS infusion or i.v. αMM infusion.

FIG. 11 shows blood glucose depression curves in non-diabetic male Yucatan minipigs equipped with dual vascular access ports (n=3 per study) following i.v. injection of conjugate IOC-52 at 0.69 nmol/kg under conditions of PBS infusion or i.v. αMM infusion.

FIG. 12 shows blood glucose depression curves in non-diabetic male Yucatan minipigs equipped with dual vascular access ports (n=3 per study) following i.v. injection of conjugate IOC-53 at 0.69 nmol/kg under conditions of PBS infusion or i.v. αMM infusion.

### DETAILED DESCRIPTION OF THE INVENTION

The present disclosure provides insulin conjugates comprising one, two, or three penta-valent sugar cluster(s). These insulin conjugates may display a pharmacokinetic (“PK”) and/or pharmacodynamic (“PD”) profile that is responsive to the systemic concentrations of a saccharide, such as glucose or alpha-methylmannose, when administered to a subject in need thereof in the absence of an exogenous multivalent saccharide-binding molecule such as the lectin Concanavalin A (“Con A”). In general, the conjugates comprise an insulin or insulin analog molecule covalently attached at its Gly^{A1}, Lys^{εB29}, or Phe^{B}1 amino acid to a linker having a penta-valent sugar cluster thereon. In particular embodiments, the conjugates comprise an insulin or insulin analog molecule covalently attached at its Lys^{εB29} amino acid to a linker having a penta-valent sugar cluster thereon.

In particular embodiments, a conjugate may have a polydispersity index of one and a molecular weight (“MW”) of less than about 20,000 Da. In particular embodiments, the conjugate is long acting (i.e., exhibits a PK profile that is more sustained than soluble recombinant human insulin (“RHI”)).

The conjugates disclosed herein may display a PD or PK profile that is sensitive to the serum concentration of a serum saccharide when administered to a subject in need thereof in the absence of an exogenous saccharide binding molecule. In particular aspects, the serum saccharide is glucose or alpha-methylmannose. In further aspects, the conjugate binds an endogenous saccharide binding molecule at a serum glucose concentration of 60 or 70 mg/dL or less when administered to a subject in need thereof. The binding of the conjugate to the endogenous saccharide binding molecule is sensitive to the serum concentration of the serum saccharide. In a further aspect, the conjugate is capable of binding the insulin receptor at a serum saccharide concentration greater than 60, 70, 80, 90, or 100 mg/dL. At serum saccharide concentration at 60 or 70 mg/dL, the conjugate preferentially binds the endogenous saccharide binding molecule over the insulin receptor, and, as the serum concentration of the serum saccharide increases from 60 or 70 mg/dL, the binding of the conjugate to the endogenous saccharide binding molecule decreases, and the binding of the conjugate to the insulin receptor increases.

The present disclosure provides a conjugate comprising an insulin or insulin analog molecule covalently attached to at least one penta-valent sugar cluster, wherein the penta-valent sugar cluster is provided by a penta-dentate linker having five arms, wherein each arm of the penta-dentate linker is independently covalently linked to a ligand comprising or consisting of a saccharide, such as a monosaccharide, disaccharide, trisaccharide, tetrasaccharide, or branched trisaccharide.

In particular embodiments of the conjugate, the conjugate comprises an insulin or insulin analog molecule conjugated to at least two penta-valent sugar clusters. In a further embodiment, the conjugate comprises an insulin or insulin analog molecule conjugated to at least three penta-valent sugar clusters.

The present disclosure provides a conjugate comprising an insulin or insulin analog molecule covalently attached to one penta-valent sugar cluster, wherein the penta-valent sugar cluster is provided by a penta-dentate linker having five arms, wherein each arm of the penta-dentate linker is independently covalently linked to a ligand comprising or consisting of a saccharide, such as a monosaccharide, disaccharide, trisaccharide, tetrasaccharide, or branched trisaccharide.

The present disclosure provides a conjugate comprising an insulin or insulin analog molecule covalently attached to two penta-valent sugar clusters, wherein each penta-valent sugar cluster is provided by a penta-dentate linker having five arms, wherein each arm of the penta-dentate linker is independently covalently linked to a ligand comprising or consisting of a saccharide, such as a monosaccharide, disaccharide, trisaccharide, tetrasaccharide, or branched trisaccharide.

The present disclosure provides a conjugate comprising an insulin or insulin analog molecule covalently attached to three penta-valent sugar clusters wherein each penta-valent sugar cluster is provided by a penta-dentate linker having five arms wherein each arm of the penta-dentate linker is independently covalently linked to a ligand comprising or consisting of a saccharide, such as a monosaccharide, disaccharide, trisaccharide, tetrasaccharide, or branched trisaccharide.

In particular embodiments of the conjugate, the ligand comprises or consists of a saccharide selected from the group consisting of fucose, mannose, glucosamine, glucose, bimannose (also referred to herein as dimannose), trimannose, tetramannose, or branched trimannose.

In particular embodiments, the ligand comprises or consists of a saccharide and amine group. In particular embodiments, the saccharide and amine group are separated by a C₁-C₆ alkyl group, e.g., a C₁-C₃ alkyl group.

In particular embodiments, the ligand comprises or consists of a saccharide selected from the group consisting of aminoethylglucose (“AEG”), aminoethylmannose (“AEM”), aminoethylbimannose (“AEBM”), aminoethyltrimannose (“AETM”), β-aminoethyl-N-acetylglucosamine (“AEGA”), and aminoethylfucose (“AEF”). In particular embodiments, the saccharide is of the “D” configuration and in other embodiments, the saccharide is of the “L” configuration.

In particular embodiments of the conjugate, the penta-valent sugar cluster is covalently linked to the side chain amino group of B29 lysine of insulin or insulin analog, in the presence or absence of small substitution on A1 and B1 amino groups, which offers a balanced binding profile against both insulin receptor and mannose receptor. These B29 conjugates demonstrate glucose lowering in preclinical insulin replacement therapy for glycemic control in diabetic patients, however, is often insufficient due to the inability of these exogenous insulins to function in response to the varying glucose concentration. Among approaches to develop glucose responsive insulins, conjugation of a cluster of sugars, e.g., D-mannose and L-fucose, to insulin has been reported in patent literature that potentially offer such glucose responsive insulins. The cluster of sugar moieties, acting as substrate of endogenous mannose receptor, potentially affect the pharmacokinetic properties of their corresponding insulin conjugates in a way that is sensitive to the endogenous glucose concentration, rendering these insulin conjugates low risk of hypoglycemia. The conjugation of a cluster of penta-valent sugar moieties onto the side chain amino group of B29 lysine of insulin or insulin analog, in the presence or absence of small substitution on A1 and B1 amino groups, offers a balanced binding profile against both insulin receptor and mannose receptor. These B29 conjugates demonstrate glucose lowering in preclinical mini-pig model in the presence of alpha-methyl mannose, a surrogate for glucose, and potentially might be used for the treatment of diabetes with lower risk of hypoglycemia.

In particular embodiments of the conjugate, the insulin analog is insulin lispro, insulin glargine, insulin aspart, insulin detemir, or insulin glulisine.

In particular embodiments of the conjugate, the conjugate displays a PD and/or PK profile that is sensitive to the serum concentration of a serum saccharide when administered to a subject in need thereof in the absence of an exogenous saccharide binding molecule.

In particular embodiments of the conjugate, the serum saccharide is glucose or alpha-methylmannose.

In particular embodiments of the conjugate, the conjugate binds an endogenous saccharide binding molecule at a serum glucose concentration of 60 mg/dL or less when administered to a subject in need thereof.

In particular embodiments of the conjugate, the endogenous saccharide binding molecule is human mannose receptor 1.

In particular embodiments, the conjugate has the general formula I:

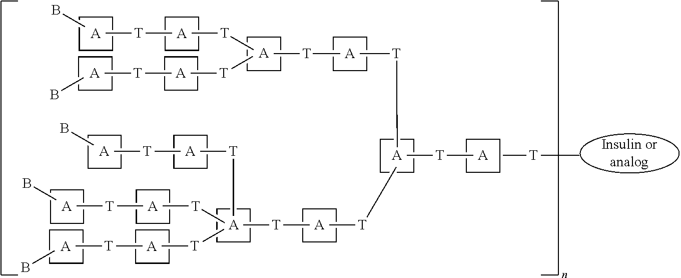

or has the general formula II:

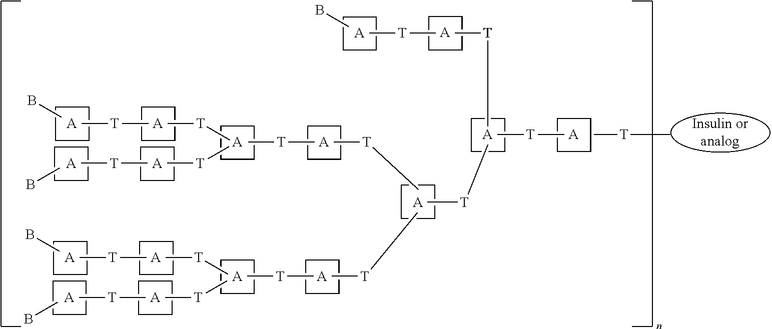

or has the general formula III:

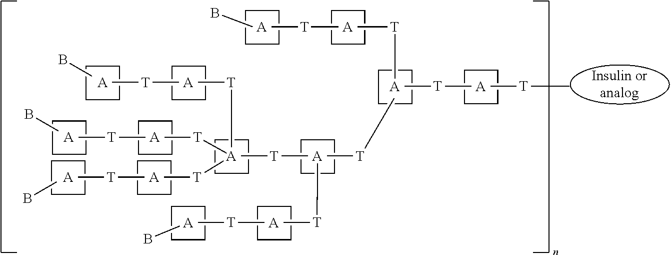

or has the general formula IV:

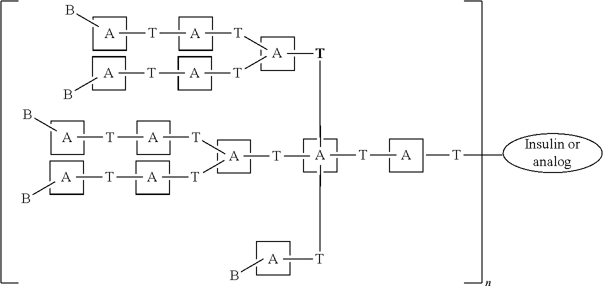

wherein
(i) T is a linker, and each occurrence of T is independently selected from a covalent bond or a bivalent, straight or branched, saturated or unsaturated, optionally substituted C₁₋₃₀ hydrocarbon chain, wherein
   (a) one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic moiety, an aryl moiety, or a heteroaryl moiety, and
   (b) each occurrence of R is independently hydrogen, a suitable protecting group, an acyl moiety, arylalkyl moiety, aliphatic moiety, aryl moiety, heteroaryl moiety, or heteroaliphatic moiety;
(ii) [symbol] is a branching point, and each occurrence of [symbol] is independently selected from a covalent bond, a carbon atom, a heteroatom, or an optionally substituted group selected from the group consisting of acyl moieties, aliphatic moieties, heteroaliphatic moieties, aryl moieties, heteroaryl moieties, and heterocyclic moieties; and
(iii) B is a sugar-containing moiety, and each occurrence of B is independently -T-L^{B}-X, wherein each occurrence of X is independently a ligand comprising or consisting of a monosaccharide, disaccharide, trisaccharide, tetrasaccharide, or branched trisaccharide, and each occurrence of L^{B} is independently a covalent bond or a group derived from the covalent conjugation of a T with an X; and,
(iv) n is 1, 2, or 3.

In further embodiments, the conjugate is a conjugate having general formula I, and the insulin or insulin analog is conjugated to a pentavalent linker selected from the group consisting of:

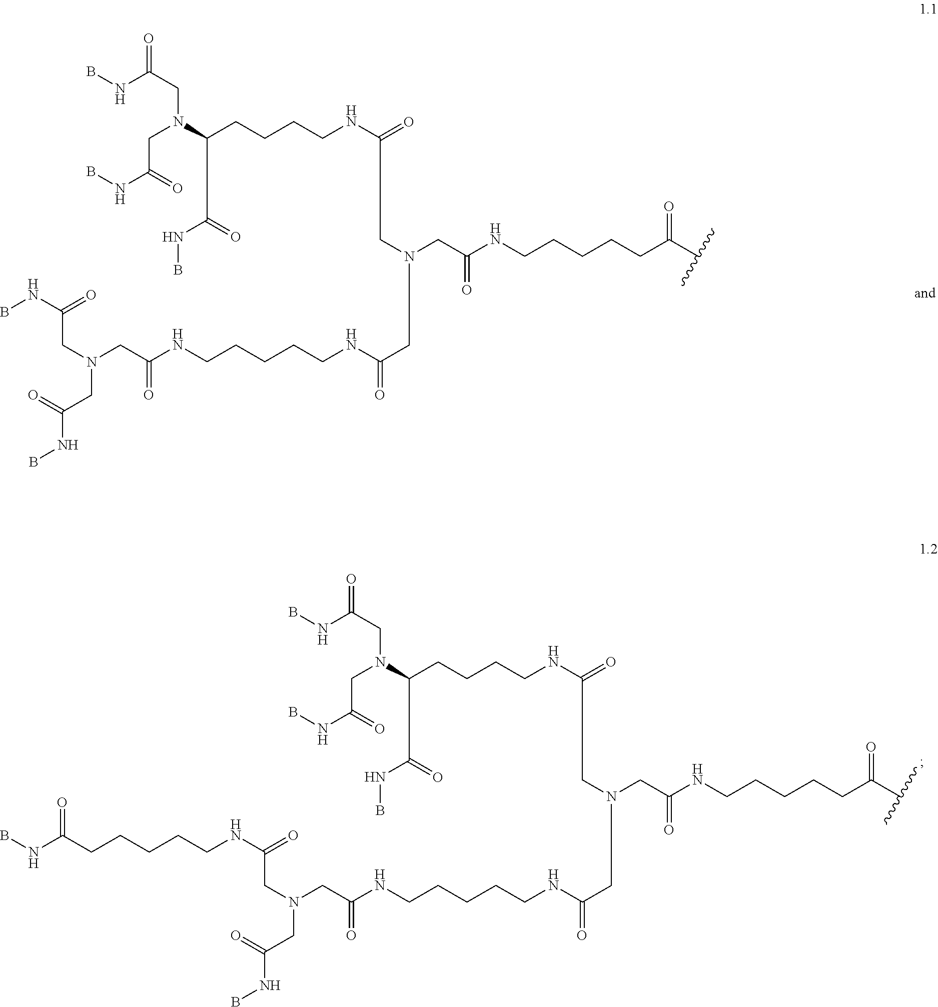

or the conjugate is a conjugate having general formula II, and the insulin or insulin analog is conjugated to a pentavalent linker selected from the group consisting of:

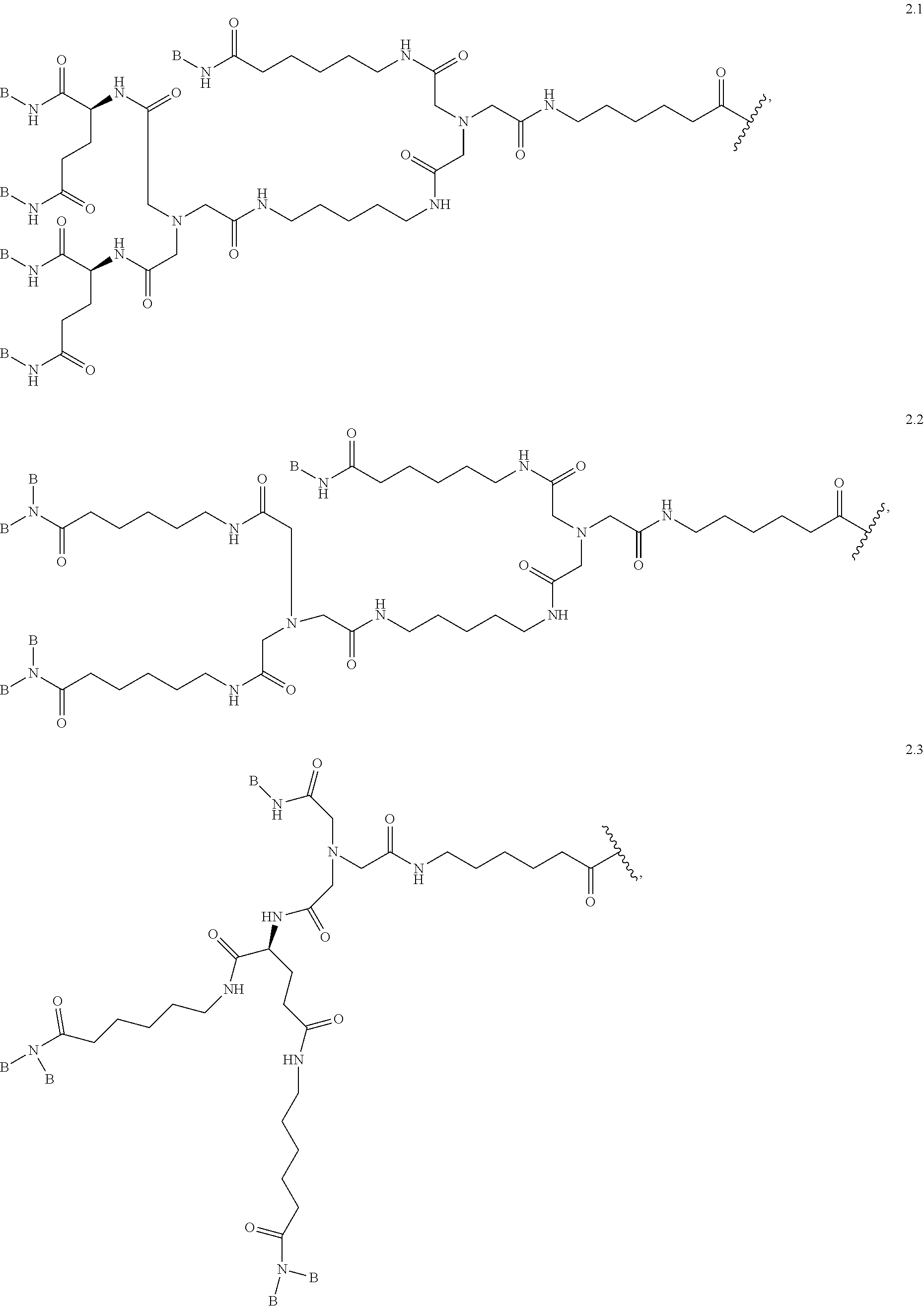

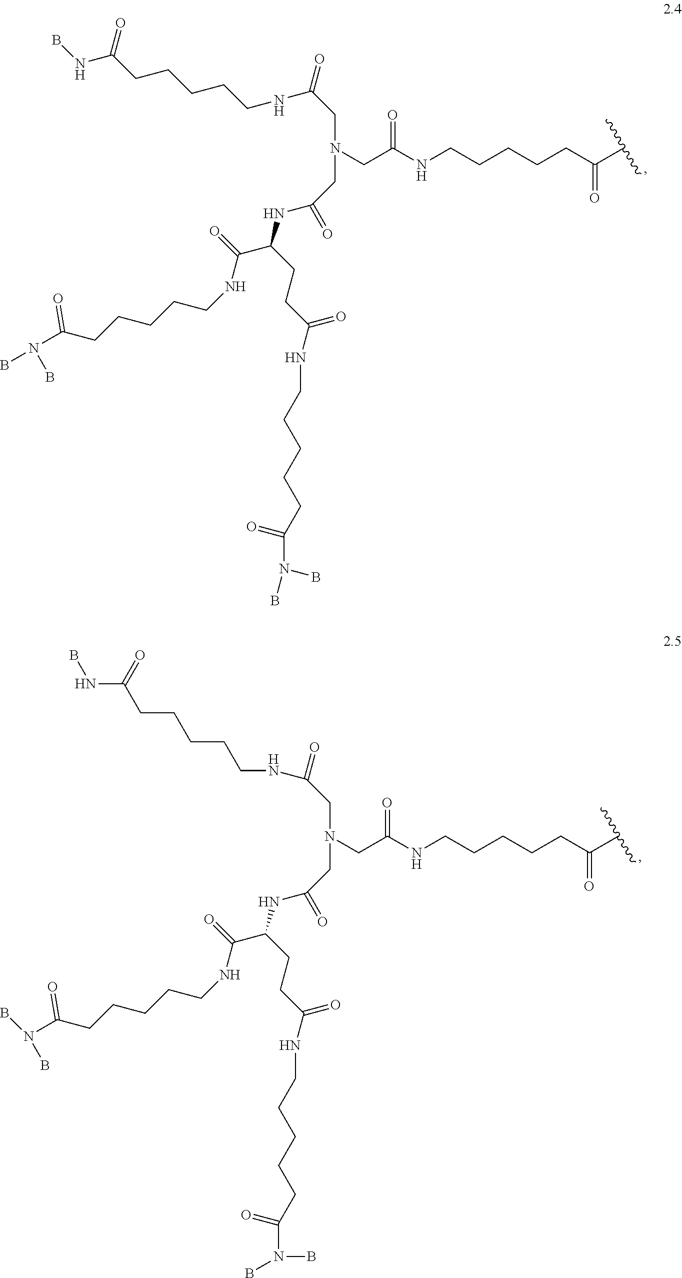

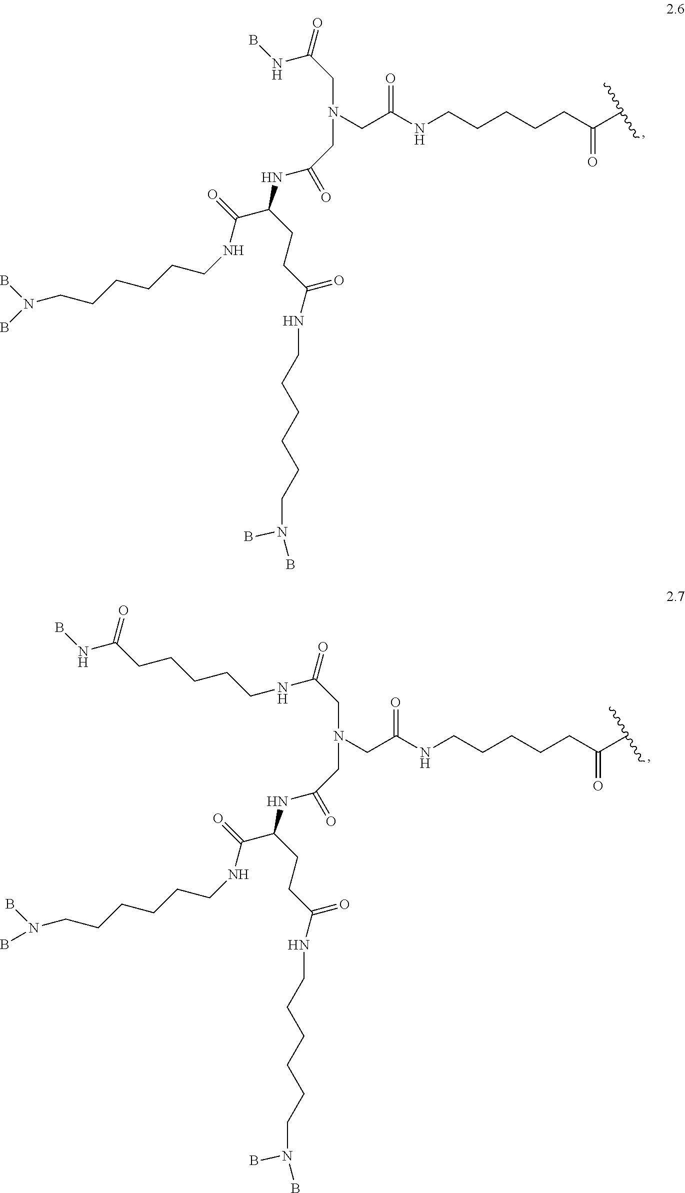

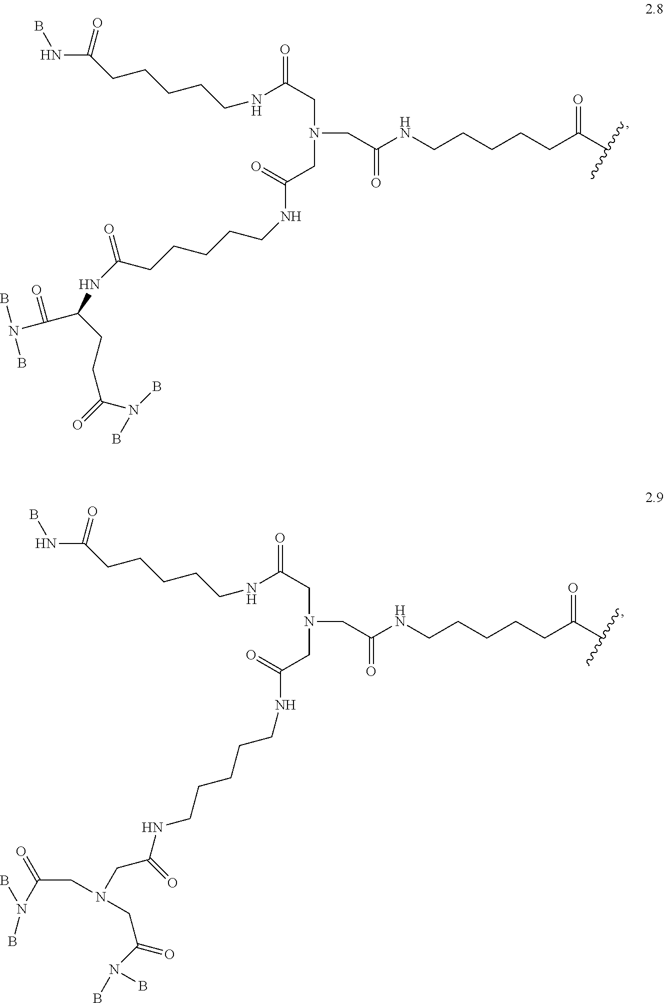

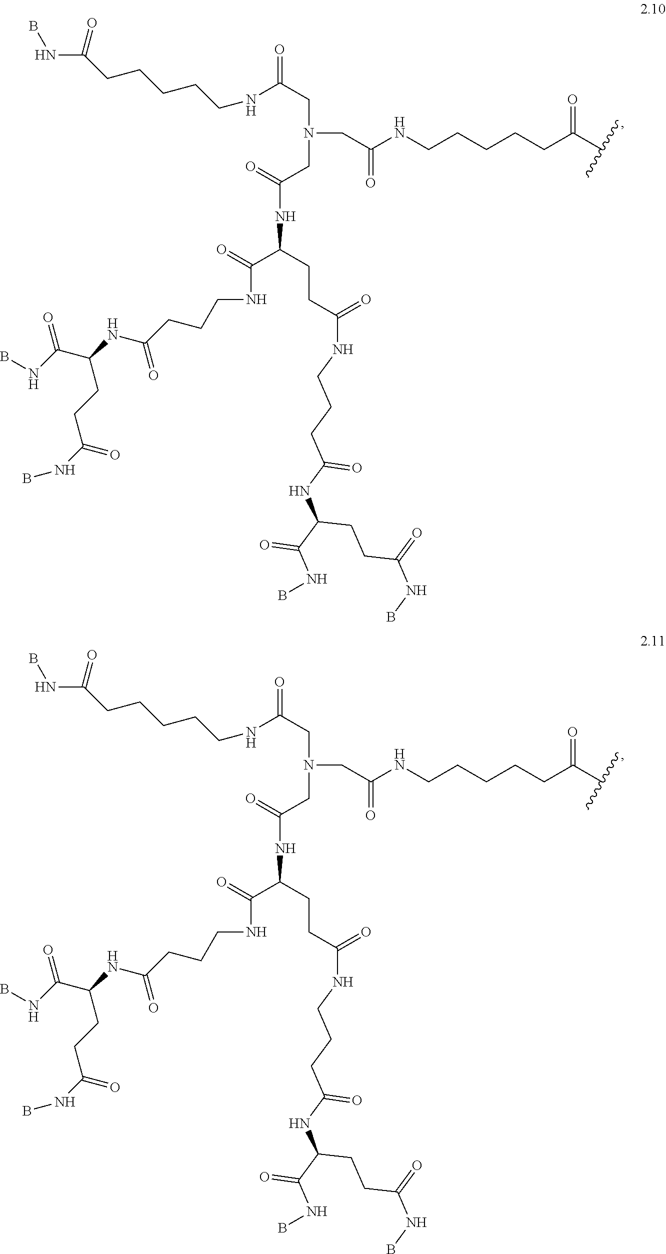

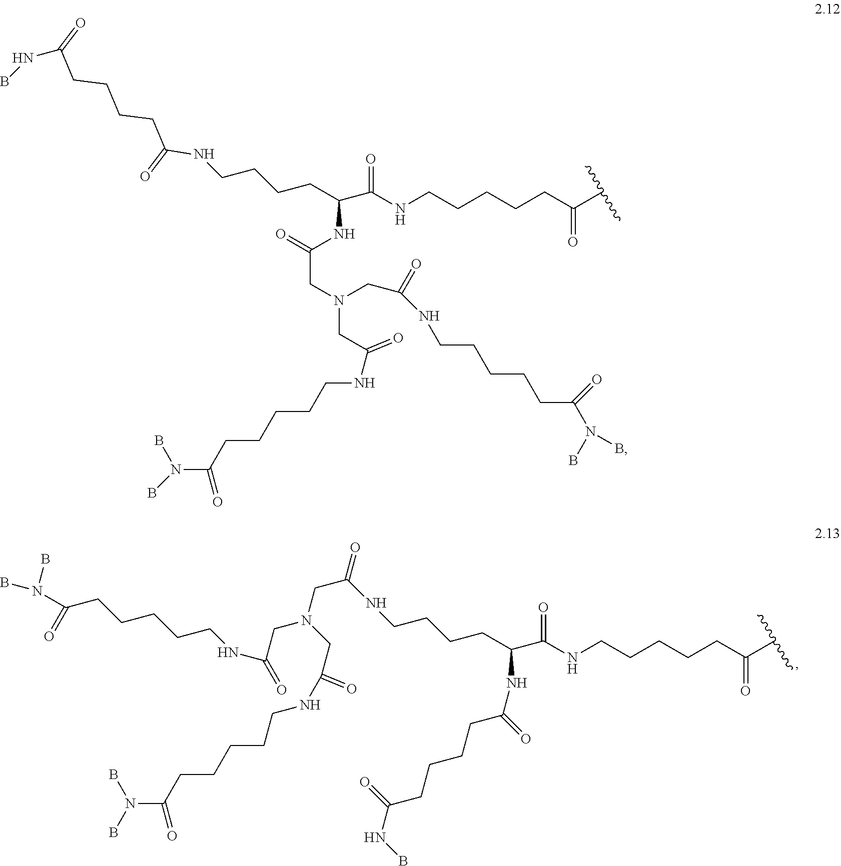

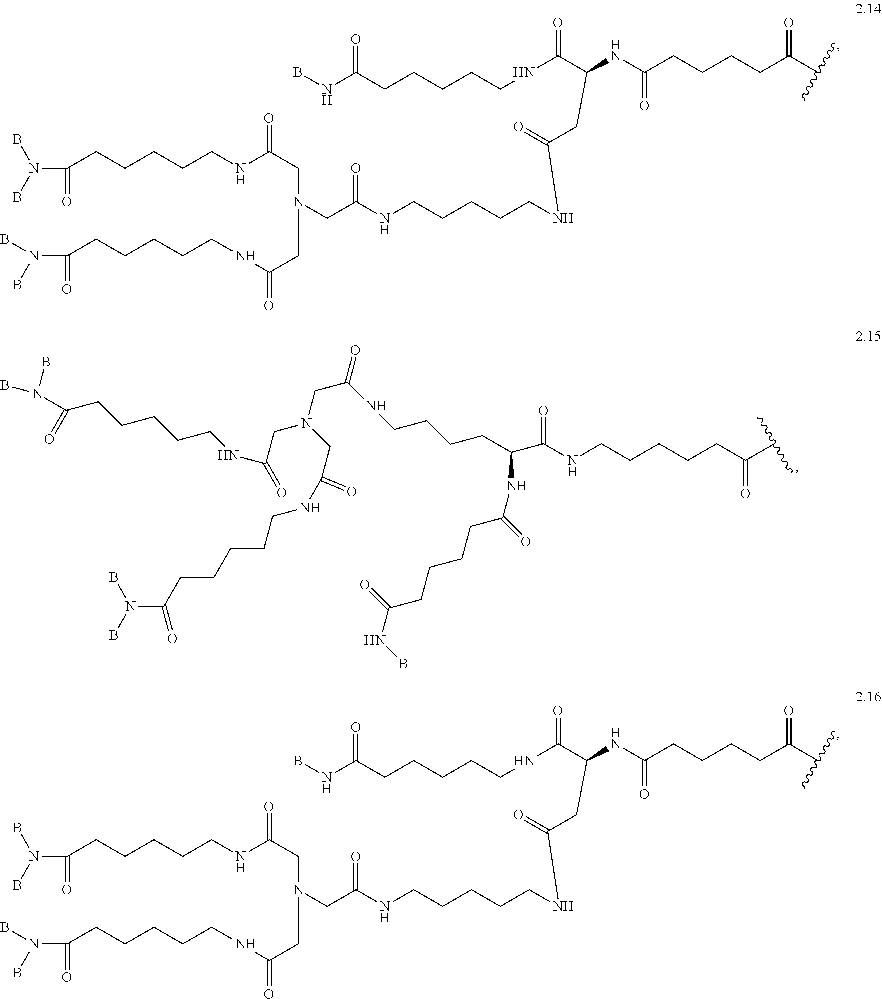

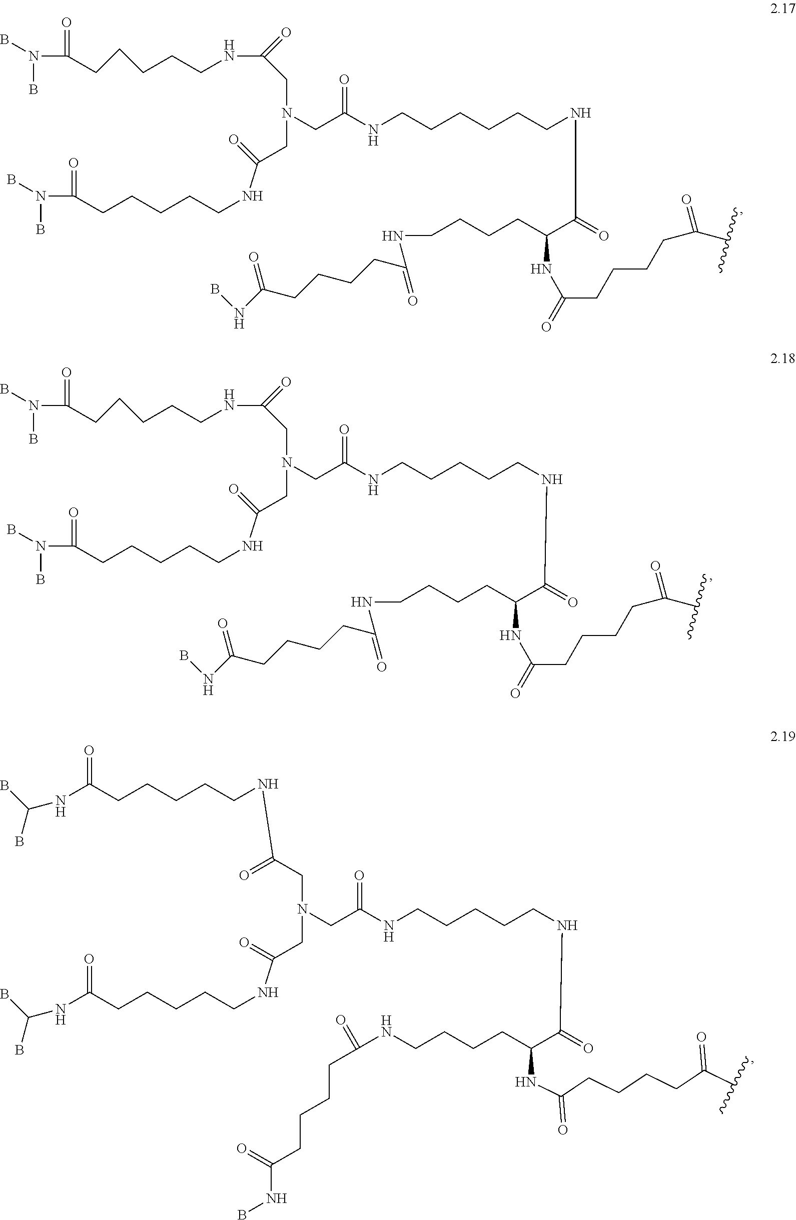

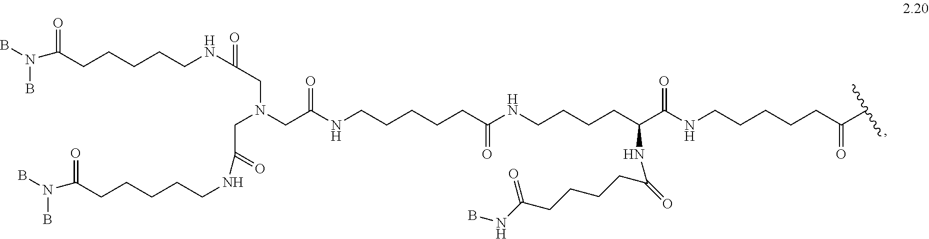

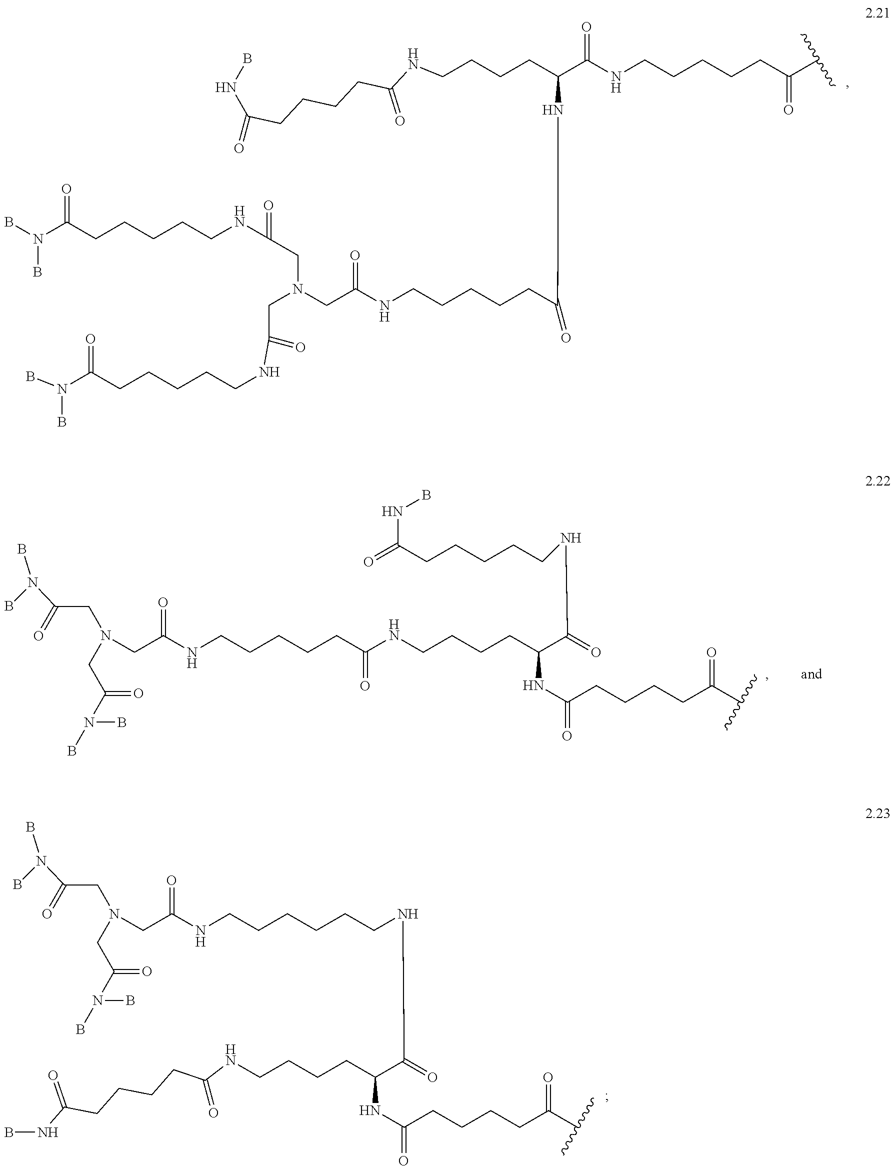

or the conjugate is a conjugate having general formula III, and the insulin or insulin analog is conjugated to a pentavalent linker selected from the group consisting of:

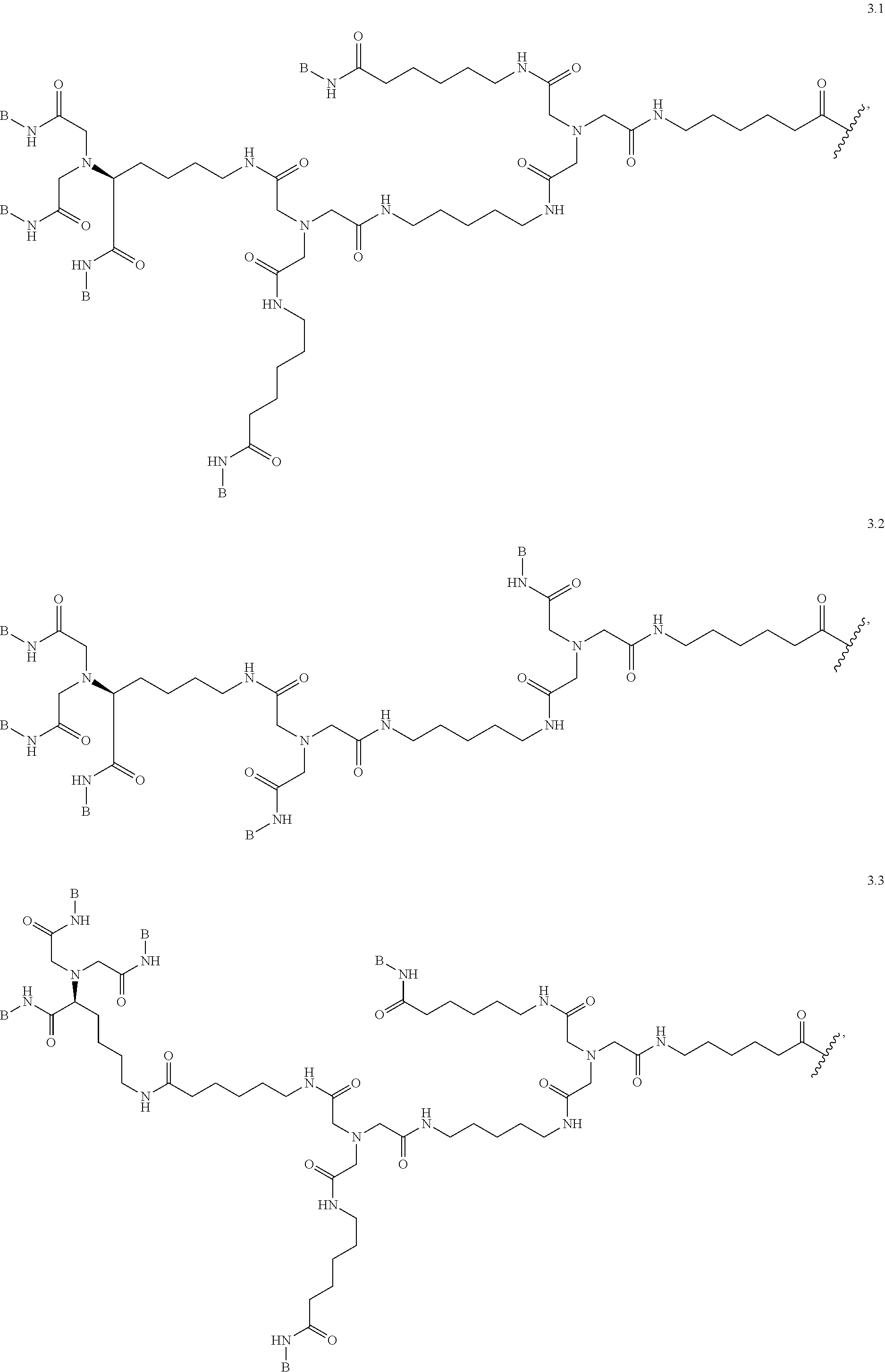

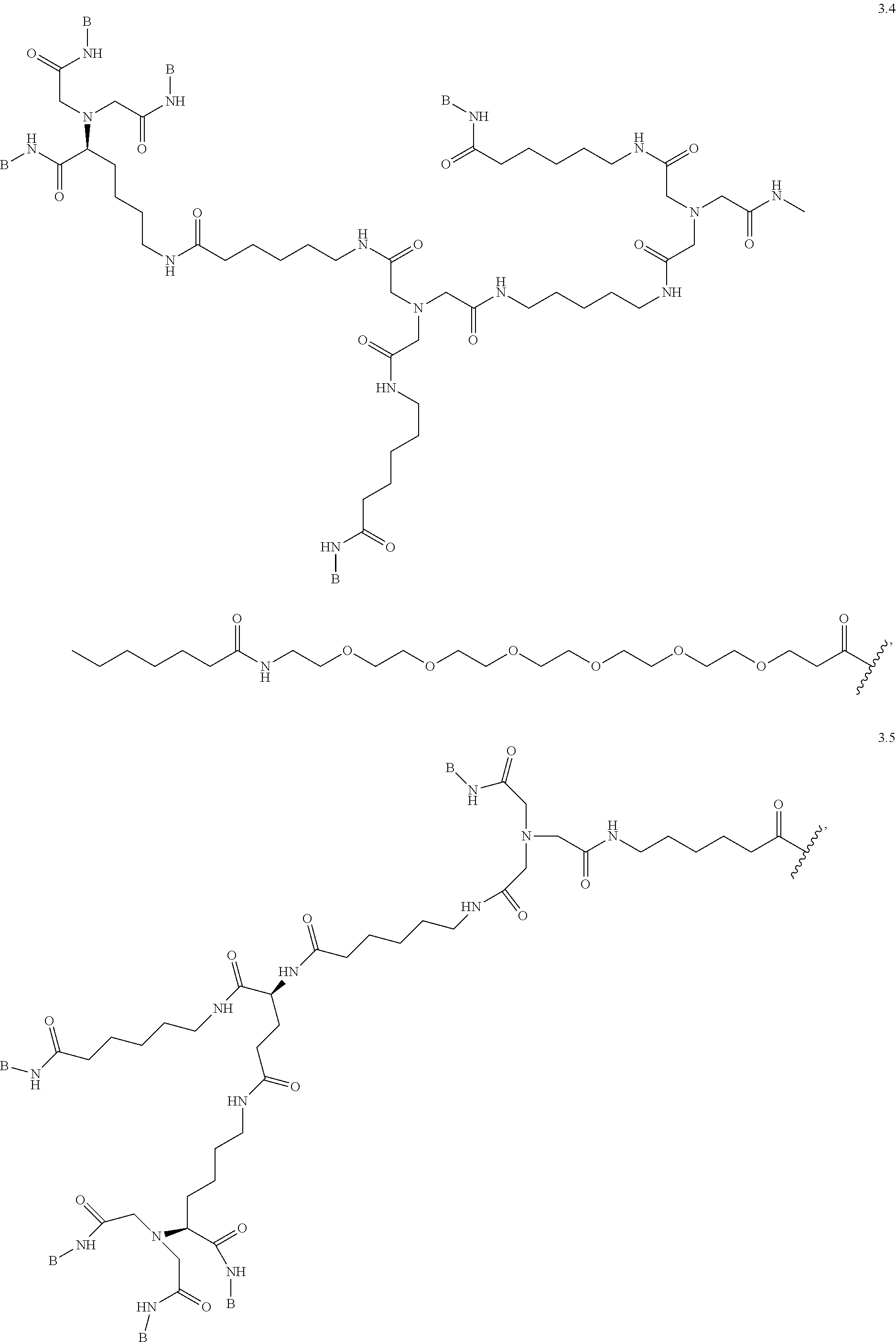

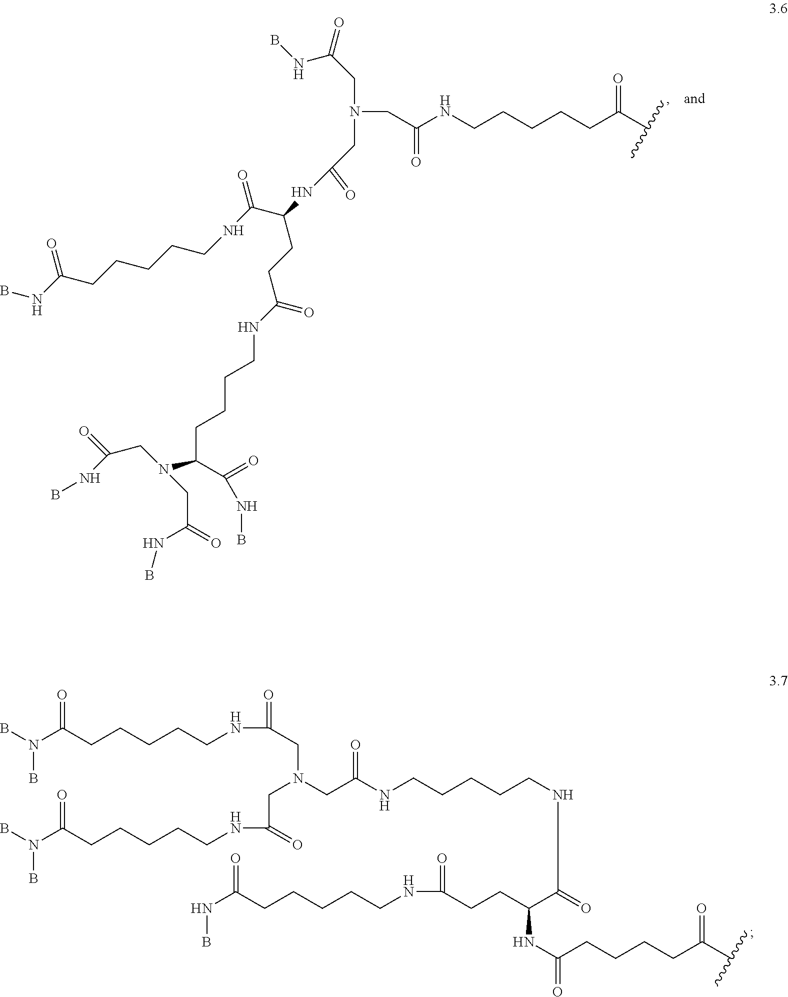

or the conjugate is a conjugate having general formula IV, and the insulin or insulin analog is conjugated to a pentavalent linker that is

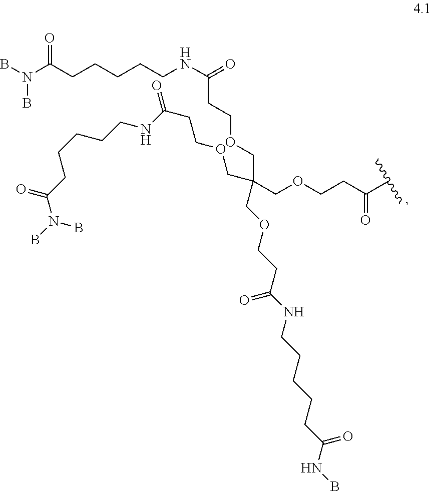

wherein a wavy line indicates the bond between the proximal end of the linker arm and amino acid on the insulin or insulin analog and wherein each B is independently -T-L^{B}-X, wherein each occurrence of X is independently the ligand and each occurrence of L^{B} is independently a covalent bond or a group derived from the covalent conjugation of a T with an X.

The present disclosure further provides a conjugate comprising an insulin or insulin analog is conjugated to a penta-valent sugar cluster that comprises a structure selected from the group consisting of ML-1, ML-2, ML-3, ML-4, ML-5, ML-6, ML-7, ML-8, ML-9, ML-10, ML-11, ML-12, ML-13, ML-14, ML-15, ML-16, ML-17. ML-18, ML-19, ML-20, ML-21, ML-22, ML-23. ML-24, ML-25, ML-26, ML-27, ML-28. ML-29, ML-30. ML-31, ML-32. ML-33, ML-34, ML-35, ML-36, ML-37, ML-38, ML-39, ML-40, ML-41, ML-42, ML-43, ML-44, ML-45, ML-46, ML-47, ML-48, ML-49, ML-50, ML-51, and ML-52.

In particular embodiments, the conjugate is selected from the group consisting of IOC-1, IOC-2, IOC-3, IOC-4, IOC-5, IOC-6, IOC-7, IOC-8, IOC-9, IOC-10, IOC-11, IOC-12, IOC-13, IOC-14, IOC-15, IOC-16, IOC-17, IOC-18, IOC-19, IOC-20, IOC-21, IOC-22, IOC-23, IOC-24, IOC-25, IOC-26. IOC-27, IOC-28, IOC-29, IOC-30, IOC-31, IOC-32, IOC-33, IOC-34, IOC-35, IOC-36. IOC-37. IOC-38, IOC-39, IOC-40, IOC-41, IOC-42. IOC-43, IOC-44, IOC-45, IOC-46, IOC-47, IOC-48, IOC-49, IOC-50, IOC-51, IOC-52, IOC-53, IOC-54, IOC-55, and IOC-56.

The present disclosure provides a composition comprising an insulin or insulin analog molecule covalently attached to at least one penta-valent sugar cluster, wherein the penta-valent sugar cluster is provided by a penta-dentate linker having four arms, wherein each arm of the penta-dentate linker is independently covalently linked to a ligand comprising or consisting of a monosaccharide, disaccharide, trisaccharide, tetrasaccharide, or branched trisaccharide, and a pharmaceutically acceptable carrier.

In particular embodiments, the ligand is selected from the group consisting of fucose, mannose, glucosamine, glucose, dimannose, trimannose, tetramannose, or branched trimannose.

In particular embodiments, the penta-valent sugar cluster is covalently linked to the side chain amino group of B29 lysine of insulin or insulin analog, in the presence or absence of small substitution on A1 and B1 amino groups.

In particular embodiments, the insulin analog is insulin lispro, insulin glargine, insulin aspart, insulin detemir, or insulin glulisine.

In particular embodiments, the conjugate displays a PD and/or PK profile that is sensitive to the serum concentration of a serum saccharide when administered to a subject in need thereof in the absence of an exogenous saccharide binding molecule.

In particular embodiments, the serum saccharide is glucose or alpha-methylmannose.

In particular embodiments, the conjugate binds an endogenous saccharide binding molecule at a serum glucose concentration of 60 mg/dL or less when administered to a subject in need thereof.

In particular embodiments, the endogenous saccharide binding molecule is human mannose receptor 1.

The present disclosure further provides a method for treating diabetes comprising administering to an individual in need thereof a therapeutically effective amount of the conjugate or composition herein to treat the diabetes. In particular aspects, the diabetes is type I diabetes, type II diabetes, or gestational diabetes.

The present disclosure further provides for the use of the conjugate or composition herein for the treatment of diabetes. In particular aspects, the diabetes is type I diabetes, type II diabetes, or gestational diabetes.

### Definitions

Definitions of specific functional groups, chemical terms, and general terms used throughout the specification are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

As used herein, the term “acyl,” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl, substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

As used herein, the term “aliphatic” or “aliphatic group” denotes an optionally substituted hydrocarbon moiety that may be straight-chain (i.e., unbranched), branched, or cyclic (“carbocycliC”) and may be completely saturated or may contain one or more units of unsaturation, but which is not aromatic. Unless otherwise specified, aliphatic groups contain 1-12 carbon atoms. In some embodiments, aliphatic groups contain 1-6 carbon atoms. In some embodiments, aliphatic groups contain 1-4 carbon atoms, and in yet other embodiments, aliphatic groups contain 1-3 carbon atoms. Suitable aliphatic groups include, but are not limited to, linear or branched, alkyl, alkenyl, and alkynyl groups, and hybrids thereof, such as (cycloalkyl)alkyl, (cycloalkenyl)alkyl, or (cycloalkyl)alkenyl.

As used herein, the term “alkenyl” denotes an optionally substituted monovalent group derived from a straight- or branched-chain aliphatic moiety having at least one carbon-carbon double bond. In particular embodiments, the alkenyl group employed in the disclosure contains 2-6 carbon atoms. In particular embodiments, the alkenyl group employed in the disclosure contains 2-5 carbon atoms. In some embodiments, the alkenyl group employed in the disclosure contains 2-4 carbon atoms. In another embodiment, the alkenyl group employed contains 2-3 carbon atoms. Alkenyl groups include, for example, ethenyl, propenyl, butenyl, 1-methyl-2-buten-1-yl, and the like.

As used herein, the term “alkyl” refers to optionally substituted saturated, straight- or branched-chain hydrocarbon radicals derived from an aliphatic moiety containing between 1-6 carbon atoms by removal of a single hydrogen atom. In some embodiments, the alkyl group employed in the disclosure contains 1-5 carbon atoms. In another embodiment, the alkyl group employed contains 1-4 carbon atoms. In still other embodiments, the alkyl group contains 1-3 carbon atoms. In yet another embodiment, the alkyl group contains 1-2 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

As used herein, the term “alkynyl” refers to an optionally substituted monovalent group derived from a straight- or branched-chain aliphatic moiety having at least one carbon-carbon triple bond by the removal of a single hydrogen atom. In particular embodiments, the alkynyl group employed in the disclosure contains 2-6 carbon atoms. In particular embodiments, the alkynyl group employed in the disclosure contains 2-5 carbon atoms. In some embodiments, the alkynyl group employed in the disclosure contains 2-4 carbon atoms. In another embodiment, the alkynyl group employed contains 2-3 carbon atoms. Representative alkynyl groups include, but are not limited to, ethynyl, 2-propynyl (propargyl), 1-propynyl, and the like.

As used herein, the term “aryl” used alone or as part of a larger moiety as in “aralkyl”, “aralkoxy”, or “aryloxyalkyl”, refers to an optionally substituted monocyclic and bicyclic ring systems having a total of five to 10 ring members, wherein at least one ring in the system is aromatic and wherein each ring in the system contains three to seven ring members. The term “aryl” may be used interchangeably with the term “aryl ring.” In particular embodiments, “aryl” refers to an aromatic ring system that includes, but not limited to, phenyl (“Ph”), biphenyl, naphthyl, anthracyl and the like, which may bear one or more substituents. Optionally, one or more heteroatoms, such as S, N, or O, may be incorporated into the aryl ring, providing a heteroaryl or heteroaromatic moiety, as defined below.

As used herein, the term “arylalkyl” refers to an alkyl group substituted with an aryl group (e.g., an aromatic or heteroaromatic group).

As used herein, the term “bivalent hydrocarbon chain” (also referred to as a “bivalent alkylene group”) is a polymethylene group, i.e., —(CH₂)_{z}—, wherein z is a positive integer from 1 to 30, from 1 to 20, from 1 to 12, from 1 to 8, from 1 to 6, from 1 to 4, from 1 to 3, from 1 to 2, from 2 to 30, from 2 to 20, from 2 to 10, from 2 to 8, from 2 to 6, from 2 to 4, or from 2 to 3.

As used herein, the term “carbonyl” refers to a monovalent or bivalent moiety containing a carbon-oxygen double bond. Non-limiting examples of carbonyl groups include aldehydes, ketones, carboxylic acids, ester, amide, enones, acyl halides, anhydrides, ureas, carbamates, carbonates, thioesters, lactones, lactams, hydroxamates, isocyanates, and chloroformates.

As used herein, the terms “cycloalkyl”, “cycloaliphatic”, “carbocycle”, or “carbocyclic”, used alone or as part of a larger moiety, refer to an optionally substituted saturated or partially unsaturated cyclic aliphatic monocyclic or bicyclic ring systems, as described herein, having from 3 to 10 members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloalkyl has 3-6 carbons.

As used herein, the term “fucose” refers to the D or L form of fucose and may refer to an oxygen or carbon linked glycoside.

As used herein, the terms “halo” and “halogen” refer to an atom selected from fluorine (fluoro, —F), chlorine (chloro, —Cl), bromine (bromo, —Br), and iodine (iodo, —I).

As used herein, the terms “heteroaliphatic” or “heteroaliphatic group”, denote an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but that is not aromatic. Unless otherwise specified, heteroaliphatic groups contain 1-6 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups.

As used herein, the term “heteroaralkyl” refers to an alkyl group substituted by a heteroaryl, wherein the alkyl and heteroaryl portions independently are optionally substituted.

As used herein, the term “heteroaryl” used alone or as part of a larger moiety, e.g., “heteroaralkyl”, or “heteroaralkoxy”, refers to an optionally substituted group having 5 to 10 ring atoms, preferably 5, 6, or 9 ring atoms; having 6, 10, or 14π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, carbocyclic, or heterocyclic rings, where the radical or point of attachment is on the heteroaromatic ring. Non limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted.

As used herein, the term “heteroatom” refers to nitrogen, oxygen, or sulfur, and includes any oxidized form of nitrogen or sulfur, and any quaternized form of a basic nitrogen. The term “nitrogen” also includes a substituted nitrogen.

As used herein, the terms “heterocycle”, “heterocyclyl”. “heterocyclic radical”, and “heterocyclic ring” are used interchangeably and refer to a stable optionally substituted 5- to 7-membered monocyclic or 7- to 10-membered bicyclic heterocyclic moiety that is either saturated or partially unsaturated, and having, in addition to carbon atoms, one or more heteroatoms, as defined above. A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle” “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or carbocyclic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “unsaturated”, means that a moiety has one or more double or triple bonds.

As used herein, the term “partially unsaturated” refers to a ring moiety that includes at least one double or triple bond. The term “partially unsaturated” is intended to encompass rings having multiple sites of unsaturation, but it is not intended to include aryl or heteroaryl moieties, as herein defined.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in particular embodiments, their recovery, purification, and use for one or more of the purposes disclosed herein.

Suitable monovalent substituents on a substitutable carbon atom of an “optionally substituted” group are independently halogen; —(CH₂)₀₋₄R^{º}; —(CH₂)₀₋₄OR^{º}; —O—(CH₂)₀₋₄C(O)OR^{º}; —(CH₂)₀₋₄CH(OR^{º})₂; —(CH₂)₀₋₄SR^{º}; —(CH₂)₀₋₄Ph, which may be substituted with R^{º}; —(CH₂)₀₋₄O(CH₂)₀₋₁Ph, which may be substituted with R^{º}; —CH═CHPh, which may be substituted with R^{º}; —NO₂; —CN; —N₃; —(CH₂)₀₋₄N(R^{º})₂; —(CH₂)₀₋₄N(R^{º})C(O)R^{º}; —N(R^{º})C(S)R^{º}; —(CH₂)₀₋₄N(R^{º})C(O)NR^{º}₂; —N(R^{º})C(S)NR^{º}₂; —(CH₂)₀₋₄N(R^{º})C(O)OR^{º}; —N(R^{º})N(R^{º})C(O)R^{º}; —N(R^{º})N(R^{º})C(O)NR^{º}₂; —N(R^{º})N(R^{º})C(O)OR^{º}; —(CH₂)₀₋₄C(O)R^{º}; —C(S)R^{º}; —(CH₂)₀₋₄C(O)OR^{º}; —(CH₂)₀₋₄C(O)SR^{º}; —(CH₂)₀₋₄C(O)OSiR^{º}₃; —(CH₂)₀₋₄OC(O)R^{º}; —OC(O)(CH₂)₀₋₄SR—, SC(S)SR^{º}; —(CH₂)₀₋₄SC(O)R^{º}; —(CH₂)₀₋₄C(O)NR^{º}₂; —C(S)NR^{º}₂; —C(S)SR^{º}; —SC(S)SR^{º}, —(CH₂)₀₋₄OC(O)NR^{º}₂; —C(O)N(OR^{º})R^{º}; —C(O)C(O)R^{º}; —C(O)CH₂C(O)R^{º}; —C(NOR^{º})R^{º}; —(CH₂)₀₋₄SSR^{º}; —(CH₂)₀₋₄S(O)₂R^{º}; —(CH₂)₀₋₄S(O)₂OR^{º}; —(CH₂)₀₋₄OS(O)₂R^{º}; —S(O)₂NR^{º}₂; —(CH₂)₀₋₄S(O)R^{º}; —N(R^{º})S(O)₂NR^{º}₂; —N(R^{º})S(O)₂R^{º}; —N(OR^{º})R^{º}; —C(NH)NR^{º}₂; —P(O)₂R^{º}; —P(O)R^{º}₂; —OP(O)R^{º}₂; —OP(O)(OR^{º})₂; SiR^{º}₃; —(C₁₋₄ straight or branched alkylene)O—N(R^{º})₂; or —(C₁₋₄ straight or branched alkylene)C(O)O—N(R^{º})₂, wherein each R^{º} may be substituted as defined below and is independently hydrogen, C₁₋₆ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur, or, notwithstanding the definition above, two independent occurrences of R^{º}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{º} (or the ring formed by taking two independent occurrences of R^{º} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur. Suitable divalent substituents on a saturated carbon atom of R^{º} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic that may be substituted as defined below, or an unsubstituted 5- or 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic, which may be substituted as defined below, or an unsubstituted 5- to 6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic that may be substituted as defined below, unsubstituted —OPh, or an unsubstituted 5- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

As used herein, the term “suitable protecting group,” refers to amino protecting groups or hydroxyl protecting groups depending on its location within the compound and includes those described in detail in PROTECTING GROUPS IN ORGANIC SYNTHESIS, T. W. Greene and P. G. M. Wuts, 3^{rd} edition, John Wiley & Sons, 1999.

In any case where a chemical variable (e.g., an R group) is shown attached to a bond that crosses a bond of the ring, this means that one or more such variables are optionally attached to the ring having the crossed bond. Each R group on such a ring can be attached at any suitable position on the ring, this is generally understood to mean that the group is attached in place of a hydrogen atom on the parent ring. This includes the possibility that two R groups can be attached to the same ring atom. Furthermore, when more than one R group is present on a ring, each may be the same or different than other R groups attached thereto, and each group is defined independently of other groups that may be attached elsewhere on the same molecule, even though they may be represented by the same identifier.

As used herein, an “exogenous” molecule is one that is not present at significant levels in a patient unless administered to the patient. In particular embodiments, the patient is a mammal, e.g., a human, a dog, a cat, a rat, a minipig, etc. As used herein, a molecule is not present at significant levels in a patient if normal serum for that type of patient includes less than 0.1 mM of the molecule. In particular embodiments, normal serum for the patient may include less than 0.08 mM, less than 0.06 mM, or less than 0.04 mM of the molecule.

As used herein, the term “treat” (or “treating”, “treated”, “treatment”, etc.) refers to the administration of a conjugate of the present disclosure to a subject in need thereof with the purpose to alleviate, relieve, alter, ameliorate, improve or affect a condition (e.g., diabetes), a symptom or symptoms of a condition (e.g., hyperglycemia), or the predisposition toward a condition. For example, as used herein the term “treating diabetes” will refer in general to maintaining glucose blood levels near normal levels and may include increasing or decreasing blood glucose levels depending on a given situation.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the US Pharmacopeia for use in animals, including humans.

As used herein, the terms “effective amount” or “therapeutically effective amount” refer to a nontoxic but sufficient amount of an insulin analog to provide the desired effect. For example, one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” However, an appropriate “effective amount” in any individual case may be determined by one of ordinary skill.

As used herein, the term “patenteral” means not through the alimentary canal but by some other route such as intranasal, inhalation, subcutaneous, intramuscular, intraspinal, or intravenous.

As used herein, the term “insulin” means the active principle of the pancreas that affects the metabolism of carbohydrates in the animal body and is of value in the treatment of diabetes mellitus. The term includes synthetic and biotechnologically derived products that are the same as, or similar to, naturally occurring insulins in structure, use, and intended effect, and that are of value in the treatment of diabetes mellitus.

As used herein, the term “insulin or insulin molecule” is a generic term that includes the 51 amino acid heterodimer comprising the A-chain peptide having the amino acid sequence shown in SEQ ID NO: 1 and the B-chain peptide having the amino acid sequence shown in SEQ ID NO: 2, wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond. As used herein, the terms “insulin” or “insulin molecule” encompasses all salt and non-salt forms of the insulin molecule. It will be appreciated that the salt form may be anionic or cationic depending on the insulin molecule. By “insulin” or “an insulin molecule”, it is intended that this disclosure encompasses both wild-type insulin and modified forms of insulin as long as they are bioactive (i.e., capable of causing a detectable reduction in glucose when administered in vivo).

The term “insulin analog” or “insulin analog” as used herein includes any heterodimer analog or single-chain analog that comprises one or more modification(s) of the native A-chain peptide and/or B-chain peptide. Modifications include but are not limited to substituting an amino acid for the native amino acid at a position selected from A4, A5, A8, A9. A10, A12, A13, A14, A15, A16, A17, A18, A19, A21, B1, B2, B3, B4, B5, B9, B10, B13, B14, B15, B16, B17, B18, B20, B21, B22, B23, B26, B27, B28, B29, and B30; deleting any or all of positions B1-4 and B26-30; adding any or all of terminal positions A1, B1, A21, and B30; or conjugating directly or by a polymeric or non-polymeric linker one or more acyl, polyethylglycine (“PEG”), or saccharide moiety (moieties); or any combination thereof. As exemplified by the N-linked glycosylated insulin analogs disclosed herein, the term further includes any insulin heterodimer and single-chain analog that has been modified to have at least one N-linked glycosylation site and in particular, embodiments in which the N-linked glycosylation site is linked to or occupied by an N-glycan. Examples of insulin analogs include but are not limited to the heterodimer and single-chain analogs disclosed in published international application WO2010/0080606, WO2009/099763, and WO2010/080609, the disclosures of which are incorporated herein by reference. Examples of single-chain insulin analogs also include but are not limited to those disclosed in published International Applications WO96/34882, WO95/516708, WO2005/054291, WO2006/097521, WO2007/104734, WO2007/104736, WO2007/104737, WO2007/104738, WO2007/096332, WO2009/132129; U.S. Pat. Nos. 5,304,473 and 6,630,348; and Kristensen et al., BIOCHEM. J. 305: 981-986 (1995), the disclosures of which are each incorporated herein by reference.

As used herein, the term “single-chain insulin or single-chain insulin analog” encompasses a group of structurally-related proteins wherein the A-chain peptide or functional analog and the B-chain peptide or functional analog are covalently linked by a peptide or polypeptide of 2 to 35 amino acids or non-peptide polymeric or non-polymeric linker and which has at least 1%, 10%, 50%, 75%, or 90% of the activity of insulin at the insulin receptor as compared to native insulin. The single-chain insulin or insulin analog further includes three disulfide bonds: the first disulfide bond is between the cysteine residues at positions 6 and 11 of the A-chain or functional analog thereof, the second disulfide bond is between the cysteine residues at position 7 of the A-chain or functional analog thereof and position 7 of the B-chain or functional analog thereof, and the third disulfide bond is between the cysteine residues at position 20 of the A-chain or functional analog thereof and position 19 of the B-chain or functional analog thereof.

As used herein, the terms “connecting peptide” or C-peptide” refer to the connection moiety “C” of the B-C-A polypeptide sequence of a single chain prepro insulin-like molecule. Specifically, in the natural insulin chain, the C-peptide connects the amino acid at position 30 of the B-chain and the amino acid at position 1 of the A-chain. The term can refer to both the native insulin C-peptide, the monkey C-peptide, and any other peptide from 3 to 35 amino acids that connects the B-chain to the A-chain thus is meant to encompass any peptide linking the B-chain peptide to the A-chain peptide in a single-chain insulin analog (see for example, U.S. Published Application Nos. US2009/0170750 and US2008/0057004 and WO96/34882) and in insulin precursor molecules such as disclosed in WO95/16708 and U.S. Pat. No. 7,105,314.

As used herein, the term “amino acid modification” refers to a substitution of an amino acid or the derivation of an amino acid by the addition and/or removal of chemical groups to/from the amino acid, and the term includes substitution with any of the 20 amino acids commonly found in human proteins, as well as atypical or non-naturally occurring amino acids.

Commercial sources of atypical amino acids include Sigma-Aldrich (Milwaukee, WI), ChemPep Inc. (Miami, FL), and Genzyme Pharmaceuticals (Cambridge, MA). Atypical amino acids may be purchased from commercial suppliers, synthesized de novo, or chemically modified or derivatized from naturally occurring amino acids.

As used herein, the term “amino acid substitution” refers to the replacement of one amino acid residue by a different amino acid residue.

As used herein, the term “conservative amino acid substitution” is defined herein as exchanges within one of the following five groups:
I. Small aliphatic, nonpolar, or slightly polar residues:
   Ala, Ser, Thr, Pro, Gly;
II. Polar, negatively charged residues and their amides:
   Asp, Asn, Glu, Gln, cysteic acid, and homocysteic acid;
III. Polar, positively charged residues:
   His, Arg, Lys; Ornithine (Om)
IV. Large, aliphatic, nonpolar residues:
   Met, Leu, Ile, Val, Cys, Norleucine (Nle), homocysteine
V. Large, aromatic residues:
   Phe, Tyr, Trp, acetyl phenylalanine

As used herein, the term “penta-dentate linker” refers to a linker comprising a linker arm having a proximal end and a distal end wherein the proximal end is covalently linked to an amino acid on an insulin molecule and the distal end is covalently linked at or near the distal end to five ligand arms, each ligand arm having a distal end and a proximal end wherein the distal end is covalently linked to a ligand and the proximal end is covalently linked to the linker arm at or near the distal end of the linker arm.

As used herein, “plasma glucose” is usually 10% to 12% higher than “blood glucose” (considering blood glucose to be plasma+all blood cells).

The present disclosure provides methods for controlling the PK and/or PD profiles of insulin in a manner that is responsive to the systemic concentrations of a saccharide such as glucose. The methods are based in part on the discovery disclosed in U.S. Published Application No. US2011/0301083 that when particular insulin conjugates are modified to include high affinity saccharide ligands, such as branched trimannose, they could be made to exhibit PK/PD profiles that responded to saccharide concentration changes even in the absence of an exogenous multivalent saccharide-binding molecule such as the lectin Con A.

In general, the insulin conjugates of the present disclosure comprise an insulin or insulin analog molecule covalently attached to a penta-valent sugar cluster at the side chain amino group of B29 lysine of insulin or insulin analog, in the presence or absence of small substitution on A1 and B1 amino groups of insulin or insulin analog. In particular embodiments, the penta-valent sugar cluster is capable of competing with a saccharide (e.g., glucose or alpha-methylmannose) for binding to an endogenous saccharide-binding molecule, such as the Macrophage Mannose Receptor 1. In particular embodiments, the penta-valent sugar cluster is capable of competing with glucose or alpha-methylmannose for binding to Con A. In particular embodiments, the linker is non-polymeric or highly branched. In particular embodiments, the conjugate may have a polydispersity index of one and a MW of less than about 20,000 Da. In particular embodiments, the conjugate is of formula I or of formula II or of formula III or of formula IV as defined and described herein. In particular embodiments, the conjugate is long acting (i.e., exhibits a PK profile that is more sustained than soluble RHI).

### Insulin Conjugates

In one aspect, the present disclosure provides an insulin or insulin analog molecule conjugated to at least one penta-valent sugar cluster wherein the penta-valent sugar cluster is provided by a branched linker having five arms (penta-dentate linker, as discussed above) wherein each arm of the penta-dentate linker is independently covalently linked to a ligand comprising or consisting of a monosaccharide, disaccharide, trisaccharide, tetrasaccharide, or branched trisaccharide. Thus, as used herein a penta-valent sugar cluster comprises or consists of five ligands conjugated to a single amino acid on the insulin or insulin analog molecule. In particular embodiments, the amino acid is the side chain amino group of B29 lysine of insulin or insulin analog, in the presence or absence of small substitution on A1 and B1 amino groups of insulin or insulin analog.

In particular aspects, the insulin or insulin analog molecule is conjugated to one, two, or three penta-dentate linkers wherein each arm of each penta-dentate linker is independently covalently linked to a ligand comprising or consisting of a saccharide. In particular aspects, each ligand independently comprises or consists of a monosaccharide, disaccharide, trisaccharide, tetrasaccharide, or branched trisaccharide. In particular aspects, each ligand comprises or consists of a monomannose, dimannose, trimannose, tetramannose, or branched trimannose. In particular aspects, at least one ligand is fucose. In particular aspects, at least one ligand is a branched trimannose. In particular aspects, at least one ligand is a dimannose. In particular aspects, at least one ligand is mannose. In particular aspects, at least two ligands are fucose, branched mannose, dimannose, or mannose. In particular aspects, at least three ligands are fucose, branched mannose, dimannose, or mannose. In particular aspects, all four ligands are fucose, branched mannose, dimannose, or mannose.

In particular aspects, the insulin or insulin analog molecule is conjugated to two penta-dentate linkers wherein each arm of each penta-dentate linker is independently covalently linked to a ligand comprising or consisting of a saccharide. In particular aspects, each ligand independently comprises or consists of a monosaccharide, disaccharide, trisaccharide, tetrasaccharide, or branched trisaccharide. In particular aspects, each ligand comprises or consists of a monomannose, dimannose, trimannose, tetramannose, or branched trimannose. In particular aspects, at least one ligand is fucose. In particular aspects, at least one ligand is a branched trimannose. In particular aspects, at least one ligand is a dimannose. In particular aspects, at least one ligand is mannose. In particular aspects, at least two ligands are fucose, branched mannose, dimannose, or mannose. In particular aspects, at least three ligands are fucose, branched mannose, dimannose, or mannose. In particular aspects, all four ligands are fucose, branched mannose, dimannose, or mannose.

In particular aspects, the insulin or insulin analog molecule is conjugated to three penta-dentate linkers wherein each arm of each penta-dentate linker is independently covalently linked to a ligand comprising or consisting of a saccharide. In particular aspects, each ligand independently comprises or consists of a monosaccharide, disaccharide, trisaccharide, tetrasaccharide, or branched trisaccharide. In particular aspects, each ligand comprises or consists of a monomannose, dimannose, trimannose, tetramannose, or branched trimannose. In particular aspects, at least one ligand is fucose. In particular aspects, at least one ligand is a branched trimannose. In particular aspects, at least one ligand is a dimannose. In particular aspects, at least one ligand is mannose. In particular aspects, at least two ligands are fucose, branched mannose, dimannose, or mannose. In particular aspects, at least three ligands are fucose, branched mannose, dimannose, or mannose. In particular aspects, all four ligands are fucose, branched mannose, dimannose, or mannose.

In particular aspects, the insulin or insulin analog molecule of the insulin conjugate disclosed herein is conjugated to a penta-dentate linker wherein each arm of each penta-dentate linker is independently covalently linked to a ligand comprising or consisting of a saccharide and is covalently attached to a linear linker that is linked to one ligand comprising or consisting of a saccharide. In particular aspects, each ligand independently comprises or consists of a monosaccharide, disaccharide, trisaccharide, tetrasaccharide, or branched trisaccharide. In particular aspects, each ligand comprises or consists of a monomannose, dimannose, trimannose, tetramannose, or branched trimannose. In particular aspects, at least one ligand is fucose. In particular aspects, at least one ligand is a branched trimannose. In particular aspects, at least one ligand is a dimannose. In particular aspects, at least one ligand is mannose. In particular aspects, at least two ligands are fucose, branched mannose, dimannose, or mannose. In particular aspects, at least three ligands are fucose, branched mannose, dimannose, or mannose. In particular aspects, at least four ligands are fucose, branched mannose, dimannose, or mannose. In particular aspects, all five ligands are fucose, branched mannose, dimannose, or mannose.

In particular aspects, the insulin or insulin analog molecule of the insulin conjugate disclosed herein is conjugated to a penta-dentate linker wherein each arm of each penta-dentate linker is independently covalently linked to a ligand comprising or consisting of a saccharide and is covalently attached to a linker having two arms, each arm independently covalently linked to a ligand comprising or consisting of a saccharide. In particular aspects, each ligand independently comprises or consists of a monosaccharide, disaccharide, trisaccharide, tetrasaccharide, or branched trisaccharide. In particular aspects, each ligand comprises or consists of a monomannose, dimannose, trimannose, tetramannose, or branched trimannose. In particular aspects, at least one ligand is fucose. In particular aspects, at least one ligand is a branched trimannose. In particular aspects, at least one ligand is a dimannose. In particular aspects, at least one ligand is mannose. In particular aspects, at least two ligands are selected from fucose, branched mannose, dimannose, or mannose. In particular aspects, at least three ligands are fucose, branched mannose, dimannose, or mannose. In particular aspects, at least four ligands are fucose, branched mannose, dimannose, or mannose. In particular aspects, all five ligands are fucose, branched mannose, dimannose, or mannose.

When the insulin conjugate is administered to a mammal at least one pharmacokinetic or pharmacodynamic property of the conjugate is sensitive to the serum concentration of a saccharide. In particular embodiments, the PK and/or PD properties of the conjugate are sensitive to the serum concentration of an endogenous saccharide such as glucose. In particular embodiments, the PK and/or PD properties of the conjugate are sensitive to the serum concentration of an exogenous saccharide, e.g., without limitation, mannose, fucose, N-acetyl glucosamine and/or alpha-methylmannose.

### Pharmacokinetic (“PK”) and Pharmacodynamic (“PD”) Properties

In various embodiments, the PK and/or PD behavior of the insulin conjugate may be modified by variations in the serum concentration of a saccharide. For example, from a PK perspective, the serum concentration curve may shift upward when the serum concentration of the saccharide (e.g., glucose) increases or when the serum concentration of the saccharide crosses a threshold (e.g., is higher than normal glucose levels).

In particular embodiments, the serum concentration curve of a conjugate disclosed herein is substantially different when administered to the mammal under fasted and hyperglycemic conditions. As used herein, the term “substantially different” means that the two curves are statistically different as determined by a student t-test (p<0.05). As used herein, the term “fasted conditions” means that the serum concentration curve was obtained by combining data from five or more fasted non-diabetic individuals. In particular embodiments, a fasted non-diabetic individual is a randomly selected 18- to 30-year old human who presents with no diabetic symptoms at the time blood is drawn and who has not eaten within 12 hours of the time blood is drawn. As used herein, the term “hyperglycemic conditions” means that the serum concentration curve was obtained by combining data from five or more fasted non-diabetic individuals in which hyperglycemic conditions (glucose Cₘₐₓ at least 100 mg/dL above the mean glucose concentration observed under fasted conditions) were induced by concurrent administration of conjugate and glucose. Concurrent administration of conjugate and glucose simply requires that the glucose Cₘₐₓ occur during the period when the conjugate is present at a detectable level in the serum. For example, a glucose injection (or ingestion) could be timed to occur shortly before, at the same time or shortly after the conjugate is administered. In particular embodiments, the conjugate and glucose are administered by different routes or at different locations. For example, in particular embodiments, the conjugate is administered subcutaneously while glucose is administered orally or intravenously.

In particular embodiments, the serum Cₘₐₓ of the conjugate is higher under hyperglycemic conditions as compared to fasted conditions. Additionally or alternatively, in particular embodiments, the serum area under the curve (“AUC”) of the conjugate is higher under hyperglycemic conditions as compared to fasted conditions. In various embodiments, the serum elimination rate of the conjugate is slower under hyperglycemic conditions as compared to fasted conditions. In particular embodiments, the serum concentration curve of the conjugates can be fit using a two-compartment bi-exponential model with one short and one long half-life. The long half-life appears to be particularly sensitive to glucose concentration. Thus, in particular embodiments, the long half-life is longer under hyperglycemic conditions as compared to fasted conditions. In particular embodiments, the fasted conditions involve a glucose Cₘₐₓ of less than 100 mg/dL (e.g., 80 mg/dL, 70 mg/dL, 60 mg/dL, 50 mg/dL, etc.). In particular embodiments, the hyperglycemic conditions involve a glucose Cₘₐₓ in excess of 200 mg/dL (e.g., 300 mg/dL, 400 mg/dL, 500 mg/dL, 600 mg/dL, etc.). It will be appreciated that other PK parameters such as mean serum residence time (“MRT”), mean serum absorption time (“MAT”), etc. could be used instead of or in conjunction with any of the aforementioned parameters.

The normal range of glucose concentrations in humans, dogs, cats, and rats is 60 to 200 mg/dL. One skilled in the art will be able to extrapolate the following values for species with different normal ranges (e.g., the normal range of glucose concentrations in miniature pigs is 40 to 150 mg/dl). Glucose concentrations below 60 mg/dL are considered hypoglycemic. Glucose concentrations above 200 mg/dL are considered hyperglycemic. In particular embodiments, the PK properties of the conjugate may be tested using a glucose clamp method, and the serum concentration curve of the conjugate may be substantially different when administered at glucose concentrations of 50 and 200 mg/dL, 50 and 300 mg/dL, 50 and 400 mg/dL, 50 and 500 mg/dL, 50 and 600 mg/dL, 100 and 200 mg/dL, 100 and 300 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc. Additionally or alternatively, the serum Tₘₐₓ, serum Cₘₐₓ, MRT, MAT, and/or serum half-life may be substantially different at the two glucose concentrations. As discussed below, in particular embodiments, 100 mg/dL and 300 mg/dL may be used as comparative glucose concentrations. It is to be understood, however, that the present disclosure encompasses each of these embodiments with an alternative pair of comparative glucose concentrations including, without limitation, any one of the following pairs: 50 and 200 mg/dL, 50 and 300 mg/dL, 50 and 400 mg/dL, 50 and 500 mg/dL, 50 and 600 mg/dL, 100 and 200 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc.

Thus, in particular embodiments, the Cₘₐₓ of the conjugate is higher when administered to the mammal at the higher of the two glucose concentrations (e.g., 300 vs. 100 mg/dL glucose). In particular embodiments, the Cₘₐₓ of the conjugate is at least 50% (e.g., at least 100%, at least 200% or at least 400%) higher when administered to the mammal at the higher of the two glucose concentrations (e.g., 300 vs. 100 mg/dL glucose).

In particular embodiments, the AUC of the conjugate is higher when administered to the mammal at the higher of the two glucose concentrations (e.g., 300 vs. 100 mg/dL glucose). In particular embodiments, the AUC of the conjugate is at least 50% (e.g., at least 100%, at least 200% or at least 400%) higher when administered to the mammal at the higher of the two glucose concentrations (e.g., 300 vs. 100 mg/dL glucose).

In particular embodiments, the serum elimination rate of the conjugate is slower when administered to the mammal at the higher of the two glucose concentrations (e.g., 300 vs. 100 mg/dL glucose). In particular embodiments, the serum elimination rate of the conjugate is at least 25% (e.g., at least 50%, at least 100%, at least 200%, or at least 400%) faster when administered to the mammal at the lower of the two glucose concentrations (e.g., 100 vs. 300 mg/dL glucose).

In particular embodiments, the serum concentration curve of conjugates may be fit using a two-compartment bi-exponential model with one short and one long half-life. The long half-life appears to be particularly sensitive to glucose concentration. Thus, in particular embodiments, the long half-life is longer when administered to the mammal at the higher of the two glucose concentrations (e.g., 300 vs. 100 mg/dL glucose). In particular embodiments, the long half-life is at least 50% (e.g., at least 100%, at least 200% or at least 400%) longer when administered to the mammal at the higher of the two glucose concentrations (e.g., 300 vs. 100 mg/dL glucose).

In particular embodiments, the present disclosure provides a method in which the serum concentration curve of a conjugate is obtained at two different glucose concentrations (e.g., 300 vs. 100 mg/dL glucose); the two curves are fit using a two-compartment bi-exponential model with one short and one long half-life; and the long half-lives obtained under the two glucose concentrations are compared. In particular embodiments, this method may be used as an assay for testing or comparing the glucose sensitivity of one or more conjugates.

In particular embodiments, the hyperglycemic conditions involve a glucose Cₘₐₓ in excess of 200 mg/dL (e.g., 300 mg/dL, 400 mg/dL, 500 mg/dL, 600 mg/dL, etc.). In particular embodiments, the fasted conditions involve a glucose Cₘₐₓ of less than 100 mg/dL (e.g., 80 mg/dL, 70 mg/dL, 60 mg/dL, 50 mg/dL, etc.). It will be appreciated that any of the aforementioned PK parameters such as serum Tₘₐₓ, serum Cₘₐₓ, AUC, MRT, MAT, and/or serum half-life could be compared.

From a PD perspective, the bioactivity of the conjugate may increase when the glucose concentration increases or when the glucose concentration crosses a threshold, e.g., is higher than normal glucose levels. In particular embodiments, the bioactivity of a conjugate is lower when administered under fasted conditions as compared to hyperglycemic conditions. In particular embodiments, the fasted conditions involve a glucose Cₘₐₓ of less than 100 mg/dL (e.g., 80 mg/dL, 70 mg/dL, 60 mg/dL, 50 mg/dL, etc.). In particular embodiments, the hyperglycemic conditions involve a glucose Cₘₐₓ in excess of 200 mg/dL (e.g., 300 mg/dL, 400 mg/dL, 500 mg/dL, 600 mg/dL, etc.).

In particular embodiments, the PD properties of the conjugate may be tested by measuring the glucose infusion rate (“GIR”) required to maintain a steady glucose concentration. According to such embodiments, the bioactivity of the conjugate may be substantially different when administered at glucose concentrations of 50 and 200 mg/dL, 50 and 300 mg/dL, 50 and 400 mg/dL, 50 and 500 mg/dL, 50 and 600 mg/dL, 100 and 200 mg/dL, 100 and 300 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc. Thus, in particular embodiments, the bioactivity of the conjugate is higher when administered to the mammal at the higher of the two glucose concentrations (e.g., 300 vs. 100 mg/dL glucose). In particular embodiments, the bioactivity of the conjugate is at least 25% (e.g., at least 50% or at least 100%) higher when administered to the mammal at the higher of the two glucose concentrations (e.g., 300 vs. 100 mg/dL glucose).

In general, it will be appreciated that any of the PK and PD characteristics discussed in this section can be determined according to any of a variety of published pharmacokinetic and pharmacodynamic methods (see e.g., Baudys et al., BIOCONJUGATE CHEM. 9:176-183, 1998, for methods suitable for subcutaneous delivery). It is also to be understood that the PK and/or PD properties may be measured in any mammal (e.g., a human, a rat, a cat, a minipig, a dog, etc.). In particular embodiments, PK and/or PD properties are measured in a human. In particular embodiments, PK and/or PD properties are measured in a rat. In particular embodiments, PK and/or PD properties are measured in a minipig. In particular embodiments, PK and/or PD properties are measured in a dog.

### Ligand(s)

In general, a ligand comprises or consists of a monosaccharide, disaccharide, trisaccharide, tetrasaccharide, or branched trisaccharide. In particular aspects, the ligand comprises or consists of a monomannose, dimannose, trimannose, tetramannose, or branched trimannose. In particular aspects, the ligand comprises or consists of fucose, glucose, or N-glucosamine.

In particular embodiments, each ligand comprises a penta-valent sugar cluster are capable of competing with a saccharide (e.g., glucose, alpha-methylmannose, or mannose) for binding to an endogenous saccharide-binding molecule (e.g., without limitation surfactant proteins A and D or members of the selectin family). In particular embodiments, the ligands are capable of competing with glucose or alpha-methylmannose for binding to the human macrophage mannose receptor 1 (“MRC1”). In particular embodiments, the ligands are capable of competing with a saccharide for binding to a non-human lectin (e.g., Con A). In particular embodiments, the ligands are capable of competing with glucose, alpha-methylmannose, or mannose for binding to a non-human lectin (e.g., Con A). Exemplary glucose-binding lectins include calnexin, calreticulin, N-acetylglucosamine receptor, selectin, asialoglycoprotein receptor, collectin (mannose-binding lectin), mannose receptor, aggrecan, versican, Pisum sativum agglutinin (“PSA”), Vicia faba lectin, Lens culinaris lectin, soybean lectin, peanut lectin, Lathyrus ochrus lectin, sainfoin lectin, Sophora japonica lectin, Bowringia mildbraedii lectin, Con A, and pokeweed mitogen.

In particular embodiments, one or more of the ligands may have the same chemical structure as glucose or may be a chemically related species of glucose, e.g., glucosamine. In various embodiments, it may be advantageous for one or more of the ligands to have a different chemical structure from glucose, e.g., in order to fine tune the glucose response of the conjugate. For example, in particular embodiments, one might use a ligand that includes glucose, mannose, fucose, or derivatives of these (e.g., alpha-L-fucopyranoside, mannosamine, beta-linked N-acetyl mannosamine, methylglucose, methylmannose, ethylglucose, ethylmannose, propylglucose, propylmannose, etc.) and/or higher order combinations of these (e.g., a dimannose, linear, and/or branched trimannose, etc.).

In particular embodiments, a ligand includes a monosaccharide. In particular embodiments, a ligand includes a disaccharide. In particular embodiments, a ligand includes a trisaccharide. In some embodiments, the ligand comprises or consists of a saccharide and one or more amine groups. In some embodiments, the ligand comprises or consists of a saccharide and ethyl group. In particular embodiments, the saccharide and amine group are separated by a C₁-C₆ alkyl group, e.g., a C₁-C₃ alkyl group. In some embodiments, the ligand is aminoethylglucose (“AEG”). In some embodiments, the ligand is aminoethylmannose (“AEM”). In some embodiments, the ligand is aminoethylbimannose (“AEBM”). In some embodiments, the ligand is aminoethyltrimannose (“AETM”). In some embodiments, the ligand is β-aminoethyl-N-acetylglucosamine (“AEGA”). In some embodiments, the ligand is aminoethylfucose (“AEF”). In particular embodiments, the saccharide is of the “D” configuration and in other embodiments, the saccharide is of the “L” configuration. Below are the structures of exemplary saccharides having an amine group separated from the saccharide by a C₂ ethyl group wherein R may be hydrogen or a carbonyl group of the linker. Other exemplary ligands will be recognized by those skilled in the art.

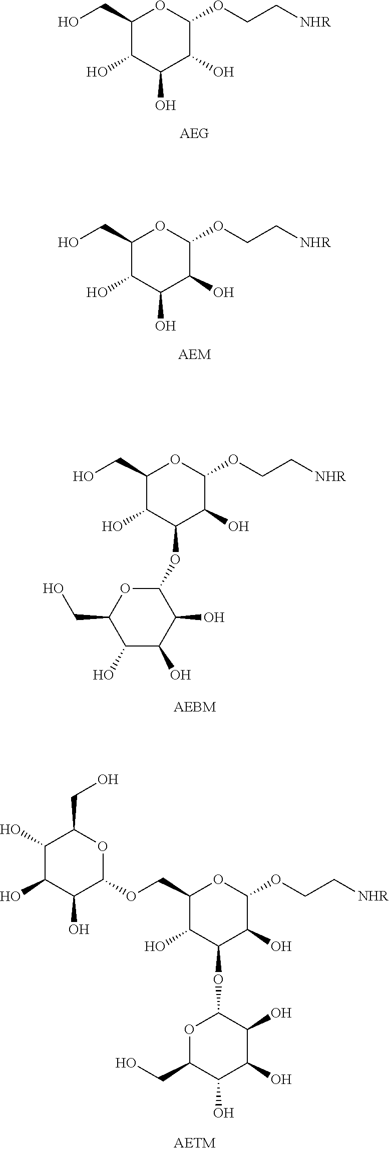

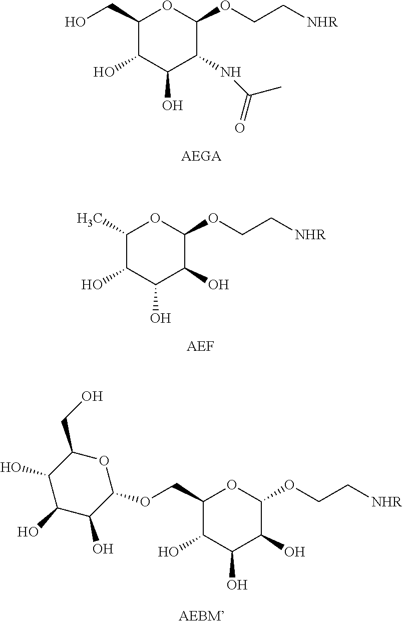

### Insulin or Insulin Analog

As used herein and as defined above, the term “insulin” or “insulin molecule” encompasses all salt and non-salt forms of the insulin molecule. It will be appreciated that the salt form may be anionic or cationic depending on the insulin molecule. By “insulin” or “an insulin molecule”, it is intended that this disclosure encompasses both wild-type insulin and modified forms of insulin as long as they are bioactive (i.e., capable of causing a detectable reduction in glucose when administered in vivo). Wild-type insulin includes insulin from any species whether in purified, synthetic, or recombinant form (e.g., human insulin, porcine insulin, bovine insulin, rabbit insulin, sheep insulin, etc.). A number of these are available commercially, e.g., from Sigma-Aldrich (St. Louis, MO). A variety of modified forms of insulin are known in the art (e.g., see Crotty and Reynolds, PEDIATR. EMERG. CARE. 23:903-905, 2007 and Gerich, AM. J. M^{ED}. 113:308-16, 2002). Modified forms of insulin (insulin analogs) may be chemically modified (e.g., by addition of a chemical moiety such as a PEG group or a fatty acyl chain as described below) and/or mutated (i.e., by addition, deletion, or substitution of one or more amino acids).

In particular embodiments, an insulin molecule of the present disclosure will differ from a wild-type insulin by 1-10 (e.g., 1-9, 1-8, 1-7, 1-6, 1-5, 1-4, 1-3, 1-2, 2-9, 2-8, 2-7, 2-6, 2-5, 2-4, 2-3, 3-9, 3-8, 3-7, 3-6, 3-5, 3-4, 4-9, 4-8, 4-7, 4-6, 4-5, 5-9, 5-8, 5-7, 5-6, 6-9, 6-8, 6-7, 7-9, 7-8, 8-9, 9, 8, 7, 6, 5, 4, 3, 2, or 1) amino acid substitutions, additions and/or deletions. In particular embodiments, an insulin molecule of the present disclosure will differ from a wild-type insulin by amino acid substitutions only. In particular embodiments, an insulin molecule of the present disclosure will differ from a wild-type insulin by amino acid additions only. In particular embodiments, an insulin molecule of the present disclosure will differ from wild-type insulin by both amino acid substitutions and additions. In particular embodiments, an insulin molecule of the present disclosure will differ from a wild-type insulin by both amino acid substitutions and deletions.

In particular embodiments, amino acid substitutions may be made on the basis of similarity in polarity, charge, solubility, hydrophobicity, hydrophilicity, and/or the amphipathic nature of the residues involved. In particular embodiments, a substitution may be conservative, that is, one amino acid is replaced with one of similar shape and charge. Conservative substitutions are well known in the art and typically include substitutions within the following groups: glycine, alanine; valine, isoleucine, leucine; aspartic acid, glutamic acid; asparagine, glutamine; serine, threonine; lysine, arginine; and tyrosine, phenylalanine. In particular embodiments, the hydrophobic index of amino acids may be considered in choosing suitable mutations. The importance of the hydrophobic amino acid index in conferring interactive biological function on a polypeptide is generally understood in the art. Alternatively, the substitution of like amino acids can be made effectively on the basis of hydrophilicity. The importance of hydrophilicity in conferring interactive biological function of a polypeptide is generally understood in the art. The use of the hydrophobic index or hydrophilicity in designing polypeptides is further discussed in U.S. Pat. No. 5,691,198.

The wild-type sequence of human insulin (A-chain and B-chain) is shown below.

A-Chain (SEQ ID NO: 1):
GIVEQCCTSICSLYQLENYCN
B-Chain (SEQ ID NO: 2):
FVNQHLCGSHLVEALYLVCGERGFFYTPKT

In various embodiments, an insulin molecule of the present disclosure may be mutated at the B28 and/or B29 positions of the B-peptide sequence. For example, insulin lispro (HUMALOG™) is a rapid acting insulin mutant having the A-Chain of wild-type human insulin and in which the penultimate lysine and proline residues on the C-terminal end of the B-peptide have been reversed (Lys^{B28}Pro^{B29}-human insulin) (SEQ ID NO: 3).

B-Chain (SEQ ID NO: 3):
FVNQHLCGSHLVEALYLVCGERGFFYTKPT

This modification blocks the formation of insulin multimers. Insulin aspart (NOVOLOG™) is another rapid acting insulin mutant having the A-Chain of wild-type human insulin and in which proline at position B28 has been substituted with aspartic acid (Asp^{B28}-human insulin) (SEQ ID NO: 4).

B-Chain (SEQ ID NO: 4):
FVNQHLCGSHLVEALYLVCGERGFFYTDKT

This mutant also prevents the formation of multimers. In some embodiments, mutation at positions B28 and/or B29 is accompanied by one or more mutations elsewhere in the insulin polypeptide. For example, insulin glulisine (APIDRA™) is yet another rapid acting insulin mutant having the A-Chain of wild-type human insulin and in which aspartic acid at position B3 has been replaced by a lysine residue and lysine at position B29 has been replaced with a glutamic acid residue (Lys^{B3}Glu^{B29}-human insulin) (SEQ ID NO: 5).

B-Chain (SEQ ID NO: 5):
FVKQHLCGSHLVEALYLVCGERGFFYTPDT

In various embodiments, an insulin molecule of the present disclosure has an isoelectric point that is shifted relative to human insulin. In some embodiments, the shift in isoelectric point is achieved by adding one or more arginine residues to the N-terminus of the insulin A-peptide and/or the C-terminus of the insulin B-peptide. Examples of such insulin polypeptides include Arg^{A0}-human insulin, Arg^{B31}Arg^{B32}-human insulin, Gly^{A2}1Arg^{B31}Arg^{B32}-human insulin, Arg^{A0}Arg^{B31}Arg^{B32}-human insulin, and Arg^{A0}Gly^{A21}Arg^{B31}Arg^{B32}-human insulin. By way of further example, insulin glargine (LANTUS™) is an exemplary long acting insulin mutant in which Asp^{A21} has been replaced by glycine (SEQ ID NO: 6), and two arginine residues have been added to the C-terminus of the B-peptide (SEQ ID NO: 7).

A-Chain (SEQ ID NO: 6):
GIVEQCCTSICSLYQLENYCG
B-Chain (SEQ ID NO: 7):
FVNQHLCGSHLVEALYLVCGERGFFYTPKTRR

The effect of these changes is to shift the isoelectric point, producing a solution that is completely soluble at pH 4. Thus, in some embodiments, an insulin molecule of the present disclosure comprises an A-peptide sequence wherein A21 is Gly and B-peptide sequence wherein B31 and B32 are Arg-Arg. It is to be understood that the present disclosure encompasses all single and multiple combinations of these mutations and any other mutations that are described herein (e.g., Gly^{A21}-human insulin, Gly^{A21}Arg^{B31}-human insulin, Arg^{B31}Arg^{B32}-human insulin, Arg^{B31}-human insulin).

In various embodiments, an insulin molecule of the present disclosure is truncated. For example, in particular embodiments, a B-peptide sequence of an insulin polypeptide of the present disclosure is missing B1, B2, B3, B26, B27, B28, B29, and/or B30. In particular embodiments, combinations of residues are missing from the B-peptide sequence of an insulin polypeptide of the present disclosure. For example, the B-peptide sequence may be missing residues B(1-2), B(1→3), B(29-30), B(28-30), B(27-30), and/or B(26-30). In some embodiments, these deletions and/or truncations apply to any of the aforementioned insulin molecules (e.g., without limitation to produce des(B30)-insulin lispro, des(B30)-insulin aspart, des(B30)-insulin glulisine, des(B30)-insulin glargine, etc.).

In some embodiments, an insulin molecule contains additional amino acid residues on the N- or C-terminus of the A or B-peptide sequences. In some embodiments, one or more amino acid residues are located at positions A0, A21, B0, and/or B31. In some embodiments, one or more amino acid residues are located at position A0. In some embodiments, one or more amino acid residues are located at position A21. In some embodiments, one or more amino acid residues are located at position 80. In some embodiments, one or more amino acid residues are located at position B31. In particular embodiments, an insulin molecule does not include any additional amino acid residues at positions A0, A21, B0, or B31.

In particular embodiments, an insulin molecule of the present disclosure is mutated such that one or more amidated amino acids are replaced with acidic forms. For example, asparagine may be replaced with aspartic acid or glutamic acid. Likewise, glutamine may be replaced with aspartic acid or glutamic acid. In particular, Asn^{A18}, Asn^{A21}, or Asn^{B3}, or any combination of those residues, may be replaced by aspartic acid or glutamic acid. Gln^{A15} or Gln^{B4}, or both, may be replaced by aspartic acid or glutamic acid. In particular embodiments, an insulin molecule has aspartic acid at position A21 or aspartic acid at position B3, or both.

One skilled in the art will recognize that it is possible to mutate yet other amino acids in the insulin molecule while retaining biological activity. For example, without limitation, the following modifications are also widely accepted in the art: replacement of the histidine residue of position B10 with aspartic acid (His^{B10}→Asp^{B10}); replacement of the phenylalanine residue at position B1 with aspartic acid (Phe^{B1}→Asp^{B1}); replacement of the threonine residue at position B30 with alanine (Thr^{B30}→Ala^{B30}); replacement of the tyrosine residue at position B26 with alanine (Tyr^{B26}→Ala^{B26}); and replacement of the serine residue at position B9 with aspartic acid (Ser^{B9}→Asp^{B9}).

In various embodiments, an insulin molecule of the present disclosure has a protracted profile of action. Thus, in particular embodiments, an insulin molecule of the present disclosure may be acylated with a fatty acid. That is, an amide bond is formed between an amino group on the insulin molecule and the carboxylic acid group of the fatty acid. The amino group may be the alpha-amino group of an N-terminal amino acid of the insulin molecule, or the amino group may be the epsilon-amino group of a lysine residue of the insulin molecule. An insulin molecule of the present disclosure may be acylated at one or more of the three amino groups that are present in wild-type human insulin or may be acylated on lysine residue that has been introduced into the wild-type human insulin sequence. In particular embodiments, an insulin molecule may be acylated at position B1. In particular embodiments, an insulin molecule may be acylated at position B29. In particular embodiments, the insulin molecule is acylated with a fatty acid molecule. In particular embodiments, the fatty acid is selected from myristic acid (C14), pentadecylic acid (C15), palmitic acid (C16), heptadecylic acid (C17) and stearic acid (C18). For example, insulin detemir (LEVEMIR™) is a long acting insulin mutant in which Thr^{B30} has been deleted, and a C14 fatty acid chain (myristic acid) has been attached to Lys^{B29}.

In some embodiments, the N-terminus of the A-peptide, the N-terminus of the B-peptide, the epsilon-amino group of Lys at position B29 or any other available amino group in an insulin molecule of the present disclosure is covalently linked to a fatty acid moiety of general formula:

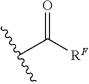

wherein R^{F} is hydrogen or a C₁₋₃₀ alkyl group. In some embodiments, R^{F} is a C₁₋₂₀ alkyl group, a C₃₋₁₉ alkyl group, a C₅₋₁₈ alkyl group, a C₆₋₁₇ alkyl group, a C₈₋₁₆ alkyl group, a C₁₀₋₁₅ alkyl group, or a C₁₂₋₁₄ alkyl group. In particular embodiments, the insulin polypeptide is conjugated to the moiety at the A1 position. In particular embodiments, the insulin polypeptide is conjugated to the moiety at the B1 position. In particular embodiments, the insulin polypeptide is conjugated to the moiety at the epsilon-amino group of Lys at position B29. In particular embodiments, position B28 of the insulin molecule is Lys and the epsilon-amino group of Lys^{B28} is conjugated to the fatty acid moiety. In particular embodiments, position B3 of the insulin molecule is Lys and the epsilon-amino group of LyS^{B3} is conjugated to the fatty acid moiety. In some embodiments, the fatty acid chain is 8-20 carbons long. In some embodiments, the fatty acid is octanoic acid (C8), nonanoic acid (C9), decanoic acid (C10), undecanoic acid (C11), dodecanoic acid (C12), or tridecanoic acid (C13). In particular embodiments, the fatty acid is myristic acid (C14), pentadecanoic acid (C15), palmitic acid (C16), heptadecanoic acid (C17), stearic acid (C18), nonadecanoic acid (C19), or arachidic acid (C20).

In various embodiments, an insulin molecule of the present disclosure includes the three wild-type disulfide bridges (i.e., one between position 7 of the A-chain and position 7 of the B-chain, a second between position 20 of the A-chain and position 19 of the B-chain, and a third between positions 6 and 11 of the A-chain). In particular embodiments, an insulin molecule is mutated such that the site of mutation is used as a conjugation point, and conjugation at the mutated site reduces binding to the insulin receptor (e.g., Lys^{A3}). In particular other embodiments, conjugation at an existing wild-type amino acid or terminus reduces binding to the insulin receptor (e.g., Gly^{A1}). In some embodiments, an insulin molecule is conjugated at position A4, A5, A8, A9, or B30. In particular embodiments, the conjugation at position A4, A5, A8, A9, or B30 takes place via a wild-type amino acid side chain (e.g., Glu^{A4}). In particular other embodiments, an insulin molecule is mutated at position A4, A5, A8, A9, or B30 to provide a site for conjugation (e.g., Lys^{A4}, Lys^{A5}, Lys^{A}8, Lys^{A9}, or Lys^{B30}).

Methods for conjugating insulin molecules are described below. In particular embodiments, an insulin molecule is conjugated to a penta-valent sugar cluster via the side chain amino group of B29 lysine of insulin or insulin analog, in the presence or absence of small substitution on A1 and B1 amino groups. It will be appreciated that different conjugation positions on the B-chain may lead to different reductions in insulin activity.

### Exemplary Insulin Conjugates

In various embodiments, the insulin conjugate of the present disclosure comprises an insulin or insulin analog molecule conjugated one penta-valent sugar cluster, wherein the penta-valent sugar cluster is provided by a branched linker having five arms (penta-dentate linker), wherein each arm of the penta-dentate linker is independently covalently linked to a ligand comprising or consisting of a monosaccharide, disaccharide, trisaccharide, tetrasaccharide, or branched trisaccharide. In particular embodiments, the ligands are independently selected from the group consisting of AEG, AEM, AEBM, AETM, AEGA, and AEF. In more particular embodiments, the insulin molecule is conjugated via the side chain amino group of B29 lysine of insulin or insulin analog, in the presence or absence of small substitution(s) on A1 and B1 amino groups.

In particular embodiments, the insulin or insulin molecule of the above insulin conjugate may be conjugated to one or more additional linkers attached to one or more ligands, each ligand independently selected from AEG, AEM, AEBM, AETM, AEGA, and AEF. The additional linkers may be linear, bi-dentate, tri-dentate, quadra-dentate, etc., wherein each arm of the linker comprises a ligand, which may independently be selected from AEG, AEM, AEBM, AETM, AEGA, and AEF.

Thus, in particular embodiments, the insulin conjugate may comprise or consist of a penta-valent sugar cluster conjugated via the side chain amino group of B29 lysine of insulin or insulin analog, in the presence or absence of small substitution(s) on A1 and B1 amino groups.

In particular embodiments, the insulin conjugate may comprise or consist of two penta-valent sugar clusters (a first sugar cluster and a second sugar cluster) wherein each ligand comprising the first penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups, and wherein each ligand comprising the second penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups.

In particular embodiments, the insulin conjugate may comprise or consist of three penta-valent sugar clusters (a first sugar cluster, a second sugar cluster, and a third sugar cluster) wherein each ligand comprising the first penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups; wherein each ligand comprising the second penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups, and wherein each ligand comprising the third penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups.

In particular embodiments, the insulin conjugate may comprise or consist of four penta-valent sugar clusters (a first sugar cluster, a second sugar cluster, a third sugar cluster, and a fourth sugar cluster) wherein each ligand comprising the first penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups; wherein each ligand comprising the second penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups and wherein each ligand comprising the third penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups, and wherein each ligand comprising the fourth penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups.

In particular embodiments, the insulin conjugate may comprise or consist of five penta-valent sugar clusters (a first sugar cluster, a second sugar cluster, a third sugar cluster, a fourth sugar cluster, and a fifth sugar cluster) wherein each ligand comprising the first penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups; wherein each ligand comprising the second penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups and wherein each ligand comprising the third penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups, wherein each ligand comprising the fourth penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups, and wherein each ligand comprising the fifth penta-valent sugar cluster is independently a ligand selected from AEG, AEM, AEBM, AETM, AEGA, and AEF is conjugated via the side chain amino group of B29 lysine of insulin or insulin analog, in the presence or absence of small substitution(s) on A1 and B1 amino groups.

In particular embodiments, the insulin or insulin analog molecule further includes an acyl group covalently linked to the A1 or both A1 and B1 N-terminal amino groups. In particular embodiments, the insulin or insulin analog molecule further includes a urea group covalently linked to the A1 and B1 N-terminal amino groups.

### Insulin Conjugates

This section describes some exemplary insulin or insulin analog conjugates.

In various embodiments, the conjugates may have the general formula I:

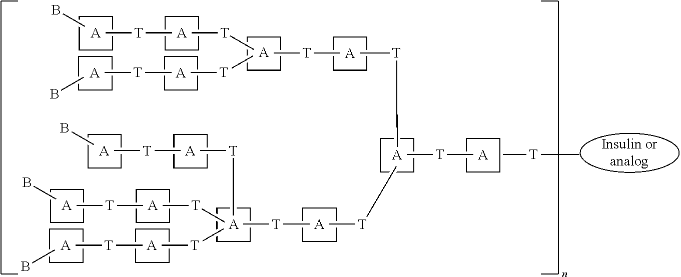

wherein
(i) T is a linker, and each occurrence of T is independently selected from a covalent bond or a bivalent, straight or branched, saturated or unsaturated, optionally substituted C₁₋₃₀ hydrocarbon chain, wherein
   (a) one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic moiety, an aryl moiety, or a heteroaryl moiety, and
   (b) each occurrence of R is independently hydrogen, a suitable protecting group, an acyl moiety, arylalkyl moiety, aliphatic moiety, aryl moiety, heteroaryl moiety, or heteroaliphatic moiety;
(ii) [symbol] is a branching point, and each occurrence of [symbol] is independently selected from a covalent bond, a carbon atom, a heteroatom, or an optionally substituted group selected from the group consisting of acyl moieties, aliphatic moieties, heteroaliphatic moieties, aryl moieties, heteroaryl moieties, and heterocyclic moieties; and
(iii) B is a sugar-containing moiety, and each occurrence of B is independently -T-L^{B}-X, wherein each occurrence of X is independently a ligand comprising or consisting of a monosaccharide, disaccharide, trisaccharide, tetrasaccharide, or branched trisaccharide, and each occurrence of L^{B} is independently a covalent bond or a group derived from the covalent conjugation of a T with an X; and,
(iv) n is 1, 2, or 3.

In various embodiments, the conjugates may have the general formula II:

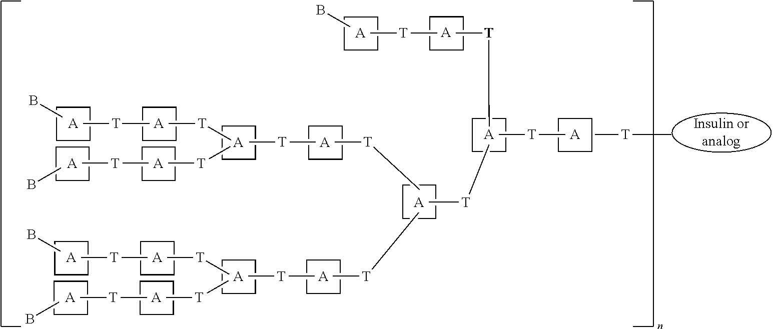

wherein
(i) T is a linker, and each occurrence of T is independently selected from a covalent bond or a bivalent, straight or branched, saturated or unsaturated, optionally substituted C₁₋₃₀ hydrocarbon chain, wherein
   (a) one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic moiety, an aryl moiety, or a heteroaryl moiety, and
   (b) each occurrence of R is independently hydrogen, a suitable protecting group, an acyl moiety, arylalkyl moiety, aliphatic moiety, aryl moiety, heteroaryl moiety, or heteroaliphatic moiety;
(ii) [symbol] is a branching point, and each occurrence of [symbol] is independently selected from a covalent bond, a carbon atom, a heteroatom, or an optionally substituted group selected from the group consisting of acyl moieties, aliphatic moieties, heteroaliphatic moieties, aryl moieties, heteroaryl moieties, and heterocyclic moieties; and
(iii) B is a sugar-containing moiety, and each occurrence of B is independently -T-L^{B}-X, wherein each occurrence of X is independently a ligand comprising or consisting of a monosaccharide, disaccharide, trisaccharide, tetrasaccharide, or branched trisaccharide, and each occurrence of L^{B} is independently a covalent bond or a group derived from the covalent conjugation of a T with an X; and,
(iv) n is 1, 2, or 3.

In various embodiments, the conjugates may have the general formula III:

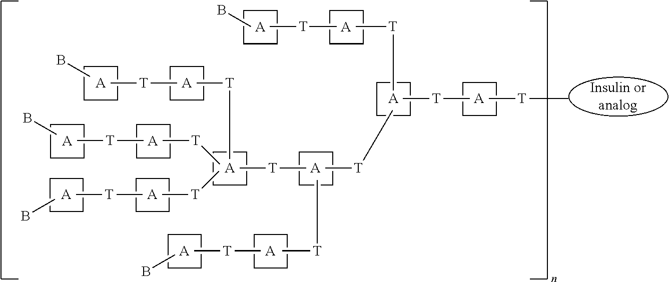

wherein
(i) T is a linker, and each occurrence of T is independently selected from a covalent bond or a bivalent, straight or branched, saturated or unsaturated, optionally substituted C₁₋₃₀ hydrocarbon chain, wherein
   (a) one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic moiety, an aryl moiety, or a heteroaryl moiety, and
   (b) each occurrence of R is independently hydrogen, a suitable protecting group, an acyl moiety, arylalkyl moiety, aliphatic moiety, aryl moiety, heteroaryl moiety, or heteroaliphatic moiety;
(ii) [symbol] is a branching point, and each occurrence of [symbol] is independently selected from a covalent bond, a carbon atom, a heteroatom, or an optionally substituted group selected from the group consisting of acyl moieties, aliphatic moieties, heteroaliphatic moieties, aryl moieties, heteroaryl moieties, and heterocyclic moieties; and
(iii) B is a sugar-containing moiety, and each occurrence of B is independently -T-L^{B}-X, wherein each occurrence of X is independently a ligand comprising or consisting of a monosaccharide, disaccharide, trisaccharide, tetrasaccharide, or branched trisaccharide, and each occurrence of L^{B} is independently a covalent bond or a group derived from the covalent conjugation of a T with an X; and,
(iv) n is 1, 2, or 3.

In various embodiments, the conjugates may have the general formula IV:

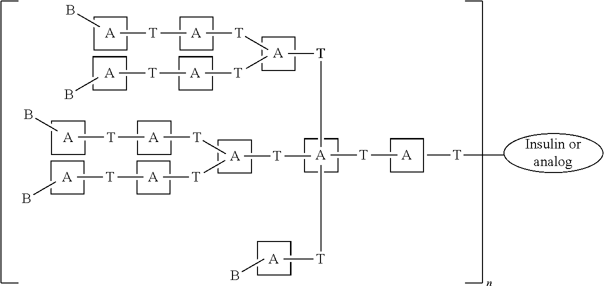

wherein
(i) T is a linker, and each occurrence of T is independently selected from a covalent bond or a bivalent, straight or branched, saturated or unsaturated, optionally substituted C₁₋₃₀ hydrocarbon chain, wherein
   (a) one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic moiety, an aryl moiety, or a heteroaryl moiety, and
   (b) each occurrence of R is independently hydrogen, a suitable protecting group, an acyl moiety, arylalkyl moiety, aliphatic moiety, aryl moiety, heteroaryl moiety, or heteroaliphatic moiety;
(ii) [symbol] is a branching point, and each occurrence of [symbol] is independently selected from a covalent bond, a carbon atom, a heteroatom, or an optionally substituted group selected from the group consisting of acyl moieties, aliphatic moieties, heteroaliphatic moieties, aryl moieties, heteroaryl moieties, and heterocyclic moieties; and
(iii) B is a sugar-containing moiety, and each occurrence of B is independently -T-L^{B}-X, wherein each occurrence of X is independently a ligand comprising or consisting of a monosaccharide, disaccharide, trisaccharide, tetrasaccharide, or branched trisaccharide, and each occurrence of L^{B} is independently a covalent bond or a group derived from the covalent conjugation of a T with an X; and,
(iv) n is 1, 2, or 3.

### Description of Exemplary Groups

[symbol]

In particular embodiments, each occurrence of [symbol] is independently an optionally substituted group selected from the group consisting of acyl moieties, aliphatic moieties, heteroaliphatic moieties, aryl moieties, heteroaryl moieties, and heterocyclic moieties. In some embodiments, each occurrence of [symbol] is the same. In some embodiments, the central [symbol] is different from all other occurrences of [symbol] . In particular embodiments, all occurrences of [symbol] are the same except for the central [symbol] .

In some embodiments, [symbol] is an optionally substituted aryl or heteroaryl group.

In some embodiments, [symbol] is a 2-, 3-, 4-, 6-, or 8-membered aryl or heteroaryl group. In some embodiments, [symbol] is a 5- or 6-membered heterocyclic group. In particular embodiments, [symbol] is a heteroatom selected from N, O, or S. In some embodiments, is nitrogen atom. In some embodiments, [symbol] is an oxygen atom. In some embodiments, [symbol] is sulfur atom. In some embodiments, [symbol] is a carbon atom. In some embodiments, [symbol] is the structure

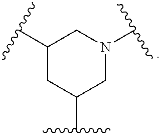 [SMILES: *C1CC(*)CN(*)C1]

### T (Linker)

In particular embodiments, each occurrence of T is independently a bivalent, straight or branched, saturated or unsaturated, optionally substituted C₁₋₂₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group. In particular embodiments, one, two, three, four, or five methylene units of T are optionally and independently replaced. In particular embodiments, T is constructed from a C₁₋₁₀, C₁₋₈, C₁₋₆, C₁₋₄, C₂₋₁₂, C₄₋₁₂, C₆₋₁₂, C₈₋₁₂, or C₁₀₋₁₂ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group. In some embodiments, one or more methylene units of T is replaced by a heterocyclic group. In some embodiments, one or more methylene units of T is replaced by a triazole moiety. In particular embodiments, one or more methylene units of T is replaced by —C(O)—. In particular embodiments, one or more methylene units of T is replaced by —C(O)N(R)—. In particular embodiments, one or more methylene units of T is replaced by —O—.

In particular embodiments of the conjugate, the conjugate is a conjugate having general formula I, and the insulin or insulin analog is conjugated to a pentavalent linker selected from the group consisting of:

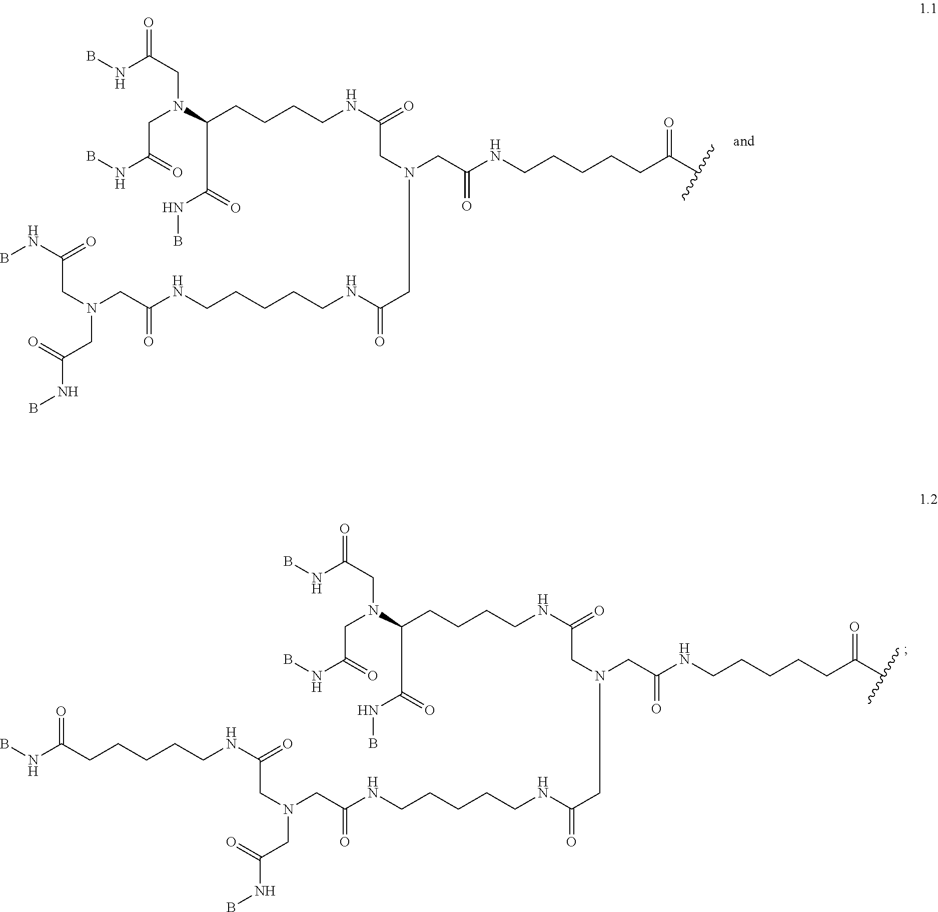

or the conjugate is a conjugate having general formula II, and the insulin or insulin analog is conjugated to a pentavalent linker selected from the group consisting of:

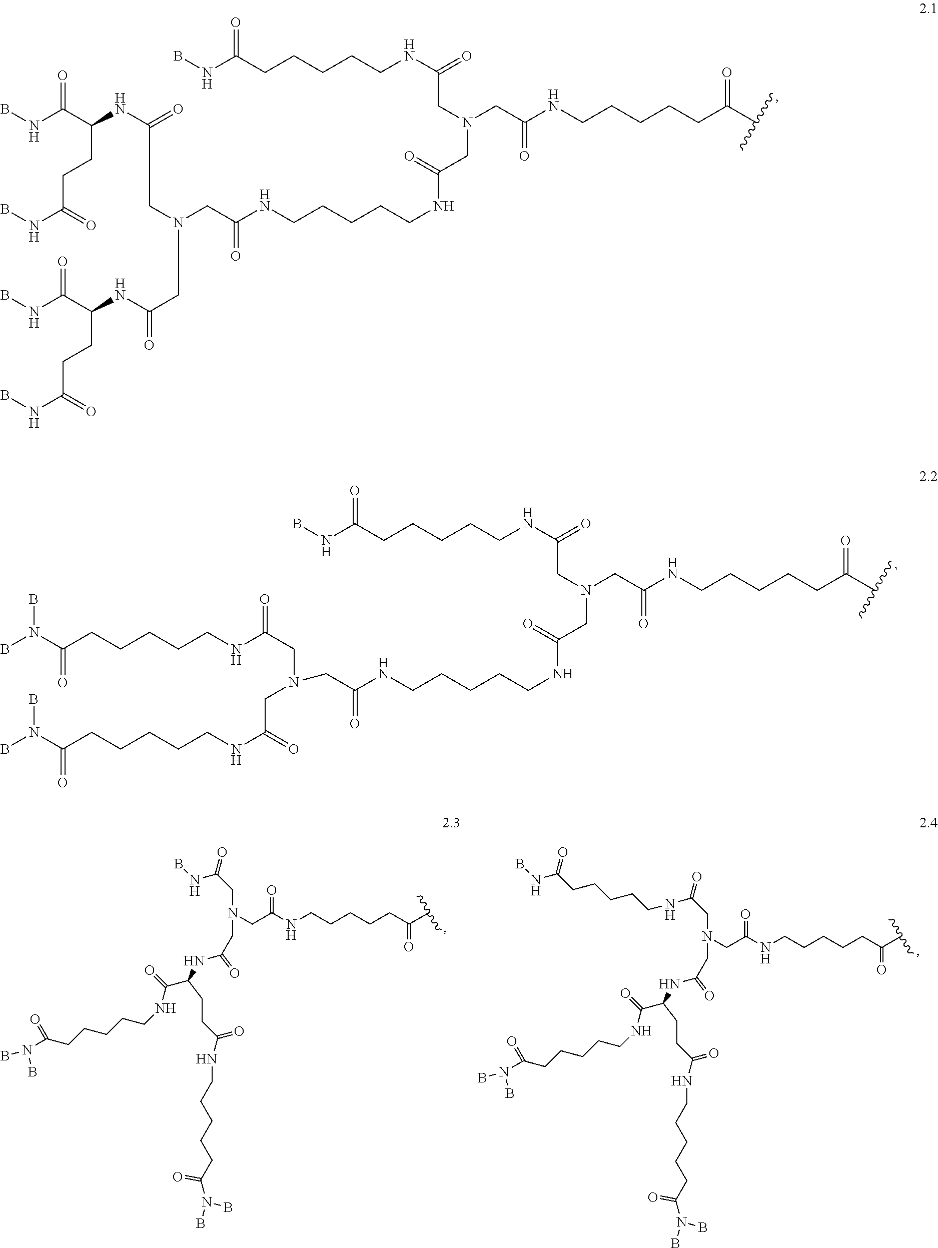

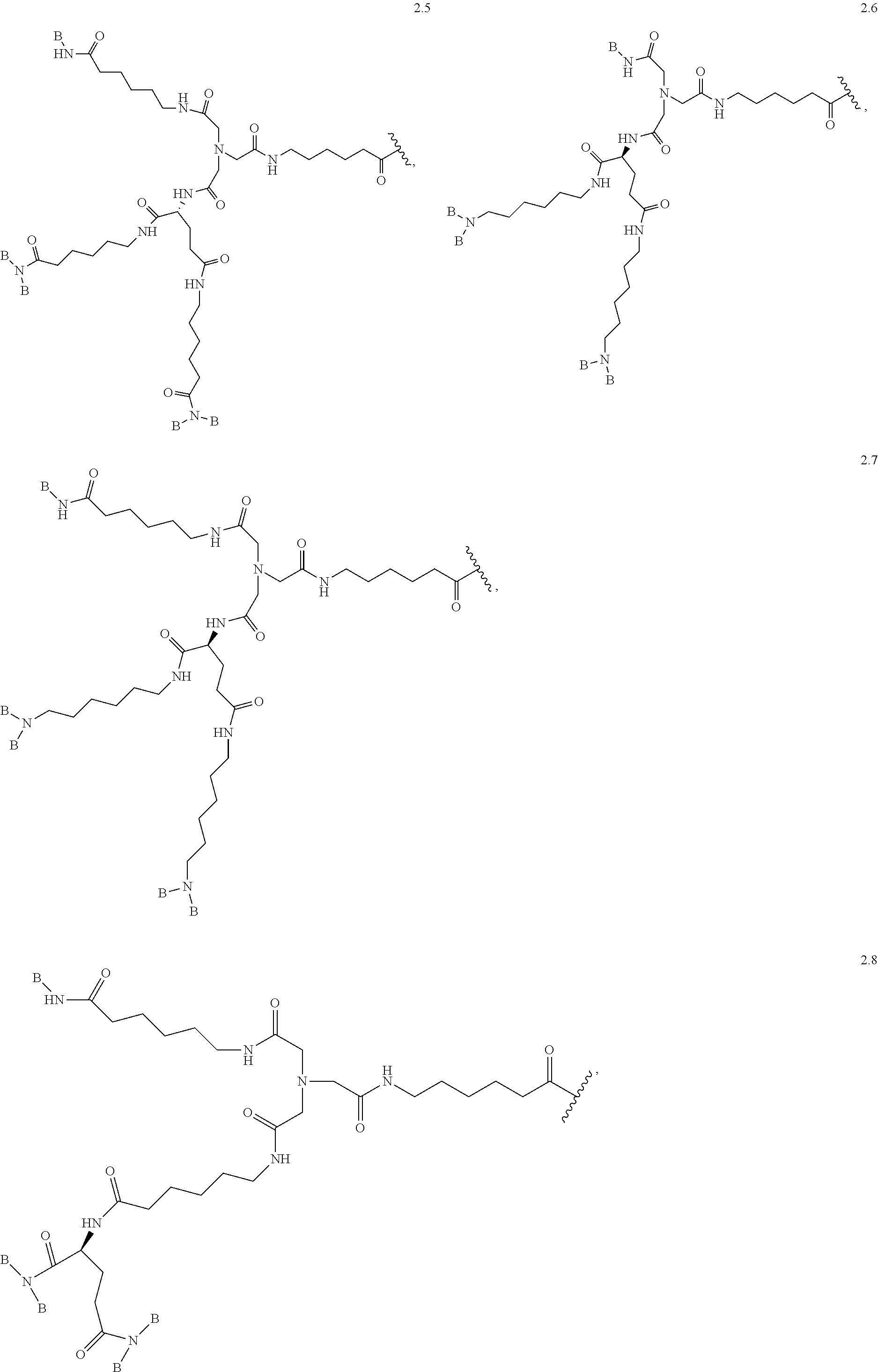

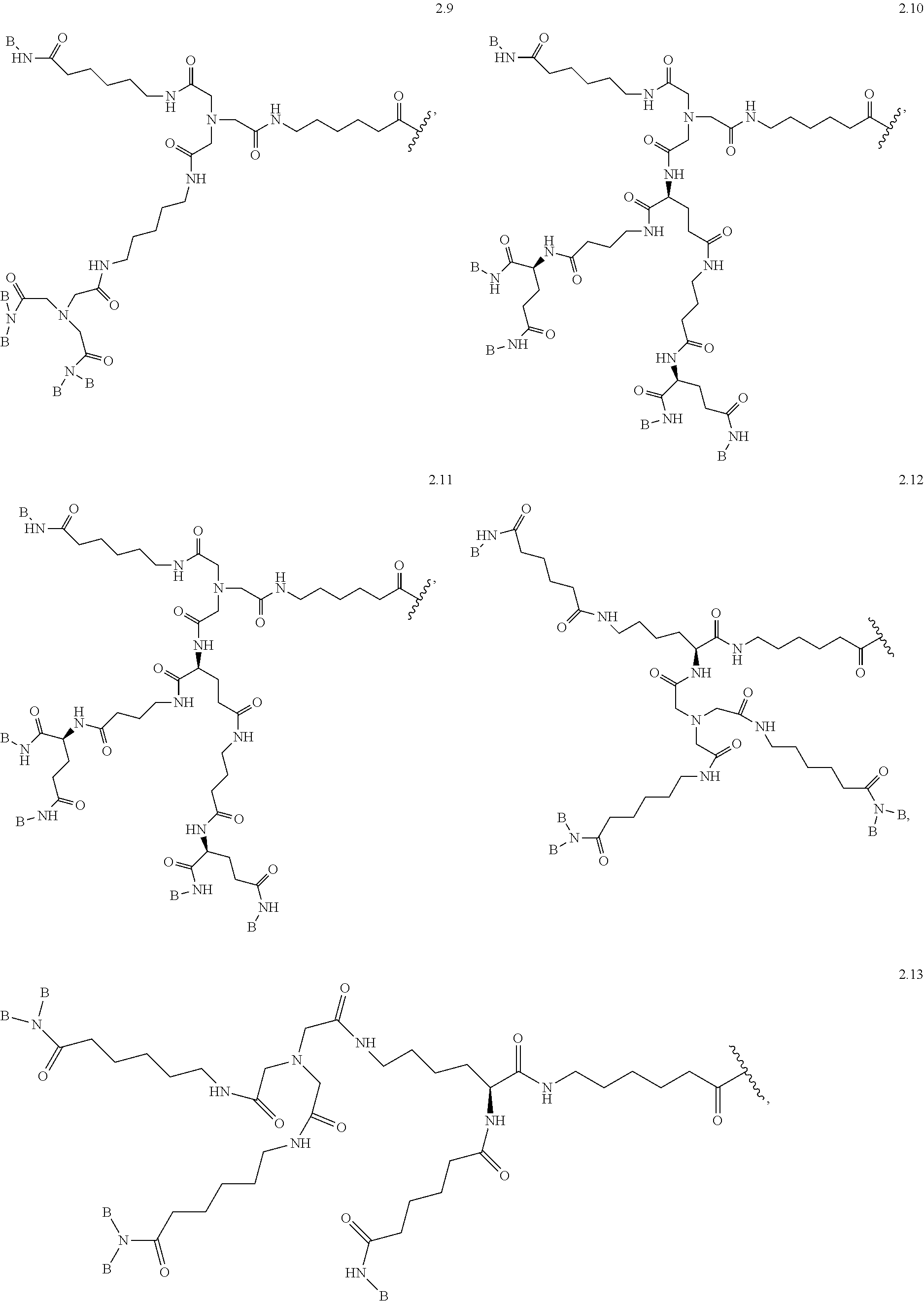

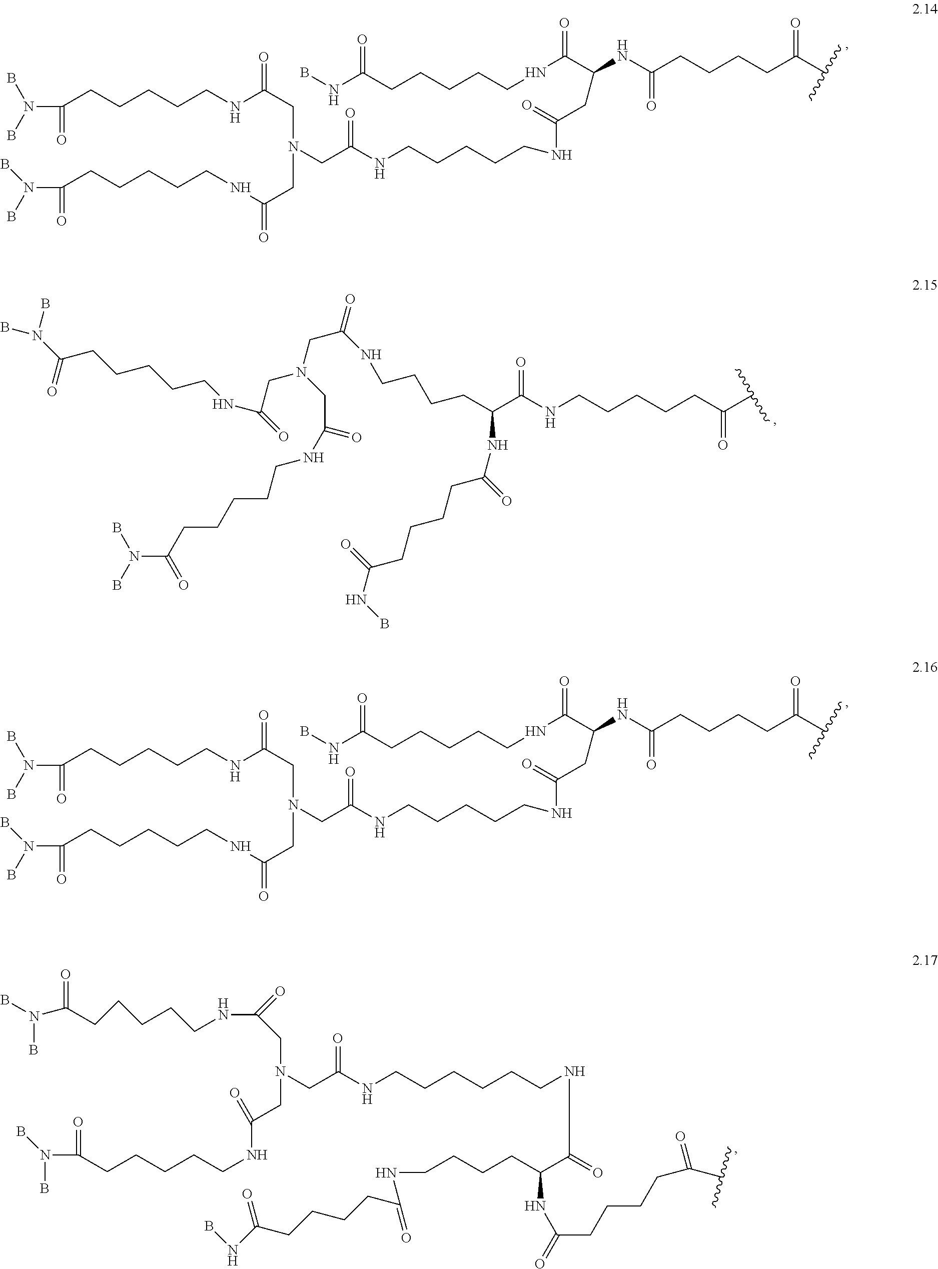

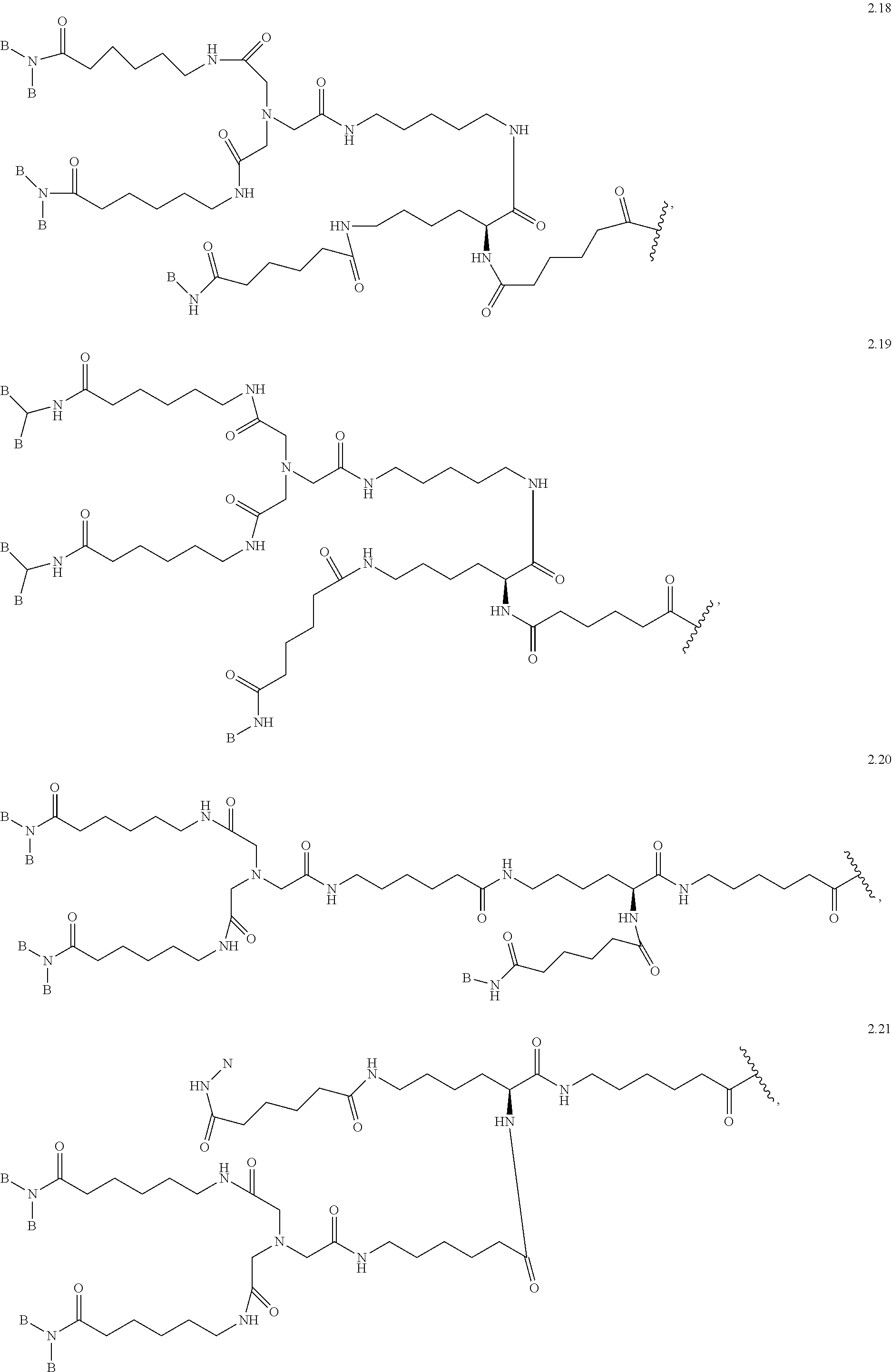

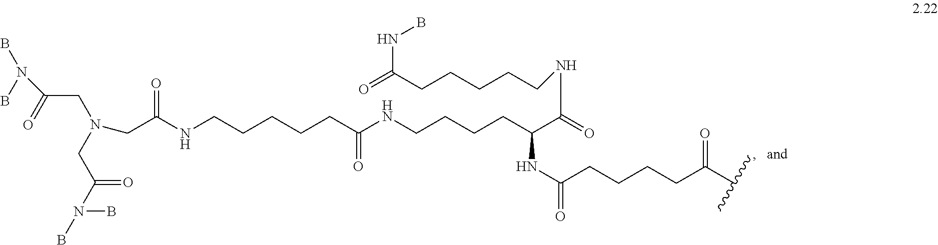

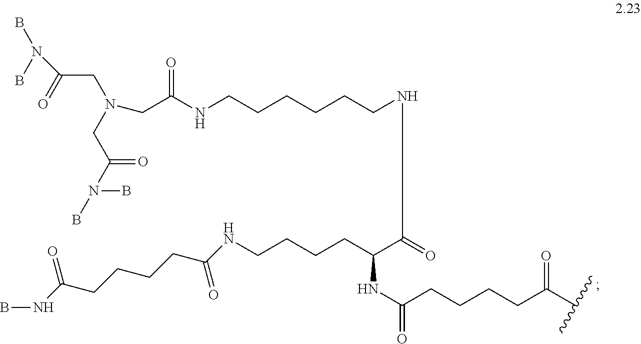

or the conjugate is a conjugate having general formula III, and the insulin or insulin analog is conjugated to a pentavalent linker selected from the group consisting of:

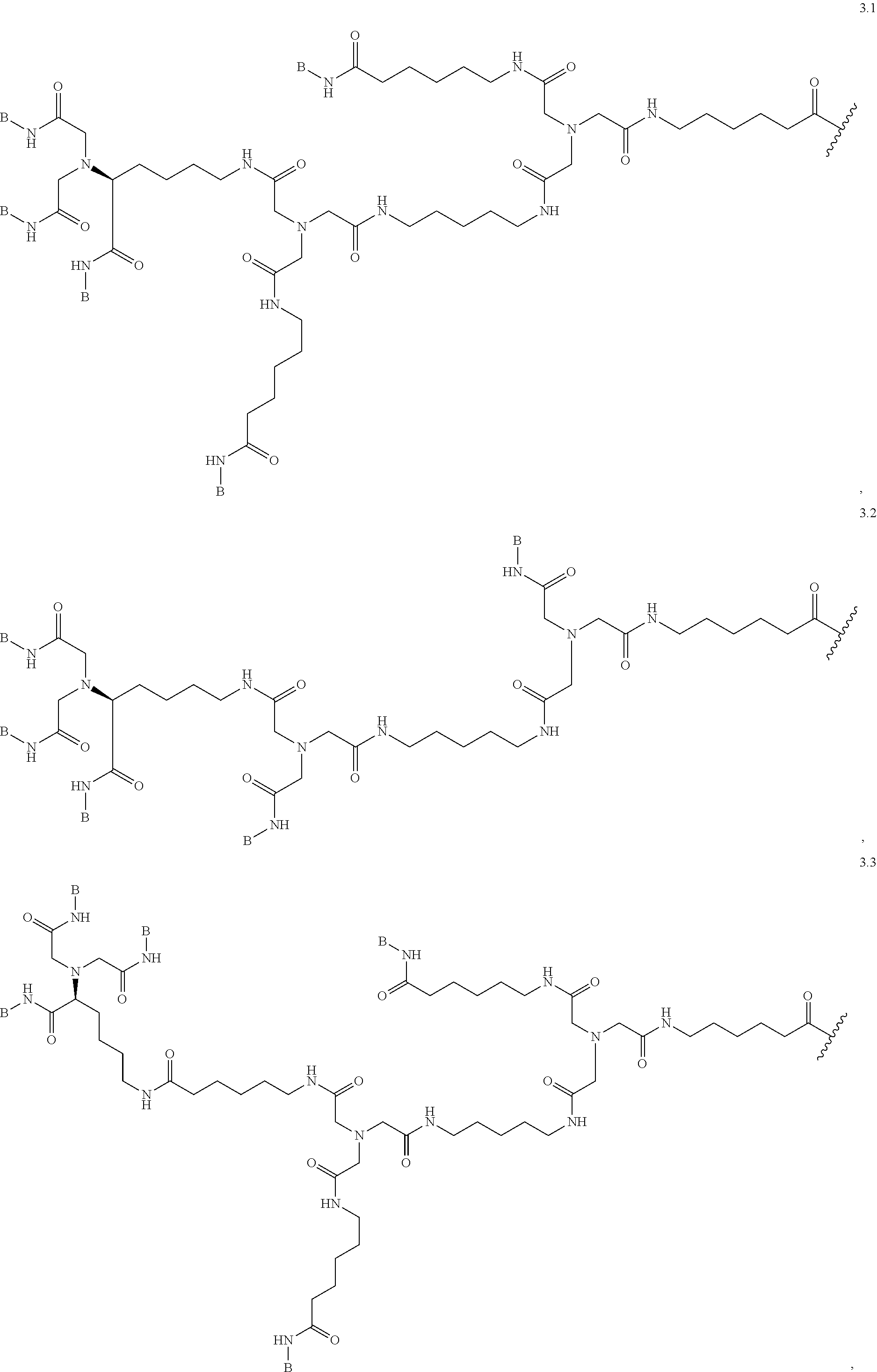

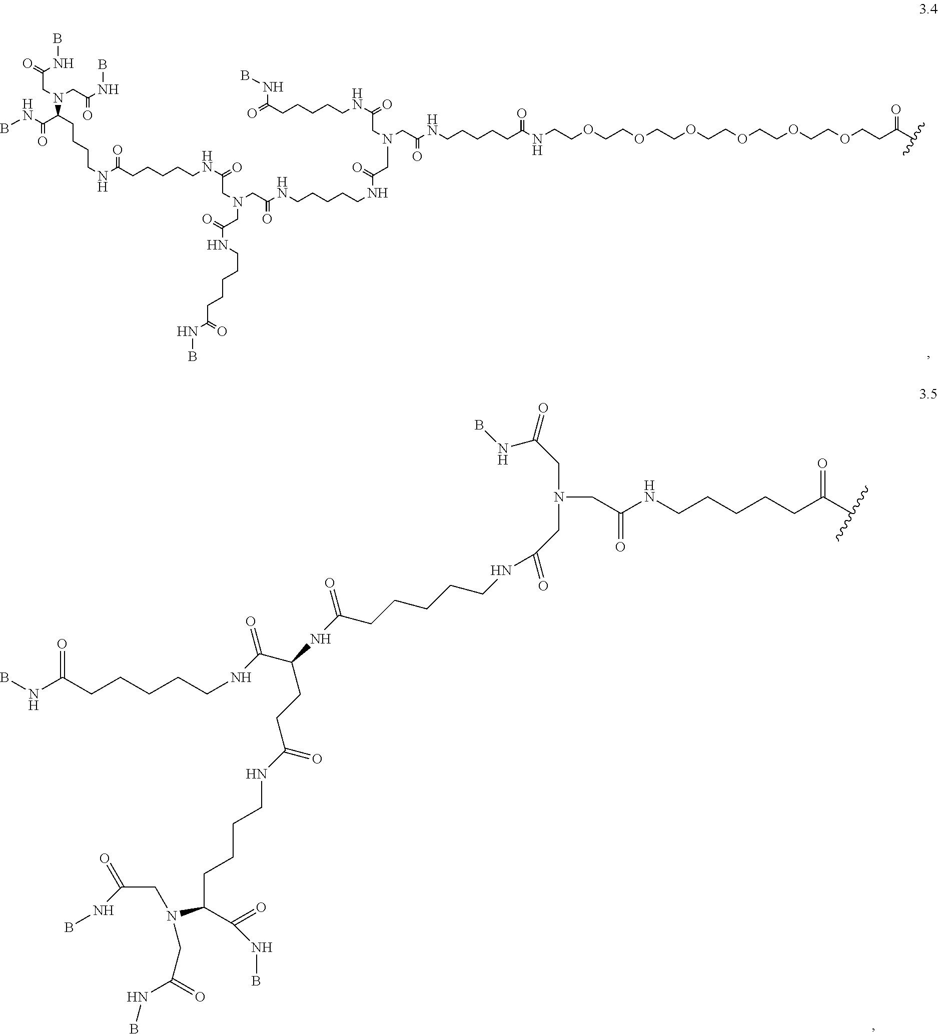

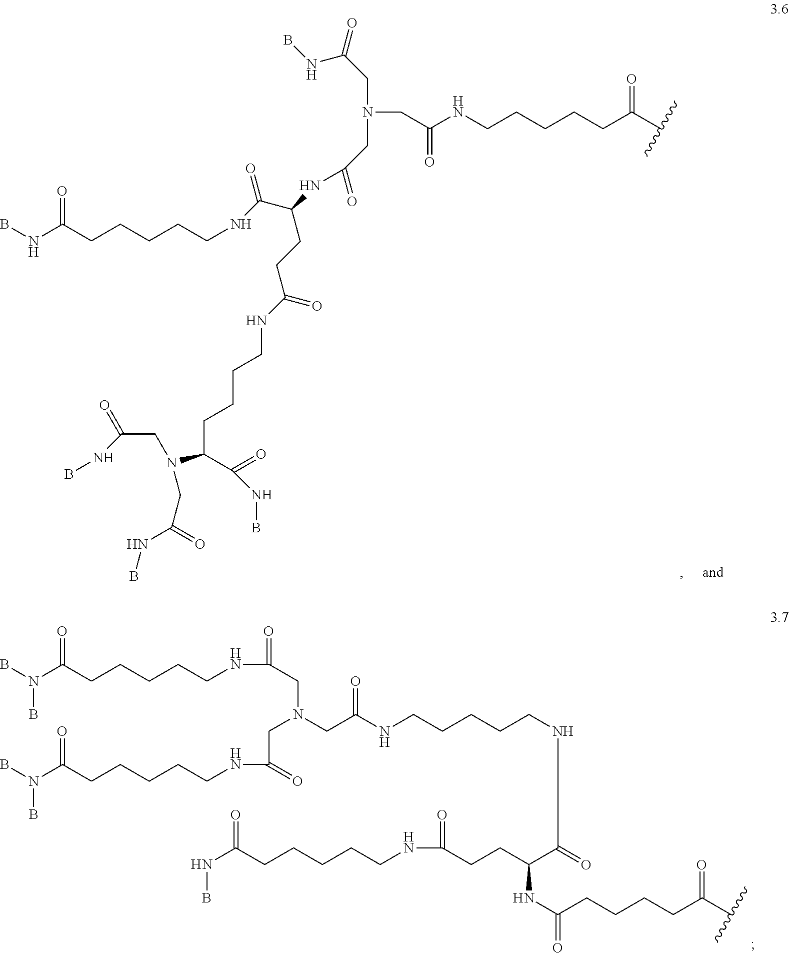

or the conjugate is a conjugate having general formula IV, and the insulin or insulin analog is conjugated to a pentavalent linker that is

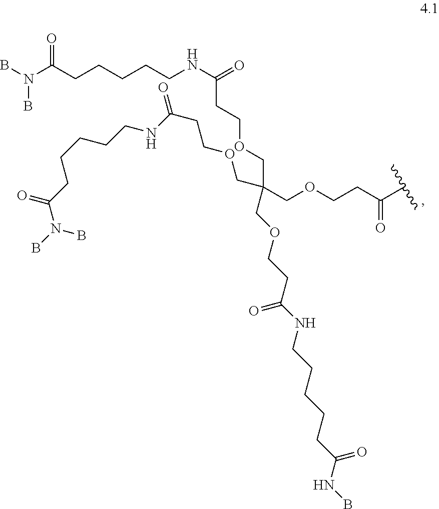

wherein a wavy line indicates the bond between the proximal end of the linker arm and amino acid on the insulin or insulin analog and wherein each B is independently -T-L^{B}-X, wherein each occurrence of X is independently the ligand and each occurrence of L^{B} is independently a covalent bond or a group derived from the covalent conjugation of a T with an X.

In particular embodiments, the insulin analog may comprise an A chain sequence comprising a sequence of GIVEQCCX₁SICSLYQLENYCX₂ (SEQ ID NO: 8); and a B chain sequence comprising a sequence of X₃LCGX₄X₅LVEALYLVCGERGFF (SEQ ID NO: 9), or X₈VNQX₃LCGX₄X₅LVEALYLVCGERGFFYTX₆X₇ (SEQ ID NO: 10), wherein
X₁ is selected from the group consisting of threonine and histidine;
X₂ is selected from the group consisting of asparagine and glycine;
X₃ is selected from the group consisting of histidine and threonine;
X₄ is selected from the group consisting of alanine, glycine, and serine;
X₅ is selected from the group consisting of histidine, aspartic acid, glutamic acid, homocysteic acid, and cysteic acid;
X₆ is selected from the group consisting of aspartate-lysine dipeptide, a lysine-proline dipeptide, and a proline-lysine dipeptide;
X₇ is selected from the group consisting of threonine, alanine, and a threonine-arginine-arginine tripeptide; and
X₈ is selected from the group consisting of phenylalanine and desamino-phenylalanine.

In particular embodiments, the A-chain may have the amino acid sequence set forth in SEQ ID NO: 1 or SEQ ID NO: 6 and the B-chain may have the amino acid sequence set forth in SEQ ID NO: 2, SEQ ID NO: 3, SEQ ID NO: 4, or SEQ ID NO: 5. In particular embodiments, the insulin analog is a des B30 insulin analog, a des B29-B30 insulin analog, a des B28-B30 insulin analog, a des B27-B30 insulin analog, or a des B26-B30 insulin analog.

In particular embodiments, the insulin or insulin analog is conjugated to one, two, or three pentavalent-valent sugar clusters selected from the group consisting of ML-1, ML-2, ML-3, ML-4, ML-5, ML-6, ML-7, ML-8, ML-9, ML-10, ML-11, ML-12, ML-13, ML-14, ML-15, ML-16, ML-17, ML-18, ML-19, ML-20, ML-21, ML-22, ML-23, ML-24, ML-25, ML-26, ML-27, ML-28, ML-29, ML-30, ML-31, ML-32, ML-33, ML-34, ML-35, ML-36, ML-37, ML-38, ML-39, ML-40, ML-41, ML-42, ML-43, ML-44, ML-45, ML-46, ML-47, ML-48, ML-49, ML-50, ML-51, and ML-52.

Exemplary human insulin oligosaccharide conjugates (IOCs) of the present disclosure include the IOCs having the following structures:

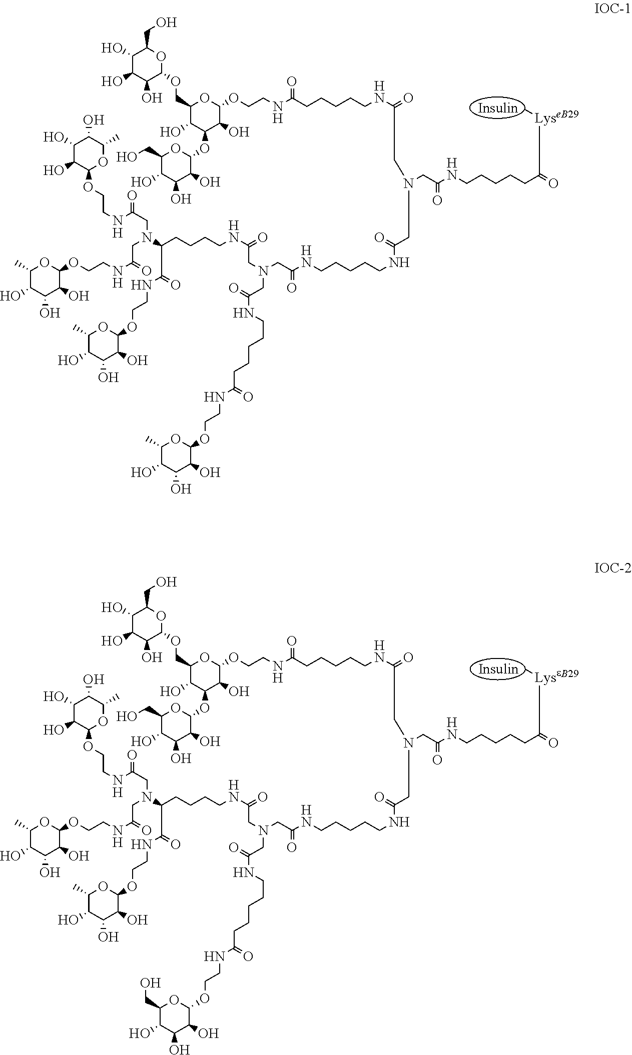

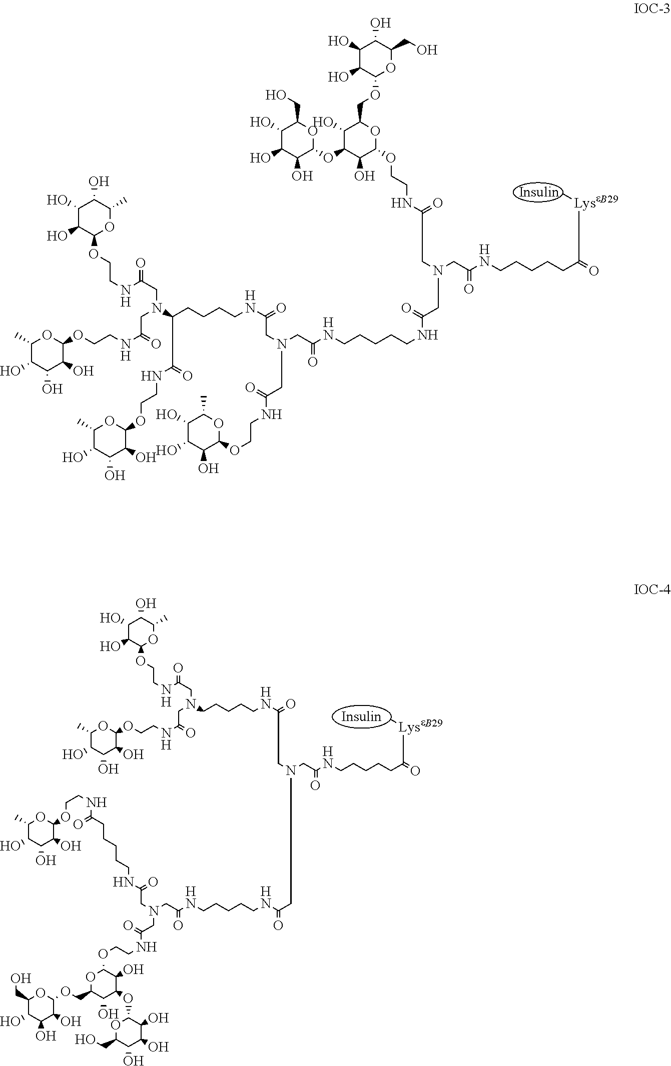

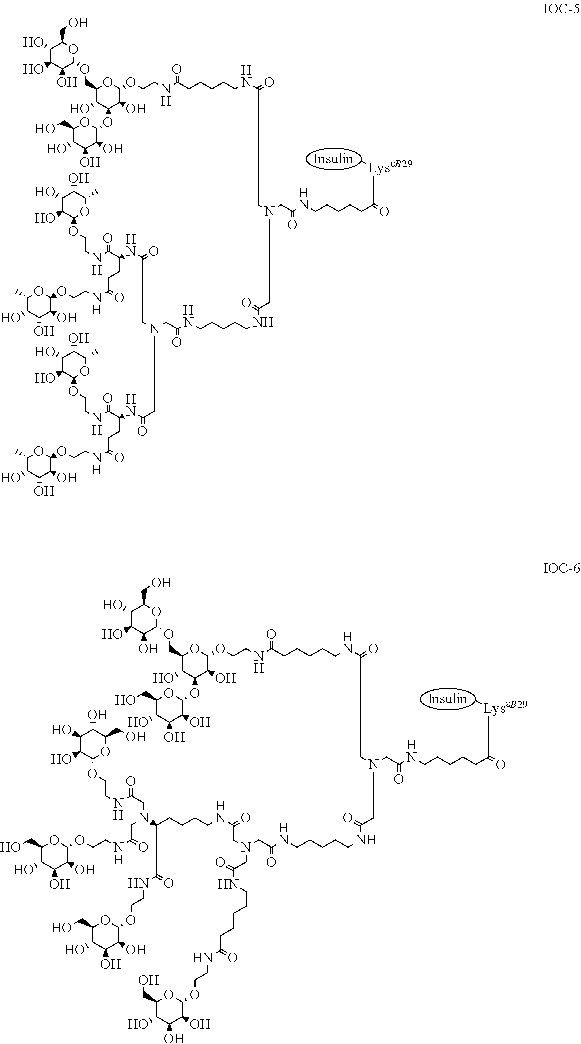

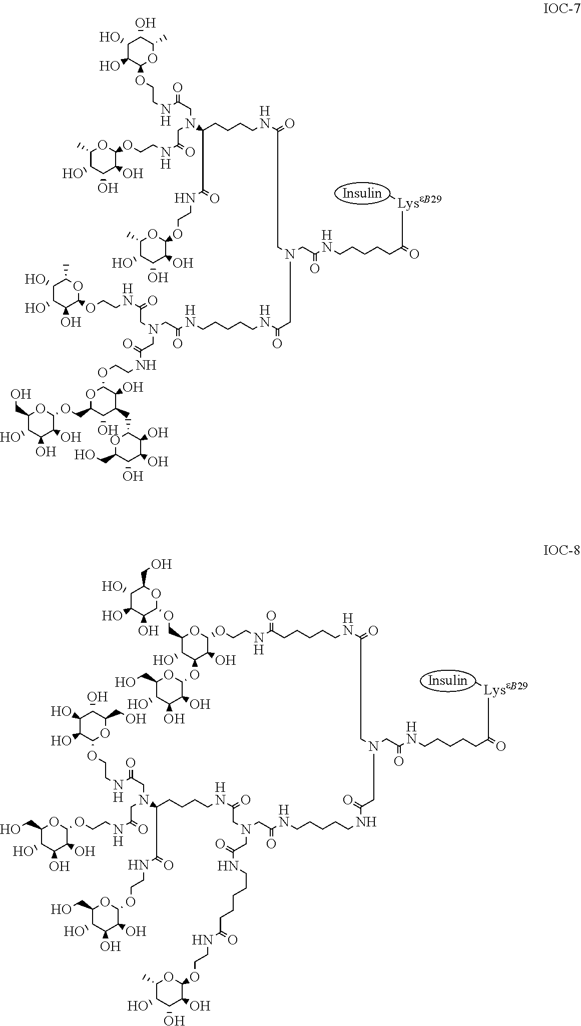

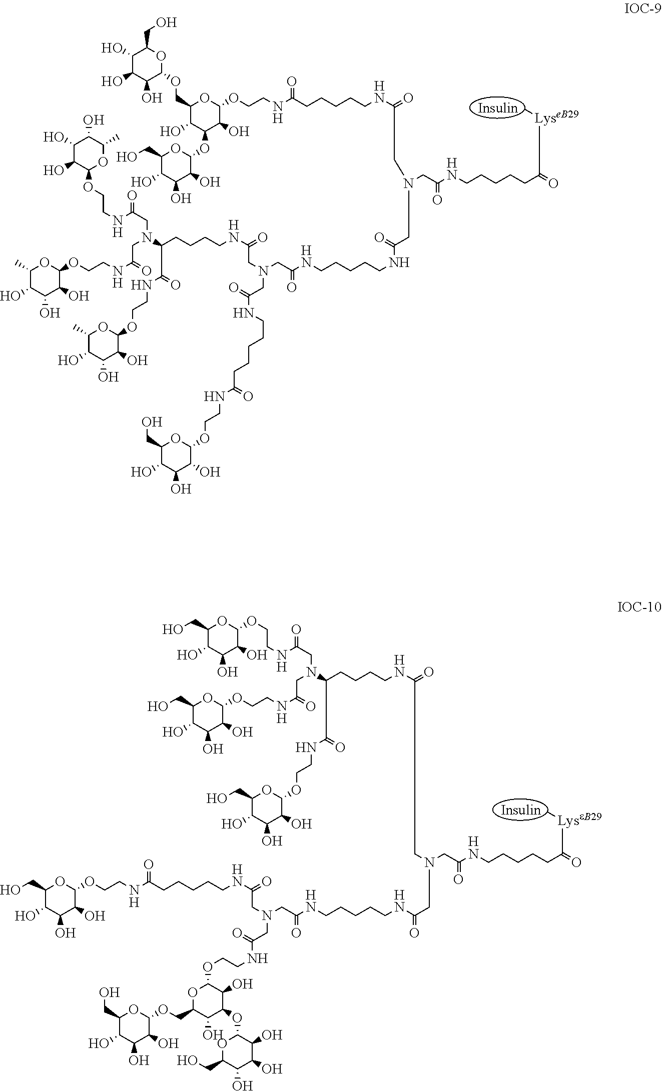

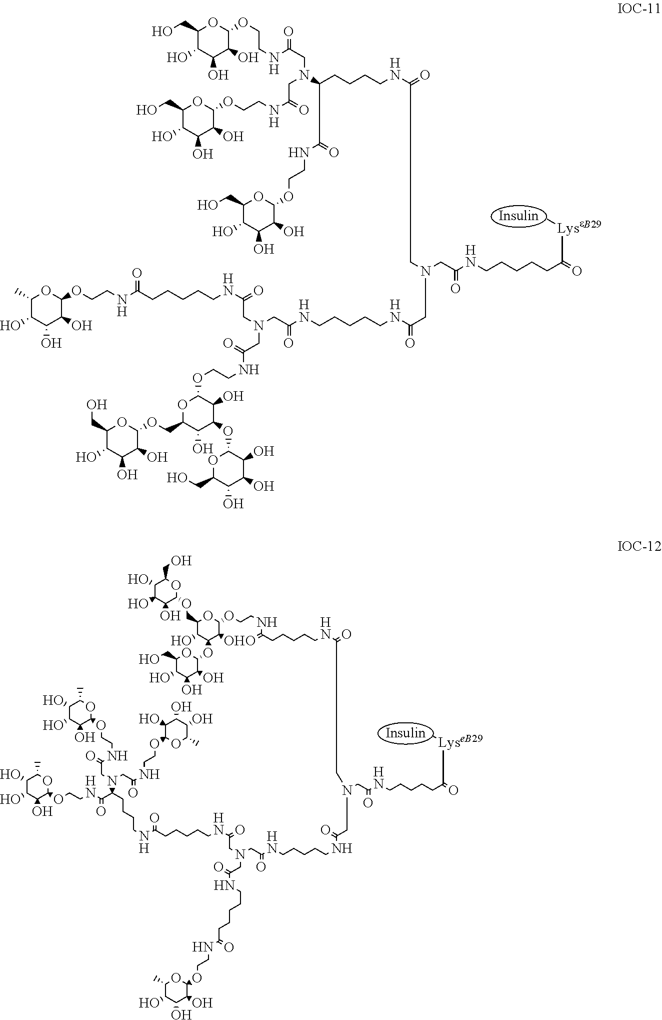

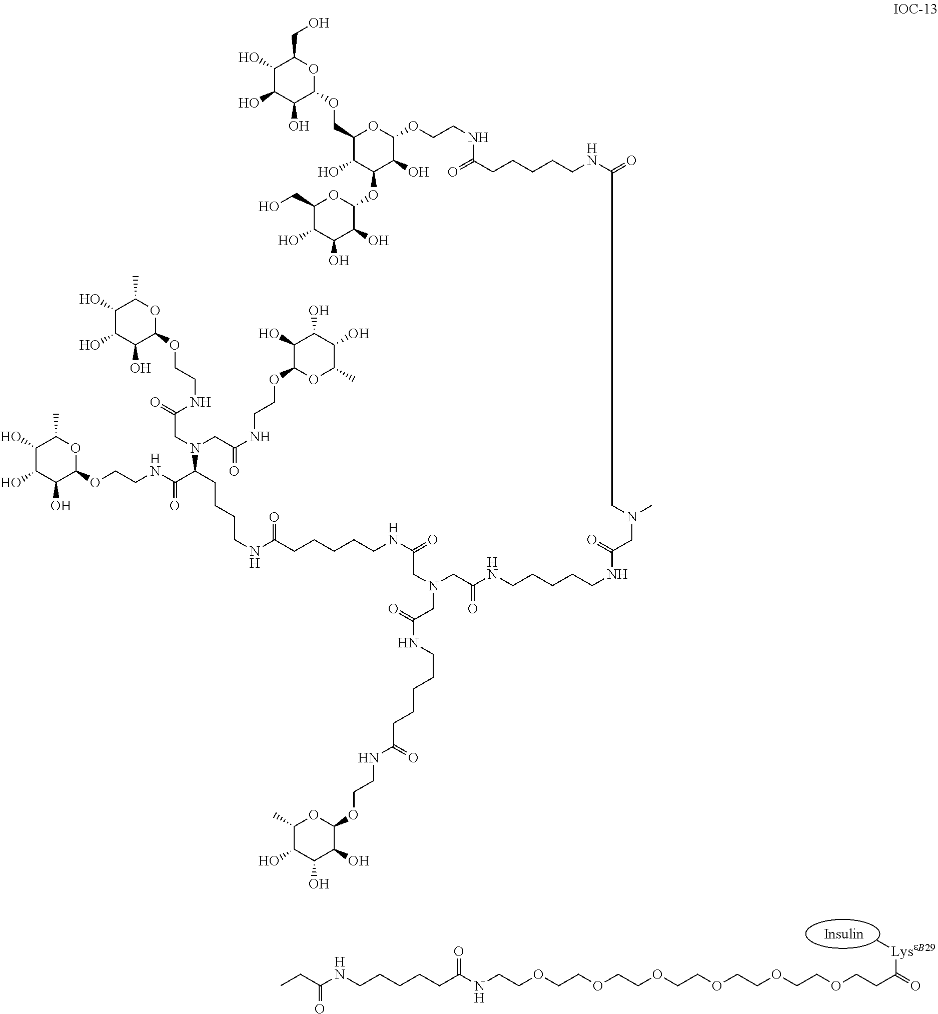

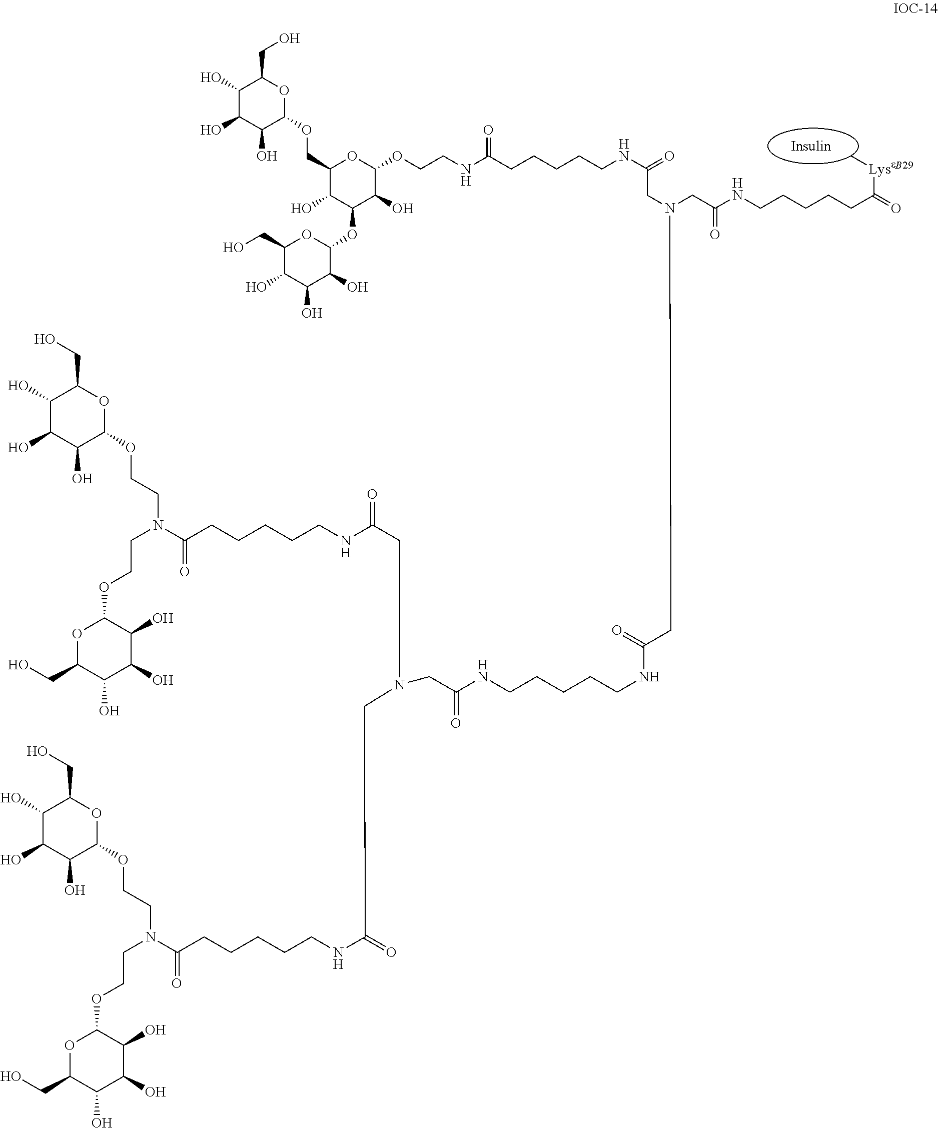

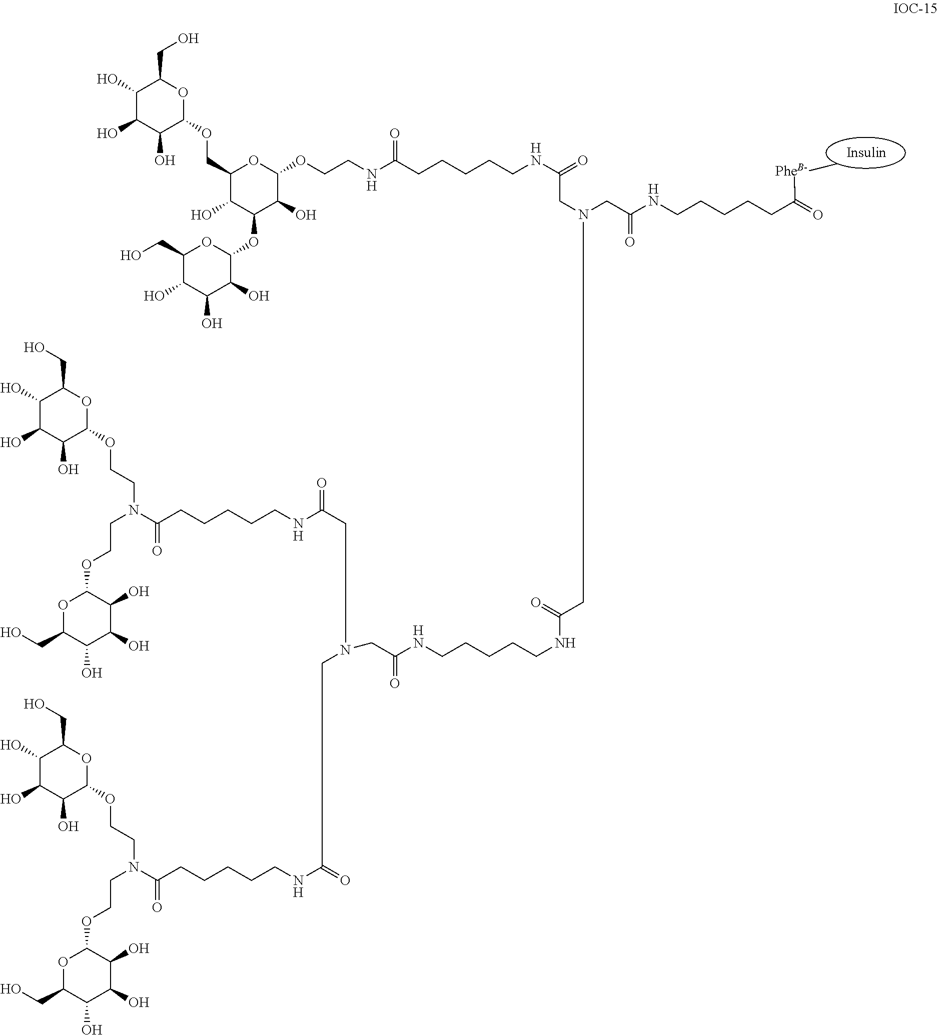

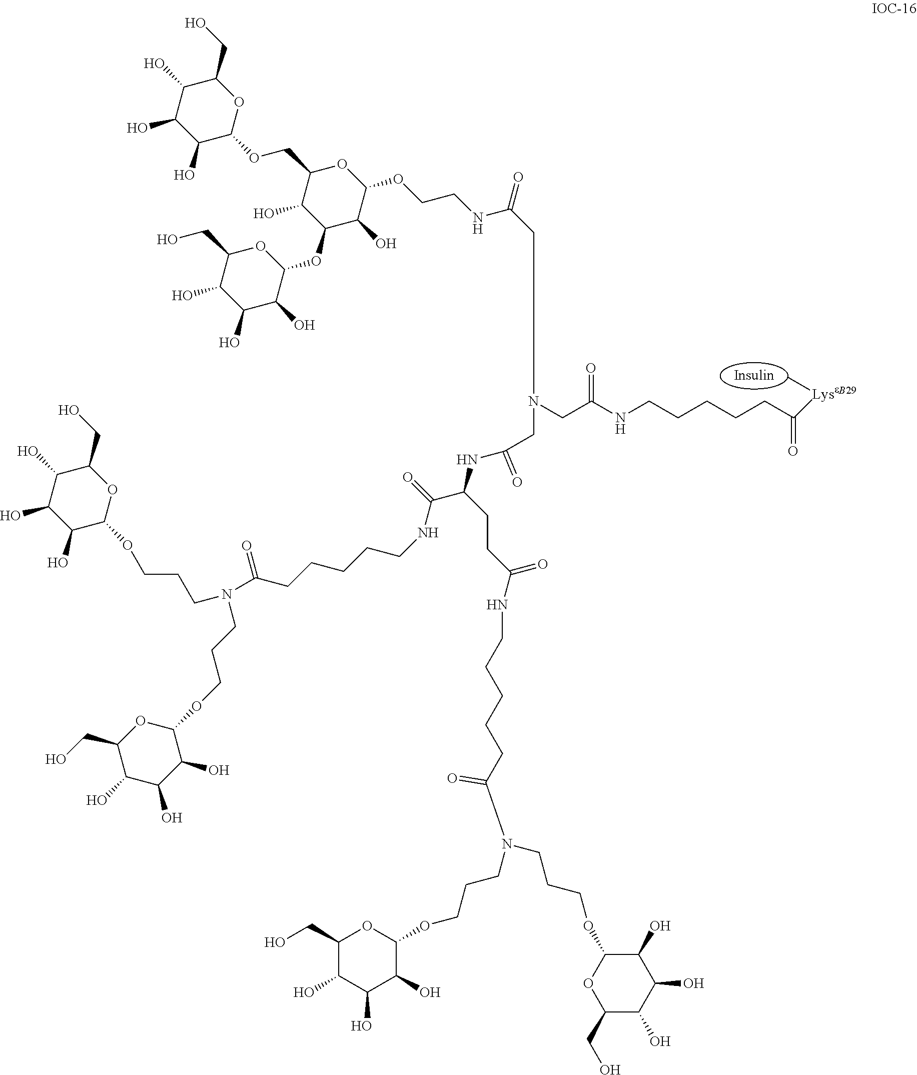

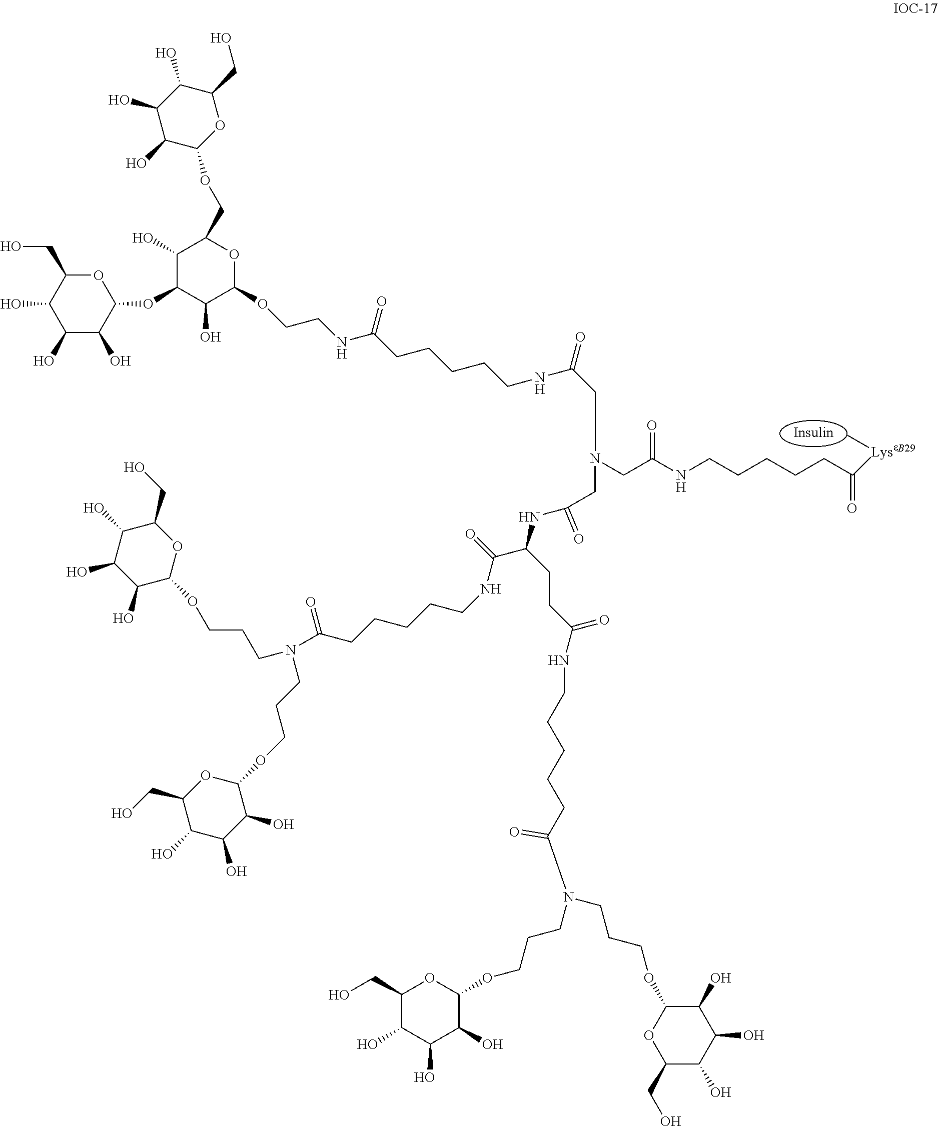

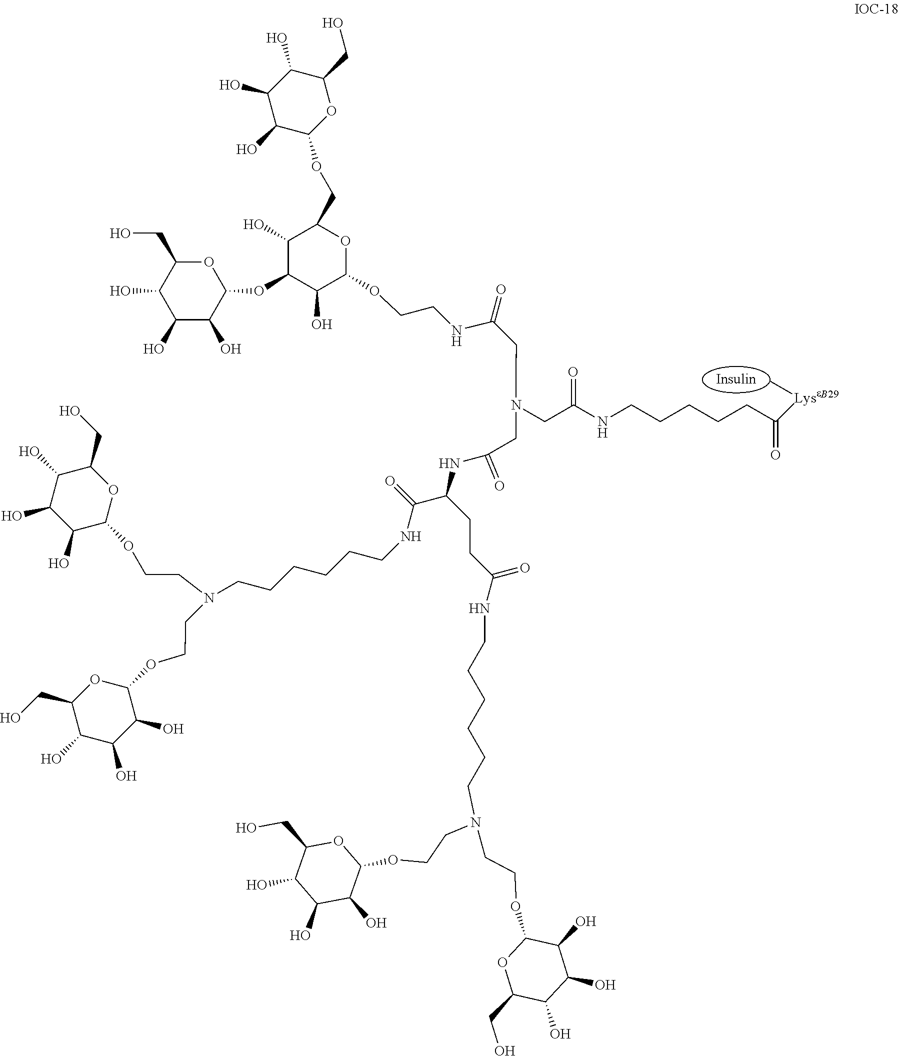

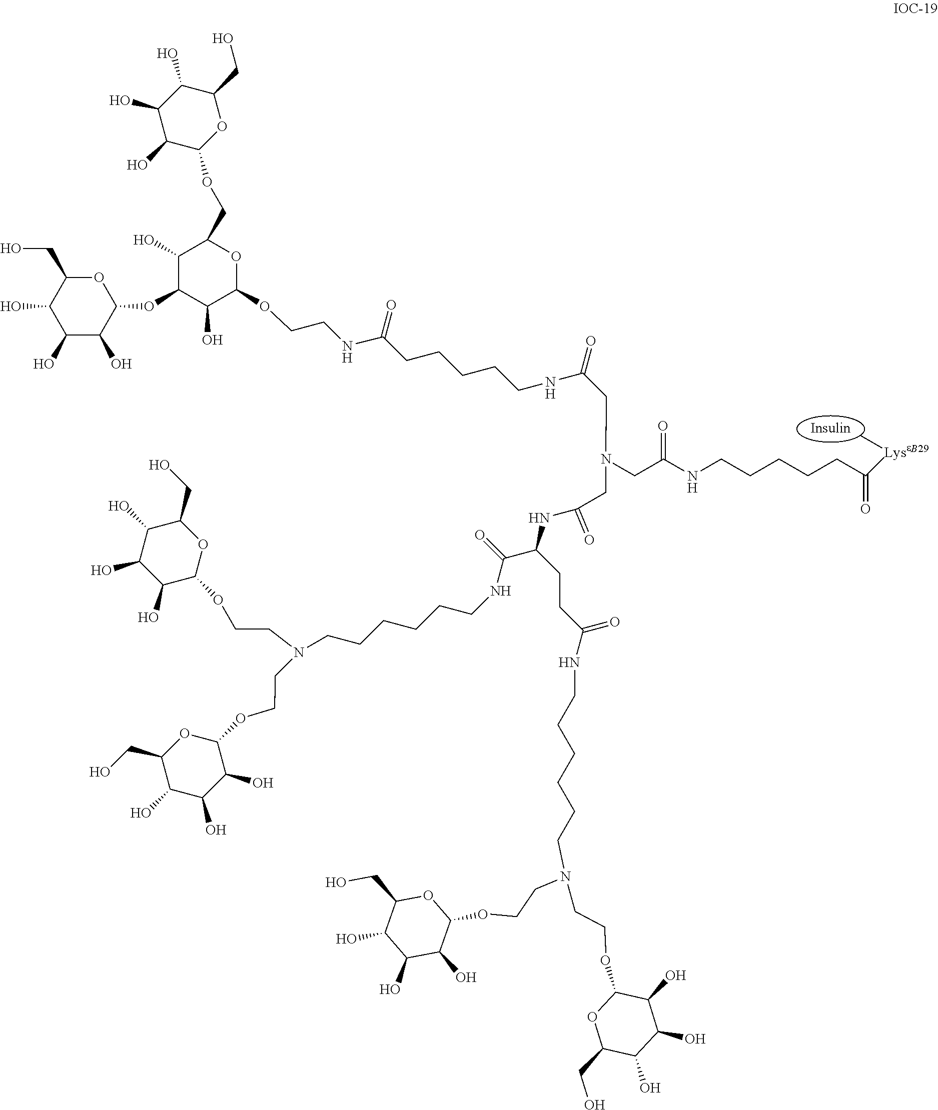

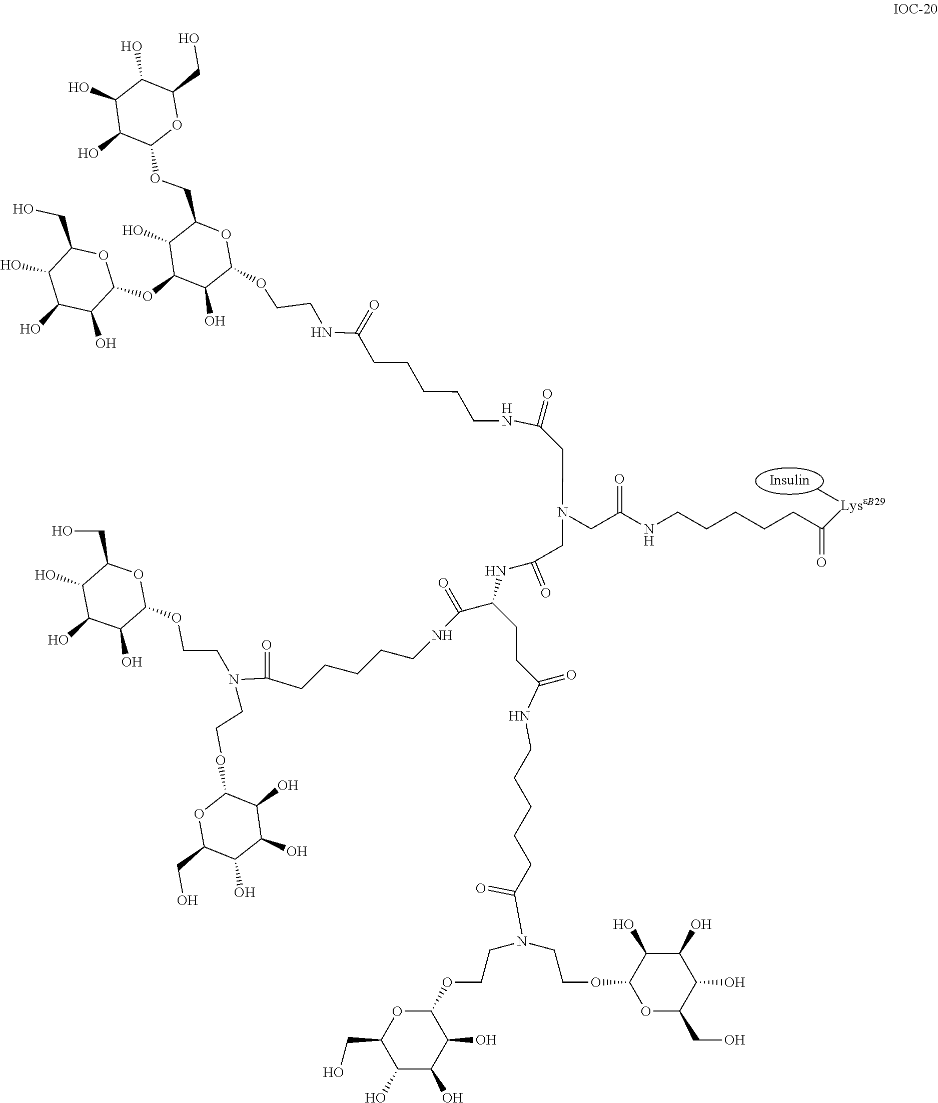

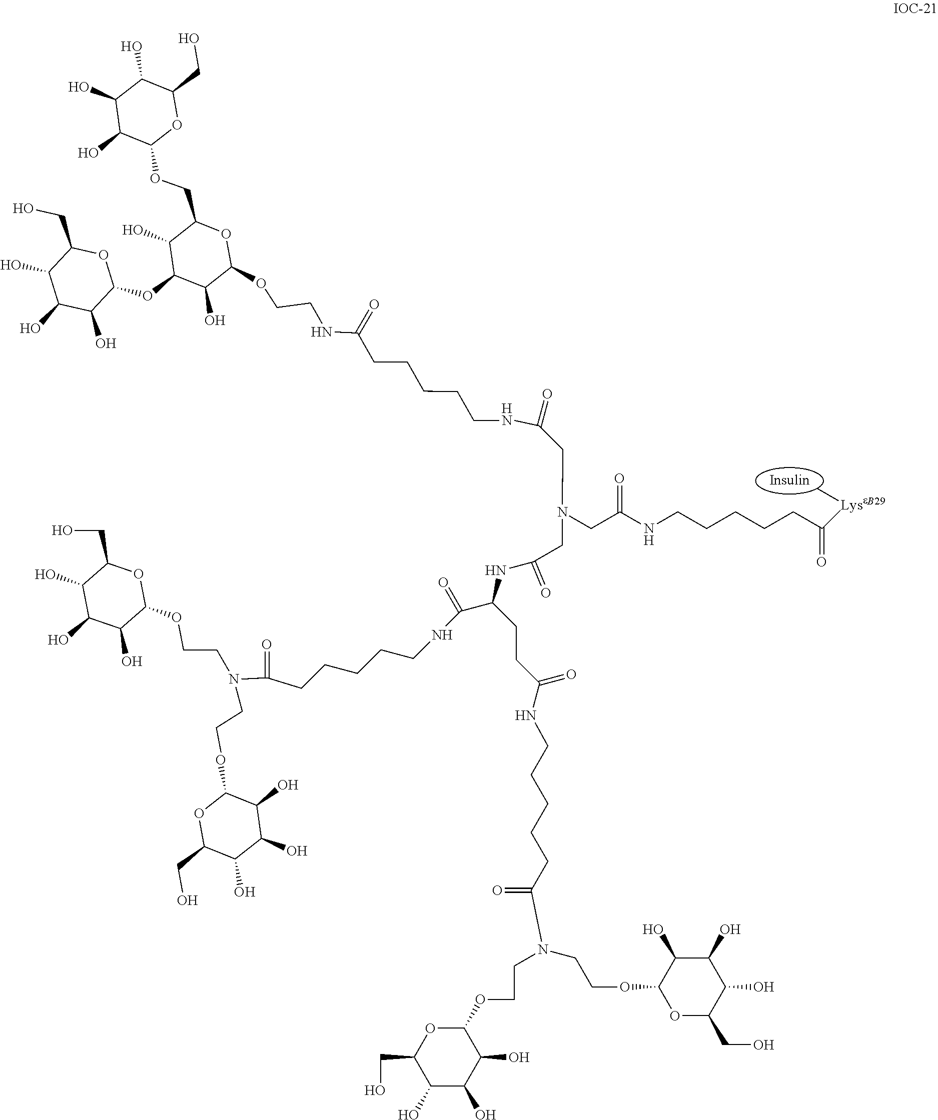

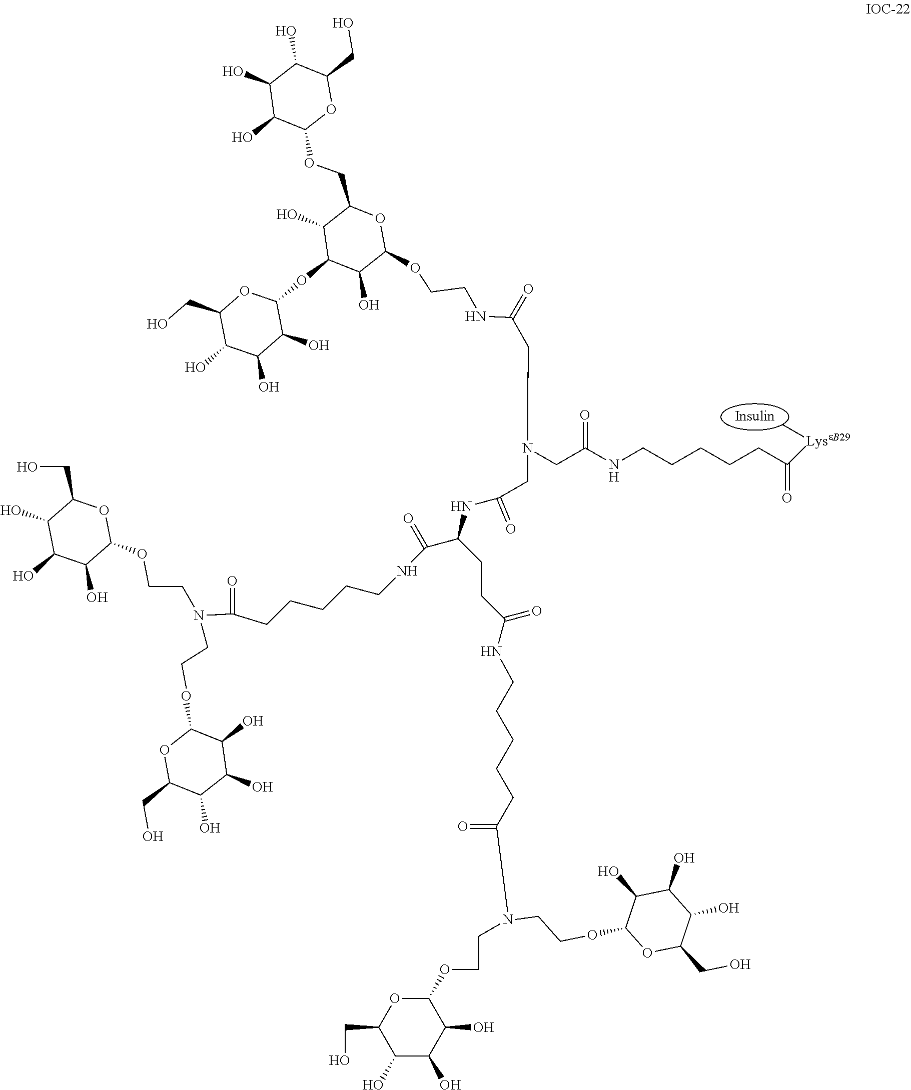

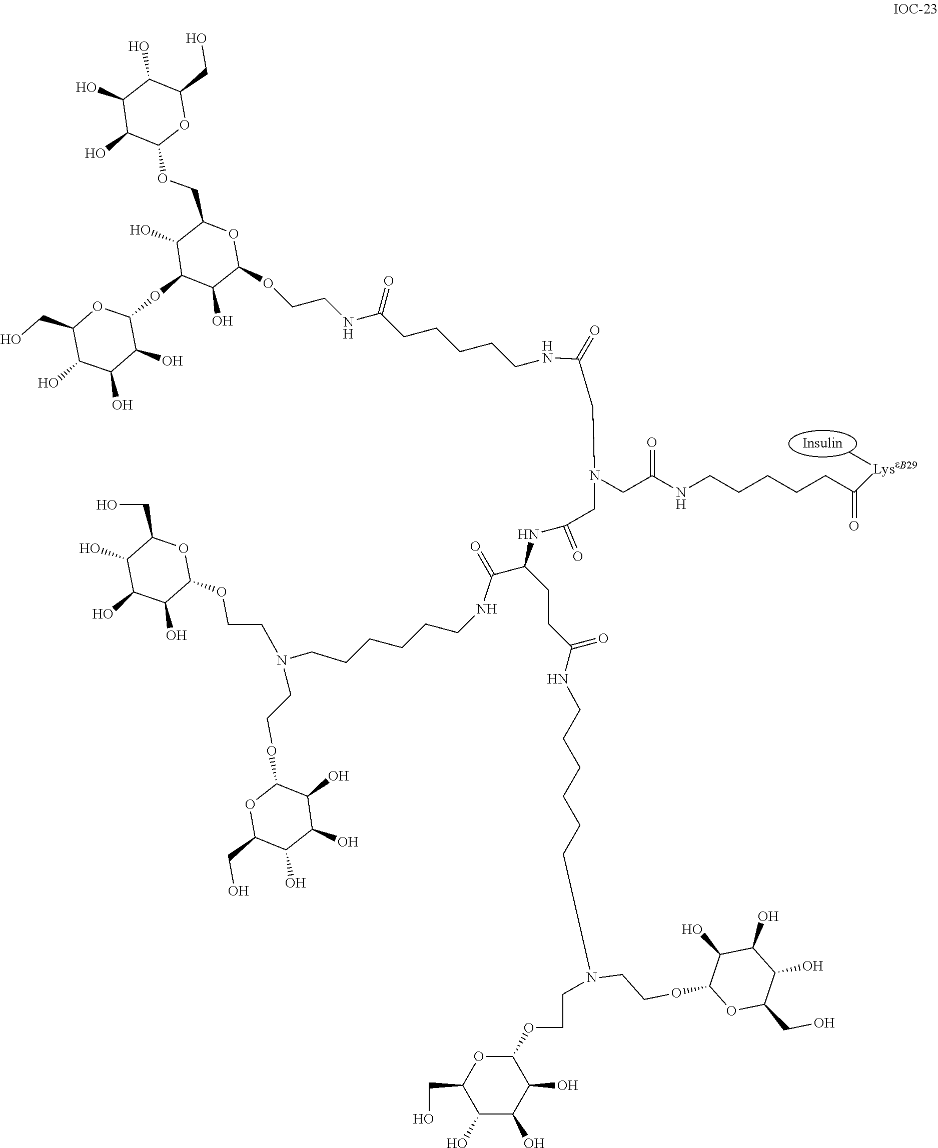

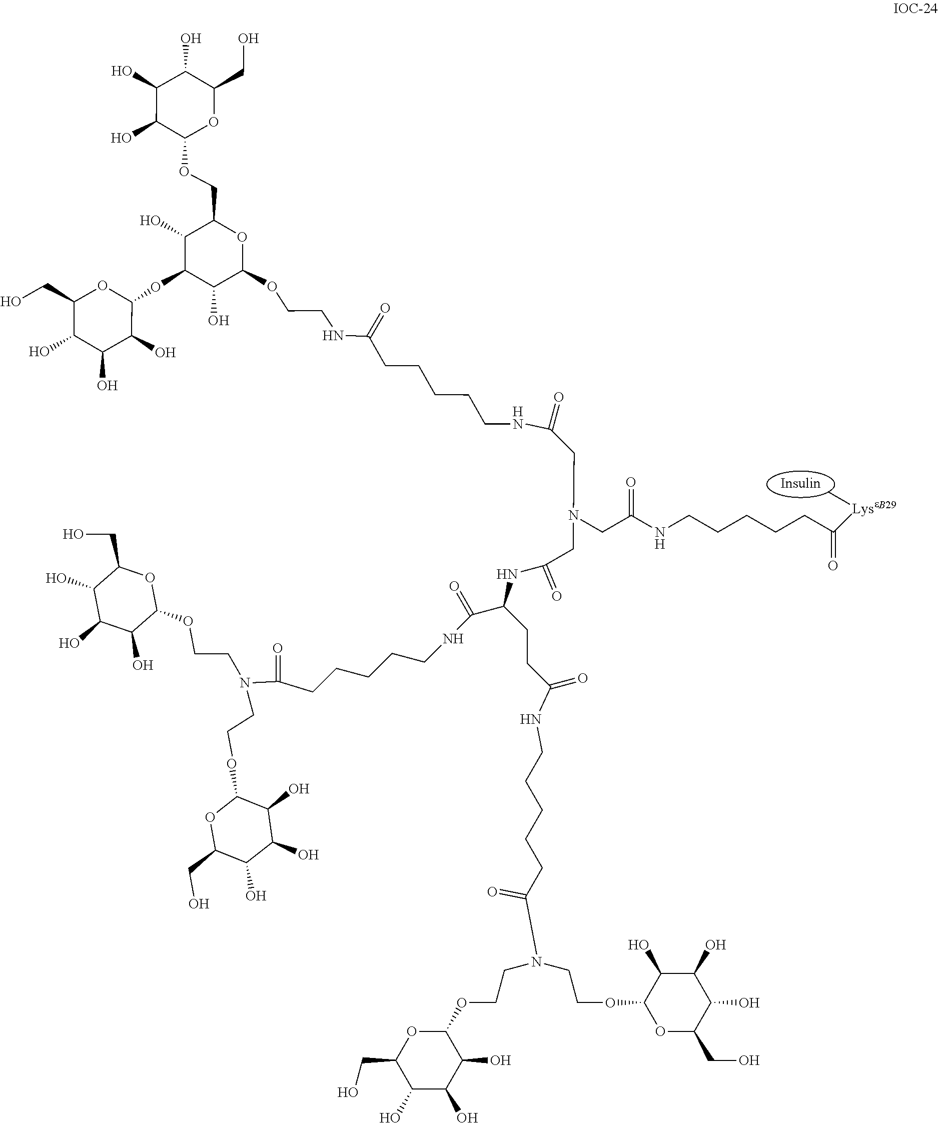

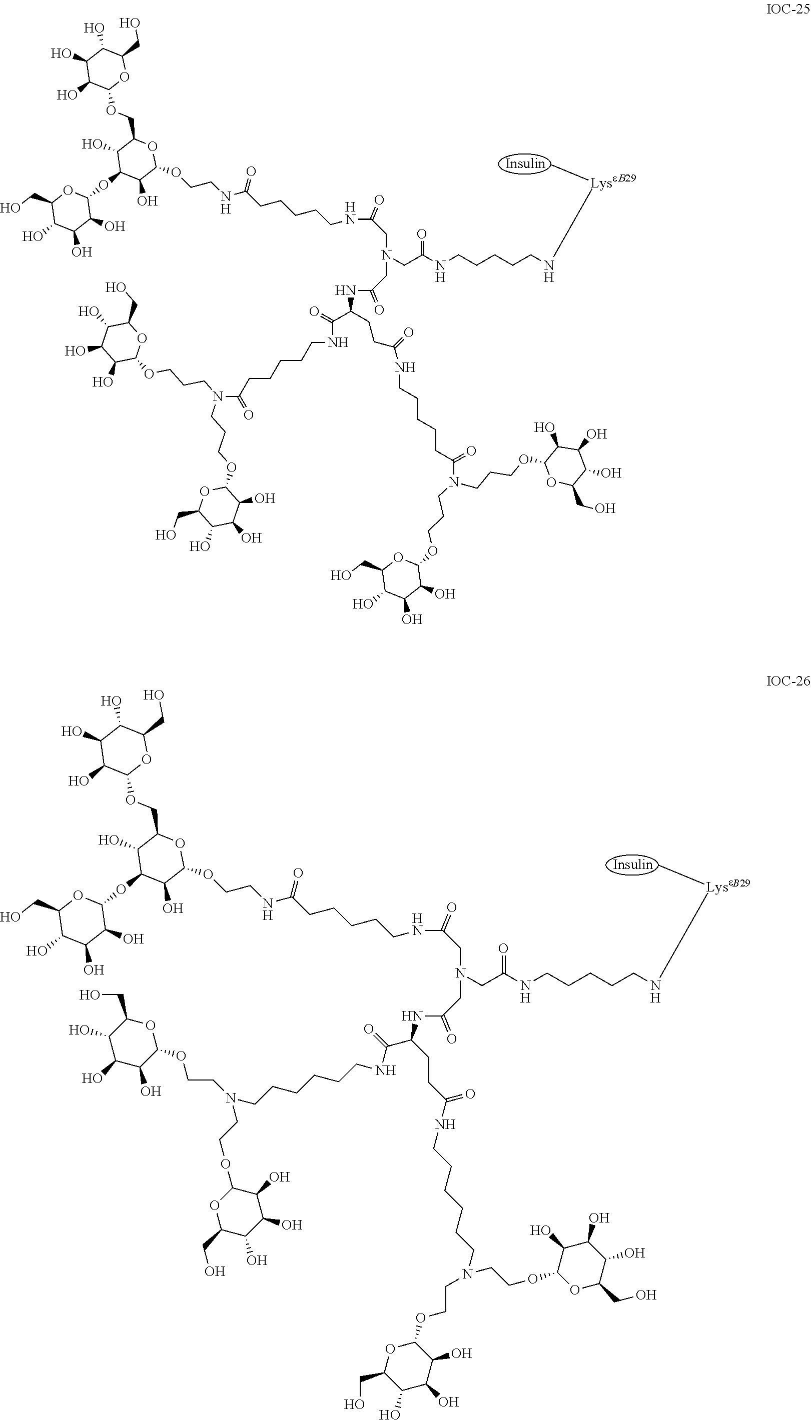

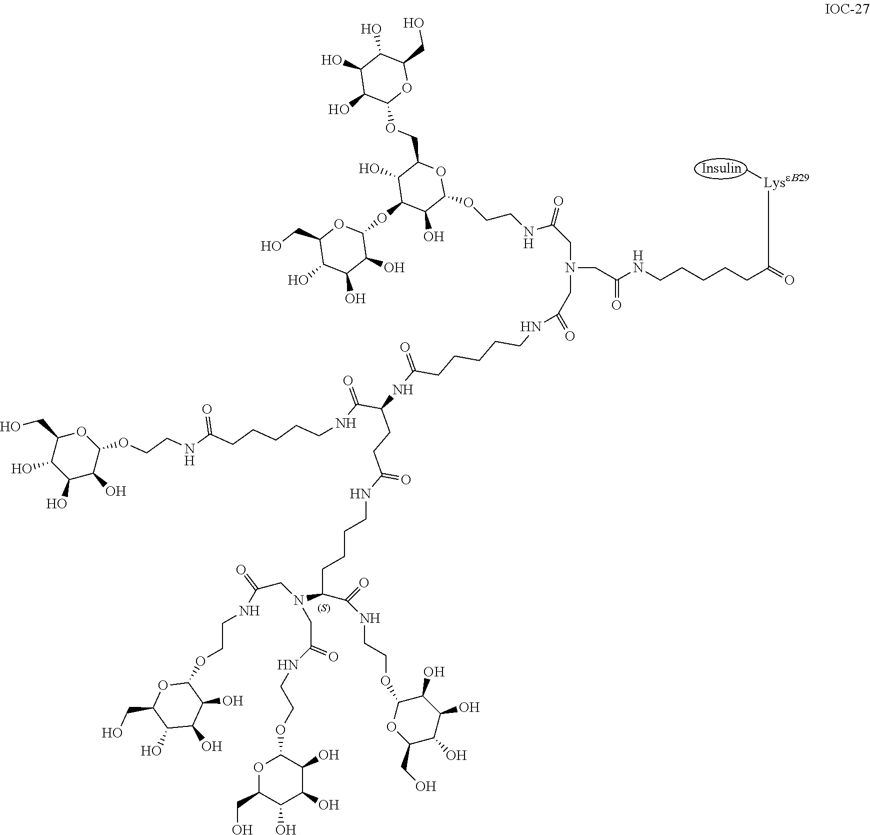

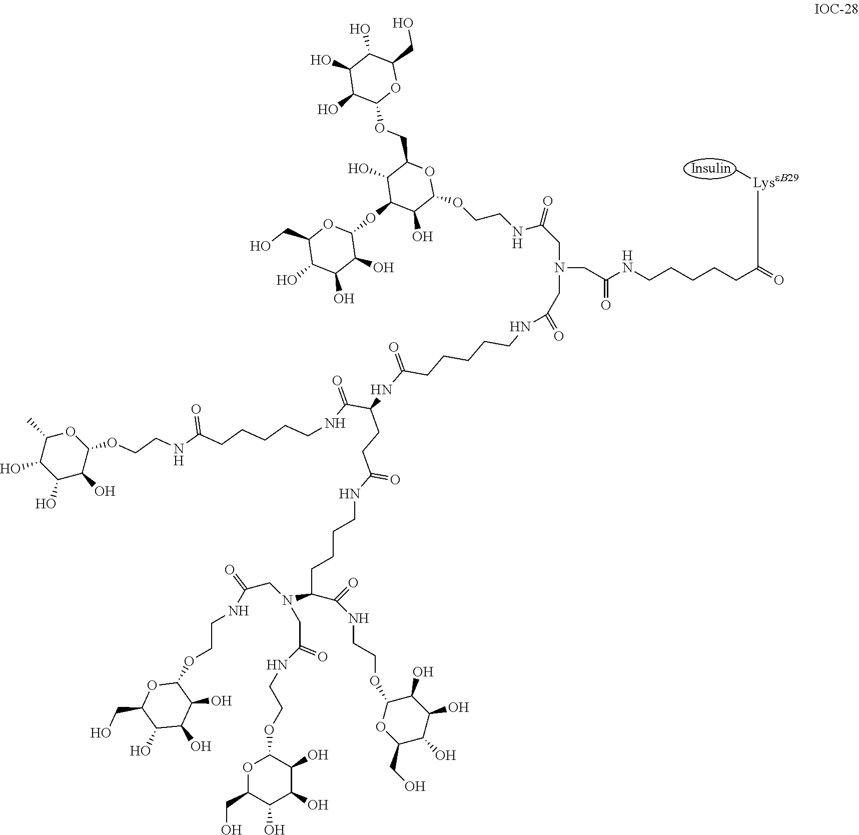

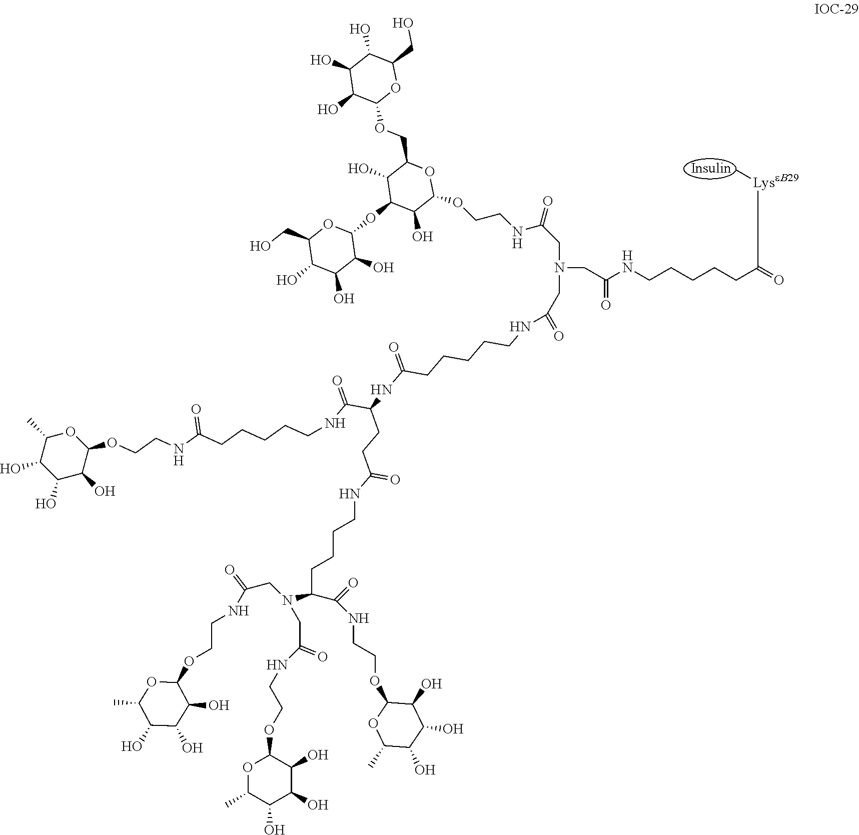

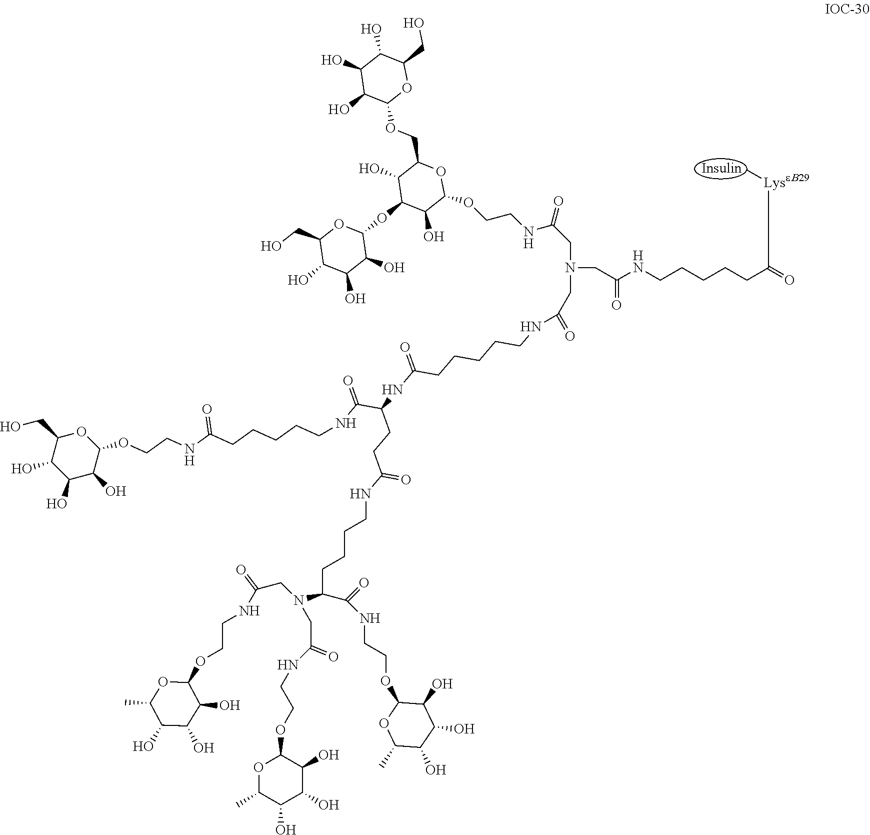

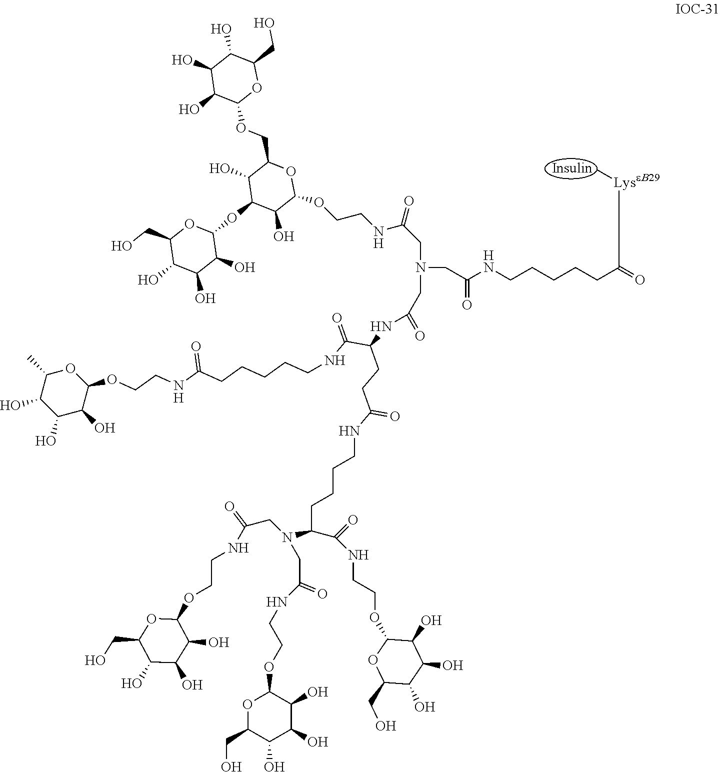

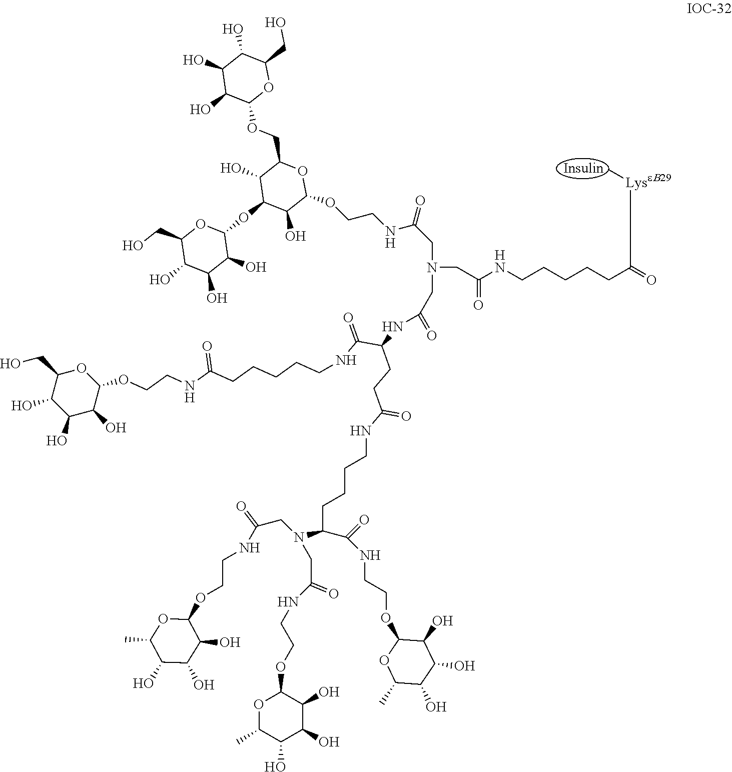

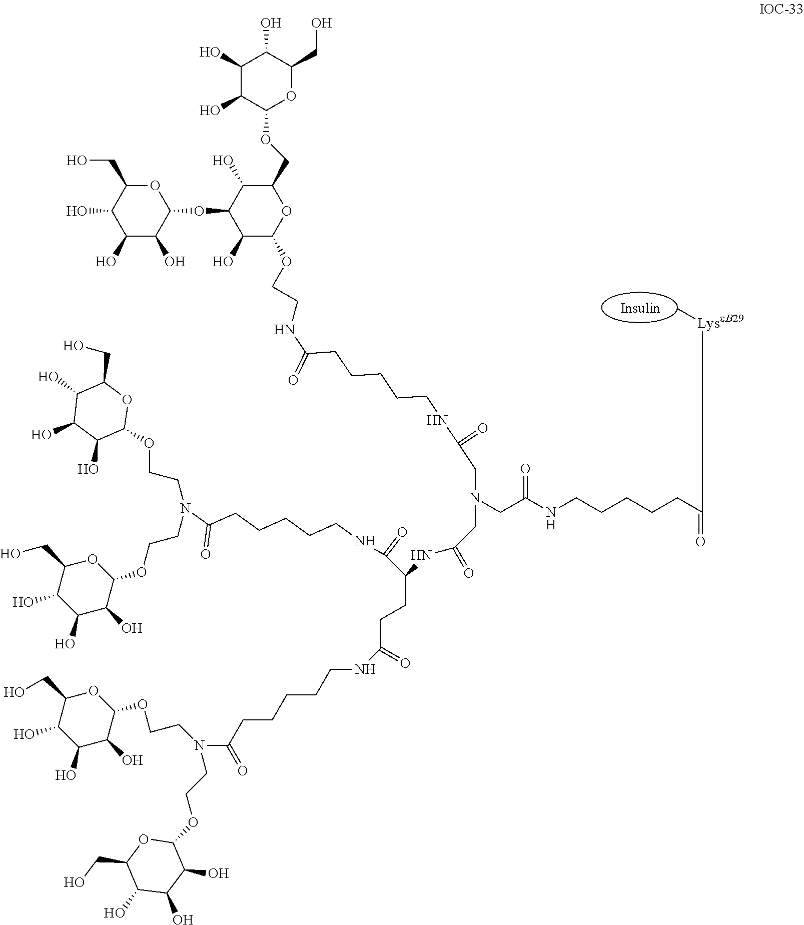

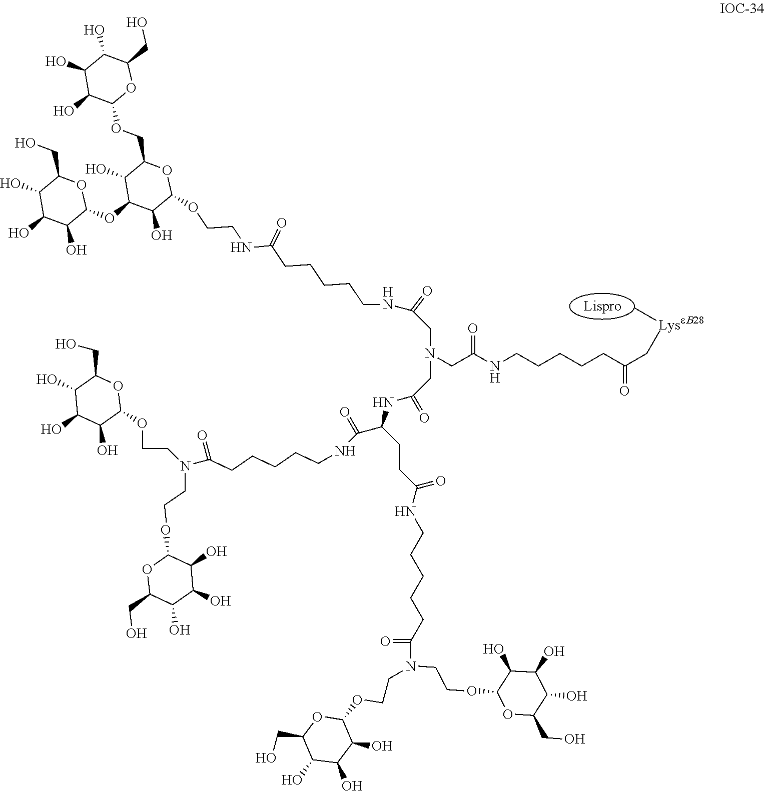

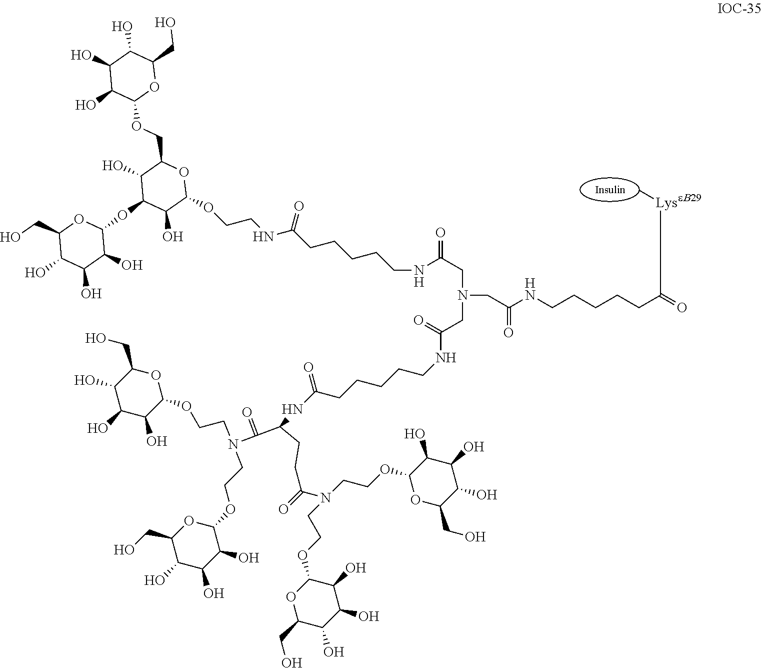

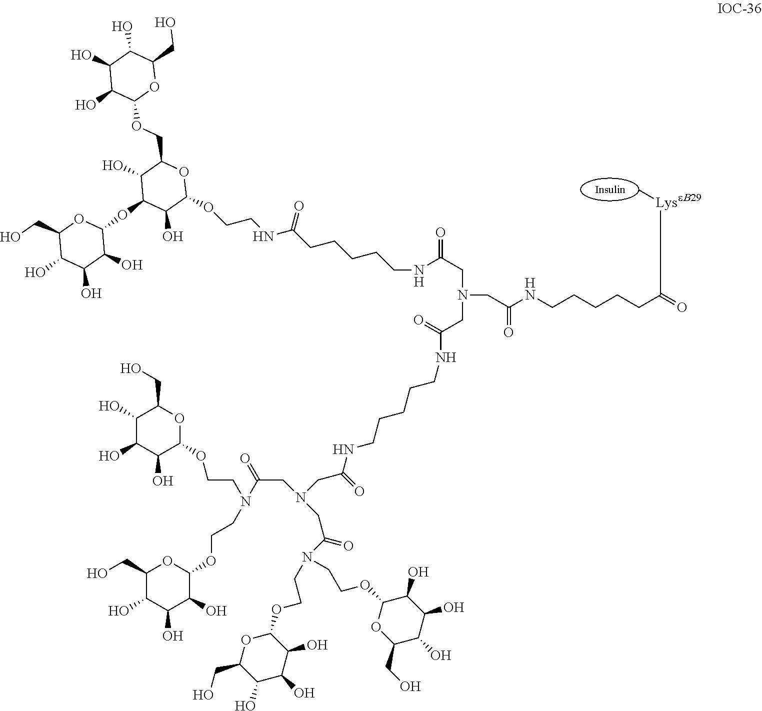

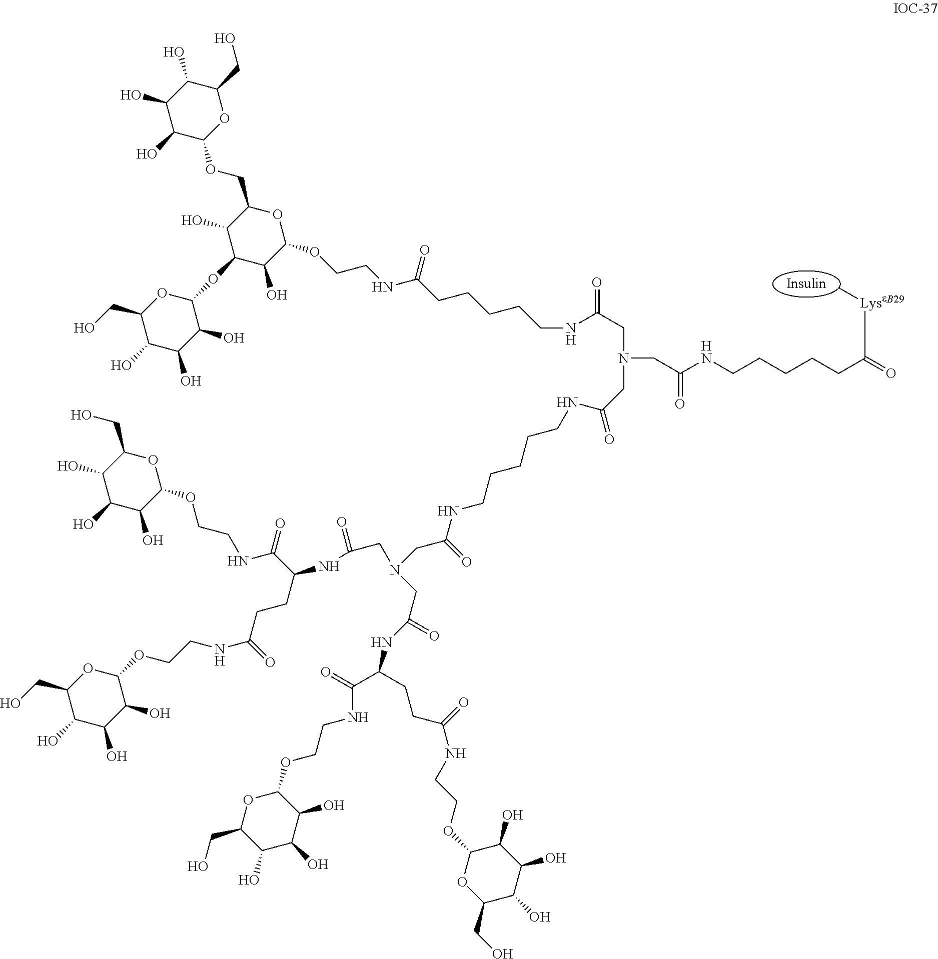

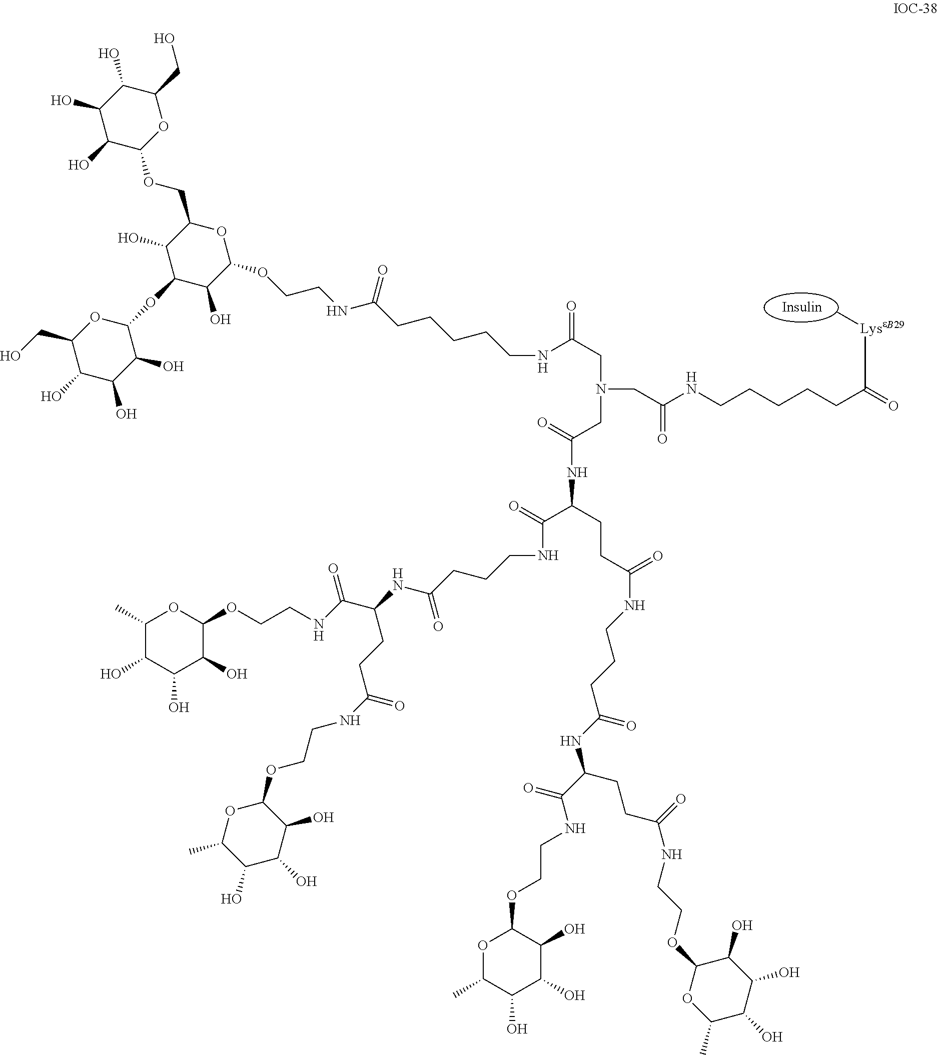

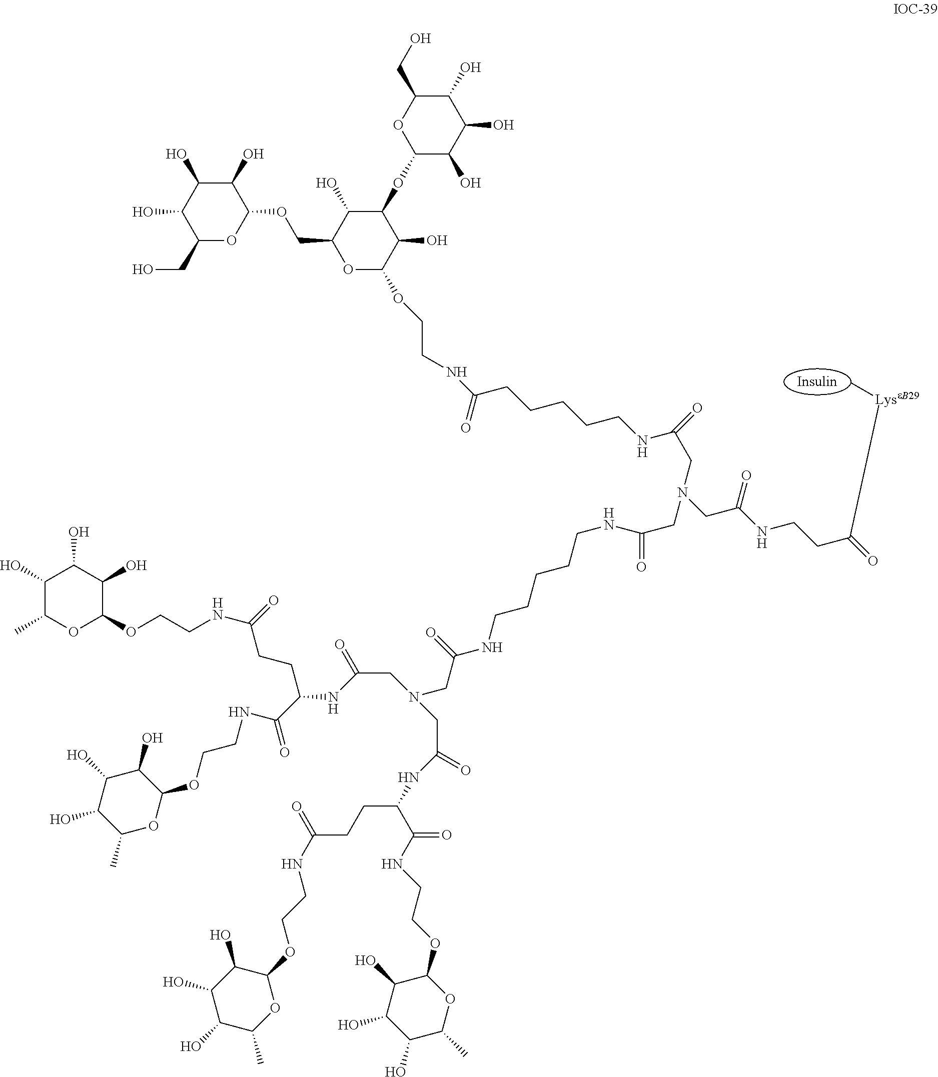

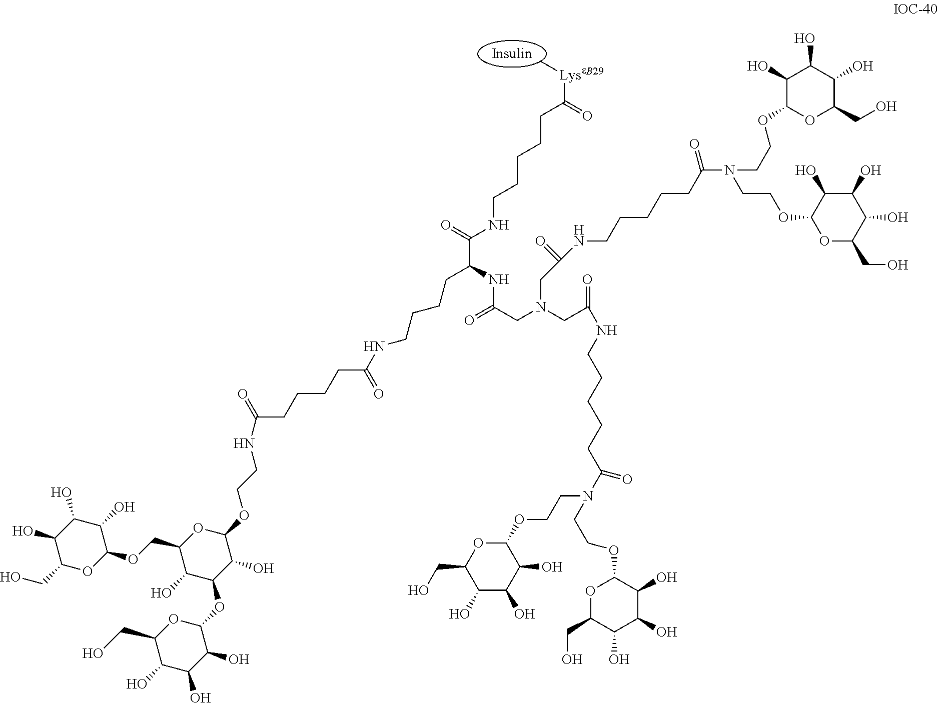

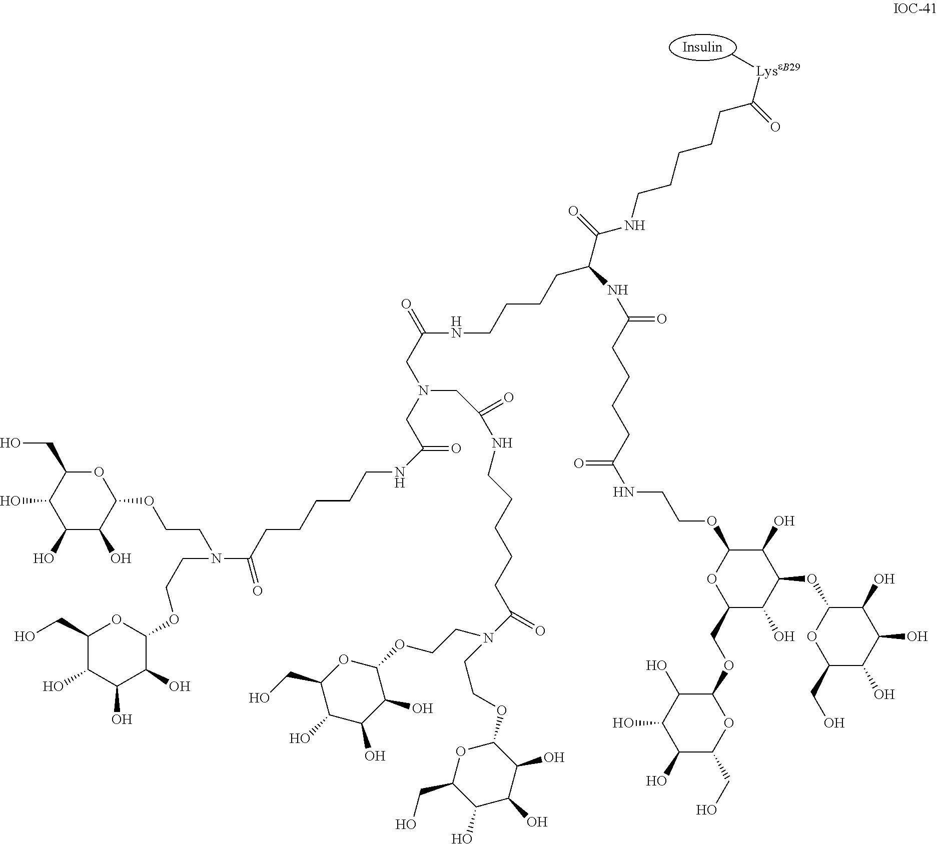

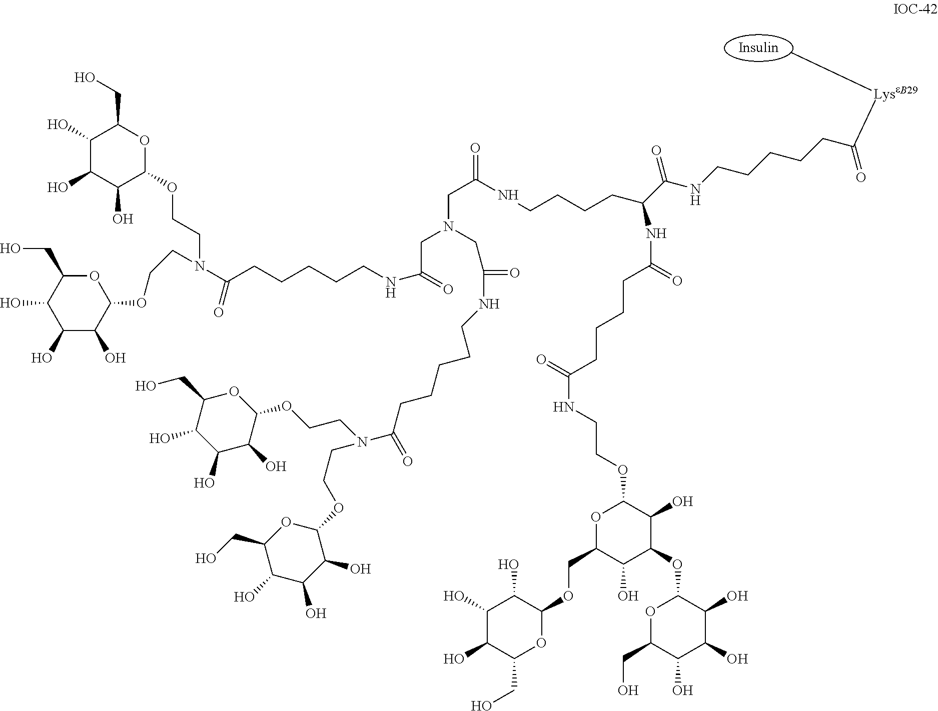

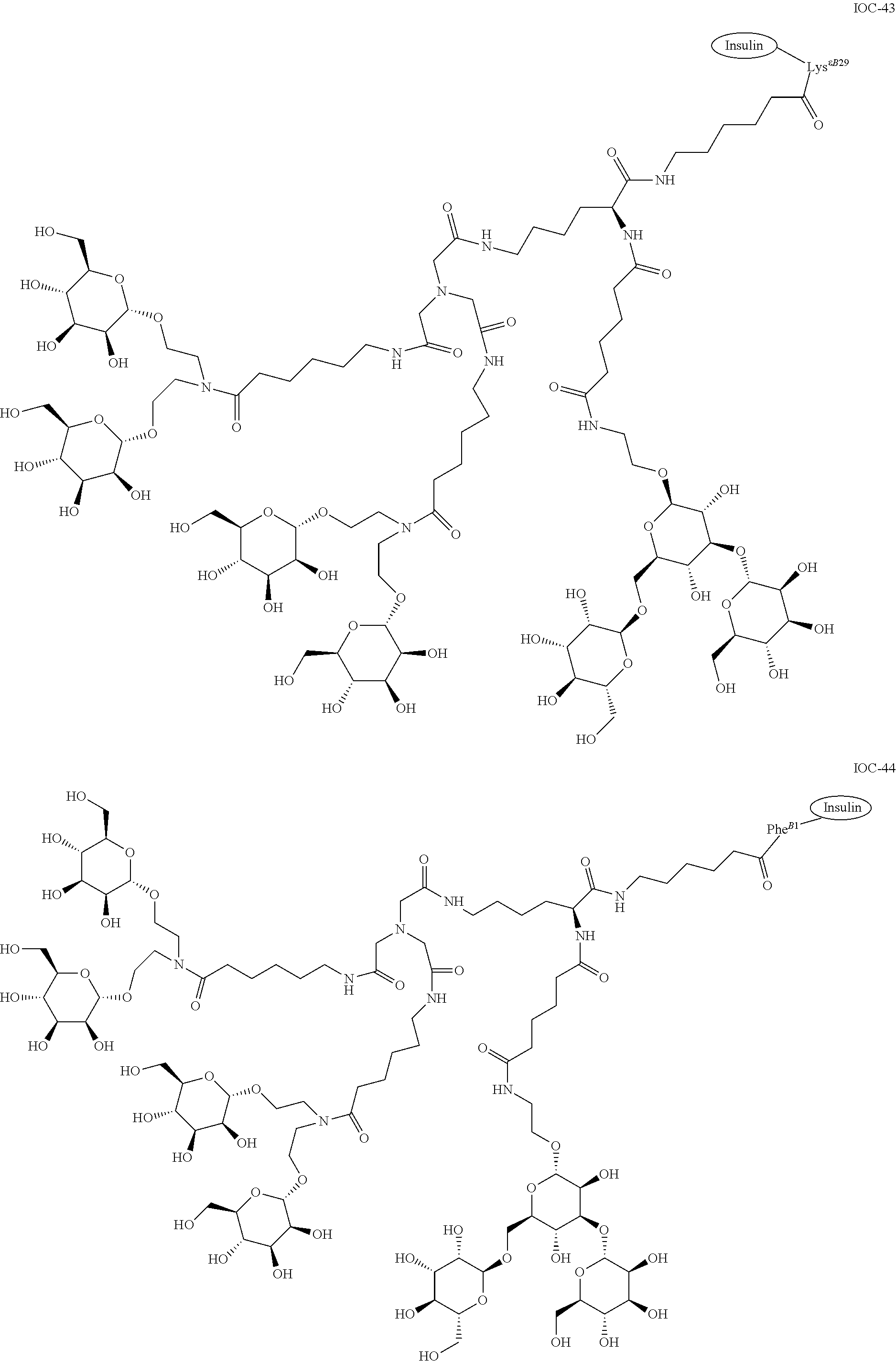

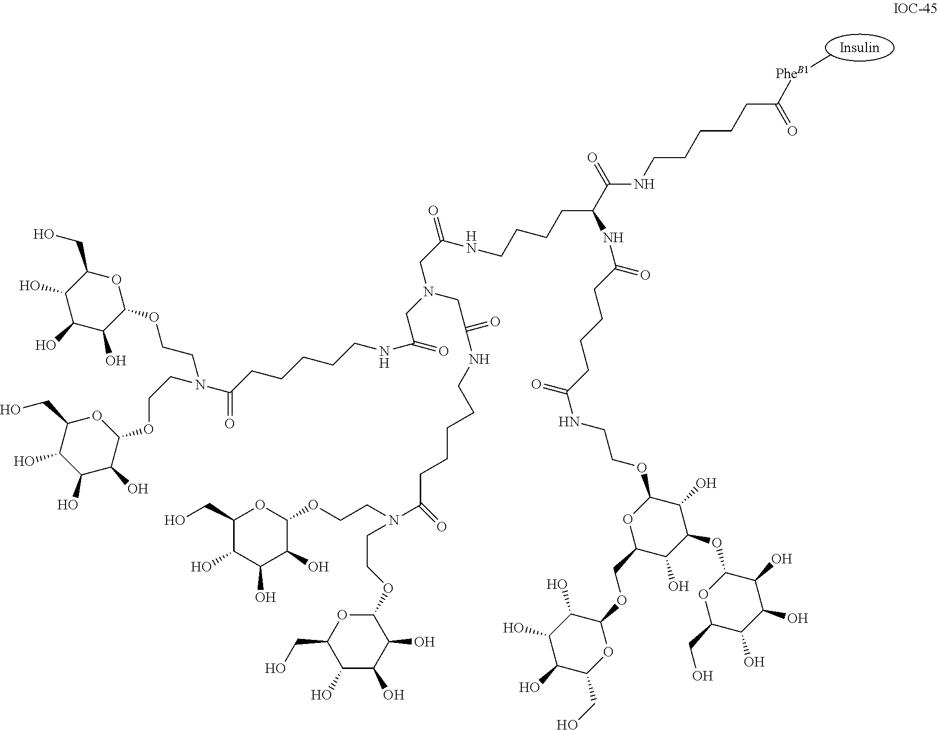

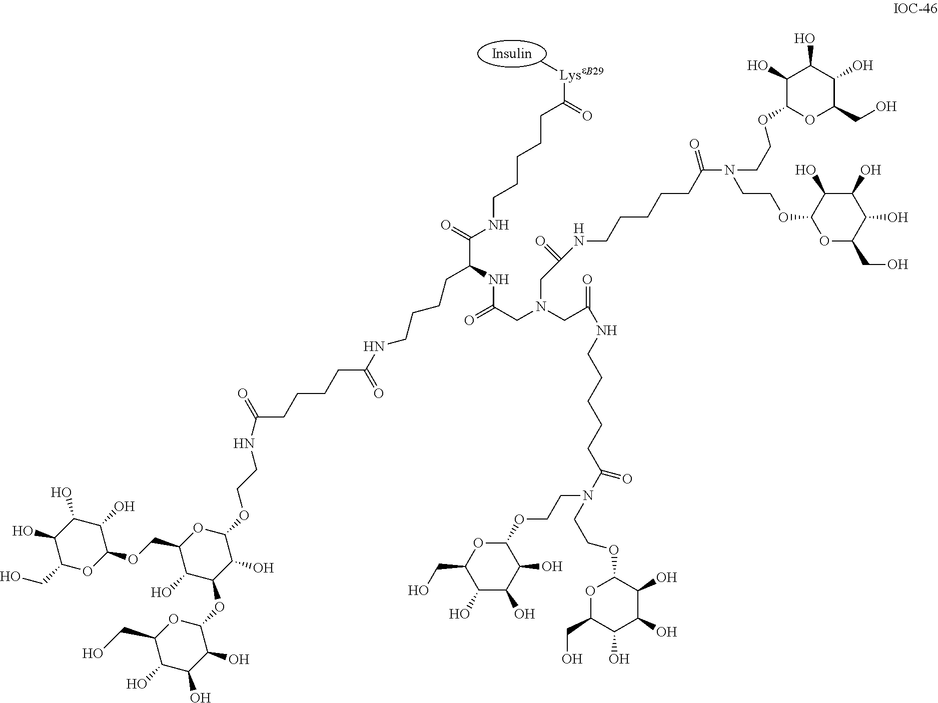

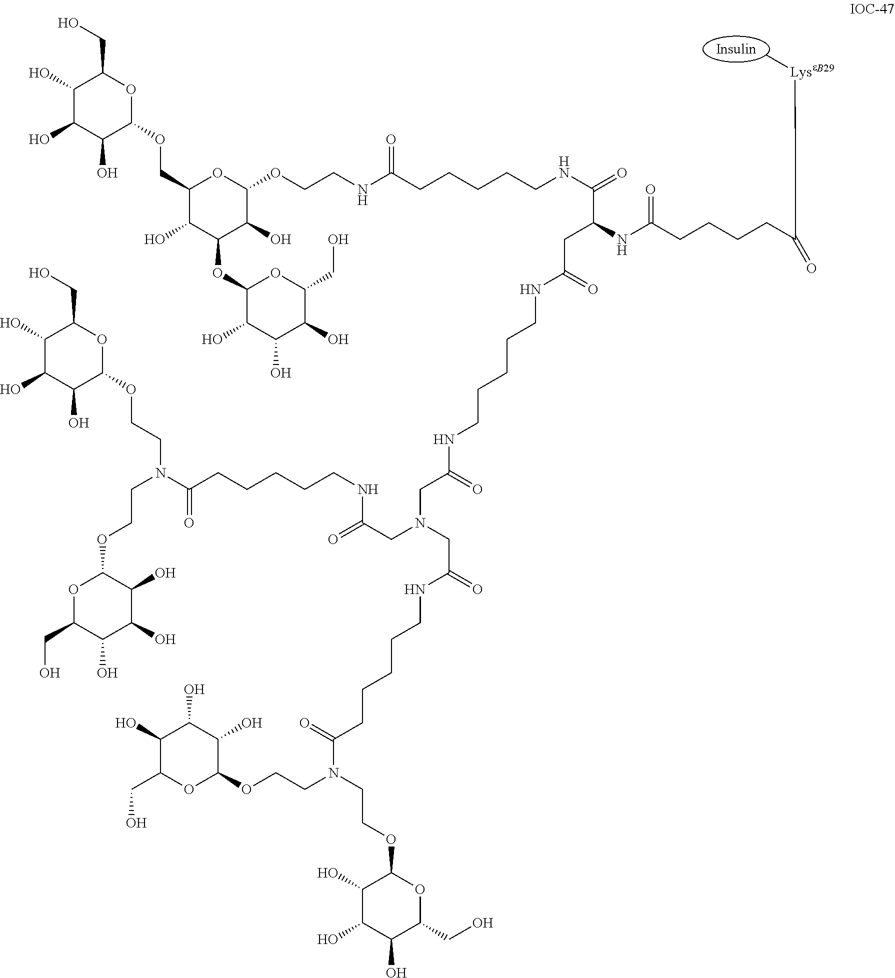

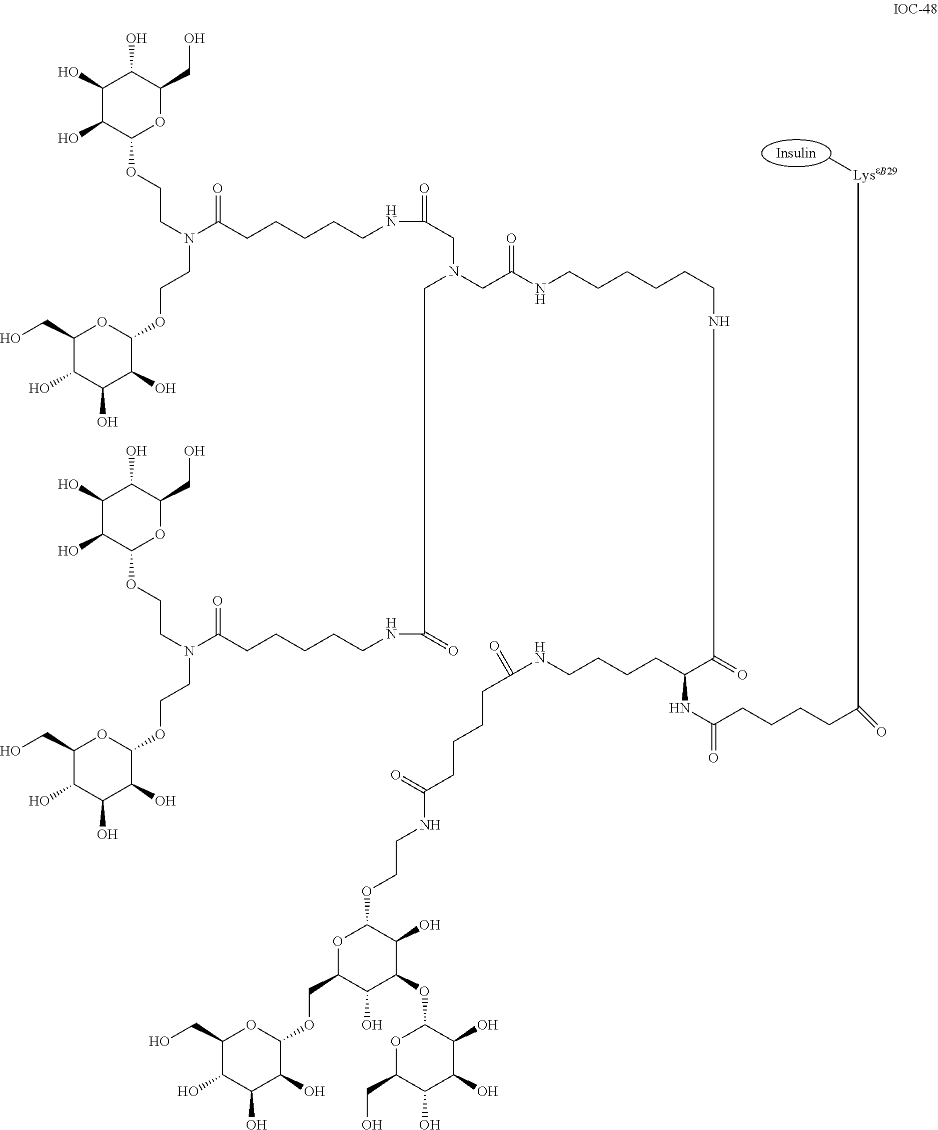

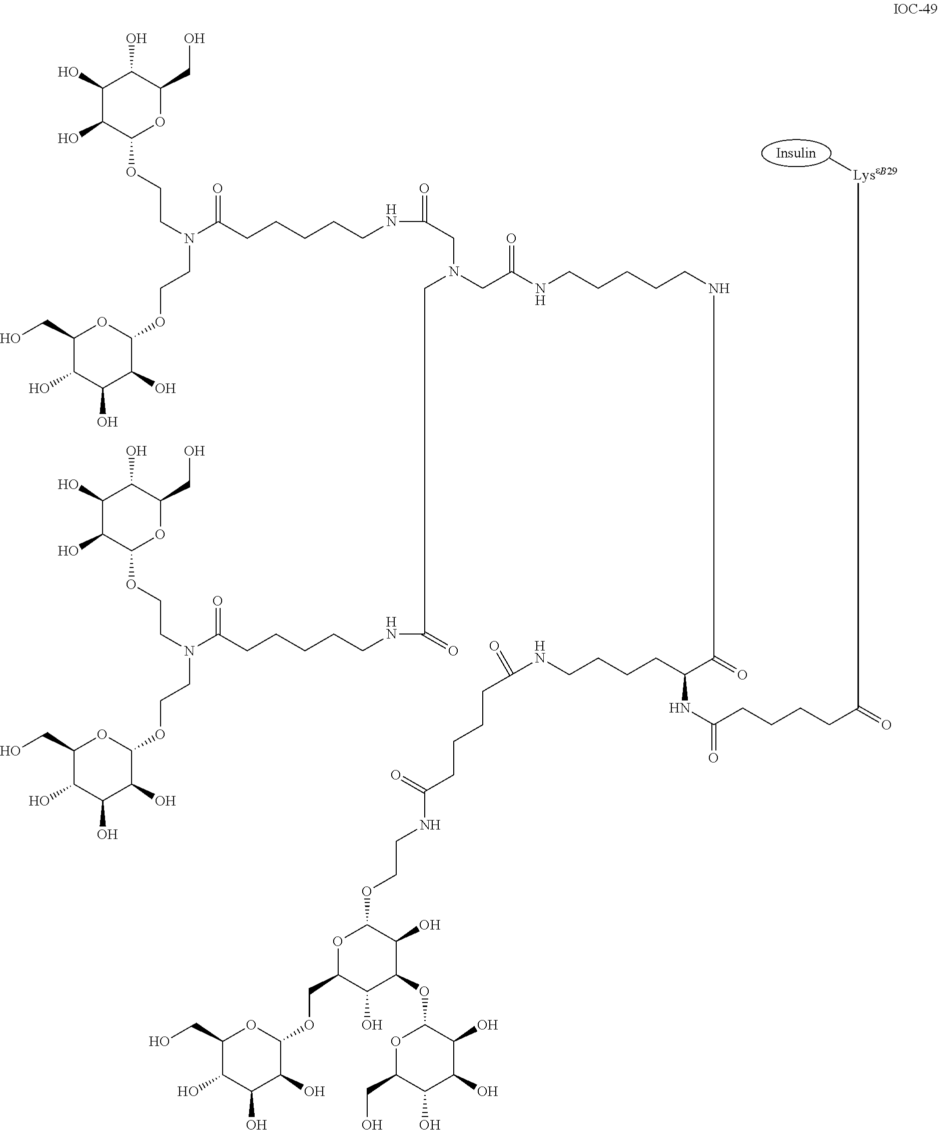

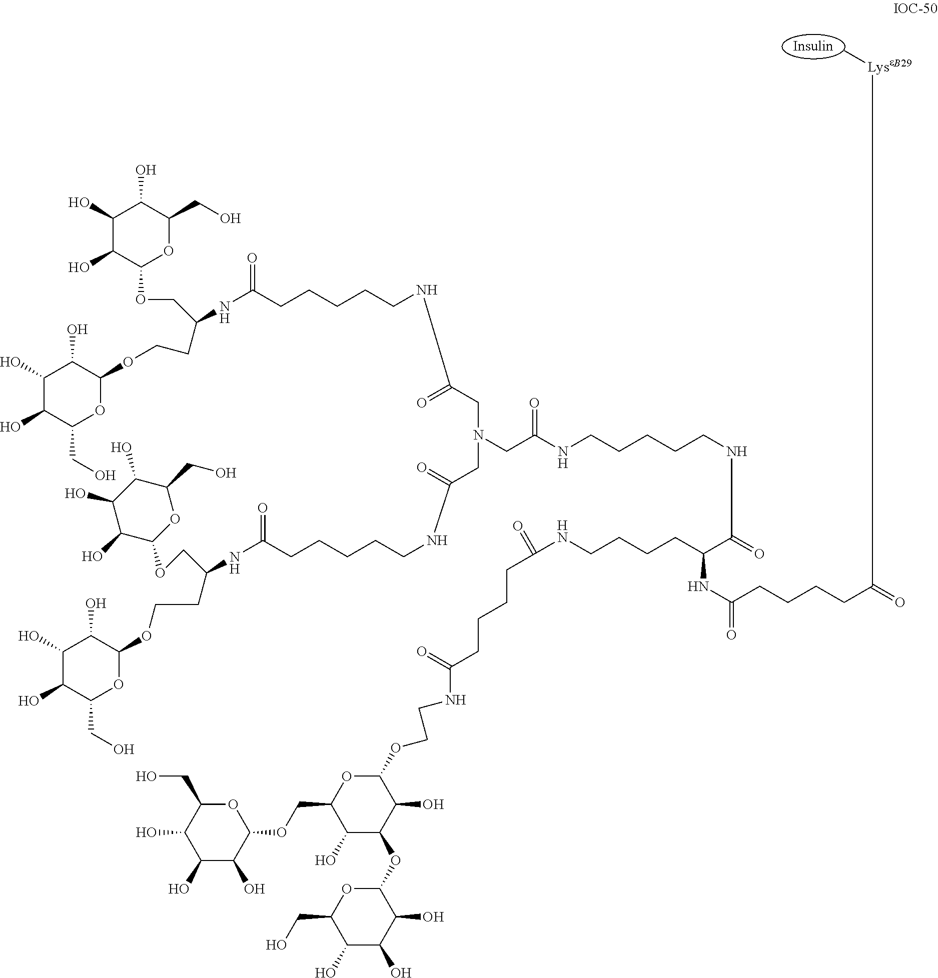

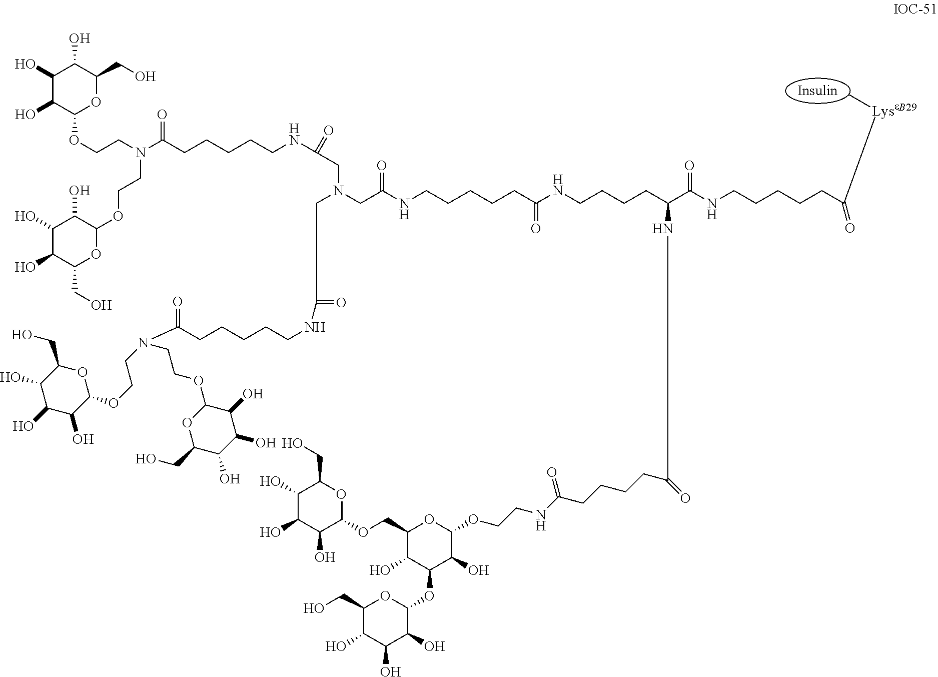

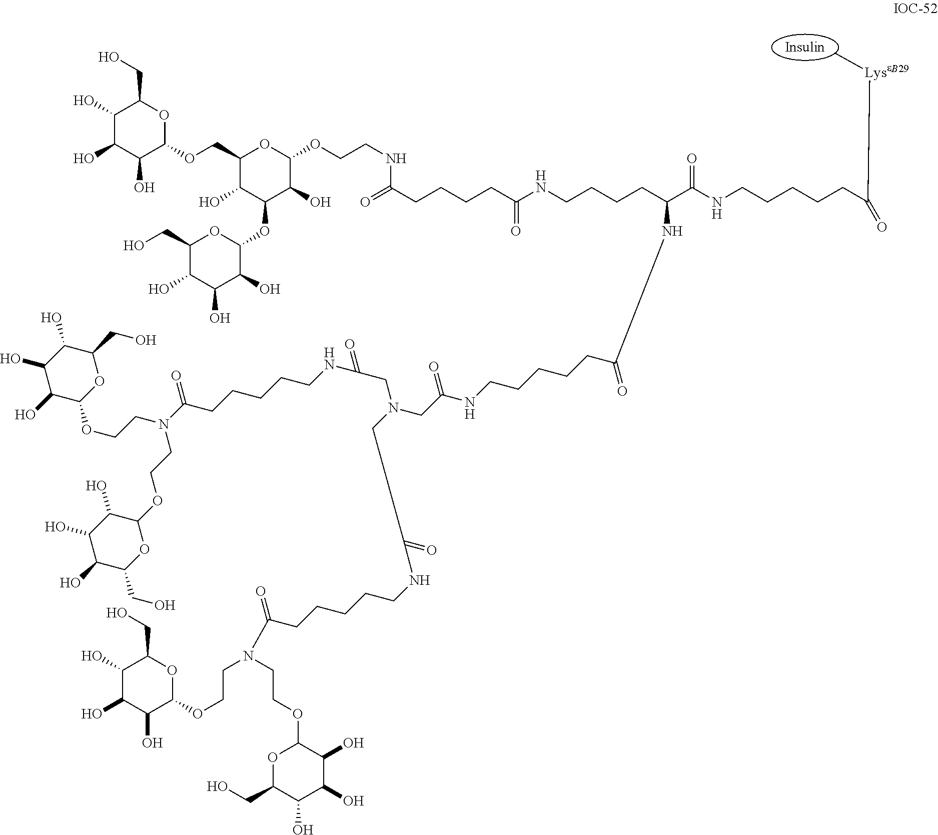

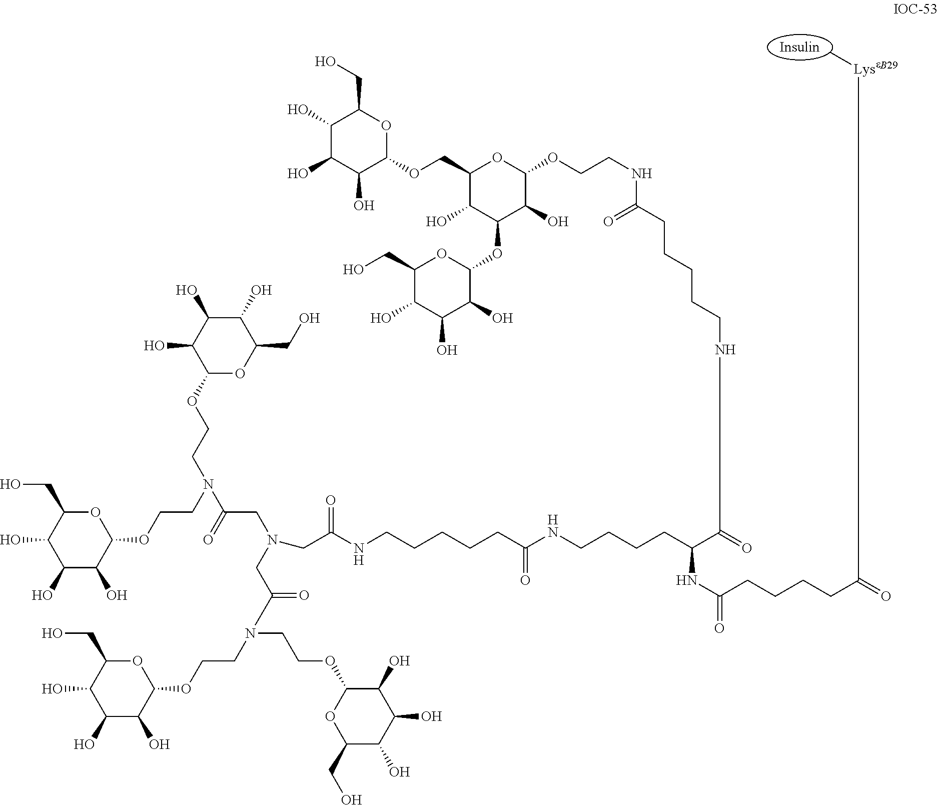

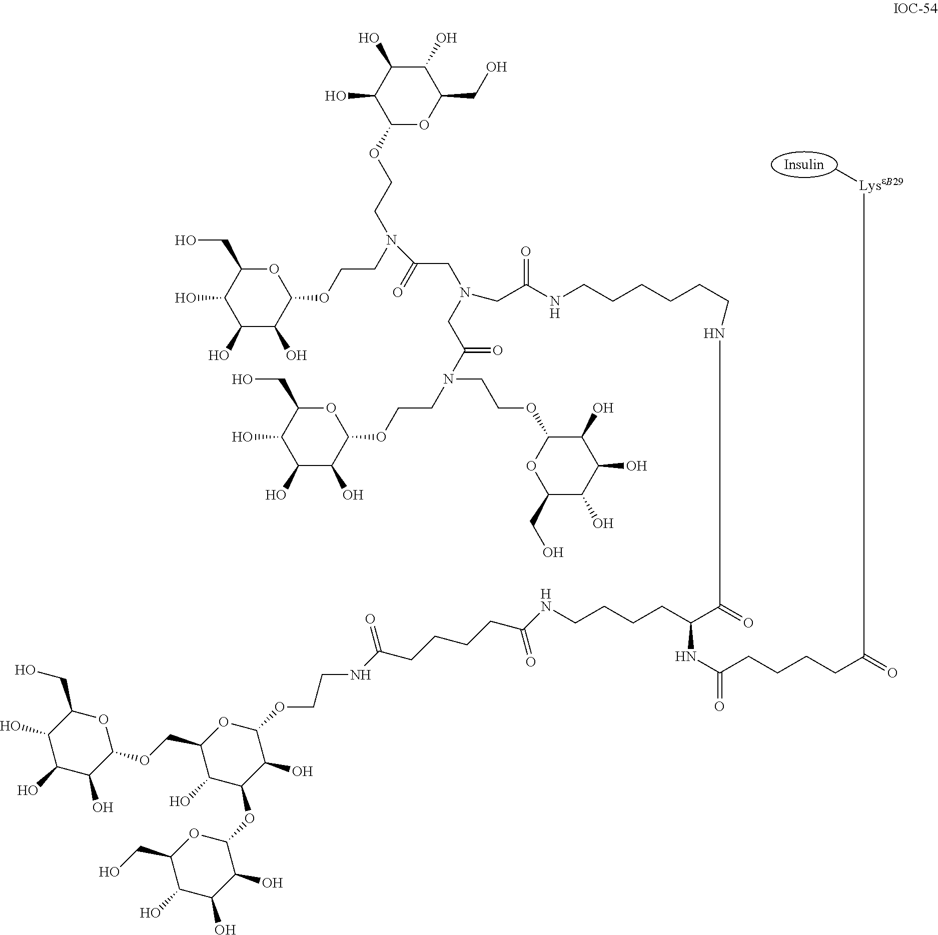

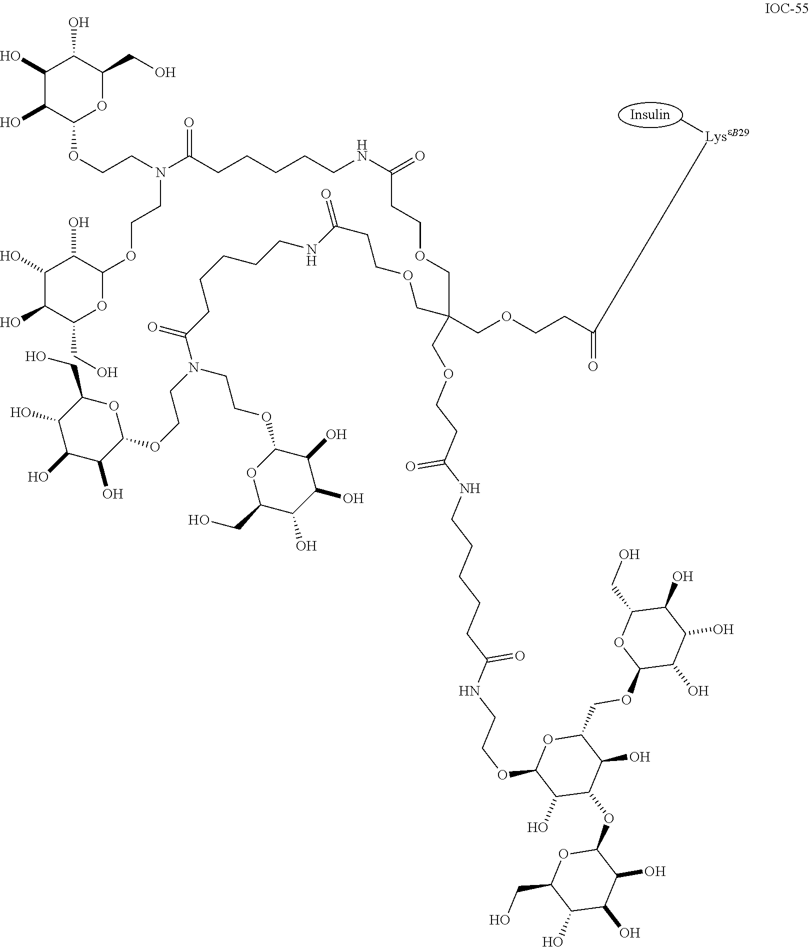

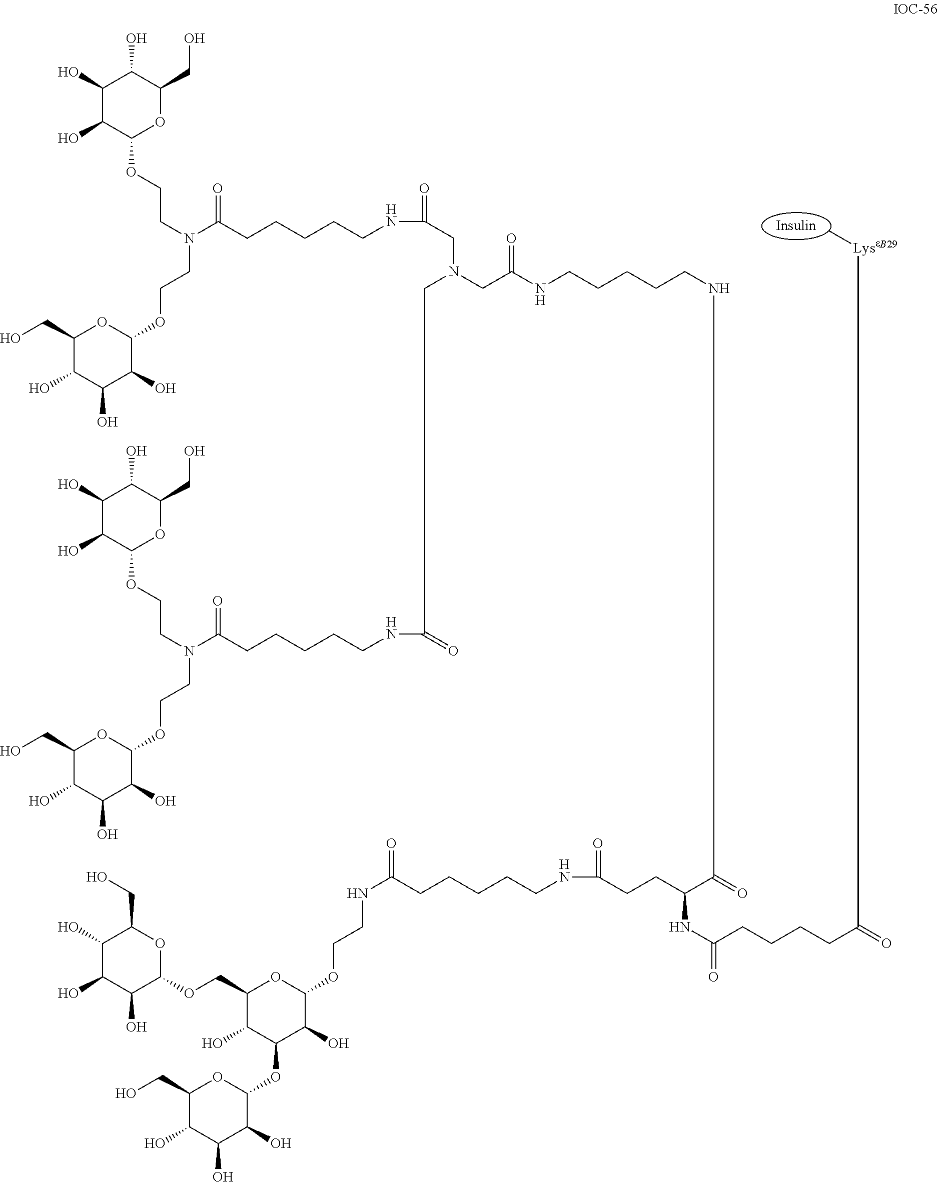

### Sustained Release Formulations

In particular embodiments, it may be advantageous to administer an insulin conjugate in a sustained fashion (i.e., in a form that exhibits an absorption profile that is more sustained than soluble recombinant human insulin). This will provide a sustained level of conjugate that can respond to fluctuations in glucose on a timescale that it more closely related to the typical glucose fluctuation timescale (i.e., hours rather than minutes). In particular embodiments, the sustained release formulation may exhibit a zero-order release of the conjugate when administered to a mammal under non-hyperglycemic conditions (i.e., fasted conditions).

It will be appreciated that any formulation that provides a sustained absorption profile may be used. In particular embodiments this may be achieved by combining the conjugate with other ingredients that slow its release properties into systemic circulation. For example, protamine zinc insulin (“PZI”) formulations may be used for this purpose. The present disclosure encompasses amorphous and crystalline forms of these PZI formulations.

Thus, in particular embodiments, a formulation of the present disclosure includes from about 0.05 to about 10 mg protamine/mg conjugate. For example, from about 0.2 to about 10 mg protamine/mg conjugate, e.g., about 1 to about 5 mg protamine/mg conjugate.

In particular embodiments, a formulation of the present disclosure includes from about 0.006 to about 0.5 mg zinc/mg conjugate. For example, from about 0.05 to about 0.5 mg zinc/mg conjugate, e.g., about 0.1 to about 0.25 mg zinc/mg conjugate.

In particular embodiments, a formulation of the present disclosure includes protamine and zinc in a ratio (w/w) in the range of about 100:1 to about 5:1, for example, from about 50:1 to about 5:1, e.g., about 40:1 to about 10:1. In particular embodiments, a PZI formulation of the present disclosure includes protamine and zinc in a ratio (w/w) in the range of about 20:1 to about 5:1, for example, about 20:1 to about 10:1, about 20:1 to about 15:1, about 15:1 to about 5:1, about 10:1 to about 5:1, about 10:1 to about 15:1.

One or more of the following components may be included in the PZI formulation: an antimicrobial preservative, an isotonic agent, and/or an unconjugated insulin molecule.

In particular embodiments, a formulation of the present disclosure includes an antimicrobial preservative (e.g., m-cresol, phenol, methylparaben, or propylparaben). In particular embodiments, the antimicrobial preservative is m-cresol. For example, in particular embodiments, a formulation may include from about 0.1 to about 1.0% v/v m-cresol. For example, from about 0.1 to about 0.5% v/v m-cresol, e.g., about 0.15 to about 0.35% v/v m-cresol.

In particular embodiments, a formulation of the present disclosure includes a polyol as isotonic agent (e.g., mannitol, propylene glycol, or glycerol). In particular embodiments, the isotonic agent is glycerol. In particular embodiments, the isotonic agent is a salt, e.g., NaCl. For example, a formulation may comprise from about 0.05 to about 0.5 M NaCl, e.g., from about 0.05 to about 0.25 M NaCl or from about 0.1 to about 0.2 M NaCl.

In particular embodiments, a formulation of the present disclosure includes an amount of unconjugated insulin molecule. In particular embodiments, a formulation includes a molar ratio of conjugated insulin molecule to unconjugated insulin molecule in the range of about 100:1 to 1:1, e.g., about 50:1 to 2:1 or about 25:1 to 2:1.

The present disclosure also encompasses the use of standard sustained (also called extended) release formulations that are well known in the art of small molecule formulation (e.g., see REMINGTON'S PHARMACEUTICAL SCIENCES, 19^{th} ed., Mack Publishing Co., Easton, PA, 1995). The present disclosure also encompasses the use of devices that rely on pumps or hindered diffusion to deliver a conjugate on a gradual basis. In particular embodiments, a long acting formulation may (additionally or alternatively) be provided by using a modified insulin molecule. For example, one could use insulin glargine (LANTUS™) or insulin detemir (LEVEMIR™) instead of wild-type human insulin in preparing the conjugate. Insulin glargine is an exemplary long acting insulin analog in which Asn at position A21 of the A-chain has been replaced by glycine and two arginine residues are at the C-terminus of the B-chain. The effect of these changes is to shift the isoelectric point, producing an insulin that is insoluble at physiological pH but is soluble at pH 4. Insulin detemir is another long acting insulin analog in which Thr at position B30 of the B-chain has been deleted and a C14 fatty acid chain has been attached to the Lys at position B29.

### Uses of Conjugates

In another aspect, the present disclosure provides methods of using the insulin conjugates. In general, the insulin conjugates can be used to controllably provide insulin to an individual in need in response to a saccharide (e.g., glucose or an exogenous saccharide such as mannose, alpha-methylmannose, L-fucose, etc.). The disclosure encompasses treating diabetes by administering an insulin conjugate of the present disclosure. Although the insulin conjugates can be used to treat any patient (e.g., dogs, cats, cows, horses, sheep, pigs, mice, etc.), they are most preferably used in the treatment of humans. An insulin conjugate may be administered to a patient by any route. In general, the present disclosure encompasses administration by oral, intravenous, intramuscular, intra-arterial, subcutaneous, intraventricular, transdermal, rectal, intravaginal, intraperitoneal, topical (as by powders, ointments, or drops), buccal, or as an oral or nasal spray or aerosol. General considerations in the formulation and manufacture of pharmaceutical compositions for these different routes may be found, for example, in REMINGTON'S PHARMACEUTICAL SCIENCES, 19^{th} ed., Mack Publishing Co., Easton, PA, 1995. In various embodiments, the conjugate may be administered subcutaneously, e.g., by injection. The insulin conjugate may be dissolved in a carrier for ease of delivery. For example, the carrier can be an aqueous solution including, but not limited to, sterile water, saline, or buffered saline.

In general, a therapeutically effective amount of the insulin conjugate will be administered. The term “therapeutically effective amount” means a sufficient amount of the insulin conjugate to treat diabetes at a reasonable benefit/risk ratio, which involves a balancing of the efficacy and toxicity of the insulin conjugate. In various embodiments, the average daily dose of insulin is in the range of 10 to 200 U, e.g., 25 to 100 U (where 1 unit of insulin (“U”) is ˜0.04 mg). In particular embodiments, an amount of conjugate with these insulin doses is administered on a daily basis. In particular embodiments, an amount of conjugate with 5 to 10 times these insulin doses is administered on a weekly basis. In particular embodiments, an amount of conjugate with 10 to 20 times these insulin doses is administered on a bi-weekly basis. In particular embodiments, an amount of conjugate with 20 to 40 times these insulin doses is administered on a monthly basis.

In particular embodiments, a conjugate of the present disclosure may be used to treat hyperglycemia in a patient (e.g., a mammalian or human patient). In particular embodiments, the patient is diabetic. However, the present methods are not limited to treating diabetic patients. For example, in particular embodiments, a conjugate may be used to treat hyperglycemia in a patient with an infection associated with impaired glycemic control. In particular embodiments, a conjugate may be used to treat diabetes.

In particular embodiments, when an insulin conjugate or formulation of the present disclosure is administered to a patient (e.g., a mammalian patient), it induces less hypoglycemia than an unconjugated version of the insulin molecule. In particular embodiments, a formulation of the present disclosure induces a lower HbA1c value in a patient (e.g., a mammalian or human patient) than a formulation comprising an unconjugated version of the insulin molecule. In particular embodiments, the formulation leads to an HbA1c value that is at least 10% lower (e.g., at least 20% lower, at least 30% lower, at least 40% lower, or at least 50% lower) than a formulation comprising an unconjugated version of the insulin molecule. In particular embodiments, the formulation leads to an HbA1c value of less than 7%, e.g., in the range of about 4 to about 6%. In particular embodiments, a formulation comprising an unconjugated version of the insulin molecule leads to an HbA1c value in excess of 7%, e.g., about 8 to about 12%.

### Exogenous Trigger

As mentioned previously, the methods, conjugates and compositions that are described herein are not limited to glucose responsive conjugates. As demonstrated in the Examples, several exemplary insulin conjugates were also responsive to exogenous saccharides such as alpha-methylmannose. It will therefore be appreciated that, in particular embodiments, an insulin conjugate may be triggered by exogenous administration of a saccharide other than glucose, such as alpha-methylmannose or any other saccharide that can alter the PK or PD properties of the conjugate.

Once a conjugate has been administered as described above (e.g., as a sustained release formulation), it can be triggered by administration of a suitable exogenous saccharide. In a particular embodiment, a triggering amount of the exogenous saccharide is administered. As used herein, a “triggering amount” of exogenous saccharide is an amount sufficient to cause a change in at least one PK and/or PD property of the conjugate (e.g., Cₘₐₓ, AUC, half-life, etc. as discussed previously). It is to be understood that any of the aforementioned methods of administration for the conjugate apply equally to the exogenous saccharide. It is also to be understood that the methods of administration for the conjugate and exogenous saccharide may be the same or different. In various embodiments, the methods of administration are different (e.g., for purposes of illustration the conjugate may be administered by subcutaneous injection on a weekly basis while the exogenous saccharide is administered orally on a daily basis). The oral administration of an exogenous saccharide is of particular value because it facilitates patient compliance. In general, it will be appreciated that the PK and PD properties of the conjugate will be related to the PK profile of the exogenous saccharide. Thus, the conjugate PK and PD properties can be tailored by controlling the PK profile of the exogenous saccharide. As is well known in the art, the PK profile of the exogenous saccharide can be tailored based on the dose, route, frequency, and formulation used. For example, if a short and intense activation of the conjugate is desired then an oral immediate release formulation might be used. In contrast, if a longer less intense activation of conjugate is desired then an oral extended release formulation might be used instead. General considerations in the formulation and manufacture of immediate and extended release formulation may be found, for example, in REMINGTON'S PHARMACEUTICAL SCIENCES, 19^{th} ed., Mack Publishing Co., Easton, PA, 1995.

It will also be appreciated that the relative frequency of administration of a conjugate of the present disclosure and of an exogenous saccharide may be the same or different. In particular embodiments, the exogenous saccharide is administered more frequently than the conjugate. For example, in particular embodiment, the conjugate may be administered daily while the exogenous saccharide is administered more than once a day. In particular embodiment, the conjugate may be administered twice weekly, weekly, biweekly, or monthly, while the exogenous saccharide is administered daily. In particular embodiments, the conjugate is administered monthly, and the exogenous saccharide is administered twice weekly, weekly, or biweekly. Other variations on these schemes will be recognized by those skilled in the art and will vary depending on the nature of the conjugate and formulation used.

The following examples are intended to promote a further understanding of the present disclosure.

### Examples

### General Procedures

All chemicals were purchased from commercial sources, unless otherwise noted. Reactions sensitive to moisture or air were performed under nitrogen or argon using anhydrous solvents and reagents. The progress of reactions was monitored by analytical thin layer chromatography (“TLC”), high performance liquid chromatography-mass spectrometry (“HPLC-MS”), or ultra-performance liquid chromatography-mass spectrometry (“UPLC-MS”). TLC was performed on E. Merck TLC plates precoated with silica gel 60F-254, layer thickness 0.25 mm. The plates were visualized using 254 nm ultraviolet radiation (“UV”) and/or by exposure to cerium ammonium molybdate (“CAM”) or p-anisaldehyde staining solutions followed by charring. High performance liquid chromatography (“HPLC”) was conducted on an Agilent 1100 series HPLC using SUPELCO™ Ascentis Express C18 2.7 μm 3.0×100 mm column with gradient 10:90-99:1 v/v CH₃CN/H₂O+v 0.05% TFA over 4.0 min then hold at 98:2 v/v CH₃CN/H₂O+v 0.05% TFA for 0.75 min; flow rate 1.0 mL/min, UV range 200-400 nm (LC-MS Method A). Mass analysis was performed on a Waters MICROMASS™ ZQTM with electrospray ionization in positive ion detection mode and the scan range of the mass-to-charge ratio was either 170-900 or 500-1500. Ultra-performance liquid chromatography (UPLC) was performed on a Waters ACQHITY™ UPLC™ system using the following methods:

UPLC-MS Method A: Waters ACQUITY™ UPLC™ BEH C18 1.7 μm 2.1×100 mm column with gradient 10:90-70:30 v/v CH₃CN/H₂O+v 0.10% TFA over 4.0 min and 70:30-95:5 v/v CH₃CN/H₂O+v 0.1% TFA over 40 sec; flow rate 0.3 mL/min, UV wavelength 200-300 nm.

UPLC-MS Method B: Waters ACQUITY™ UPLC™ BEH C18 1.7 μm 2.1×100 mm column with gradient 60:40-100:0 v/v CH₃CN/H₂O+v 0.1% TFA over 4.0 min and 100:0-95:5 v/v CH₃CN/H₂O+v 0.1% TFA over 40 sec; flow rate 0.3 mL/min, UV wavelength 200-300 nm.

UPLC-MS Method C: Waters ACQUITY™ UPLC™ HSS T3 1.7 μm 2.1×100 mm column with gradient 0:100-40:60 v/v CH₃CN/H₂O+v 0.05% TFA over 8.0 min and 40:60-10:90 v/v CH₃CN/H₂O+v 0.05% TFA over 2.0 min; flow rate 0.3 mL/min, UV wavelength 200-300 nm.

UPLC-MS Method D: Waters ACQUITY™ UPLC™ BEH C18 1.7 μm 2.1×100 mm column with gradient 0:100-60:40 v/v CH₃CN/1120+v 0.1% TFA over 8.0 min and 60:40-90:10 v/v CH₃CN/H₂O+v 0.1% TFA over 3.0 min and hold at 100:0 v/v CH₃CN/H₂O+v 0.1% TFA for 2 min; flow rate 0.3 mL/min, UV wavelength 200-300 nm.

UPLC-MS Method E: Waters ACQUITY™ UPLC™ BEH C8 1.7 μm 2.1×100 mm column with gradient 10:90-55:45 v/v CH₃CN/1120+v 0.1% TFA over 4.2 min and 100:0-95:5 v/v CH₃CN/H₂O+v 0.1% TFA over 0.4 min; flow rate 0.3 mL/min, UV wavelength 200-300 nm.

UPLC-MS Method F: Waters ACQUITY™ UPLC™ BEH C8 1.7 μm 2.1×100 mm column with gradient 10:90-90:10 v/v CH₃CN/H₂O+v 0.1% TFA over 4.2 min and 90:10-95:5 v/v CH₃CN/H₂O+v 0.1% TFA over 0.4 min; flow rate 0.3 mL/min, UV wavelength 200-300 nm.

UPLC-MS Method G: Waters ACQUITY™ UPLC™ BEH300 C4 1.7 μm 2.1×100 mm column with gradient 10:90-90:10 v/v CH₃CN/H₂O+v 0.1% TFA over 4.0 min and 90:10-95:5 v/v CH₃CN/H₂O+v 0.1% TFA over 0.5 min; flow rate 0.3 mL/min, UV wavelength 200-300 nm.

Mass analysis was performed on a Waters MICROMASS™ LCT PREMIER™ XE with electrospray ionization in positive ion detection mode, and the scan range of the mass-to-charge ratio was 300-2000. The identification of the produced insulin conjugates was confirmed by comparing the theoretical molecular weight to the experimental value that was measured using UPLC-MS. For the determination of the position of sugar modification(s), specifically, insulin conjugates were subjected to DTT treatment (for a/b chain) or Glu-C digestion (with reduction and alkylation), and then the resulting peptides were analyzed by LC-MS. Based on the measured masses, the sugar positions were deduced.

Flash chromatography was performed using either a Biotage Flash Chromatography apparatus (Dyax Corp.) or a COMBIFLASH™ Rf instrument (Teledyne Isco). Normal-phase chromatography was carried out on silica gel (20-70 μm, 60 Å pore size) in pre-packed cartridges of the size noted. Reverse-phase chromatography was carried out on C18-bonded silica gel (20-60 μm, 60-100 Å pore size) in pre-packed cartridges of the size noted. Preparative scale HPLC was performed on Gilson 333-334 binary system using Waters DELTA-PAK™ C4 15 μm, 300 Å, 50×250 mm column or KROMASIL™ C8 10 μm, 100 Å, 50×250 mm column, flow rate 85 mL/min, with gradient noted. Concentration of solutions was carried out on a rotary evaporator under reduced pressure or freeze-dried on a VirTis Freezemobile Freeze Dryer (SP Scientific).

¹H-NMR spectra were acquired at 500 MHz (or otherwise specified) spectrometers in deuterated solvents noted. Chemical shifts were reported in parts per million (“ppm”). Tetramethylsilane (“TMS”) or residual proton peak of deuterated solvents was used as an internal reference. Coupling constant (“J”) were reported in hertz (“Hz”).

### ABBREVIATIONS

αMM alpha-methylmannose
Å Angstrom(s)
ACN, MeCN Acetonitrile, CH₃CN
AcOH Acetic acid
aq Aqueous
BEH Ethylene bridged hybrid technology
BOC, Boc tert-butoxycarbonyl protecting group
Brine Saturated aqueous sodium chloride solution
BSA Bovine serum albumin
C18 Carbon 18
CAM Cerium ammonium molybdate
Cbz Carboxybenzyl
CHO Chinese hamster ovary
Cₘₐₓ The highest concentration of a drug in the blood
conc. Concentrated
CuI Copper iodide
CV Column volume
Da Dalton(s)
DCC Dicyclohexylcarbodiimide
DCM Dichloromethane
DDT Dichlorodiphenyltrichloroethane
DELFH Eu-N1-ITC DELFIA™ Europium N1-isocyanate
DIPEA N,N-diisopropylethylamine or Hünig's Base
dL Deciliter(s)
DMA N,N-dimethylacetamide
DMAP (4-dimethylamino)pyridine
DMF N,N-dimethylformamide
DMSO Dimethylsulfoxide
DTT Dithiothreitol
EDC N-(3-dimethylaminopropyl)-N′-ethylcarbodiimide hydrochloride
Et₂O, ether Diethyl ether
EtOAc Ethyl acetate
FBS Fasting blood sugar
FR Flow rate
g Gram(s)
G418 GENETICIN™, aminoglycoside antibiotic
h, hr Hour(s)
HATU O-(7-azabenzotriazol-1-yl)-N,N,N′,N′-tetramethyluronium hexafluorophosphate)
HbA1c Hemoglobin A1c
Hex Hexanes
HOBt, HOBT 1-hydroxybenzotriazole hydrate
HPLC High performance liquid chromatography
HPLC-MS High performance liquid chromatography-mass spectroscopy
HSPyU 1-(((2,5-dioxopyrrolidin-1-yl)oxy)(pyrrolidin-1-yl)methylene)pyrrolidin-1-ium hexafluorophosphate(V)
Hz Hertz
IGF Insulin-like growth factor
IPA Isopropyl alcohol
IR Insulin receptor
IR(B) Insulin receptor (type B)
J Coupling constant
kg Kilogram(s)
LC-MS Liquid chromatography-mass spectroscopy
M Molar, moles per liter
m/e Mass/electron
m/z Mass-to-charge ratio
Me Methyl, CH₃—
mg Milligram(s)
MHz Megahertz(s)
mm Minute(s)
mL, ml Milliliter(s)
mm Millimeter(s)
mmol Millimole(s)
MRC1 Mannose receptor C type 1
MS, ms Mass spectrum
MSD Meso scale discovery
MW Molecular weight
N Normality, number of mole equivalents per liter of solution
NaOMe Sodium methoxide
N6-Cbz-L-Lysine N6-((benzyloxy)carbonyl)-L-lysine
NH₂-PEG(6)-CO₂H 1-Amino-3,6,9,12,15,18-hexaoxahenicosan-21-oic acid
NHS N-Hydroxysuccinimide
nm Nanometer(s)
nM Nanomolar
NMM N-methylmorpholine
nmol Nanomole(s)
OBD Optimum bed density
OtBu Tert-butyl ester
PBS Phosphate buffered saline
Pd/C, Pd—C Palladium on carbon
PE Petroleum ether
PFTU Pentafluorphenol-tetramethyluronium hexafluorophosphate
pIR Phosphorus insulin receptor
ppm Parts per million
psi Pounds per square inch
PZI Protamine zinc insulin
RPM, rpm Revolutions per minute
RT, rt Room temperature (˜25° C.)
sat., sat'd Saturated
sec Second(s)
SiO₂ Silicon dioxide
TEA Triethylamine
TFA Trifluoroacetic acid
TFAA Trifluoroacetic anhydride
THF Tetrahy drofuran
TLC Analytical thin layer chromatography
Tₘₐₓ The amount of time that a drug is present at the maximum concentration in serum
TMS Tetramethylsilane
t_{R} Retention time
TSTU N,N,N′,N′-tetramethyl-O—(N-succinimidyl)uronium tetrafluoroborate
UPLC Ultra-performance liquid chromatography
UPLC-MS Ultra-performance liquid chromatography-mass spectroscopy
UV Ultraviolet
V, v volume
v/v Volume per volume
w/w Weight ratio
WGA PVT PEI SPA Wheat germ agglutinin polyvinyltoluene polyethyleneimine scintillation proximity assay
Wt, wtt, w Weight
z charge
Z-ASP(OBZL)-OH N-Cbz-L-aspartic acid 4-benzyl ester
Z-GLU-OBZL N-Cbz-L-glutamic acid benzyl ester
Z-GLU-OH N-(Carbobenzyloxy)-L-glutamic acid
Z-LYS(BOC)—OH (Carbobenzoxyl)(carbo-tert-butoxyl)lysine
Z-LYS-OH Nα-(Carbobenzyloxy)-L-lysine
μL, μl, uL, ul, Microliter(s)
μm, um Micrometer(s)
μmol, umol Micromole(s)

### Intermediate 1: 13-(Carboxymethyl)-3,11-dioxo-1-phenyl-2-oxa-4,10,13-triazapentadecan-15-oic acid

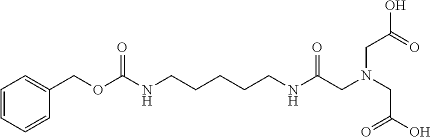 [SMILES: O=C(O)CN(CC(=O)O)CC(=O)NCCCCCNC(=O)OCc1ccccc1]

To a solution of benzyl (5-aminopentyl)carbamate hydrochloride (10 g, 36.7 mmol) in DMF (44 mL) at 0° C. was added K₂CO₃ (5.07 g, 36.7 mmol), and the mixture was stirred at ice bath temperature for 2 h. The resulting suspension was filtered through CELITE™ diatomaceous earth, and the filtrate added to a solution of 2-(2,6-dioxomorpholino) acetic acid (6.35 g, 36.7 mmol) in DMF (44 mL) at 0° C. for 30 min. The resulting mixture was warmed to rt and stirred for 16 h. The liquid was allowed to evaporate from the mixture, and water (20 mL) was added. The resulting mixture was stirred at rt for 30 min. A precipitate formed and was removed by filtration, washed with a small amount of ACN to isolate title compound. UPLC-MS Method A: m/z=410.2 (z=1); t_{R}=3.39 min.

### Intermediate 2: 2,2′-(((S)-6-Amino-1-oxo-1-((2-(((2R,3S,4R,5S,6S)-3,4,5-trihydroxy-6-methyltetrahydro-2H-pyran-2-yl)oxy)ethyl)amino)hexan-2-yl)azanediyl)bis(N-(2-(((2R,3S,4R,5S,6S)-3,4,5-trihydroxy-6-methyltetrahydro-2H-pyran-2-yl)oxy)ethyl)acetanmide)

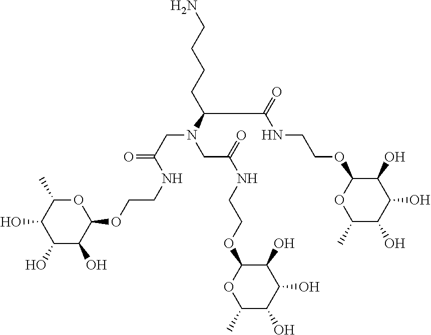 [SMILES: C[C@@H]1O[C@@H](OCCNC(=O)CN(CC(=O)NCCO[C@@H]2O[C@@H](C)[C@@H](O)[C@@H](O)[C@@H]2O)[C@@H](CCCCN)C(=O)NCCO[C@@H]2O[C@@H](C)[C@@H](O)[C@@H](O)[C@@H]2O)[C@@H](O)[C@H](O)[C@@H]1O]

### Step 1: Benzyl ((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)carbamate

To a solution of (S)-2,2′-((5-(((benzyloxy)carbonyl)amino)-1-carboxypentyl)azanediyl) diacetic acid (3000 mg, 0.579 mmol) in DMF (100 mL) at rt was added EDC (4385 mg, 22.87 mmol), HOBT (3503 mg, 22.87 mmol), and TEA (6.18 mL, 44.4 mmol). After stirring for 30 min, a solution of AEF in DMF (10 mL) was added dropwise to the reaction mixture. After stirring for 16 h, the mixture was concentrated, and the residue was purified by C18 reverse phase chromatography to provide the title compound. UPLC-MS Method A: m/z=964.5 (z=1); t_{R}=2.00 min.

### Step 2: 2, 2′-(((S)-6-Amino-1-oxo-1-((2-(((2R,3S,4R,5S,6S)-3,4,5-trihydroxy-6-methyltetrahydro-2H-pyran-2-yl)oxy)ethyl)amino)hexan-2-yl)azanediyl)bis(N-(2-(((2R,3S,4R,5S,6S)-3,4,5-trihydroxy-6-methyltetrahydro-2H-pyran-2-yl)oxy)ethyl)acetamide)

A mixture of benzyl ((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)carbamate (10 g, 10.37 mmol) and Pd/C (0.331 g, 3.11 mmol) in water (100 mL) was hydrogenated for 1 h. The solution was filtered through CELITE™ diatomaceous earth and washed with water. The filtrate was freeze-dried to isolate title compound. UPLC-MS Method A: m/z=830.2 (z=1); t_{R}=3.70.

### Intermediate 3: (S)-2,2′-((5-(((Benzyloxy)carbonyl)amino)-1-carboxypentyl)azanediyl)diacetic acid

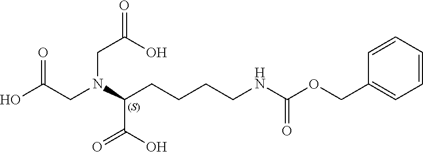

To a solution of bromoacetic acid (39.7 g, 285 mmol) in 2M NaOH (178 mL, 357 mmol) at 0° C. was added N⁶-Cbz-L-Lysine (20 g, 71.3 mmol) dissolved in 2M NaOH (71.3 mL, 143 mmol) dropwise. Upon completion of the addition, the solution was heated to 50° C. for 16 h. The solution was cooled to 0° C. and acidified with concentrated HCl until a precipitate formed. The solid was removed by filtration and dried to isolate title compound.

### Intermediate 4: 6-Amino-N-(2-(((2R,3S,4R,5S,6S)-3,4,5-trihydroxy-6-methyltetrahydro-2H-pyran-2-yl)oxy)ethyl)hexananide

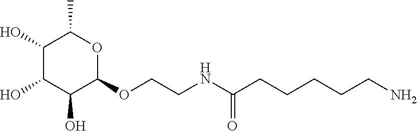 [SMILES: C[C@@H]1O[C@@H](OCCNC(=O)CCCCCN)[C@@H](O)[C@H](O)[C@@H]1O]

### Step 1: Benzyl (6-oxo-6-((2-(((2R,3S,4R,5S,6S)-3,4,5-trihydroxy-6-methyltetrahydro-2H-pyran-2-yl)oxy)ethyl)amino)hexyl)carbamate

2,5-dioxopyrrolidin-1-yl 6-(((benzyloxy)carbonyl)amino)hexanoate (3.15 g, 8.69 mmol) was added to a solution of AEF (1.5 g, 7.24 mol) in DMF (80 mL), and the solution was stirred. After an hour, TEA (2.220 mL, 15.92 mmol) was added, and the reaction was stirred for 48 h. The reaction mixture was concentrated and purified by C18 reverse phase chromatography to isolate title compound. UPLC-MS Method A: m/z=455.2 (z=1); t_{R}=4.82.

### Step 2: 6-Amino-N-(2-(((2R,3S,4R,5S,6S)-3,4,5-trihydroxy-6-methyltetrahydro-2H-pyran-2-yl)oxy)ethyl)hexanamide

A mixture of benzyl (6-oxo-6-((2-(((2R,3S,4R,5S,6S)-3,4,5-trihydroxy-6-methyltetrahydro-2H-pyran-2-yl)oxy)ethyl)amino)hexyl)carbamate (3.29 g, 7.24 mmol) and Pd/C (0.023 g, 0.217 mmol) in water (20 mL) was placed under N₂. The solution was hydrogenated for 1 h. The solution was filtered through CELITE™ diatomaceous earth and washed with water. The filtrate was freeze-dried to isolate the title compound. UPLC-MS Method A: m/z=321.2 (z=1); t_{R}=4.28 min.

### Intermediate 5: 6-Amino-N-(2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)hexanamide

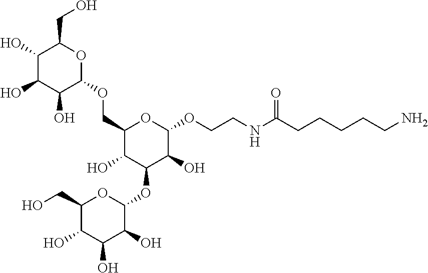 [SMILES: NCCCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)carbamate

2,5-dioxopyrrolidin-1-yl 6-(((benzyloxy)carbonyl)amino)hexanoate (1.98 g, 5.48 mmol) was added to a solution of AETM (2.0 g, 3.65 mmol) in DMF (50 mL), and the solution was stirred. After 1 h, TEA (1.273 mL, 9.13 mmol) was added, and the reaction was stirred for 48 h. The reaction was concentrated and purified by C18 reverse phase chromatography to isolate title compound. UPLC-MS Method A: m/z=455.2 (z=1); t_{R}=4.82.

### Step 2: 6-Amino-N-(2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) hexanamide

A mixture of benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate (1.6 g, 2.05 mmol) and Pd/C (6.5 mg, 0.062 mmol) in water (20 mL) was placed under N₂. The solution was hydrogenated for 1 h, filtered through CELITE™ diatomaceous earth and washed with water. The filtrate was freeze-dried to isolate title compound. UPLC-MS Method A: m/z=661.3 (z=1); t_{R}=1.15 min.

### Intermediate 6: N-(2-((6-(benzyloxy)-6-oxohexyl)amino)-2-oxoethyl)-N-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl)amino)-2-oxoethyl)glycine

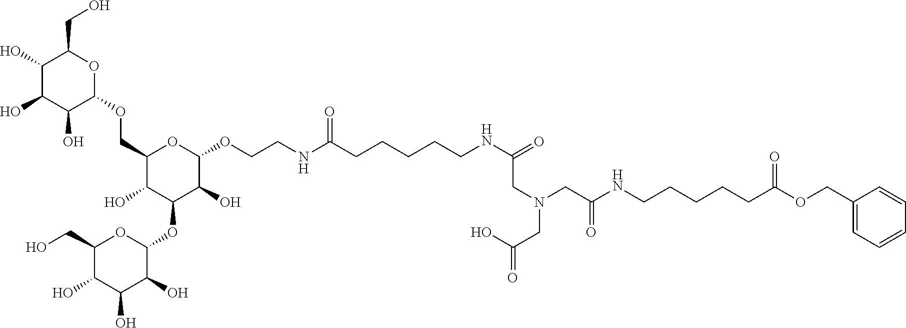 [SMILES: O=C(O)CN(CC(=O)NCCCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CC(=O)NCCCCCC(=O)OCc1ccccc1]

To a suspension of 2,2′-((2-((6-(benzyloxy)-6-oxohexyl)amino)-2-oxoethyl)azanediyl) diacetic acid (1.5 g, 3.66 mmol) in DCM (16 mL) at 0° C. was added TFAA (0.394 mL, 2.79 mmol). After stirring at 0° C. for 3 h, the mixture was chilled to −30° C. To the resulting solution was added as solution of TEA (0.848 mL, 6.08 mmol) in DMF (12 mL) dropwise over 30 min. To the resulting mixture was added a solution of 6-amino-N-(2-(((2S,3S,4S,5R,6R)-3,5-dihydroxy-4-(((2R,3S,4S,5S,6R)-3,4,5-trihydroxy-6-(hydroxymethyltetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)hexanamide (1.256 g, 1.90 mmol) in DMF (16 mL). After stirring at rt for 16 h, the mixture was concentrated, and the residue was purified by C18 reverse phase chromatography to isolate title compound. UPLC-MS Method A: m/z=1037.5 (z=1); t_{R}=3.26 min.

### Intermediate 7: 6-amino-N-(2-(((2S,3S,4S,5S,6R)-3,4,5-trihydroxy-6-(hydroxymethyl) tetrahydro-2H-pyran-2-yl)oxy)ethyl)hexanamide

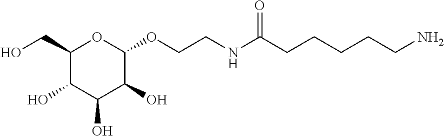 [SMILES: NCCCCCC(=O)NCCO[C@H]1O[C@H](CO)[C@@H](O)[C@H](O)[C@@H]1O]

### Step 1: Benzyl (6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)carbamate

The title compound was prepared using procedures analogous to those described for Intermediate 4, Step 1, substituting AEM for AEF to isolate title compound. UPLC-MS Method A: m/z=471.2 (z=1); t_{R}=2.69 min.

### Step 2: 6-Amino-N-(2-(((2S,3S,4S,5S,6R)-3,4,5-trihydroxy-6-(hydroxymethyl)tetrahydro-2H-pyran-2-yl)oxy)ethyl)hexanamide

The title compound was prepared using procedures analogous to those described for Intermediate 4, Step 2, substituting benzyl (6-oxo-6-((2-(((2R,3S,4R,5S,6S)-3,4,5-trihydroxy-6-methyltetrahydro-2H-pyran-2-yl)oxy)ethyl)amino)hexyl)carbamate for benzyl (6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)carbamate to isolate title compound. UPLC-MS Method A: m/z=337.2 (z=1); t_{R}=0.84 min.

### Intermediate 8: (S)-3-(2-((6-(Benzyloxy)-6-oxohexyl)amino)-2-oxoethyl)-5,13,17,26-tetraoxo-24-(2-oxo-2-((2-(((2R,3S,4R,5S,6S)-3,4,5-trihydroxy-6-methyltetrahydro-2H-pyran-2-yl)oxy)ethyl) amino)ethyl)-15-(2-oxo-2-((2-(((2S,3R,4S,5R,6R)-3,4,5-trihydroxy-6-methyltetrahydro-2H-pyran-2-yl)oxy)ethyl)amino)ethyl)-29-(((2R,3S,4R,5S,6S)-3,4,5-trihydroxy-6-methyltetrahydro-2H-pyran-2-yl)oxy)-23-((2-(((2R,3S,4R,5S,6S)-3,4,5-trihydroxy-6-methyltetrahydro-2H-pyran-2-yl)oxy)ethyl)carbamoyl)-3,6,12,15,18,24,27-heptaazanonacosanoic acid

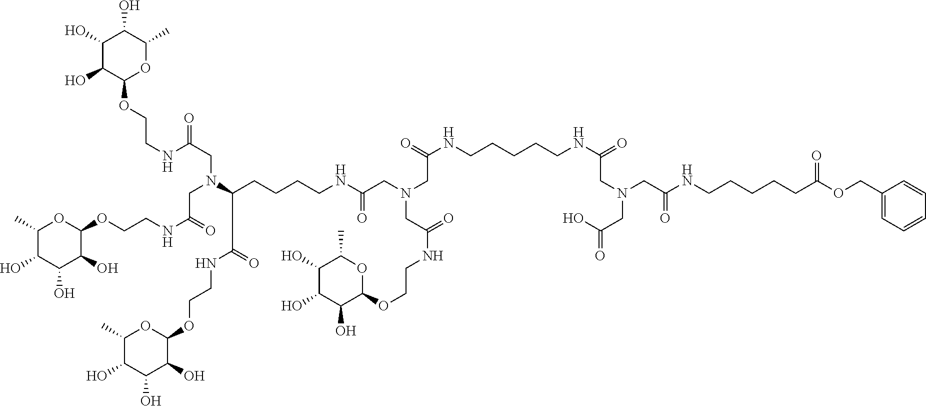 [SMILES: C[C@@H]1O[C@@H](OCCNC(=O)CN(CC(=O)NCCCCCNC(=O)CN(CC(=O)O)CC(=O)NCCCCCC(=O)OCc2ccccc2)CC(=O)NCCCC[C@@H](C(=O)NCCO[C@@H]2O[C@@H](C)[C@@H](O)[C@@H](O)[C@@H]2O)N(CC(=O)NCCO[C@@H]2O[C@@H](C)[C@@H](O)[C@@H](O)[C@@H]2O)CC(=O)NCCO[C@@H]2O[C@@H](C)[C@@H](O)[C@@H](O)[C@@H]2O)[C@@H](O)[C@H](O)[C@@H]1O]

### Step 1: Benzyl ((S)-4,13,17-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((2-(((2S,3R,4S,5R,6R)-3,4,5-trihydroxy-6-methyltetrahydro-2H-pyran-2-yl)oxy)ethyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18-pentaazatricosan-23-yl)carbamate

To a solution of 3,11-dioxo-13-(2-oxo-2-((2-(((2S,3R,4S,5R,6R)-3,4,5-trihydroxy-6-methyltetrahydro-2H-pyran-2-yl)oxy)ethyl)amino)ethyl)-1-phenyl-2-oxa-4,10,13-triazapentadecan-15-oic acid (100 mg, 0.167 mmol) in DMF (20 mL) was added HATU (95 mg, 0.251 mmol), and TEA (0.058 mL, 0.418 mmol) was added to the solution. Then, 2,2′-(((S)-6-amino-1-oxo-1-((2-(((2R,3S,4R,5S,6S)-3,4,5-trihydroxy-6-methyltetrahydro-2H-pyran-2-yl)oxy)ethyl)amino)hexan-2-yl)azanediyl)bis(N-(2-(((2R,3S,4R,5S,6S)-3,4,5-trihydroxy-6-methyltetrahydro-2H-pyran-2-yl)oxy)ethyl)acetamide) (208 mg, 0.251 mmol) was added, and the reaction was stirred for 16 h. The reaction mixture was concentrated and purified on C18 reverse phase chromatography to isolate title compound. UPLC-MS Method A: m/z=1411.5 (z=1); t_{R}=3.38 min.

### Step 2: 2,2′-(((S)-21-Amino-4,11,15-trioxo-13-(2-oxo-2-((2-(((2S,3R,4S,5R,6R)-3,4,5-trihydroxy-6-methyltetrahydro-2H-pyran-2-yl)oxy)ethyl)amino)ethyl)-1-(((2R,3S,4R,5S,6S)-3,4,5-trihydroxy-6-methyltetrahydro-2H-pyran-2-yl)oxy)-3,10,13,16-tetraazahenicosan-5-yl)azanediyl)bis(N-(2-(((2R,3S,4R,5S,6S)-3,4,5-trihydroxy-6-methyltetrahydro-2H-pyran-2-yl)oxy)ethyl)acetamide)

The title compound was prepared using procedures analogous to those described for Intermediate 5, Step 2, substituting benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate for benzyl ((S)-4,13,17-trioxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((2-(((2S,3R,4S,5R,6R)-3,4,5-trihydroxy-6-methyltetrahydro-2H-pyran-2-yl)oxy)ethyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18-pentaazatricosan-23-yl)carbamate to isolate title compound. UPLC-MS Method A: m/z=1276.5 (z=1); t_{R}=1.10 min.

### Step 3: (S)-3-(2-((6-(Benzyloxy)-6-oxohexyl)amino)-2-oxoethyl)-5,13,17,26-tetraoxo-24-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((2-(((2S,3R,4S,5R,6R)-3,4,5-trihydroxy-6-methyltetrahydro-2H-pyran-2-yl)oxy)ethyl) amino)ethyl)-29-(((2R,3S,4R,5S,6S)-3,4,5-trihydroxy-6-methyltetrahydro-2H-pyran-2-yl)oxy)-23-((2-(((2R,3S,4R,5S,6S)-3,4,5-trihydroxy-6-methyltetrahydro-2H-pyran-2-yl)oxy)ethyl)carbamoyl)-3,6,12,15,18,24,27-heptaazanonacosanoic acid

The title compound was prepared from 2,2′-((2-((6-(benzyloxy)-6-oxohexyl) amino)-2-oxoethyl)azanediyl)diacetic acid and 2,2′-(((S)-21-amino-4,11,15-trioxo-13-(2-oxo-2-((2-(((2S,3R,4S,5R,6R)-3,4,5-trihydroxy-6-methyltetrahydro-2H-pyran-2-yl)oxy)ethyl)amino) ethyl)-1-(((2R,3S,4R,5S,6S)-3,4,5-trihydroxy-6-methyltetrahydro-2H-pyran-2-yl)oxy)-3,10,13,16-tetraazahenicosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) using the procedure described for Intermediate 6. UPLC-MS Method A: m/z=1652.6 (z=1); t_{R}=3.68 min.

### Intermediate 9: 3,11-Dioxo-13-(2-oxo-2-((2-(((2R,3S,4R,5S,6S)-3,4,5-trihydroxy-6-methyltetrahydro-2H-pyran-2-yl)oxy)ethyl)amino)ethyl)-1-phenyl-2-oxa-4,10,13-triazapentadecan-15-oic acid

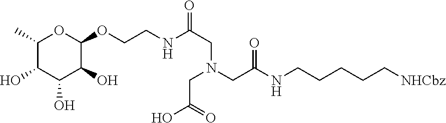 [SMILES: C[C@@H]1O[C@@H](OCCNC(=O)CN(CC(=O)O)CC(=O)NCCCCCNC(=O)OCc2ccccc2)[C@@H](O)[C@H](O)[C@@H]1O]

To a suspension of 13-(carboxymethyl)-3,11-dioxo-1-phenyl-2-oxa-4,10,13-triazapentadecan-15-oic acid (0.5 g, 1.221 mmol) in DCM (8 mL) at 0° C. was added TFAA (0.216 mL, 1.526 mmol). After stirring at 0° C. for 3 h, the mixture was cooled to −30° C. To the solution was added dropwise a solution of TEA (0.409 mL, 2.93 mmol) in DMF (6 mL) over 30 min. To the resulting mixture was added a solution of AEF (3.04 g, 3.66 mmol) in DMF (8 mL). After stirring at rt for 16 h, the mixture was concentrated, and the residue was purified by C18 reverse phase chromatography to isolate title compound. UPLC-MS Method A: m/z=599.1 (z1); t_{R}=3.69 min.

### Intermediate 10: (S)-3-(2-((6-(Benzyloxy)-6-oxohexyl)amino)-2-oxoethyl)-5,14-dioxo-12-(2-oxo-2-((2-(((2R,3S,4R,5S,6S)-3,4,5-trihydroxy-6-methyltetrahydro-2H-pyran-2-yl)oxy)ethyl)amino) ethyl)-17-(((2R,3S,4R,5S,6S)-3,4,5-trihydroxy-6-methyltetrahydro-2H-pyran-2-yl)oxy)-11-((2-(((2R,3S,4R,5S,6S)-3,4,5-trihydroxy-6-methyltetrahydro-2H-pyran-2-yl)oxy)ethyl)carbamoyl)-3,6,12,15-tetraazaheptadecanoic acid

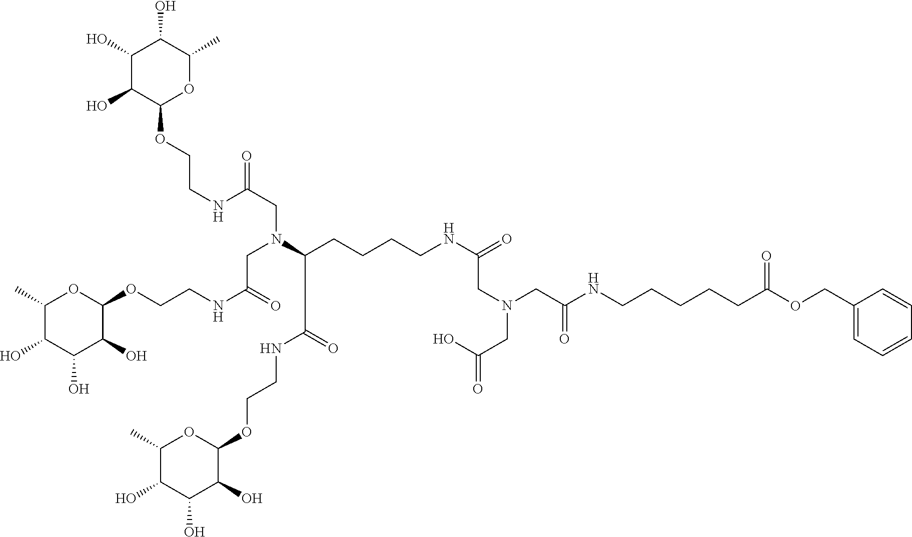 [SMILES: C[C@@H]1O[C@@H](OCCNC(=O)CN(CC(=O)NCCO[C@@H]2O[C@@H](C)[C@@H](O)[C@@H](O)[C@@H]2O)[C@@H](CCCCNC(=O)CN(CC(=O)O)CC(=O)NCCCCCC(=O)OCc2ccccc2)C(=O)NCCO[C@@H]2O[C@@H](C)[C@@H](O)[C@@H](O)[C@@H]2O)[C@@H](O)[C@H](O)[C@@H]1O]

The title compound was prepared using procedures analogous to those described for Intermediate 6, substituting 2,2′-(((S)-6-amino-1-oxo-1-((2-(((2R,3S,4R,5S,6S)-3,4,5-trihydroxy-6-methyltetrahydro-2H-pyran-2-yl)oxy)ethyl)amino)hexan-2-yl)azanediyl)bis(N-(2-(((2R,3S,4R,5S,6S)-3,4,5-trihydroxy-6-methyltetrahydro-2H-pyran-2-yl)oxy)ethyl)acetamide) for 6-amino-N-(2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl) hexanamide to isolate the title compound. UPLC-MS Method A: m/z=1206.5 (z=1); t_{R}=3.74 min.

### Intermediate 11: 3,11-Dioxo-13-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-phenyl-2-oxa-4,10,13-triazapentadecan-15-oic acid

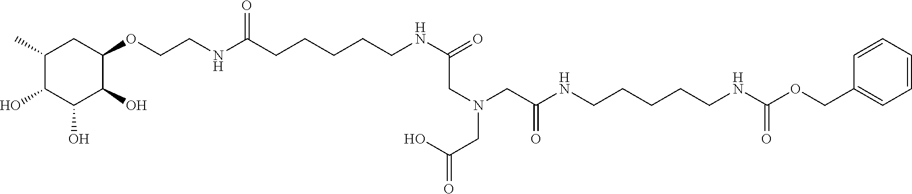 [SMILES: C[C@@H]1C[C@@H](OCCNC(=O)CCCCCNC(=O)CN(CC(=O)O)CC(=O)NCCCCCNC(=O)OCc2ccccc2)[C@@H](O)[C@H](O)[C@@H]1O]

The title compound was prepared using procedures analogous to those described for Intermediate 9, substituting 6-amino-N-(2-(((2R,3S,4R,5S,6S)-3,4,5-trihydroxy-6-methyltetrahydro-2H-pyran-2-yl)oxy)ethyl)hexanamide for AEF to isolate title compound. Method A: m/z=712.3 (z=1); t_{R}=4.18 min.

### Intermediate 12: 6-(2-((2-((5-Aminopentyl)amino)-2-oxoethyl)(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)amino)acetamido)-N-(2-(((2R,3S,4R,5S,6S)-3,4,5-trihydroxy-6-methyltetrahydro-2H-pyran-2-yl)oxy)ethyl)hexanamide

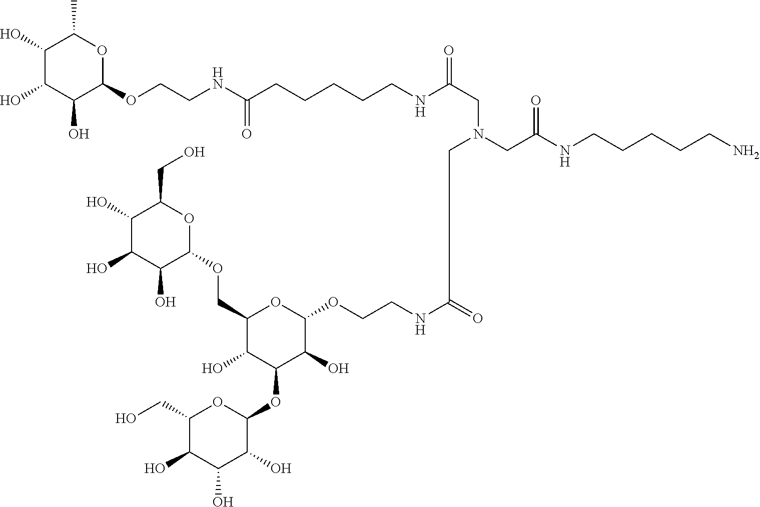 [SMILES: C[C@@H]1O[C@@H](OCCNC(=O)CCCCCNC(=O)CN(CC(=O)NCCCCCN)CC(=O)NCCO[C@H]2O[C@H](CO[C@H]3O[C@H](CO)[C@@H](O)[C@H](O)[C@@H]3O)[C@@H](O)[C@H](O[C@@H]3O[C@@H](CO)[C@H](O)[C@@H](O)[C@H]3O)[C@@H]2O)[C@@H](O)[C@H](O)[C@@H]1O]

### Step 1: Benzyl (13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,11,15-trioxo-1-(((2R,3S,4R,5S,6S)-3,4,5-trihydroxy-6-methyltetrahydro-2H-pyran-2-yl)oxy)-3,10,13,16-tetraazahenicosan-21-yl)carbamate

To a solution of 3,11-dioxo-13-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-phenyl-2-oxa-4,10,13-triazapentadecan-15-oic acid (136 mg, 0.191 mmol) in DMF (2 mL) at rt was added EDC (47.6 mg, 0.248 mmol). HOBT (38.0 mg, 0.248 mmol), and TEA (66.6 μL, 0.478 mmol). After stirring 30 min, a solution of AETM (136 mg, 0.248 mmol) in DMF (2 mL) was added dropwise. After stirring for 16 h, the mixture was concentrated, and the residue was purified by C18 reverse phase chromatography to provide the title compound. UPLC-MS Method A: m/z=1241.4 (z=1); t_{R}=3.60.

### Step 2: 6-(2-((2-((5-Aminopentyl)amino)-2-oxoethyl)(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)amino)acetamido)-N-(2-(((2R,3S,4R,5S,6S)-3,4,5-trihydroxy-6-methyltetrahydro-2H-pyran-2-yl)oxy)ethyl)hexanamide

The title compound was prepared using procedures analogous to those described for Intermediate 5, Step 2, substituting benzyl (13-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-2-oxoethyl)-4,11,15-trioxo-1-(((2R,3S,4R,5S,6S)-3,4,5-trihydroxy-6-methyltetrahydro-2H-pyran-2-yl)oxy)-3,10,13,16-tetraazahenicosan-21-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) carbamate to isolate title compound. UPLC-MS Method A: m/z=1107.8 (z=1); t_{R}=0.67 min.

### Intermediate 13: (S)-2-Amino-N1,N5-bis(2-(((2R,3S,4R,5S,6S)-3,4,5-trihydroxy-6-methyltetralhydro-2H-pyran-2-yl)oxy)ethyl)pentanediamide

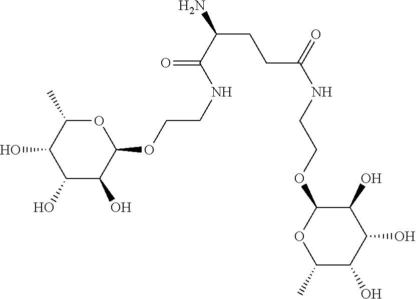 [SMILES: C[C@@H]1O[C@@H](OCCNC(=O)CC[C@H](N)C(=O)NCCO[C@@H]2O[C@@H](C)[C@@H](O)[C@@H](O)[C@@H]2O)[C@@H](O)[C@H](O)[C@@H]1O]

### Step 1: Benzyl ((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)carbamate

To a solution of Z-GLU-OH (500 mg, 1.778 mmol) in DMF (10 mL) at rt was added EDC (1022 mg, 5.33 mmol), HOBT (817 mg, 5.33 mmol), and TEA (743 μL, 5.33 mmol). After stirring 30 min, to the reaction mixture was added a solution of AEF (257 mg, 0.488 mmol) in DMF (10 mL) was added dropwise. After stirring for 16 h, the mixture was concentrated, and the residue was purified by C18 reverse phase chromatography to provide the title compound. UPLC-MS Method A: m/z=660.2 (z=1); t_{R}=3.08 min.

### Step 2: (S)-2-amino-N1,N5-bis(2-(((2R,3S,4R,5S,6S)-3,4,5-trihydroxy-6-methyltetrahydro-2H-pyran-2-yl)oxy)ethyl)pentanediamide

The title compound was prepared using procedures analogous to those described for Intermediate 5, Step 2, substituting benzyl ((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)carbamate to isolate title compound. UPLC-MS Method A: m/z=526.2 (z=1); t_{R}=1.03 min.

### Intermediate 14: (2S,2′S)-2,2′-((2,2′-((2-((5-Aminopentyl)amino)-2-oxoethyl)azanediyl) bis(acetyl))bis(azanediyl))bis(N1,N5-bis(2-(((2R,3S,4R,5S,6S)-3,4,5-trihydroxy-6-methyltetrahydro-2H-pyran-2-yl)oxy)ethyl)pentanediamide)

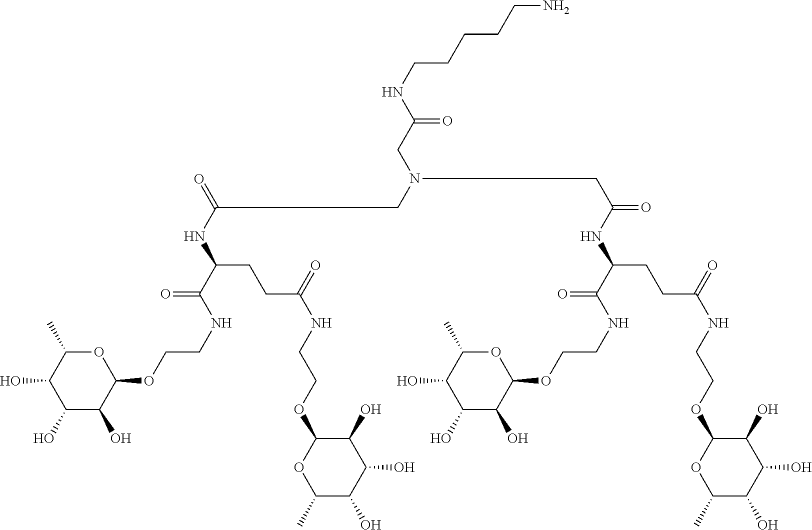 [SMILES: C[C@@H]1O[C@@H](OCCNC(=O)CC[C@H](NC(=O)CN(CC(=O)NCCCCCN)CC(=O)N[C@@H](CCC(=O)NCCO[C@@H]2O[C@@H](C)[C@@H](O)[C@@H](O)[C@@H]2O)C(=O)NCCO[C@@H]2O[C@@H](C)[C@@H](O)[C@@H](O)[C@@H]2O)C(=O)NCCO[C@@H]2O[C@@H](C)[C@@H](O)[C@@H](O)[C@@H]2O)[C@@H](O)[C@H](O)[C@@H]1O]

### Step 1: Benzyl ((S)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-2-oxoethyl)-4,9,13-trioxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,11,14-tetraazanonadecane-19-yl)carbamate

To a solution of 13-(carboxymethyl)-3,11-dioxo-1-phenyl-2-oxa-4,10,13-triazapentadecan-15-oic acid (100 mg, 0.488 mmol) in DMF (10 mL) at rt was added EDC (103 mg, 0.537 mmol), HOBT (82.0 mg, 0.537 mmol), and TEA (102 μL, 0.733 mmol). After stirring 30 min. a solution of (S)-2-amino-N1,N5-bis(2-(((2R,3S,4R,5S,6S)-3,4,5-trihydroxy-6-methyltetrahydro-2H-pyran-2-yl)oxy)ethyl)pentane diamide (257 mg, 0.488 mmol) in DMF (2 mL) was added to the reaction mixture dropwise. After stirring for 16 h, the mixture was concentrated, and the residue was purified by C18 reverse phase chromatography to provide the title compound. UPLC-MS Method A: m/z=1424.4 (z=1); t_{R}=3.38 min.

### Step 2: (2S,2′S)-2,2′-((2,2′-((2-((5 Aminopentyl)amino)-2-oxoethyl)azanediyl)bis(acetyl)) bis(azanediyl))bis(N1,N5-bis(2-(((2R,3S,4R,5S,6S)-3,4,5-trihydroxy-6-methyltetrahydro-2H-pyran-2-yl)oxy)ethyl)pentanediamide)

The title compound was prepared using procedures analogous to those described for Intermediate 5, Step 2, substituting benzyl ((S)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-2-oxoethyl)-4,9,13-trioxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,11,14-tetraazanonadecan-19-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl) carbamate to isolate title compound. UPLC-MS Method A: m/z=1290.5 (z=1); t_{R}=2.81 mm.

### Intermediate 15: 2,2′—(((R)-6-Amino-1-oxo-1-((2-(((2S,3S,4S,5S,6R)-3,4,5-trihydroxy-6-(hydroxymethyl)tetrahydro-2H-pyran-2-yl)oxy)ethyl)amino)hexan-2-yl)azanediyl)bis(N-(2-(((2S,3S,4S,5S,6R)-3,4,5-trihydroxy-6-(hydroxymethyl)tetrahydro-2H-pyran-2-yl)oxy)ethyl) acetamide)

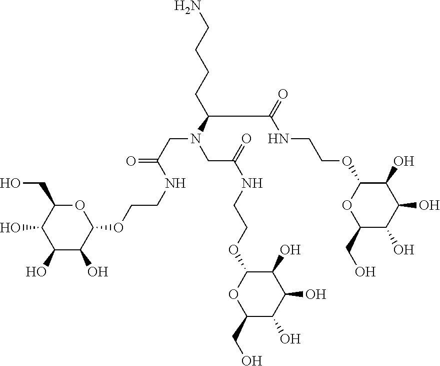 [SMILES: NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O]

### Step 1: Benzyl ((S)-5-(bis(2-oxo-2-((2-(((2S,3S,4S,5S,6R)-3,4,5-trihydroxy-6-(hydroxymethyl) tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)carbamate

The title compound was prepared using procedures analogous to those described for Intermediate 2, Step 1, substituting AEM for AEF AEM to isolate the title compound. UPLC-MS Method A: m/z=1012.3 (z=1); t_{R}=2.87 min.

### Step 2: 2, 2′-(((S)-6-Amino-1-oxo-1-((2-(((2S,3S,4S,5S,6R)-3,4,5-trihydroxy-6-(hydroxymethyl) tetrahydro-2H-pyran-2-yl)oxy)ethyl)amino)hexan-2-yl)azanediyl)bis(N-(2-(((2S,3S,4S,5S,6R)-3,4,5-trihydroxy-6-(hydroxymethyl)tetrahydro-2H-pyran-2-yl)oxy)ethyl)acetamide)

The title compound was prepared using procedures analogous to those described for Intermediate 5, Step 2, substituting benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)carbamate for benzyl ((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)carbamate to isolate title compound. UPLC-MS Method A: m/z=878.3 (z=1); t_{R}=1.00 min.

### Intermediate 16: 3,11-Dioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)ethyl)-1-phenyl-2-oxa-4,10,13-triazapentadecan-15-oic acid

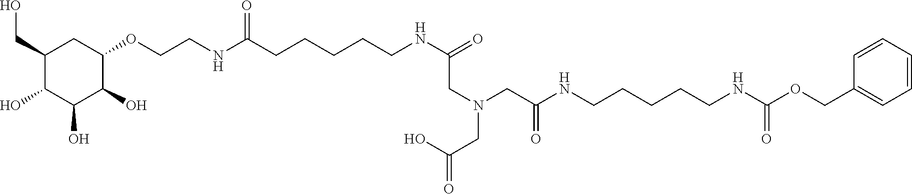 [SMILES: O=C(O)CN(CC(=O)NCCCCCNC(=O)OCc1ccccc1)CC(=O)NCCCCCC(=O)NCCO[C@H]1C[C@H](CO)[C@@H](O)[C@H](O)[C@@H]1O]

The title compound was prepared using procedures analogous to those described for Intermediate 9, substituting 6-amino-N-(2-(((2S,3S,4S,5S,6R)-3,4,5-trihydroxy-6-(hydroxymethyl)tetrahydro-2H-pyran-2-yl)oxy)ethyl)hexanamide for AEF to isolate the title compound. UPLC-MS Method A: m/z=727.8 (z=1); t_{R}=4.84 min.

### Intermediate 17: 2,2′-(((S)-13-(2-((5-Aminopentyl)amino)-2-oxoethyl)-4,11,15,22-tetraoxo-1,25-bis(((2S,3S,4S,5S,6R)-3,4,5-trihydroxy-6-(hydroxymethyl)tetrahydro-2H-pyran-2-yl)oxy)-3,10,13,16,23-pentaazapentacosan-5-yl)azanediyl)bis(N-(2-(((2S,3S,4S,5S,6R)-3,4,5-trihydroxy-6-(hydroxymethyl)tetrahydro-2H-pyran-2-yl)oxy)ethyl)acetamide)

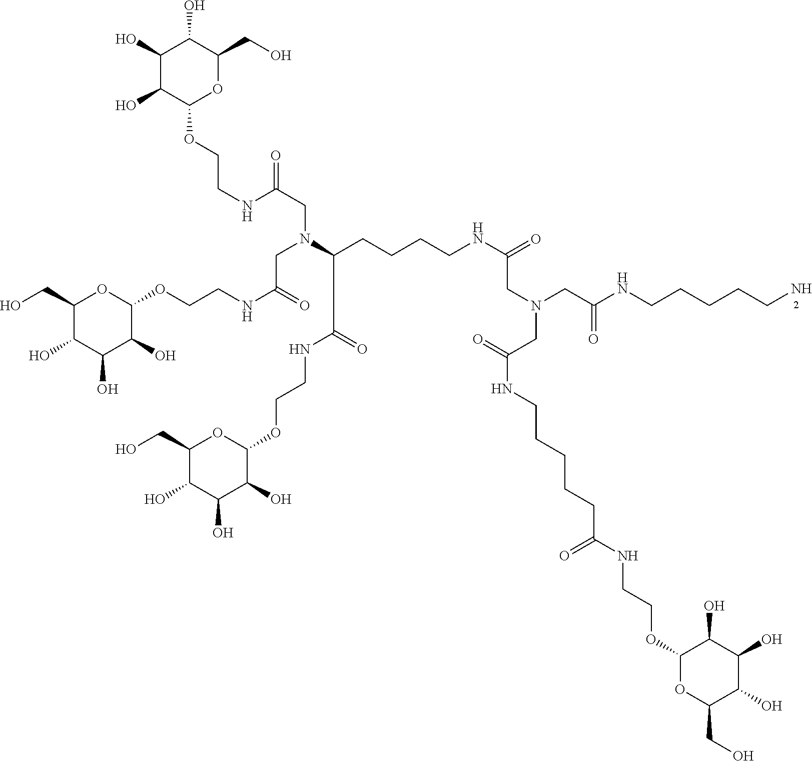 [SMILES: NCCCCCNC(=O)CN(CC(=O)NCCCCCC(=O)NCCO[C@H]1O[C@H](CO)[C@@H](O)[C@H](O)[C@@H]1O)CC(=O)NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O]

### Step 1: Benzyl ((S)-4,13,17-trioxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-15-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,15,18-pentaazatricosan-23-yl)carbamate

To a solution of 3,11-dioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)ethyl)-1-phenyl-2-oxa-4,10,13-triazapentadecan-15-oic acid (190 mg, 0.392 mmol) in DMF (2 mL) at rt was added EDC (50 mg, 0.261 mmol), HOBT (40.0 mg, 0.261 mmol), and TEA (36.4 μL, 0.261 mmol). After stirring 30 min. a solution of 2,2′-(((S)-6-amino-1-oxo-1-((2-(((2S,3S,4S,5S,6R)-3,4,5-trihydroxy-6-(hydroxymethyl)tetrahydro-2H-pyran-2-yl)oxy)ethyl) amino)hexan-2-yl)azanediyl)bis(N-(2-(((2S,3S,4S,5S,6R)-3,4,5-trihydroxy-6-(hydroxymethyl) tetrahydro-2H-pyran-2-yl)oxy)ethyl)acetamide) (344 mg, 0.392 mmol) in DMF (2 mL) was added dropwise. After stirring for 16 h, the mixture was concentrated, and the residue was purified by C18 reverse phase chromatography to provide the title compound. UPLC-MS Method A: m/z=1588.5 (z=1); t_{R}=4.00 min.

### Step 2: 2, 2′-(((S)-13-(2-((5-Aminopentyl)amino)-2-oxoethyl)-4,11,15,22-tetraoxo-1,25-bis(((2S,3S,4S,5S,6R)-3,4,5-trihydroxy-6-(hydroxymethyl)tetrahydro-2H-pyran-2-yl)oxy)-3,10,13,16,23-pentaazapentacosan-5-yl)azanediyl)bis(N-(2-(((2S,3S,4S,5S,6R)-3,4,5-trihydroxy-6-(hydroxymethyl)tetrahydro-2H-pyran-2-yl)oxy)ethyl)acetamide)

The title compound was prepared using procedures analogous to those described for Intermediate 5, Step 2, substituting benzyl ((S)-4,13,17-trioxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-15-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,15,18-pentaazatricosan-23-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate to isolate title compound. UPLC-MS Method A: m/z=1453.5 (z=1); t_{R}=2.09 min.

### Intermediate 18: (S)-3-(2-((5-(((Benzyloxy)carbonyl)amino)pentyl)amino)-2-oxoethyl)-5,14-dioxo-12-(2-oxo-2-((2-(((2R,3S,4R,5S,6S)-3,4,5-trihydroxy-6-methyltetrahydro-2H-pyran-2-yl)oxy)ethyl)amino)ethyl)-17-(((2R,3S,4R,5S,6S)-3,4,5-trihydroxy-6-methyltetrahydro-2H-pyran-2-yl)oxy)-11-((2-(((2R,3S,4R,5S,6S)-3,4,5-trihydroxy-6-methyltetrahydro-2H-pyran-2-yl)oxy)ethyl)carbamoyl)-3,6,12,15-tetraazakeptadecanoic acid

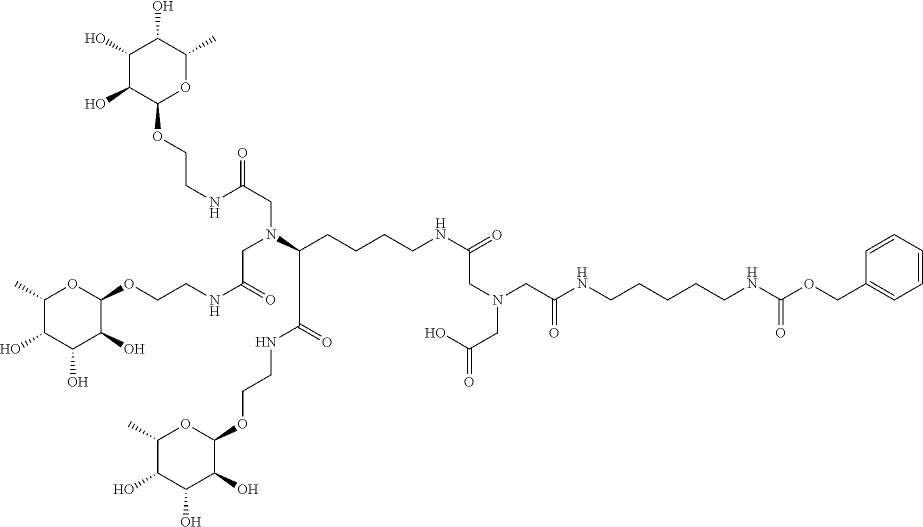 [SMILES: C[C@@H]1O[C@@H](OCCNC(=O)CN(CC(=O)NCCO[C@@H]2O[C@@H](C)[C@@H](O)[C@@H](O)[C@@H]2O)[C@@H](CCCCNC(=O)CN(CC(=O)O)CC(=O)NCCCCCNC(=O)OCc2ccccc2)C(=O)NCCO[C@@H]2O[C@@H](C)[C@@H](O)[C@@H](O)[C@@H]2O)[C@@H](O)[C@H](O)[C@@H]1O]

The title compound was prepared using a procedures analogous to those described for Intermediate 9, substituting 2,2′-(((S)-6-amino-1-oxo-1-((2-(((2R,3S,4R,5S,6S)-3,4,5-trihydroxy-6-methyltetrahydro-2H-pyran-2-yl)oxy)ethyl)amino)hexan-2-yl)azanediyl)bis(N-(2-(((2R,3S,4R,5S,6S)-3,4,5-trihydroxy-6-methyltetrahydro-2H-pyran-2-yl)oxy)ethyl)acetamide) for AEF to isolate title compound. Method A: m/z=1221.7 (z=1); t_{R}=4.70 min.

### Intermediate 19: 6-Amino-N-(2-(((2S,3R,4S,5S,6R)-3,4,5-trihydroxy-6-(hydroxymethyl) tetrahydro-2H-pyran-2-yl)oxy)ethyl)hexanamide

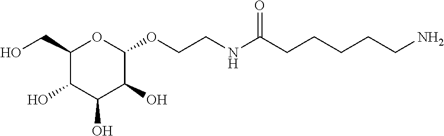 [SMILES: NCCCCCC(=O)NCCO[C@H]1O[C@H](CO)[C@@H](O)[C@H](O)[C@@H]1O]

### Step 1: Benzyl (6-oxo-6-((2-(((2S,3R,4S,5S,6R)-3,4,5-trihydroxy-6-(hydroxymethyl)tetrahydro-2H-pyran-2-yl)oxy)ethyl)amino)hexyl)carbamate

The title compound was prepared using procedures analogous to those described for Intermediate 4, Step 1, substituting AEG for AEM to isolate title compound. UPLC-MS Method A: m/z=471.2 (z=1); t_{R}=2.69 min.

### Step 2: 6-Amino-N-(2-(((2S,3R,4S,5S,6R)-3,4,5-trihydroxy-6-(hydroxymethyl)tetrahydro-2H-pyran-2-yl)oxy)ethyl)hexanamide

The title compound was prepared using procedures analogous to those described for Intermediate 5, Step 2, substituting benzyl (6-oxo-6-((2-(((2S,3R,4S,5S,6R)-3,4,5-trihydroxy-6-(hydroxymethyl)tetrahydro-2H-pyran-2-yl)oxy)ethyl)amino)hexyl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl)carbamate to isolate title compound. Method A: m/z=337.3 (z=1); t_{R}=0.84 min.

### Intermediate 20: (S)-3-(2-((6-(Benzyloxy)-6-oxohexyl)amino)-2-oxoethyl)-5,14-dioxo-12-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-17-(((2S,3S,4S,5S,6R)-3,4,5-trihydroxy-6-(hydroxymethyl) tetrahydro-2H-pyran-2-yl)oxy)-11-((2-(((2S,3S,4S,5S,6R)-3,4,5-trihydroxy-6-(hydroxymethyl) tetrahydro-2H-pyran-2-yl)oxy)ethyl)carbamoyl)-3,6,12,15-tetraazaheptadecanoic acid

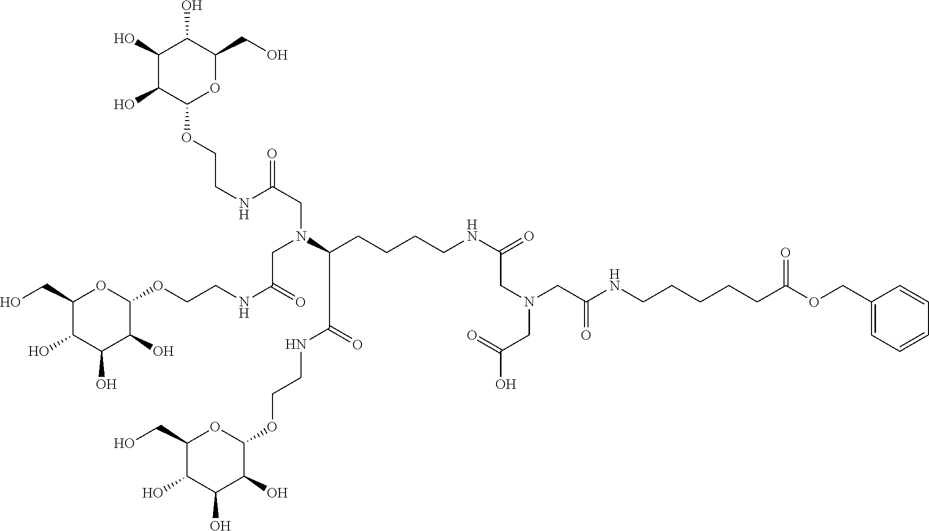 [SMILES: O=C(O)CN(CC(=O)NCCCCCC(=O)OCc1ccccc1)CC(=O)NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O]

The title compound was prepared using procedures analogous to those described for Intermediate 6, substituting 2,2′-(((S)-6-amino-1-oxo-1-((2-(((2S,3S,4S,5S,6R)-3,4,5-trihydroxy-6-(hydroxymethyl)tetrahydro-2H-pyran-2-yl)oxy)ethyl)amino)hexan-2-yl)azanediyl) bis(N-(2-(((2S,3S,4S,5S,6R)-3,4,5-trihydroxy-6-(hydroxymethyl)tetrahydro-2H-pyran-2-yl)oxy)ethyl)acetamide) for 6-amino-N-(2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)hexanamide to isolate the title compound. UPLC-MS Method A: m/z=1254.6 (z=1); t_{R}=4.68 min.

### Intermediate 21: N-(2-((6-(Benzyloxy)-6-oxohexyl)amino)-2-oxoethyl)-N-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)glycine

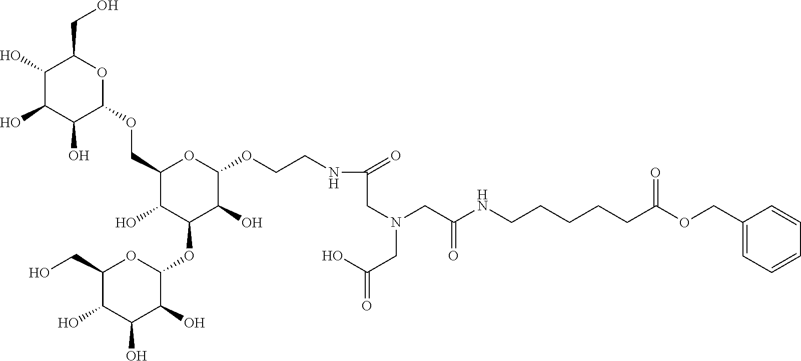 [SMILES: O=C(O)CN(CC(=O)NCCCCCC(=O)OCc1ccccc1)CC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

To a solution of 2,2′-((2-((6-(benzyloxy)-6-oxohexyl)amino)-2-oxoethyl)azanediyl) diacetic acid (750 mg, 1.9 mmol) in anhydrous DCM (8 mL) cooled in an ice bath was added trifluoroacetic anhydride (0.336 mL, 2.38 mmol), and the mixture was stirred at ice bath temperature for 3 h. The mixture was cooled to −15° C., and a solution of TEA (0.636 mL, 4.56 mmol) in DMF (4 mL) added slowly over 20 min. After complete addition, the mixture was stirred at −15° C. for a further 30 min, then warmed to rt and a solution of AETM (1.25 g, 2.28 mmol) in DMF (8 mL) added and stirred for 1 h. The mixture evaporated and purified by C18 reverse phase chromatography to isolate the title material. UPLC-MS Method A: m/z=924.4 (z=1); t_{R}=4.68 min.

### Intermediate 22: 6-Amino-N-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)hexanamide

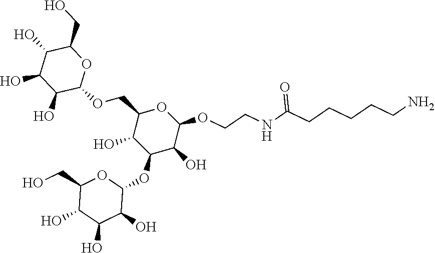 [SMILES: NCCCCCC(=O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

The title compound was prepared using procedures analogous to those described for Intermediate 5, using beta-AETM instead of AETM. UPLC-MS Method A: m/z=661.3 (z=1); t_{R}=1.14 min.

### Intermediate 23: N²-((Benzyloxy)carbonyl)-N⁵—((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)-L-glutamine

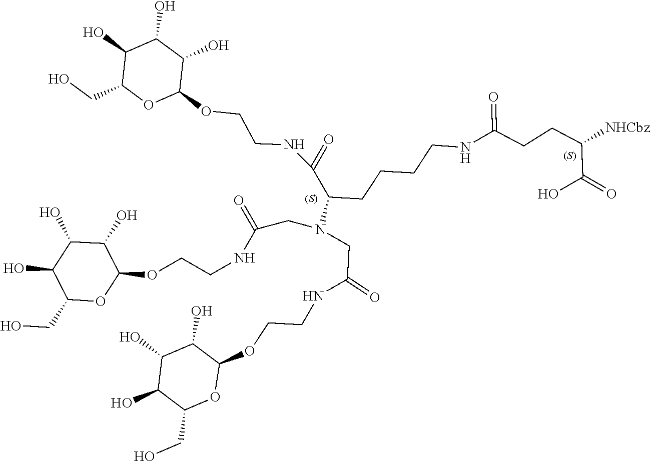

### Step 1: Benzyl N²-((benzyloxy)carbonyl)-N⁵—((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)-L-glutaminate

To a solution of 2,2′-(((S)-6-amino-1-oxo-1-((2-(((2S,3S,4S,5S,6R)-3,4,5-trihydroxy-6-(hydroxymethyl)tetrahydro-2H-pyran-2-yl)oxy)ethyl)amino)hexan-2-yl)azanediyl)bis(N-(2-(((2S,3S,4S,5S,6R)-3,4,5-trihydroxy-6-(hydroxymethyl)tetrahydro-2H-pyran-2-yl)oxy)ethyl) acetamide) (2.84 g, 3.23 mmol) and (S)-5-(benzyloxy)-4-(((benzyloxy)carbonyl)amino)-5-oxopentanoic acid (1 g, 2.69 mmol) in anhydrous DMF (8 mL) was added EDC (774 mg, 4.04 mmol), HOBT (41 mg, 0.269 mmol) and DIPEA (0.047 mL, 0.269 mmol), and the resulting mixture was stirred at rt overnight. The mixture was evaporated, and the residue was purified by C18 reverse phase chromatography to isolate title compound. UPLC-MS Method A: m/z=1231.6 (z=1); t_{R}=3.68 min.

### Step 2: N²-((Benzyloxy)carbonyl)-N⁵—((S)-5-(bis(2-oxo-2-((2-(((2S,3S4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)-L-glutamine

To a solution of benzyl N²-((benzyloxy)carbonyl)-N⁵—((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl) amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)-L-glutaminate (2.8 g, 2.27 mmol) in water (15 mL) was added 1N aq. NaOH solution (2.274 mL, 2.27 mmol), and the mixture was stirred at rt for 1 h. AcOH (0.169 mL, 2.96 mmol) added, and the mixture was purified directly by C18 reverse phase chromatography to isolate title compound. UPLC-MS Method A: m/z=1141.1 (z=1); t_{R}=3.81 min.

### Intermediate 24: N²-((Benzyloxy)carbonyl)-N—((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)-L-glutamine

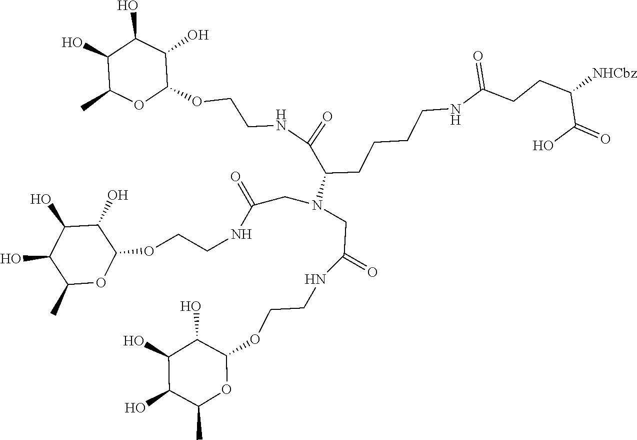 [SMILES: C[C@@H]1O[C@@H](OCCNC(=O)CN(CC(=O)NCCO[C@@H]2O[C@@H](C)[C@@H](O)[C@@H](O)[C@@H]2O)[C@@H](CCCCNC(=O)CC[C@H](NC(=O)OCc2ccccc2)C(=O)O)C(=O)NCCO[C@@H]2O[C@@H](C)[C@@H](O)[C@@H](O)[C@@H]2O)[C@@H](O)[C@H](O)[C@@H]1O]

### Step 1: Benzyl N²-((benzyloxy)carbonyl)-N⁵—((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)-L-glutaminate

To a solution of 2,2′-(((S)-6-amino-1-oxo-1-((2-(((2R,3S,4R,5S,6S)-3,4,5-trihydroxy-6-methyltetrahydro-2H-pyran-2-yl)oxy)ethyl)amino)hexan-2-yl)azanediyl)bis(N-(2-(((2R,3S,4R,5S,6S)-3,4,5-trihydroxy-6-methyltetrahydro-2H-pyran-2-yl)oxy)ethyl)acetamide) (2.68 g, 3.23 mmol) and (S)-5-(benzyloxy)-4-(((benzyloxy)carbonyl)amino)-5-oxopentanoic acid (1 g, 2.69 mmol) in anhydrous DMF (8 mL) was added EDC (774 mg, 4.04 mmol), HOBT (41 mg, 0.269 mmol) and DIPEA (0.047 mL, 0.269 mmol), and the resulting mixture was stirred at rt for 3 days. The mixture was evaporated, and the residue was purified by C18 reverse phase chromatography to isolate title compound. UPLC-MS Method A: m/z=1183 (z=1); t_{R}=3.67 min.

### Step 2: N²-((Benzyloxy)carbonyl)-N⁵—((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)-L-glutamine

To a solution of benzyl N²-((benzyloxy)carbonyl)-N⁵—((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl) amino)hexyl)-L-glutaminate (1.61 g, 1.36 mmol) in water (10 mL) was added 1N aq. NaOH solution (1.36 mL, 1.36 mmol), and the mixture was stirred at rt for 90 min. AcOH (0.093 mL, 1.63 mmol) was added, and the mixture was purified by C18 reverse phase chromatography to isolate title compound. UPLC-MS Method A: m/z=1093.5 (z=1); t_{R}=4.24 min.

### Intermediate 25: 6-Amino-N—((S)-1,4-bis(((2S,3S,4S,5S,6R)-3,4,5-trihydroxy-6-(hydroxymethyl) tetrahydro-2H-pyran-2-yl)oxy)butan-2-yl)hexanamide

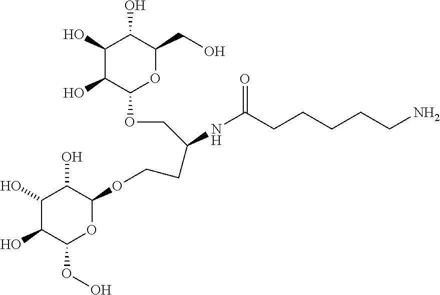 [SMILES: NCCCCCC(=O)N[C@@H](CCO[C@H]1O[C@H](OO)[C@@H](O)[C@H](O)[C@@H]1O)CO[C@H]1O[C@H](CO)[C@@H](O)[C@H](O)[C@@H]1O]

### Step 1: Benzyl (6-(((S)-1,4-bis(((2S,3S,4S,5S,6R)-3,4,5-trihydroxy-6-(hydroxymethyl)tetrahydro-2H-pyran-2-yl)oxy)butan-2-yl)amino)-6-oxohexyl)carbamate

(2R,2′R,3S,3′S,4S,4′S,5S,5′S,6S,6′S)-6,6′-(((S)-2-aminobutane-1,4-diyl)bis(oxy))bis(2-(hydroxymethyl)tetrahydro-2H-pyran-3,4,5-triol) (WO2015051052A2, 1.2 g, 2.79 mmol) was dissolved in anhydrous DMF (16 mL) under N_{2(g)}, and the solution was cooled to 0° C. To the solution was added 2,5-dioxopyrrolidin-1-yl 6-(((benzyloxy) carbonyl)amino)hexanoate (1.013 g, 2.79 mmol) in DMF (8 mL) followed by dropwise addition of TEA (0.382 mL, 2.79 mmol) over 10 min. The mixture was stirred at rt under N_{2(g)} overnight. DMF was removed by rotary evaporation under reduced pressure at 40° C. The residue was purified by column chromatography on 130 g C18 reverse phase column, gradient 0%-30% ACN/water. The fractions were combined and lyophilized to yield the title compound. UPLC-MS Method B: m/z=677.3 (z=1); t_{R}=2.60 min.

### Step 2: 6-Amino-N—((S)-1,4-bis(((2S,3S,4S,5S,6R)-3,4,5-trihydroxy-6-(hydroxymethyl)tetrahydro-2H-pyran-2-yl)oxy)butan-2-yl)hexanamide

Benzyl (6-(((S)-1,4-bis(((2S,3S,4S,5S,6R)-3,4,5-trihydroxy-6-(hydroxymethyl) tetrahydro-2H-pyran-2-yl)oxy)butan-2-yl)amino)-6-oxohexyl)carbamate (1.50 g, 2.22 mmol) was dissolved in 20 mL of water and hydrogenated over 0.595 g of 20% Pd/C using a H_{2(g)} balloon over a period of 18 h. The catalyst was removed by filtration, and the title material was isolated by lyophilization. UPLC-MS Method B: m/z=543.3 (z=1); t_{R}=0.90 min.

### Example 1: 2,5-Dioxopyrrolidin-1-yl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate (ML-1)

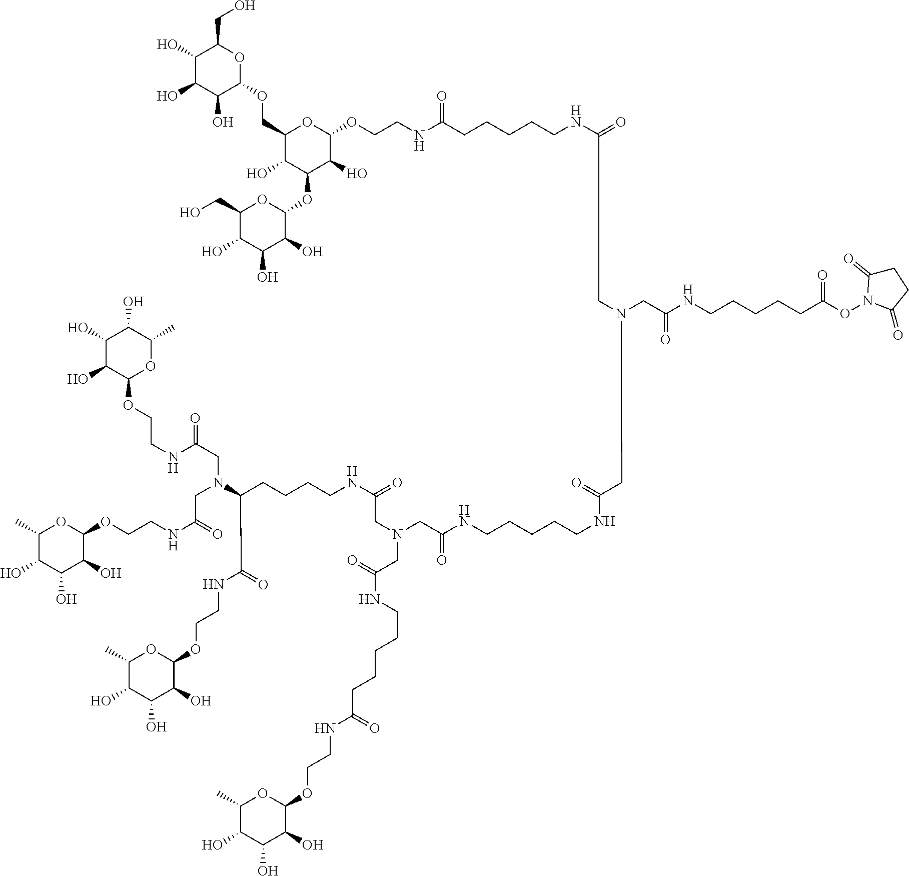 [SMILES: C[C@@H]1O[C@@H](OCCNC(=O)CCCCCNC(=O)CN(CC(=O)NCCCCCNC(=O)CN(CC(=O)NCCCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)CC(=O)NCCCC[C@@H](C(=O)NCCO[C@@H]2O[C@@H](C)[C@@H](O)[C@@H](O)[C@@H]2O)N(CC(=O)NCCO[C@@H]2O[C@@H](C)[C@@H](O)[C@@H](O)[C@@H]2O)CC(=O)NCCO[C@@H]2O[C@@H](C)[C@@H](O)[C@@H](O)[C@@H]2O)[C@@H](O)[C@H](O)[C@@H]1O]

### Step 1: (S)-3-(2-((5-(((Benzyloxy)carbonyl)amino)pentyl)amino)-2-oxoethyl)-5,14-dioxo-12-(2-oxo-2-((2-(((2R,3S,4R,5S,6S)-3,4,5-trihydroxy-6-methyltetrahydro-2H-pyran-2-yl)oxy)ethyl) amino)ethyl)-17-(((2R,3S,4R,5S,6S)-3,4,5-trihydroxy-6-methyltetrahydro-2H-pyran-2-yl)oxy)-11-((2-(((2R,3S,4R,5S,6S)-3,4,5-trihydroxy-6-methyltetrahydro-2H-pyran-2-yl)oxy)ethyl) carbamoyl)-3,6,12,15-tetraazaheptadecanoic acid

To a suspension of 13-(carboxymethyl)-3,11-dioxo-1-phenyl-2-oxa-4,10,13-triazapentadecan-15-oic acid (1.5 g, 3.66 mmol) in DCM (20 mL) at 0° C. was added TFAA (0.776 mL, 5.50 mmol). After stirring at 0° C. for 3 h, the mixture was taken to −30° C. To the resulting solution was added a solution of TEA (1.277 mL, 9.16 mmol) in DMF (20 mL) dropwise over 30 min. To the resulting mixture was added a solution of 2,2′-(((S)-6-amino-1-oxo-1-((2-(((2R,3S,4R,5S,6S)-3,4,5-trihydroxy-6-methyltetrahydro-2H-pyran-2-yl)oxy)ethyl)amino)hexan-2-yl)azanediyl)bis(N-(2-(((2R,3S,4R,5S,6S)-3,4,5-trihydroxy-6-methyltetrahydro-2H-pyran-2-yl)oxy)ethyl)acetamide) (3.04 g, 3.66 mmol) in DMF (20 mL). After stirring at rt for 16 h, the mixture was concentrated, and the residue was purified by C18 reverse phase chromatography to isolate title compound. UPLC-MS Method A: m/z=1221.6 (z=1); t_{R}=4.50 min.

### Step 2: Benzyl ((S)-4,13,17-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18-pentaazatricosan-23-yl)carbamate

To a solution of (S)-3-(2-((5-(((benzyloxy)carbonyl)amino)pentyl)amino)-2-oxoethyl)-5,14-dioxo-12-(2-oxo-2-((2-(((2R,3S,4R,5S,6S)-3,4,5-trihydroxy-6-methyltetrahydro-2H-pyran-2-yl)oxy)ethyl)amino)ethyl)-17-(((2R,3S,4R,5S,6S)-3,4,5-trihydroxy-6-methyltetrahydro-2H-pyran-2-yl)oxy)-11-((2-(((2R,3S,4R,5S,6S)-3,4,5-trihydroxy-6-methyltetrahydro-2H-pyran-2-yl)oxy)ethyl)carbamoyl)-3,6,12,15-tetraazaheptadecanoic acid (1.1289 g, 0.924 mmol) in DMF (2 mL) at rt was added EDC (213 mg, 1.109 mmol), HOBT (170 mg, 1.109 mmol), and TEA (0.283 mL, 2.034 mmol). After stirring for 30 min, a solution of 6-amino-N-(2-(((2R,3S,4R,5S,6S)-3,4,5-trihydroxy-6-methyltetrahydro-2H-pyran-2-yl)oxy)ethyl)hexanamide (444 mg, 1.387 mmol) in DMF (2 mL) was added to the reaction mixture dropwise. After stirring for 16 h, the mixture was concentrated, and the residue was purified by C18 reverse phase chromatography to provide the title compound. UPLC-MS Method A: m/z=1524.8 (z=1); t_{R}=3.24 min.

### Step 3: 2,2′-(((S)-13-(2-((5-Aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-vi)oxy)ethyl)acetamide)

A mixture of benzyl ((S)-4,13,17-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl) oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18-pentaazatricosan-23-yl)carbamate (1.0 g, 0.656 mmol) and Pd/C (70 mg, 0.656 mmol) in water (10 mL) was placed under N_{2(g)}. The solution was hydrogenated for 1 h, filtered through CELITE™ diatomaceous earth and washed with water. The filtrate was freeze-dried to isolate title compound. UPLC-MS Method A: m/z=1390.8 (z=1); t_{R}=4.18 min.

### Step 4: Benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl) amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate

To a solution of N-(2-((6-(benzyloxy)-6-oxohexyl)amino)-2-oxoethyl)-N-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)glycine (600 mg, 0.579 mmol) in DMF (1 mL) at rt was added EDC (166 mg, 0.868 mmol). HOBT (133 mg, 0.868 mmol), and DIPEA (0.253 mL, 1.446 mmol). After stirring for 30 min, a solution of 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) (856 mg, 0.616 mmol) in DMF (10 mL) was added dropwise. After stirring for 16 h, the mixture was concentrated, and the residue was purified by C18 reverse phase chromatography to provide the title compound. UPLC-MS Method F: m/z=1205.2 (z=2); t_{R}=3.96 min.

### Step 5: (S)-27-(2-((6-((2-(((2S,3S,4S5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl) amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid

A mixture of benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl) amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate (58 mg, 0.243 mmol) and Pd/C (7.77 mg, 0.073 mmol) in water (2 mL) was placed under N_{2(g)}. The solution was hydrogenated for 1 h, filtered through CELITE™ diatomaceous earth and washed with water. The filtrate was freeze-dried to isolate title compound. UPLC-MS Method A: m/z=1208.5 (z=2); t_{R}=4.06 min.

### Step 6: 2,5-Dioxopyrrolidin-1-yl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate

To a solution of (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy) ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oic acid (570 mg, 0.246 mmol) in DMF (5 mL) was added DIPEA (0.198 mL, 1.131 mmol), followed by the addition of HSPyU (121 mg, 0.295 mmol). After stirring 1 h, to the reaction mixture was added TFA (0.125 mL, 1.623 mmol) and stirred an additional 15 min. The solution was triturated with EtOAc and centrifuged at 3500 rpm for 15 min, and the product was isolated by decanting. UPLC-MS Method F: m/z=1208.5 (z=2); t_{R}=4.16 min.

### Example 2: 2,5-Dioxopyrrolidin-1-yl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3R,4R,5R,6S)-3,4,5-trihydroxy-6-(hydroxymethyl)tetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriaconetan-36-oate (ML-2)

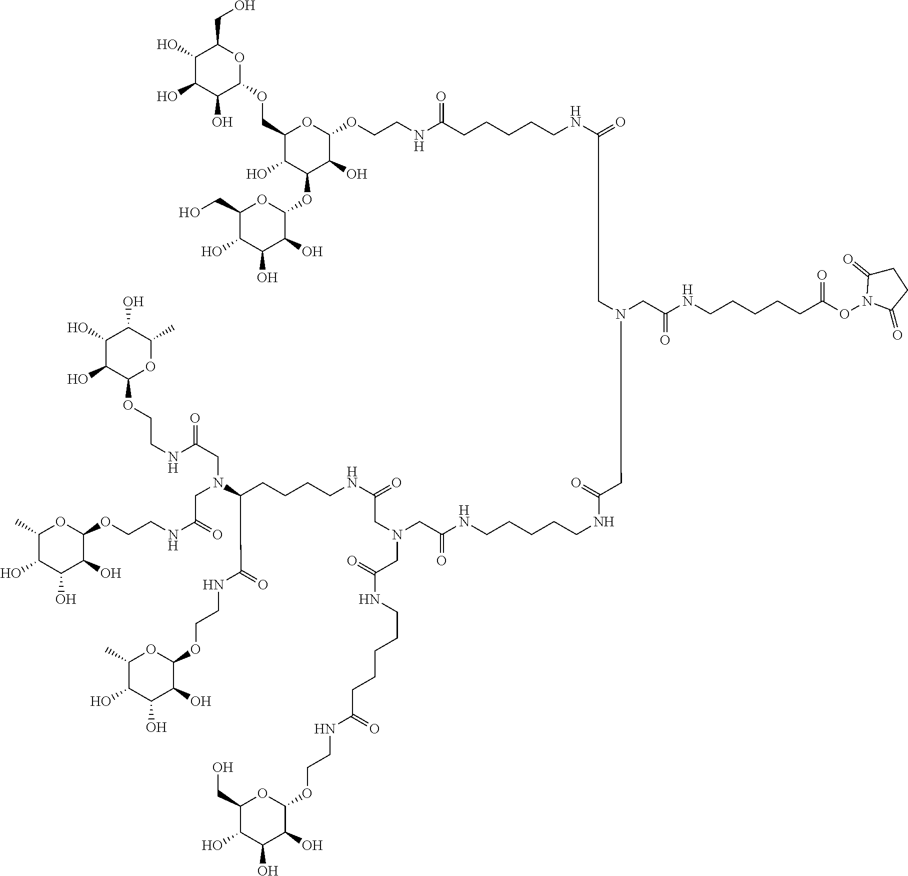 [SMILES: C[C@@H]1O[C@@H](OCCNC(=O)CN(CC(=O)NCCO[C@@H]2O[C@@H](C)[C@@H](O)[C@@H](O)[C@@H]2O)[C@@H](CCCCNC(=O)CN(CC(=O)NCCCCCNC(=O)CN(CC(=O)NCCCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)CC(=O)NCCCCCC(=O)NCCO[C@H]2O[C@H](CO)[C@@H](O)[C@H](O)[C@@H]2O)C(=O)NCCO[C@@H]2O[C@@H](C)[C@@H](O)[C@@H](O)[C@@H]2O)[C@@H](O)[C@H](O)[C@@H]1O]

### Step 1: Benzyl ((S)-4,13,17-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3R,4R,5R,6S)-3,4,5-trihydroxy-6-(hydroxymethyl)tetrahydro-2H-pyran-2-yl)oxy)ethyl)amino)hexyl) amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18-pentaazatricosan-23-yl)carbamate

The title compound was prepared using procedures analogous to those described for Example 1, Step 2, substituting 6-amino-N-(2-(((2R,3R,4R,5R,6S)-3,4,5-trihydroxy-6-(hydroxymethyl) tetrahydro-2H-pyran-2-yl)oxy)ethyl)hexanamide for 6-amino-N-(2-(((2R,3S,4R,5S,6S)-3,4,5-trihydroxy-6-methyltetrahydro-2H-pyran-2-yl)oxy)ethyl)hexanamide to isolate the title compound. UPLC-MS Method A: m/z=1539.6 (z=1); t_{R}=3.29 min.

### Step 2: 2,2′-(((S)-13-(2-((5-Aminopentyl)amino)-2-oxoethyl)-4,11,15,22-tetraoxo-25-(((2R,3R,4R,5R,6S)-3,4,5-trihydroxy-6-(hydroxymethyl)tetrahydro-2H-pyran-2-yl)oxy)-1-(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide)

The title compound was prepared using procedures analogous to those described for Example 1, Step 3, substituting benzyl ((S)-4,13,17-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3R,4R,5R,6S)-3,4,5-trihydroxy-6-(hydroxymethyl)tetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18-pentaazatricosan-23-yl) carbamate for benzyl ((S)-4,13,17-trioxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18-pentaazatricosan-23-yl)carbamate to isolate title compound. UPLC-MS Method A: m/z=1405.5 (z=1); t_{R}=1.96 mm.

### Step 3: Benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3R,4R,5R,6S)-3,4,5-trihydroxy-6-(hydroxymethyl)tetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate

The title compound was prepared using procedures analogous to those described for Example 1, Step 4, substituting 2,2′-(((S)-13-(2-((5-aminopentyl)amino)-2-oxoethyl)-4,11,15,22-tetraoxo-25-(((2R,3R,4R,5R,6S)-3,4,5-trihydroxy-6-(hydroxymethyl)tetrahydro-2H-pyran-2-yl)oxy)-1-(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) for 2,2′-(((S)-13-(2-((5-aminopentyl) amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) to isolate the title compound. UPLC-MS Method A: m/z=1212.9 (z=2); t_{R}=3.0 min.

### Step 4: (S)-27-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3R,4R,5R,6S)-3,4,5-trihydroxy-6-(hydroxymethyl)tetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid

The title compound was prepared using procedures analogous to those described for Example 1, Step 5, substituting benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3 S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3R,4R,5R,6S)-3,4,5-trihydroxy-6-(hydroxymethyl)tetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1169.9 (z=2); t_{R}=2.16 min.

### Step 5: 2,5-Dioxopyrrolidin-1-yl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3R,4R,5R,6S)-3,4,5-trihydroxy-6-(hydroxymethyl) tetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate

The title compound was prepared using procedures analogous to those described for Example 1, Step 6, substituting (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3R,4R,5R,6S)-3,4,5-trihydroxy-6-(hydroxymethyl)tetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy-)ethyl)carbamoyl)-3,6,12,15,18,24,27,30-octaazahexatriacontan-36-oic acid to isolate title compound. UPLC-MS Method A: m/z=1261.9.6 (z=2); t_{R}=2.43 min.

### Example 3: 2,5-Dioxopyrrolidin-1-yl (S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((2-(((2S,3R,4S,5R,6R)-3,4,5-trihydroxy-6-methyltetrahydro-2H-pyran-2-yl)oxy)ethyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate (ML-3)

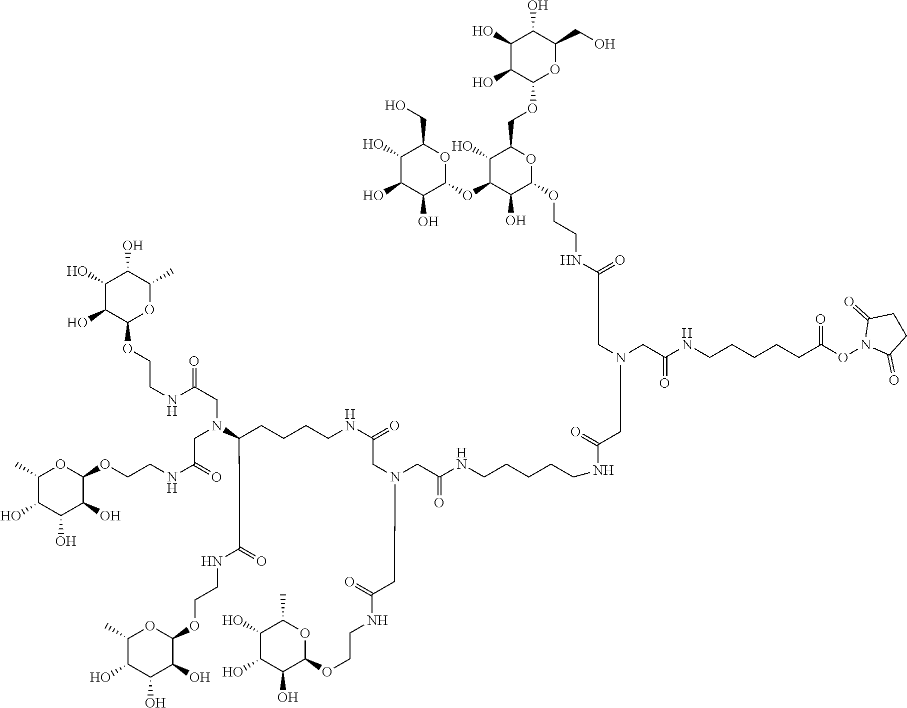 [SMILES: C[C@@H]1O[C@@H](OCCNC(=O)CN(CC(=O)NCCCCCNC(=O)CN(CC(=O)NCCCCCC(=O)ON2C(=O)CCC2=O)CC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCC[C@@H](C(=O)NCCO[C@@H]2O[C@@H](C)[C@@H](O)[C@@H](O)[C@@H]2O)N(CC(=O)NCCO[C@@H]2O[C@@H](C)[C@@H](O)[C@@H](O)[C@@H]2O)CC(=O)NCCO[C@@H]2O[C@@H](C)[C@@H](O)[C@@H](O)[C@@H]2O)[C@@H](O)[C@H](O)[C@@H]1O]

### Step 1: Benzyl (S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((2-(((2S,3R,4S,5R,6R)-3,4,5-trihydroxy-6-methyltetrahydro-2H-pyran-2-yl)oxy)ethyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate

To a solution of (S)-3-(2-((6-(benzyloxy)-6-oxohexyl)amino)-2-oxoethyl)-5,13,17,26-tetraoxo-24-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((2-(((2S,3R,4S,5R,6R)-3,4,5-trihydroxy-6-methyltetrahydro-2H-pyran-2-yl)oxy)ethyl)amino)ethyl)-29-(((2R,3S,4R,5S,6S)-3,4,5-trihydroxy-6-methyltetrahydro-2H-pyran-2-yl)oxy)-23-((2-(((2R,3S,4R,5S,6S)-3,4,5-trihydroxy-6-methyltetrahydro-2H-pyran-2-yl)oxy)ethyl)carbamoyl)-3,6,12,15,18,24,27-heptaazanonacosanoic acid (76 mg, 0.046 mmol) in DMF (2 mL) at rt was added EDC (35.3 mg, 0.184 mmol), HOBT (28.2 mg, 0.184 mmol), and TEA (0.035 mL, 0.253 mmol). After stirring for 30 min, a solution of AETM-NH₂ (856 mg, 0.616 mmol) in DMF (10 mL) was added dropwise. After stirring for 16 h, the mixture was concentrated, and the residue was purified by C18 reverse phase chromatography to provide the title compound. UPLC-MS Method A: m/z=1091.4 (z=2); t_{R}=3.28 min.

### Step 2: (S)-27-(2-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((2-(((2S,3R,4S,5R,6R)-3,4,5-trihydroxy-6-methyltetrahydro-2H-pyran-2-yl)oxy)ethyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid

The title compound was prepared using procedures analogous to those described for Example 1, Step 5, substituting benzyl (S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((2-(((2S,3R,4S,5R,6R)-3,4,5-trihydroxy-6-methyltetrahydro-2H-pyran-2-yl)oxy) ethyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,15,18,24,27,30-octaazahexatriacontan-36-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl) amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1046.4 (z=2); t_{R}=0.95 min.

### Step 3: 2,5-Dioxopyrrolidin-1-yl (S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((2-(((2S,3R,4S,5R,6R)-3,4,5-trihydroxy-6-methyltetrahydro-2H-pyran-2-yl)oxy)ethyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate

The title compound was prepared using procedures analogous to those described for Example 1, Step 6, substituting (S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((2-(((2S,3R,4S,5R,6R)-3,4,5-trihydroxy-6-methyltetrahydro-2H-pyran-2-yl)oxy)ethyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1094.8 (z=2); t_{R}=2.35 min.

### Example 4: 2,5-Dioxopyrrolidin-1-yl 25-(2-(((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-4,11,15,23,27-pentaoxo-1-(((2R,3S,4R,5S,6S)-3,4,5-trihydroxy-6-methyltetrahydro-2H-pyran-2-yl)oxy)-3,10,13,16,22,25,28-heptaazatetratriacontan-34-oate (ML-4)

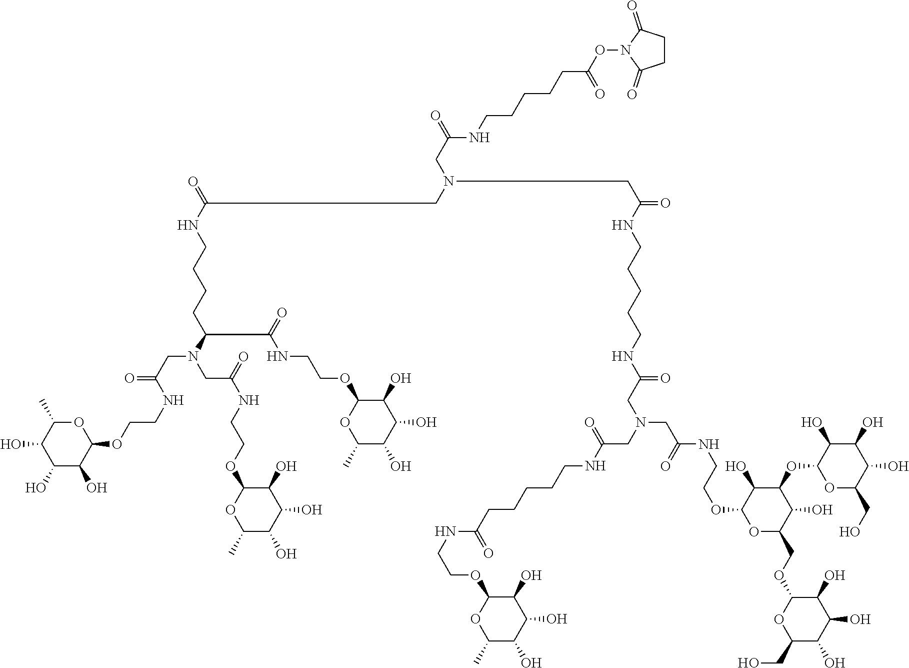 [SMILES: C[C@@H]1O[C@@H](OCCNC(=O)CCCCCNC(=O)CN(CC(=O)NCCCCCNC(=O)CN(CC(=O)NCCCCCC(=O)ON2C(=O)CCC2=O)CC(=O)NCCCC[C@@H](C(=O)NCCO[C@@H]2O[C@@H](C)[C@@H](O)[C@@H](O)[C@@H]2O)N(CC(=O)NCCO[C@@H]2O[C@@H](C)[C@@H](O)[C@@H](O)[C@@H]2O)CC(=O)NCCO[C@@H]2O[C@@H](C)[C@@H](O)[C@@H](O)[C@@H]2O)CC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)[C@@H](O)[C@H](O)[C@@H]1O]

### Step 1: Benzyl 25-(2-(((S)-5-(bis(2-oxo-2-((2-(((2R,3S,4R,5S,6S)-3,4,5-trihydroxy-6-methyltetrahydro-2H pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,11,15,23,27-pentaoxo-1-(((2R,3S,4R,5S,6S)-3,4,5-trihydroxy-6-methyltetrahydro-2H-pyran-2-yl)oxy)-3,10,13,16,22,25,28-heptaazatetratriacontan-34-oate

To a solution of (S)-3-(2-((6-(benzyloxy)-6-oxohexyl)amino)-2-oxoethyl)-5,14-dioxo-12-(2-oxo-2-((2-(((2R,3S,4R,5S,6S)-3,4,5-trihydroxy-6-methyltetrahydro-2H-pyran-2-yl)oxy)ethyl) amino)ethyl)-17-(((2R,3S,4R,5S,6S)-3,4,5-trihydroxy-6-methyltetrahydro-2H-pyran-2-yl)oxy)-11-((2-(((2R,3S,4R,5S,6S)-3,4,5-trihydroxy-6-methyltetrahydro-2H-pyran-2-yl)oxy)ethyl) carbamoyl)-3,6,12,15-tetraazaheptadecanoic acid (100 mg, 0.083 mmol) in DMF (2 mL) at rt was added EDC (15.9 mg, 0.0.083 mmol), HOBT (12.7 mg, 0.083 mmol), and TEA (11.55 μL, 0.083 mmol). After stirring 30 min. a solution of 6-(2-((2-((5-aminopentyl)amino)-2-oxoethyl)(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-2-oxoethyl)amino)acetamido)-N-(2-(((2R,3S,4R,5S,6S)-3,4,5-trihydroxy-6-methyltetrahydro-2H-pyran-2-yl)oxy)ethyl)hexanamide (92 mg, 0.083 mmol) in DMF (10 mL) was added dropwise. After stirring for 16 h, the mixture was concentrated, and the residue was purified by C18 reverse phase chromatography to provide the title compound. UPLC-MS Method F: m/z=1148.4 (z=2); t_{R}=3.35 min.

### Step 2: 25-(2-(((S)-5-(Bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methy)tetrahydro-2H-pyran-2-yl)oxy)ethyl)amino)-2-oxoethyl)-4,11,15,23,27-pentaoxo-1-(((2R,3S,4R,5S,6S)-3,4,5-trihydroxy-6-methyltetrahydro-2H-pyran-2-yl)oxy)-3,10,13,16,22,25,28-heptaazatetratriacontan-34-oic acid

The title compound was prepared using procedures analogous to those described for Example 1, Step 5, substituting benzyl 25-(2-(((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,11,15,23,27-pentaoxo-1-(((2R,3S,4R,5S,6S)-3,4,5-trihydroxy-6-methyltetrahydro-2H-pyran-2-yl)oxy)-3,10,13,16,22,25,28-heptaazatetratriacontan-34-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1103.4 (z=2); t_{R}=2.78 min.

### Step 3: 2,5-Dioxopyrrolidin-1-yl 25-(2-(((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,11,15,23,27-pentaoxo-1-(((2R,3S,4R,5.6S)-3,4,5-trihydroxy-6-methyltetrahydro-2H-pyran-2-yl)oxy)-3,10,13,16,22,25,28-heptaazatetratriacontan-34-oate

The title compound was prepared using procedures analogous to those described for Example 1, Step 6, substituting 25-(2-(((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)-4,11,15,23,27-pentaoxo-1-(((2R,3S,4R,5S,6S)-3,4,5-trihydroxy-6-methyltetrahydro-2H-pyran-2-yl)oxy)-3,10,13,16,22,25,28-heptaazatetratriacontan-34-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1151.9 (z=2); t_{R}=3.22 min.

### Example 5: 2,5-Dioxopyrrolidin-1-yl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl) amino)pentan-2-yl)amino)-2-oxoethyl)-4,9,13,21,25-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,11,14,20,23,26-heptaazadotriacontan-32-oate (ML-5)

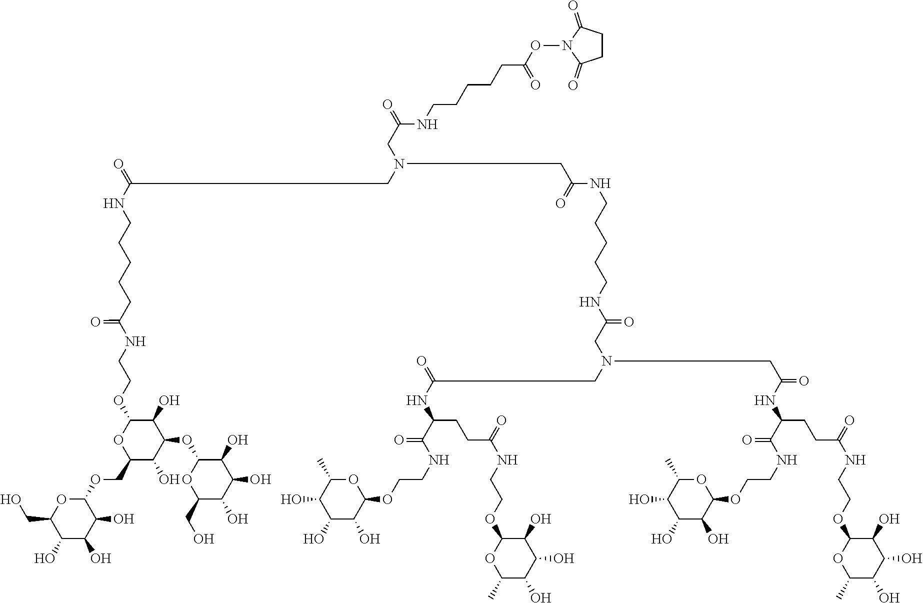 [SMILES: C[C@@H]1O[C@@H](OCCNC(=O)CC[C@H](NC(=O)CN(CC(=O)NCCCCCNC(=O)CN(CC(=O)NCCCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)CC(=O)N[C@@H](CCC(=O)NCCO[C@@H]2O[C@@H](C)[C@@H](O)[C@@H](O)[C@@H]2O)C(=O)NCCO[C@@H]2O[C@@H](C)[C@@H](O)[C@@H](O)[C@H]2O)C(=O)NCCO[C@@H]2O[C@@H](C)[C@@H](O)[C@@H](O)[C@@H]2O)[C@@H](O)[C@H](O)[C@@H]1O]

### Step 1: Benzyl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-2-oxoethyl)-4,9,13,21,25-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,8,11,14,20,23,26-heptaazadotriacontan-32-oate

The title compound was prepared using procedures analogous to those described for Example 1, Step 4, substituting (2S,2′S)-2,2′-((2,2′-((2-((5-aminopentyl)amino)-2-oxoethyl) azanediyl)bis(acetyl))bis(azanediyl))bis(N1,N5-bis(2-(((2R,3S,4R,5S,6S)-3,4,5-trihydroxy-6-methyltetrahydro-2H-pyran-2-yl)oxy)ethyl)pentanediamide) for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide to isolate title compound. UPLC-MS Method A: m/z=1155.4 (z=2); t_{R}=3.91 min.

### Step 2: (S)-23-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-2-oxoethyl)-4,9,13,21,25-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,11,14,20,23,26-heptaazadotriacontan-32-oic acid

The title compound was prepared using procedures analogous to those described for Example 1, Step 5, substituting benzyl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-2-oxoethyl)-4,9,13,21,25-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,8,11,14,20,23,26-heptaazadotriacontan-32-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1110.4 (z=2); t_{R}=3.03 min.

### Step 3: 2,5-Dioxopyrrolidin-1-yl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl) amino)pentan-2-yl)amino)-2-oxoethyl)-4,9,13,21,25-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,11,14,20,23,26-heptaazadotriacontan-32-oate

The title compound was prepared using procedures analogous to those described for Example 1, Step 6, substituting (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-2-oxoethyl)-4,9,13,21,25-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,8,11,14,20,23,26-heptaazadotriacontan-32-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3 S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: t_{R}=3.27 min; m/z=1158.9 (z=2).

### Example 6: 2,5-Dioxopyrrolidin-1-yl (S)-27-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-15-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl) carbamoyl)-3,6,12,15,18,24,27,30-octaazahexatriacontan-36-oate (ML-6)

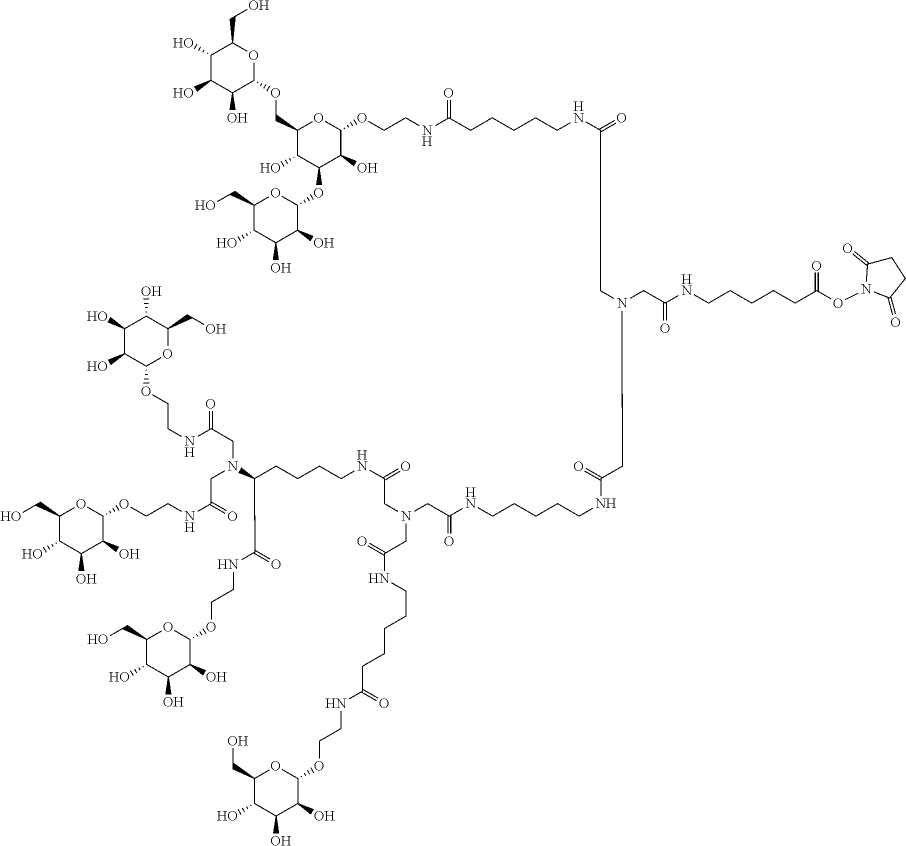 [SMILES: O=C(CCCCCNC(=O)CN(CC(=O)NCCCCCNC(=O)CN(CC(=O)NCCCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CC(=O)NCCCCCC(=O)ON1C(=O)CCC1=O)CC(=O)NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O)NCCO[C@H]1O[C@H](CO)[C@@H](O)[C@H](O)[C@@H]1O]

### Step 1: Benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-15-(2-oxo-2-((6-oxo-6-((2-(((2S,3S,4S,5S,6R)-3,4,5-trihydroxy-6-(hydroxymethyl) tetrahydro-2H-pyran-2-yl)oxy) ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,15,18,24,27,30-octaazahexatriacontan-36-oate

The title compound was prepared using procedures analogous to those described for Example 1, Step 4, substituting 2,2′-(((S)-13-(2-((5-aminopentyl)amino)-2-oxoethyl)-4,11,15,22-tetraoxo-1,25-bis(((2S,3S,4S,5S,6R)-3,4,5-trihydroxy-6-(hydroxymethyl)tetrahydro-2H-pyran-2-yl)oxy)-3,10,13,16,23-pentaazapentacosan-5-yl)azanediyl)bis(N-(2-(((2S,3S,4S,5S,6R)-3,4,5-trihydroxy-6-(hydroxymethyl)tetrahydro-2H-pyran-2-yl)oxy)ethyl)acetamide) for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl) azanediyl)bis(N-(2-(((2R,3S,4R,5S,6S)-3,4,5-trihydroxy-6-methyltetrahydro-2H-pyran-2-yl)oxy) ethyl)acetamide to isolate title compound. UPLC-MS Method A: m/z=1236.9 (z=2); t_{R}=3.81 min.

### Step 2: (S)-27-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-15-(2-oxo-2-((6-oxo-6-((2-(((2S,3S,4S,5S,6R)-3,4,5-trihydroxy-6-(hydroxymethyl) tetrahydro-2H-pyran-2-yl)oxy) ethyl)amino)hexyl)amino)ethyl)-1-(((2S,3S,4S,5S,6R)-3,4,5-trihydroxy-6-(hydroxymethyl) tetrahydro-2H-pyran-2-yl)oxy)-7-((2-(((2S,3S,4S,S, 6R)-3,4,5-trihydroxy-6-(hydroxymethyl) tetrahydro-2H-pyran-2-yl)oxy)ethyl)carbamoyl)-3,6,12,15,18,24,27,30-octaazahexatriacontan-36-oic acid

The title compound was prepared using procedures analogous to those described for Example 1, Step 5, substituting benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3 S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-15-(2-oxo-2-((6-oxo-6-((2-(((2S,3S,4S,5S,6R)-3,4,5-trihydroxy-6-(hydroxymethyl) tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl) carbamoyl)-3,6,12,15,18,24,27,30-octaazahexatriacontan-36-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,15,18,24,27,30-octaazahexatriacontan-36-oate to isolate title compound. UPLC-MS Method A: m/z=1191.9 (z=2); t_{R}=3.03 min.

### Step 3: 2,5-Dioxopyrrolidin-1-yl (S)-27-(2-((6-((2-(((2S,3S,4S,5R,66R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino) ethyl)-15-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl) carbamoyl)-3,6,12,15,18,24,27,30-octaazahexatriacontan-36-oate

The title compound was prepared using procedures analogous to those described for Example 1, Step 6, substituting (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-15-(2-oxo-2-((6-oxo-6-((2-(((2S,3S,4S,5S,6R)-3,4,5-trihydroxy-6-(hydroxymethyl) tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,15,18,24,27,30-octaazahexatriacontan-36-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1240.5 (z=2); t_{R}=3.14 min.

### Example 7: 2,5-Dioxopyrrolidin-1-yl18-(2-(((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)amino)-2-oxoethyl)-1-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)-4,8,16,20-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-3,6,9,15,18,21-hexaazakeptacosan-27-oate (ML-7)

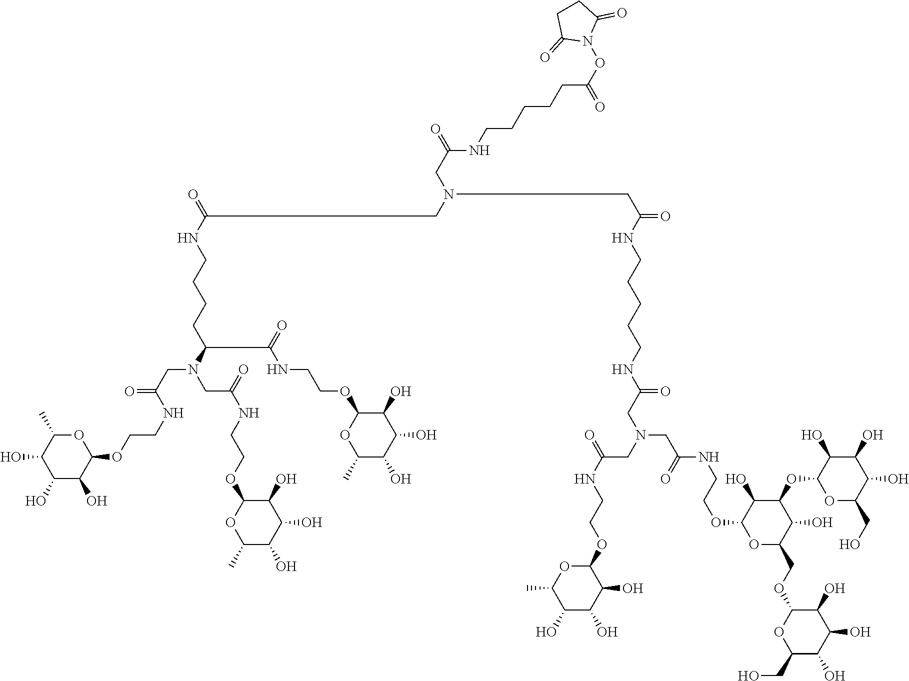 [SMILES: C[C@@H]1O[C@@H](OCCNC(=O)CN(CC(=O)NCCCCCNC(=O)CN(CC(=O)NCCCCCC(=O)ON2C(=O)CCC2=O)CC(=O)NCCCC[C@@H](C(=O)NCCO[C@@H]2O[C@@H](C)[C@@H](O)[C@@H](O)[C@@H]2O)N(CC(=O)NCCO[C@@H]2O[C@@H](C)[C@@H](O)[C@@H](O)[C@@H]2O)CC(=O)NCCO[C@@H]2O[C@@H](C)[C@@H](O)[C@@H](O)[C@@H]2O)CC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)[C@@H](O)[C@H](O)[C@@H]1O]

### Step 1: 3,11-Dioxo-13-(2-oxo-2-((2-(((2R,3S,4R,5S,6S)-3,4,5-trihydroxy-6-methyltetrahydro-2H-pyran-2-yl)oxy)ethyl)amino)ethyl)-1-phenyl-2-oxa-4,10,13-triazapentadecan-15-oic acid

To a suspension of 13-(carboxymethyl)-3,11-dioxo-1-phenyl-2-oxa-4,10,13-triazapentadecan-15-oic acid (600 mg, 1.465 mmol) in DMF (20 mL) was added HATU (1226 mg, 3.22 mmol) and TEA (0.613 mL, 4.40 mmol). The reaction was concentrated under reduced pressure and purified by C18 reverse phase chromatography to isolate the title compound.

### Step 2: Benzyl (5-(2-((2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)(2-oxo-2-((2-(((2R,3S,4R,5S,6S)-3,4,5-trihydroxy-6-methyltetrahydro-2H-pyran-2-yl)oxy)ethyl)amino)ethyl)amino)acetamido)pentyl)carbamate

To a solution of 3,11-dioxo-13-(2-oxo-2-((2-(((2R,3S,4R,5S,6S)-3,4,5-trihydroxy-6-methyltetrahydro-2H-pyran-2-yl)oxy)ethyl)amino)ethyl)-1-phenyl-2-oxa-4,10,13-triazapentadecan-15-oic acid (390 mg, 0.651 mmol) in DMF (2 mL) at rt was added EDC (150 mg, 0.782 mmol), HOBT (120 mg, 0.782 mmol), and TEA (0.227 mL, 1.629 mmol). After stirring 30 mm, to the reaction mixture was added a solution of AETM-NH₂ (428 mg, 0.651 mmol) in DMF (2 mL) dropwise. After stirring for 16 h, additional EDC (150 mg, 0.782 mmol), TEA (0.227 mL, 1.629 mmol) and AETM-NH₂ (428 mg, 0.651 mmol) were added to the reaction, which was stirred for an additional 1 h. The mixture was concentrated, and the residue was purified by C18 reverse phase chromatography to provide the title compound. UPLC-MS Method A: m/z=1128.4 (z=2); t_{R}=3.76 min.

### Step 3: N-(5-Aminopentyl)-2-((2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)(2-oxo-2-((2-(((2R,3S,4R,5S,6S)-3,4,5-trihydroxy-6-methyltetrahydro-2H-pyran-2-yl)oxy)ethyl)amino)ethyl)amino)acetamide

The title compound was prepared using procedures analogous to those described for Intermediate 5, Step 2, substituting benzyl (5-(2-((2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)(2-oxo-2-((2-(((2R,3S,4R,5S,6S)-3,4,5-trihydroxy-6-methyltetrahydro-2H-pyran-2-yl)oxy)ethyl)amino)ethyl)amino)acetamido)pentyl) carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate to isolate title compound. UPLC-MS Method A: m/z=994.4 (z=1); t_{R}=0.95 min.

### Step 4: Benzyl 18-(2-(((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)amino)-2-oxoethyl)-1-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)-4,8,16,20-tetraoxo-6-(2-oxo-2-((2-(((2R,3S,4R,5S,6S)-3,4,5-trihydroxy-6-methyltetrahydro-2H-pyran-2-yl)oxy)ethyl) amino)ethyl)-3,6,9,15,18,21-hexaazaheptacosan-27-oate

The title compound was prepared using procedures analogous to those described for Example 4, Step 1, substituting N-(5-aminopentyl)-2-((2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)(2-oxo-2-((2-(((2R,3S,4R,5S,6S)-3,4,5-trihydroxy-6-methyltetrahydro-2H-pyran-2-yl)oxy)ethyl)amino)ethyl)amino)acetamide for 6-(2-((2-((5-aminopentyl)amino)-2-oxoethyl)(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl) oxy)ethyl)amino)-2-oxoethyl)amino)acetamido)-N-(2-(((2R,3S,4R,5S,6S)-3,4,5-trihydroxy-6-methyltetrahydro-2H-pyran-2-yl)oxy)ethyl)hexanamide to isolate title compound. UPLC-MS Method A: m/z=1091.9 (z=2); t_{R}=4.12 min.

### Step 5: 18-(2-(((S)-5-(Bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)amino)-2-oxoethyl)-1-(((2S,354S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy) methyl)tetrahydro-2H-pyran-2-yl)oxy)-4,8,16,20-tetraoxo-6-(2-oxo-2-((2-(((2R,3S,4R,5S,6S)-3,4,5-trihydroxy-6-methyltetrahydro-2H-pyran-2-yl)oxy)ethyl) amino)ethyl)-3,6,9,15,18,21-hexaazaheptacosan-27-oic acid

The title compound was prepared using procedures analogous to those described for Example 1, Step 5, substituting benzyl 18-(2-(((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)amino)-2-oxoethyl)-1-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)-4,8,16,20-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-3,6,9,15,18,21-hexaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1046.9 (z=2); t_{R}=2.95 min.

### Step 6: 2,5-Dioxopyrrolidin-1-yl 18-(2-(((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl) amino)-2-oxoethyl)-1-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)-4,8,16,20-tetraoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-3,6,9,15,18,21-hexaazaheptacosan-27-oate

The title compound was prepared using procedures analogous to those described for Example 1, Step 6, substituting 18-(2-(((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)amino)-2-oxoethyl)-1-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)-4,8,16,20-tetraoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-3,6,9,15,18,21-hexaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1095.6 (z=2); t_{R}=1.62 min.

### Example 8: 2,5-Dioxopyrrolidin-1-yl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,15,18,24,27,30-octaazahexatriacontan-36-oate (ML-8)

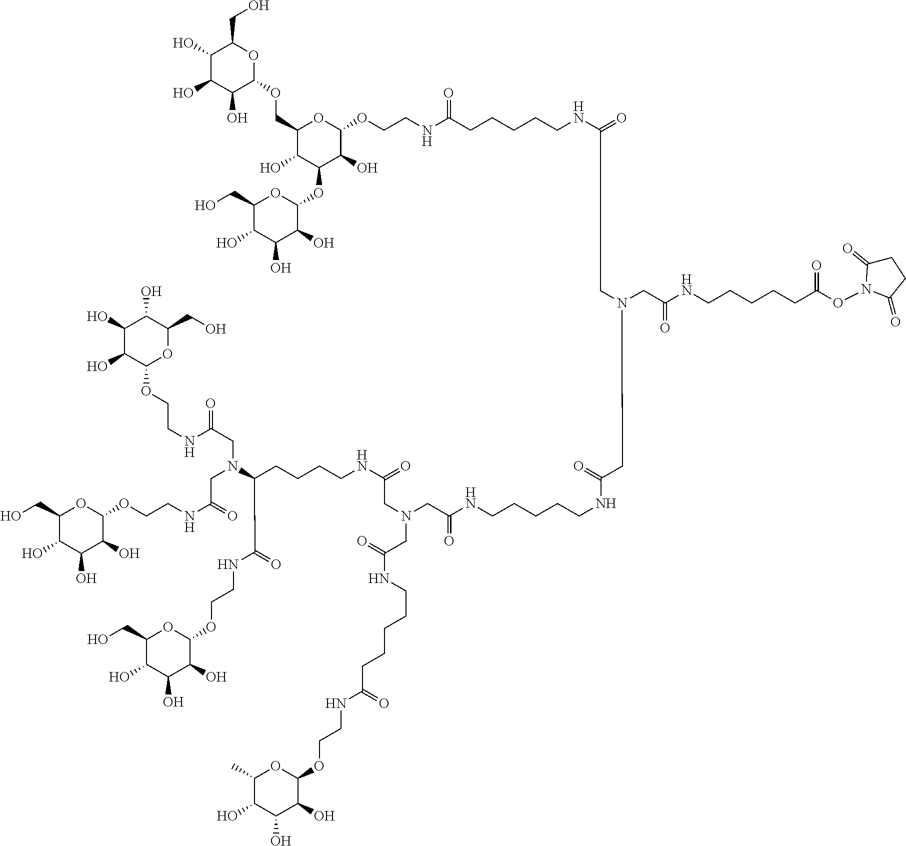 [SMILES: C[C@@H]1O[C@@H](OCCNC(=O)CCCCCNC(=O)CN(CC(=O)NCCCCCNC(=O)CN(CC(=O)NCCCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)CC(=O)NCCCC[C@@H](C(=O)NCCO[C@H]2O[C@H](CO)[C@@H](O)[C@H](O)[C@@H]2O)N(CC(=O)NCCO[C@H]2O[C@H](CO)[C@@H](O)[C@H](O)[C@@H]2O)CC(=O)NCCO[C@H]2O[C@H](CO)[C@@H](O)[C@H](O)[C@@H]2O)[C@@H](O)[C@H](O)[C@@H]1O]

### Step 1: Benzyl ((S)-4,13,17-trioxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl) amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,15,18-pentaazatricosan-23-yl)carbamate

The title compound was prepared using procedures analogous to those described for Intermediate 12, Step 1, substituting 2,2′-(((S)-6-amino-1-oxo-1-((2-(((2S,3S,4S,5S,6R)-3,4,5-trihydroxy-6-(hydroxymethyl)tetrahydro-2H-pyran-2-yl)oxy)ethyl) amino)hexan-2-yl)azanediyl) bis(N-(2-(((2S,3S,4S,5S,6R)-3,4,5-trihydroxy-6-(hydroxymethyl)tetrahydro-2H-pyran-2-yl)oxy)ethyl)acetamide) for AETM to isolate the title compound. UPLC-MS Method A: m/z=1572.5 (z=1); t_{R}=1.21 min.

### Step 2: 2,2′-(((S)-13-(2-((5-Aminopentyl)amino)-2-oxoethyl)-4,11,15,22-tetraoxo-1-(((2S,3S,4S,5S,6R)-3,4,5-trihydroxy-6-(hydroxymethyl)tetrahydro-2H-pyran-2-yl)oxy)-25-(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2S,3S,4S,5S,6R)-3,4,5-trihydroxy-6-(hydroxymethyl)tetrahydro-2H-pyran-2-yl)oxy)ethyl)acetamide)

The title compound was prepared using procedures analogous to those described for Intermediate 5, Step 2, substituting benzyl ((S)-4,13,17-trioxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,15,18-pentaazatricosan-23-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate to isolate title compound. UPLC-MS Method A: m/z=1438.0 (z=1); t_{R}=1.21 min.

### Step 3: Benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,15,18,24,27,30-octaazahexatriacontan-36-oate

The title compound was prepared using procedures analogous to those described for Example 1, Step 4, substituting 2,2′-(((S)-13-(2-((5-aminopentyl)amino)-2-oxoethyl)-4,11,15,22-tetraoxo-1-(((2S,3S,4S,5S,6R)-3,4,5-trihydroxy-6-(hydroxymethyl)tetrahydro-2H-pyran-2-yl)oxy)-25-(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2S,3S,4S,5S,6R)-3,4,5-trihydroxy-6-(hydroxymethyl)tetrahydro-2H-pyran-2-yl)oxy)ethyl)acetamide) for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide to isolate title compound. UPLC-MS Method A: m/z=1229.5 (z=2); t_{R}=2.27 min.

### Step 4: (S)-27-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,15,18,24,27,30-octaazahexatriacontan-36-oic acid

The title compound was prepared using procedures analogous to those described for Example 1, Step 5, substituting benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl) carbamoyl)-3,6,12,15,18,24,27,30-octaazahexatriacontan-36-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,15,18,24,27,30-octaazahexatriacontan-36-oate to isolate title compound. UPLC-MS Method A: m/z=1184.3 (z=2); t_{R}=1.64 min.

### Step 5: 2,5-Dioxopyrrolidin-1-yl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl) tetrahydro-2H-pyran-2-yl)oxy)ethyl)carbamoyl)-3,6,12,15,18,24,27,30-octaazahexatriacontan-36-oate

The title compound was prepared using procedures analogous to those described for Example 1, Step 6, substituting (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2S,3S,4S,5S,6R)-3,4,5-trihydroxy-6-(hydroxymethyl)tetrahydro-2H-pyran-2-yl) oxy)ethyl) amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,15,18,24,27,30-octaazahexatriacontan-36-oic acid for (S)-27-(2-((6-((2-(((2S,3S,4S,5R, 6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1232.6 (z=2); t_{R}=3.81 min.

### Example 9: 2,5-Dioxopyrrolidin-1-yl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2S,3R,4S,5S,6R)-3,4,5-trihydroxy-6-(hydroxymethyl)tetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate (ML-9)

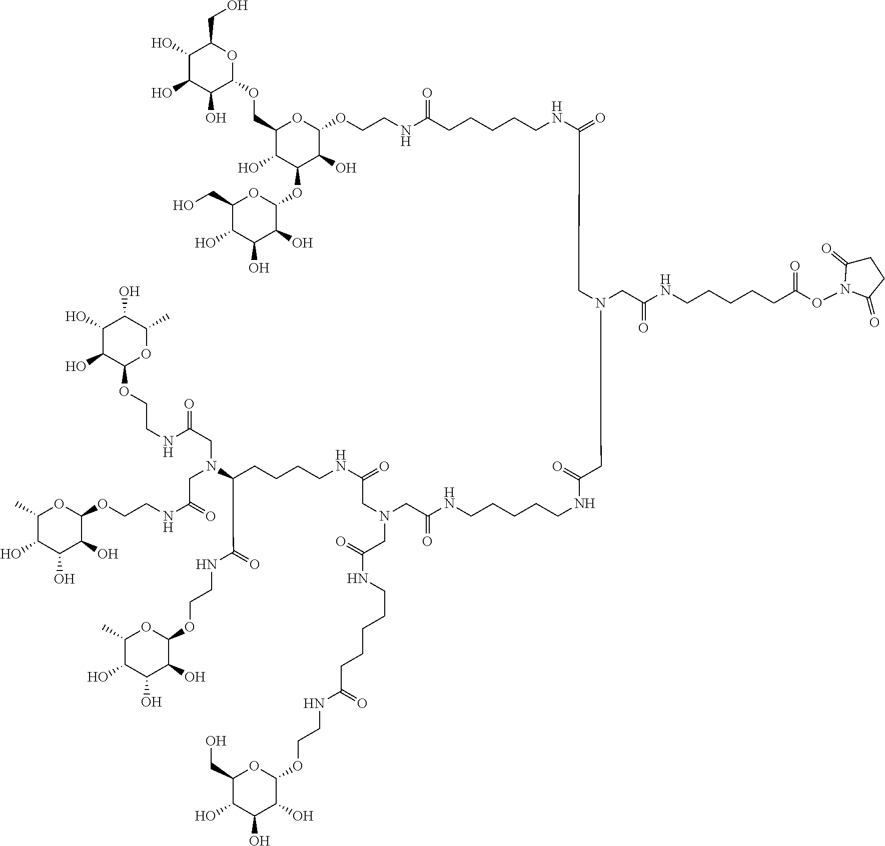 [SMILES: C[C@@H]1O[C@@H](OCCNC(=O)CN(CC(=O)NCCO[C@@H]2O[C@@H](C)[C@@H](O)[C@@H](O)[C@@H]2O)[C@@H](CCCCNC(=O)CN(CC(=O)NCCCCCNC(=O)CN(CC(=O)NCCCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)CC(=O)NCCCCCC(=O)NCCO[C@H]2O[C@H](CO)[C@@H](O)[C@H](O)[C@H]2O)C(=O)NCCO[C@@H]2O[C@@H](C)[C@@H](O)[C@@H](O)[C@@H]2O)[C@@H](O)[C@H](O)[C@@H]1O]

### Step 1: Benzyl ((S)-4,13,17-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl-15-(2-oxo-2-(6-oxo-6-((2-(((2S,3R,4S5,6R)-3,4,5-trihydroxy-6-(hydroxymethyl)tetrahydro-2H-pyran-2-yl)oxy)ethyl) amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18-pentaazatricosan-23-yl)carbamate

To a solution of (S)-3-(2-((5-(((benzyloxy)carbonyl)amino)pentyl)amino)-2-oxoethyl)-5,14-dioxo-12-(2-oxo-2-((2-(((2R,3S,4R,5S,6S)-3,4,5-trihydroxy-6-methyltetrahydro-2H-pyran-2-yl)oxy)ethyl)amino)ethyl)-17-(((2R,3S,4R,5S,6S)-3,4,5-trihydroxy-6-methyltetrahydro-2H-pyran-2-yl)oxy)-11-((2-(((2R,3S,4R,5S,6S)-3,4,5-trihydroxy-6-methyltetrahydro-2H-pyran-2-yl)oxy)ethyl) carbamoyl)-3,6,12,15-tetraazaheptadecanoic acid (200 mg, 0.164 mmol) in DMF (2 mL) at rt was added EDC (37.7 mg, 0.197 mmol), HOBT (30.1 mg, 0.782 mmol), and TEA (0.050 mL, 1.629 mmol). After stirring for 30 min, a solution of 6-amino-N-(2-(((2S,3R,4S,5S,6R)-3,4,5-trihydroxy-6-(hydroxymethyl)tetrahydro-2H-pyran-2-yl)oxy)ethyl)hexanamide (83 mg, 0.246 mmol) in DMF (2 mL) was added dropwise. After stirring for 16 h, the mixture was concentrated, and the residue was purified by C18 reverse phase chromatography to provide the title compound. UPLC-MS Method A: m/z=1540.8 (z=); t_{R}=4.48 min.

### Step 2: 2,2′-(((S)-13-(2-((5-Aminopentyl)amino)-2-oxoethyl)-4,11,15,22-tetraoxo-25-(((2S,3R,4S,5S,6R)-3,4,5-trihydroxy-6-(hydroxymethyl)tetrahydro-2H-pyran-2-yl)oxy)-1-(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide)

The title compound was prepared using procedures analogous to those described for Intermediate 5, Step 2, substituting benzyl ((S)-4,13,17-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2S,3R,4S,5S,6R)-3,4,5-trihydroxy-6-(hydroxymethyl)tetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18-pentaazatricosan-23-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate to isolate title compound. UPLC-MS Method A: m/z=1406.7 (z=1); t_{R}=4.60 min.

### Step 3: Benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2S,3R,4S,5S,6R)-3,4,5-trihydroxy-6-(hydroxymethyl)tetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate

The title compound was prepared using procedures analogous to those described for Example 1, Step 4, substituting 2,2′-(((S)-13-(2-((5-aminopentyl)amino)-2-oxoethyl)-4,11,15,22-tetraoxo-25-(((2S,3R,4S,5S,6R)-3,4,5-trihydroxy-6-(hydroxymethyl)tetrahydro-2H-pyran-2-yl)oxy)-1-(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) to isolate the title compound. UPLC-MS Method A: m/z=1213.2 (z=2); t_{R}=3.93 min.

### Step 4: (S)-27-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2S,3R,4S,5S,6R)-3,4,5-trihydroxy-6-(hydroxymethyl)tetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid

The title compound was prepared using procedures analogous to those described for Example 1, Step 5, substituting benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2S,3R,4S,5S,6R)-3,4,5-trihydroxy-6-(hydroxymethyl) tetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy-)ethyl)carbamoyl)-3,6,12,15,18,24,27,30-octaazahexatriacontan-36-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,15,18,24,27,30-octaazahexatriacontan-36-oate to isolate title compound. UPLC-MS Method A: m/z=1168.1 (z=2); t_{R}=3.80 min.

### Step 5: 2,5-Dioxopyrrolidin-1-yl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2S,3R,4S,5S,6R)-3,4,5-trihydroxy-6-(hydroxymethyl) tetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate

The title compound was prepared using procedures analogous to those described for Example 1, Step 6, substituting (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl) amino)ethyl)-15-(2-oxo-2-((6-oxo-6-((2-(((2S,3R,4S,5S,6R)-3,4,5-trihydroxy-6-(hydroxymethyl)tetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1216.5 (z=2); t_{R}=4.10 min.

### Example 10: 2,5-Dioxopyrrolidin-1-yl 25-(2-(((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy) ethyl)amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3,10,13,16,22,25,28-heptaazatetratriacontan-34-oate (ML-10)

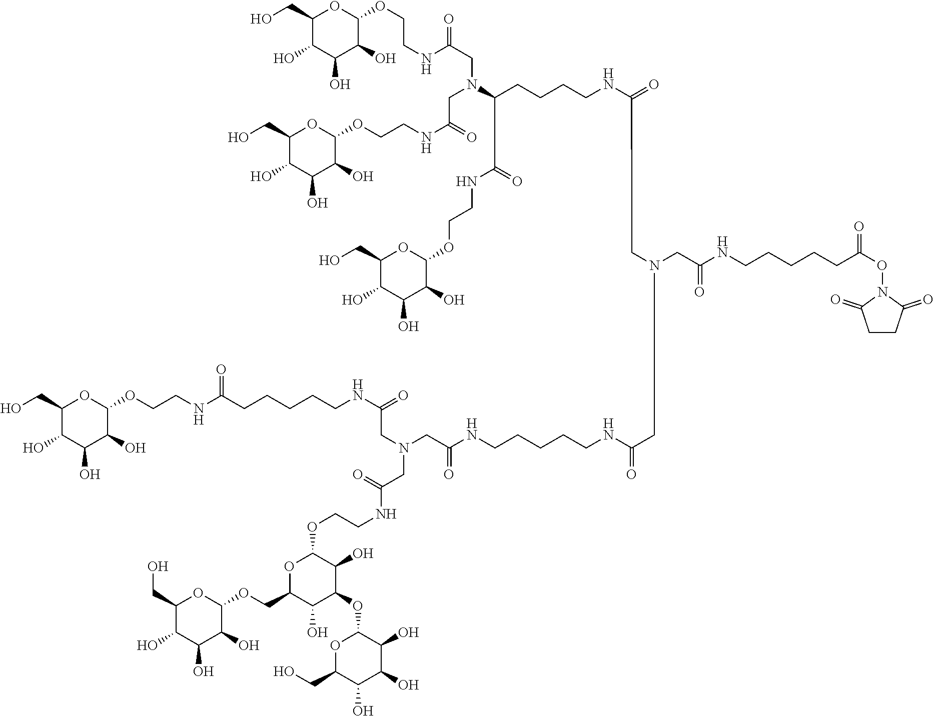 [SMILES: O=C(CCCCCNC(=O)CN(CC(=O)NCCCCCNC(=O)CN(CC(=O)NCCCCCC(=O)ON1C(=O)CCC1=O)CC(=O)NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O)CC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NCCO[C@H]1O[C@H](CO)[C@@H](O)[C@H](O)[C@@H]1O]

### Step 1: 3,11-Dioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)ethyl)-1-phenyl-2-oxa-4,10,13-triazapentadecan-15-oic acid

To 13-(carboxymethyl)-3,11-dioxo-1-phenyl-2-oxa-4,10,13-triazapentadecan-15-oic acid (1.5 g, 3.66 mmol) in anhydrous DCM (20 mL) cooled in an ice-bath was added TFAA (0.647 mL, 4.58 mmol)), and the mixture was stirred at ice-bath temperature for 3 h. The mixture was reduced to −30° C., and a solution of TEA (1.226 mL, 8.79 mmol) in anhydrous DMF (20 mL) was added dropwise over 30 min. Stirring at −30° C. was continued for 30 min, after which a mixture of 6-amino-N-(2-(((2S,3S,4S,5S,6R)-3,4,5-trihydroxy-6-(hydroxymethyl)tetrahydro-2H-pyran-2-yl)oxy)ethyl)hexanamide (1.232 g, 3.66 mmol) in DMF (20 mL) was added, and the resulting mixture was stirred for 16 h. The reaction was concentrated and purified with C18 reverse phase chromatography to isolate the title compound. UPLC-MS Method A: m/z=728.4 (z=1); t_{R}=4.84 min.

### Step 2: 2,5-Dioxopyrrolidin-1-yl 3,11-dioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)ethyl)-1-phenyl-2-oxa-4,10,13-triazapentadecan-15-oate

To a solution of 3,11-dioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)ethyl)-1-phenyl-2-oxa-4,10,13-triazapentadecan-15-oic acid (200 mg, 0.275 mmol) in DMF (6 mL) was added dipyrrolidino (N-succinimidyloxy)carbenium hexafluorophosphate (136 mg, 0.330 mmol) and DIPEA (58 μL), and the mixture was stirred for 5 h. The reaction was neutralized with TFA (47 μL) and concentrated to isolate the title compound. UPLC-MS Method A: m/z=825.4 (z=1); t_{R}=2.29 min.

### Step 3: Benzyl (13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,11,15-trioxo-1-(((2S,3S,4S,5S,6R)-3,4,5-trihydroxy-6-(hydroxymethyl)tetrahydro-2H-pyran-2-yl)oxy)-3,10,13,16-tetraazahenicosan-21-yl)carbamate

To a solution of 2,5-dioxopyrrolidin-1-yl 3,11-dioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl) amino)hexyl) amino)ethyl)-1-phenyl-2-oxa-4,10,13-triazapentadecan-15-oate (227 mg, 0.275 mmol) in DMF (1.38 mL) was added AETM (226 mg, 0.413 mmol). The reaction was stirred for 16 h. The reaction was concentrated and purified on C18 reverse phase chromatography to isolate title compound. UPLC-MS Method A: m/z=1257.6 (z=1); t_{R}=4.49 min.

### Step 4: 6-(2-((2-((5-Aminopentyl)amino)-2-oxoethyl)(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)amino)acetamido)-N-(2-(((2S,3S,4S,5S,6R)-3,4,5-trihydroxy-6-(hydroxymethyl)tetrahydro-2H-pyran-2-yl)oxy)ethyl)hexanamide

The title compound was prepared using procedures analogous to those described for Intermediate 5, Step 2, substituting benzyl (13-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy) methyl)tetrahydro-2H-pyran-2-yl)oxy)ethyl)amino)-2-oxoethyl)-4,11,15-trioxo-1-(((2S,3S,4S,5S,6R)-3,4,5-trihydroxy-6-(hydroxymethyl)tetrahydro-2H-pyran-2-yl)oxy)-3,10,13,16-tetraaza-henicosan-21-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)carbamate to isolate title compound. UPLC-MS Method A: m/z=1123.6 (z=1); t_{R}=4.05 min.

### Step 5: Benzyl 25-(2-(((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl) amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,11,15,23,27-pentaoxo-1-(((2S,3S,4S,5S,6R)-3,4,5-trihydroxy-6-(hydroxymethyl) tetrahydro-2H-pyran-2-yl)oxy)-3,10,13,16, 22, 25,28-heptaazatetratriacontan-34-oate

To a solution of (S)-3-(2-((6-(benzyloxy)-6-oxohexyl)amino)-2-oxoethyl)-5,14-dioxo-12-(2-oxo-2-((2-(((2S,3S,4S,5S,6R)-3,4,5-trihydroxy-6-(hydroxymethyl)tetrahydro-2H-pyran-2-yl) oxy)ethyl)amino)ethyl)-17-(((2S,3S,4S,5S,6R)-3,4,5-trihydroxy-6-(hydroxymethyl)tetrahydro-2H-pyran-2-yl)oxy)-11-((2-(((2S,3S,4S,5S,6R)-3,4,5-trihydroxy-6-(hydroxymethyl)tetrahydro-2H-pyran-2-yl) oxy)ethyl)carbamoyl)-3,6,12,15-tetraazaheptadecanoic acid (60 mg, 0.048 mmol) in DMF (10 mL) at rt was added EDC (9.2 mg, 0.048 mmol), HOBT (7.3 mg, 0.048 mmol), and TEA (10 μL, 0.072 mmol). After stirring for 30 min, a solution of 6-(2-((2-((5-aminopentyl) amino)-2-oxoethyl)(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)amino)acetamido)-N-(2-(((2S,3S,4S,5S,6R)-3,4,5-trihydroxy-6-(hydroxymethyl)tetrahydro-2H-pyran-2-yl)oxy)ethyl) hexanamide (81 mg, 0.072 mmol) in DMF (2 mL) was added dropwise. After stirring for 16 h, the mixture was concentrated, and the residue was purified by C18 reverse phase chromatography to provide the title compound. UPLC-MS Method A: m/z=1180.6 (z=2); t_{R}=3.95 min.

### Step 6: 25-(2-(((S)-5-(Bis(2-oxo-2-((2-(((2S,3S,4S,5S,6R)-3,4,5-trihydroxy-6-(hydroxymethyl) tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-4,11,15,23,27-pentaoxo-1-(((2S,3S,4S,5S,6R)-3,4,5-trihydroxy-6-(hydroxymethyl) tetrahydro-2H-pyran-2-yl)oxy)-3,10,13,16,22,25,28-heptoazatetratriacontan-34-oic acid

The title compound was prepared using procedures analogous to those described for Example 1, Step 5, substituting benzyl 25-(2-(((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy) ethyl)amino)-2-oxoethyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3,10,13,16,22,25,28-heptaazatetratriacontan-34-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1135.0 (z=2); t_{R}=3.80 min.

### Step 7: 2,5-Dioxopyrrolidin-1-yl 25-(2-(((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,11,15,23,27-pentaoxo-1-(((2S,3S,4S,5S,6R)-3,4,5-trihydroxy-6-(hydroxymethyl) tetrahydro-2H-pyran-2-yl)oxy)-3,10,13,16,22,25,28-heptaazatetratriacontan-34-oate

The title compound was prepared using procedures analogous to those described for Example 1, Step 6, substituting 25-(2-(((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl) amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy) ethyl)amino)-2-oxoethyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3,10,13,16,22,25,28-heptaazatetratriacontan-34-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy) ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1184.0 (z=2); t_{R}=4.23 min.

### Example 11: 2,5-Dioxopyrrolidin-1-yl 25-(2-(((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy) ethyl)amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,11,15,23,27-pentaoxo-1-(((2R,3S,4R,5S,6S)-3,4,5-trihydroxy-6-methyltetrahydro-2H-pyran-2-yl)oxy)-3,10,13,16,22,25,28-heptaazatetratriacontan-34-oate (ML-11)

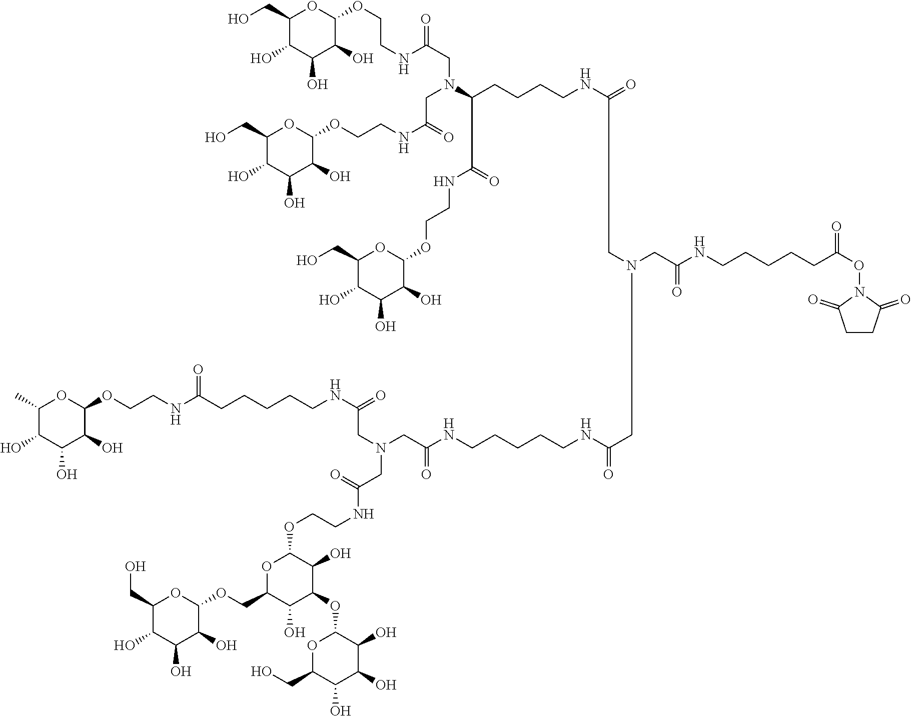 [SMILES: C[C@@H]1O[C@@H](OCCNC(=O)CCCCCNC(=O)CN(CC(=O)NCCCCCNC(=O)CN(CC(=O)NCCCCCC(=O)ON2C(=O)CCC2=O)CC(=O)NCCCC[C@@H](C(=O)NCCO[C@H]2O[C@H](CO)[C@@H](O)[C@H](O)[C@@H]2O)N(CC(=O)NCCO[C@H]2O[C@H](CO)[C@@H](O)[C@H](O)[C@@H]2O)CC(=O)NCCO[C@H]2O[C@H](CO)[C@@H](O)[C@H](O)[C@@H]2O)CC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)[C@@H](O)[C@H](O)[C@@H]1O]

### Step 1: 3,11-Dioxo-13-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-phenyl-2-oxa-4,10,13-triazapentadecan-15-oic acid

To 13-(carboxymethyl)-3,11-dioxo-1-phenyl-2-oxa-4,10,13-triazapentadecan-15-oic acid (1.5 g, 3.66 mmol) in anhydrous DCM (20 mL), cooled in an ice-bath, was added TFAA (0.647 mL, 4.58 mmol)), and the mixture was stirred at ice-bath temperature for 3 h. The temperature was reduced to −30° C., and a solution of TEA (1.226 mL, 8.79 mmol) in anhydrous DMF (20 mL) was added dropwise over 30 min. Stirring at −30° C. was continued for 30 min, then a mixture of 6-amino-N-(2-(((2R,3S,4R,5S,6S)-3,4,5-trihydroxy-6-methyltetrahydro-2H-pyran-2-yl)oxy)ethyl) hexanamide in DMF (20 mL) was added, and the mixture was stirred for 16 h. The reaction was concentrated and purified with C18 reverse phase chromatography to isolate the title compound. UPLC-MS Method A: m/z=712.4 (z=1); t_{R}=4.95 min.

### Step 2: 2,5-Dioxopyrrolidin-1-yl 3,11-dioxo-13-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-phenyl-2-oxa-4,10,13-triazapentadecan-15-oate

To a solution 3,11-dioxo-13-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-phenyl-2-oxa-4,10,13-triazapentadecan-15-oic acid (200 mg, 0.281 mmol) in DMF (6 mL) was added dipyrrolidino(N-succinimidyloxy)carbenium hexafluorophosphate (139 mg, 0.337 mmol) and DIPEA (59 μL, 0.337 mmol), and the mixture was stirred for 5 h. The reaction is neutralized with TFA (47 L) and concentrated to isolate the title compound. UPLC-MS Method A: m/z=809.4 (z=1); t_{R}=3.03 min.

### Step 3: Benzyl (13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,11,15-trioxo-1-(((2R,3S,4R,5S,6S)-3,4,5-trihydroxy-6-methyltetrahydro-2H-pyran-2-yl)oxy)-3,10,13,16-tetraazahenicosan-21-yl)carbamate

To a solution of 2,5-dioxopyrrolidin-1-yl 3,11-dioxo-13-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-phenyl-2-oxa-4,10,13-triazapentadecan-15-oate (227 mg, 0.275 mmol) in DMF (6 mL) was added AETM (230 mg, 0.421 mmol). The reaction was stirred for 16 h. The reaction was concentrated and purified on C18 reverse phase chromatography to isolate title compound. UPLC-MS Method A: m/z=1241.6 (z=1); t_{R}=4.55 min.

### Step 4: 6-(2-((2-((5-Aminopentyl)amino)-2-oxoethyl)(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)amino)acetamido)-N-(2-(((2R,3S,4R,5S,6S)-3,4,5-trihydroxy-6-methyltetrahydro-2H-pyran-2-yl)oxy)ethyl)hexanamide

The title compound was prepared using procedures analogous to those described for Intermediate 5, Step 2, substituting benzyl (13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,11,15-trioxo-1-(((2R,3S,4R,5S,6S)-3,4,5-trihydroxy-6-methyltetrahydro-2H-pyran-2-yl)oxy)-3,10,13,16-tetraazahenicosan-21-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate to isolate title compound. UPLC-MS Method A: m/z=1107.5 (z=1); t_{R}=4.03 min.

### Step 5: Benzyl 25-(2-(((S)-5-(bis(2-oxo-2-((2-(((2S,3S,4S,5S,6R)-3,4,5-trihydroxy-6-(hydroxymethyl) tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,11,15,23,27-pentaoxo-1-(((2R,3S,4R,5S,6S)-3,4,5-trihydroxy-6-methyltetrahydro-2H-pyran-2-yl)oxy)-3,10,13,16,22,25,28-heptaazatetratriacontan-34-oate

To a solution of (S)-3-(2-((6-(benzyloxy)-6-oxohexyl)amino)-2-oxoethyl)-5,14-dioxo-12-(2-oxo-2-((2-(((2S,3S,4S,5S,6R)-3,4,5-trihydroxy-6-(hydroxymethyl)tetrahydro-2H-pyran-2-yl)oxy) ethyl)amino)ethyl)-17-(((2S,3S,4S,5S,6R)-3,4,5-trihydroxy-6-(hydroxymethyl) tetrahydro-2H-pyran-2-yl)oxy)-11-((2-(((2S,3S,4S,5S,6R)-3,4,5-trihydroxy-6-(hydroxymethyl) tetrahydro-2H-pyran-2-yl)oxy)ethyl)carbamoyl)-3,6,12,15-tetraazaheptadecanoic acid (60 mg, 0.048 mmol) in DMF (10 mL) at rt was added EDC (9.2 mg, 0.048 mmol), HOBT (7.3 mg, 0.048 mmol), and TEA (10 μL, 0.072 mmol). After stirring the mixture for 30 min, 6-(2-((2-((5-aminopentyl)amino)-2-oxoethyl)(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)amino)acetamido)-N-(2-(((2R,3S,4R,5S,6S)-3,4,5-trihydroxy-6-methyltetrahydro-2H-pyran-2-yl)oxy)ethyl)hexanamide (81 mg, 0.072 mmol) was added. After stirring for 16 h, the mixture was concentrated, and the residue was purified by C18 reverse phase chromatography to provide the title compound. UPLC-MS Method A: m/z=1172.6 (z=2); t_{R}=3.96 min.

### Step 6: 25-(2-(((S)-5-(Bis(2-oxo-2-((2-(((2S,3S,4S,5S,6R)-3,4,5-trihydroxy-6-(hydroxymethyl) tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-4,11,15,23,27-pentaoxo-1-(((2R,3S,4R,5S,6 S)-3,4,5-trihydroxy-6-methyltetrahydro-2H-pyran-2-yl)oxy)-3,10,13,16, 22, 25, 28-heptaazatetratriacontan-34-oic acid

The title compound was prepared using procedures analogous to those described for Example 1, Step 5, substituting benzyl 25-(2-(((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy) ethyl)amino)-2-oxoethyl)-4,11,15,23,27-pentaoxo-1-(((2R,3S,4R,5S,6S)-3,4,5-trihydroxy-6-methyltetrahydro-2H-pyran-2-yl)oxy)-3,10,13,16,22,25,28-heptaazatetratriacontan-34-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1127.5 (z=2); t_{R}=3.80 min.

### Step 7: 2,5-Dioxopyrrolidin-1-yl 25-(2-(((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,11,15,23,27-pentaoxo-1-(((2R,3S,4R,5S,6S)-3,4,5-trihydroxy-6-methyltetrahydro-2H-pyran-2-yl)oxy)-3,10,13,16,22,25,28-heptaazatetratriacontan-34-oate

The title compound was prepared using procedures analogous to those described for Example 1, Step 6, substituting 25-(2-(((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)amino)-2-oxoethyl)-13-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy) ethyl)amino)-2-oxoethyl)-4,11,15,23,27-pentaoxo-1-(((2R,3S,4R,5S,6S)-3,4,5-trihydroxy-6-methyltetrahydro-2H-pyran-2-yl)oxy)-3,10,13,16,22,25,28-heptaazatetratriacontan-34-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oic acid to isolate title compound. UPLC-MS Method A: m/z=1184.0 (z=2); t_{R}=4.23 min.

### Example 12: 2,5-Dioxopyrrolidin-1-yl (S)-34-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetralhydro-2H-pyran-2-yl)oxy)methyl) tetrahydro-2H-pyran-2-yl)oxy)ethyl)amino)-6-oxohexyl)amino)-2-oxoethyl)-4,13,20,24,32,36-hexaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-22-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,19,22,25,31,34,37-nonaazatritetracontan-43-oate (ML-12)

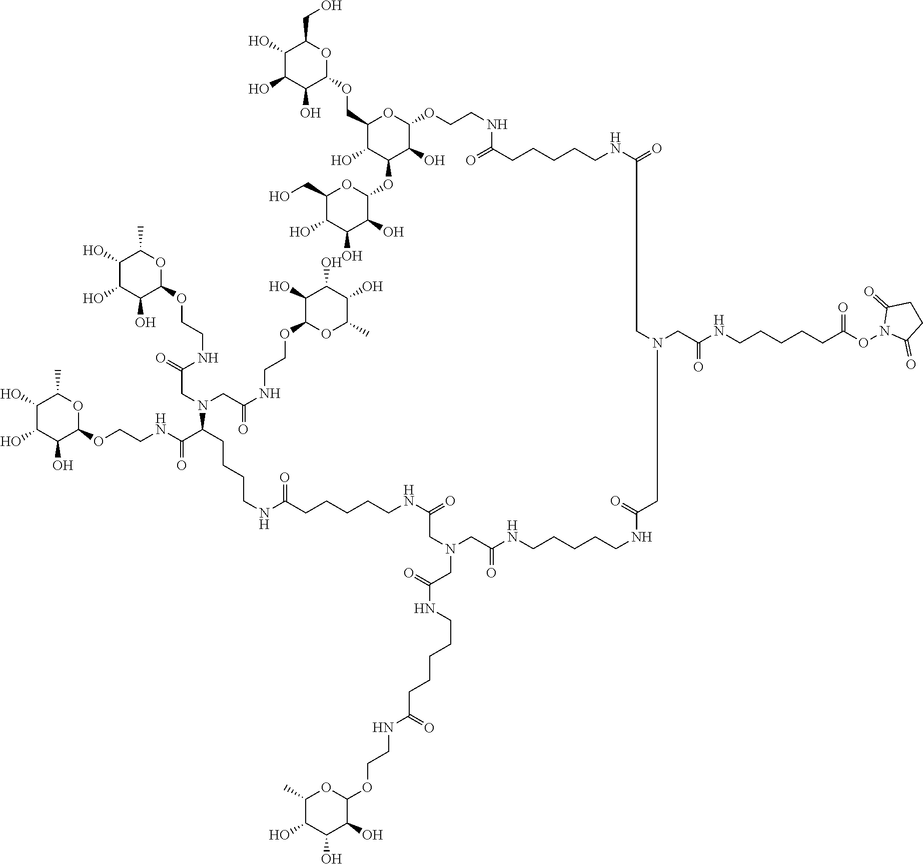 [SMILES: C[C@@H]1O[C@@H](OCCNC(=O)CN(CC(=O)NCCO[C@@H]2O[C@@H](C)[C@@H](O)[C@@H](O)[C@@H]2O)[C@@H](CCCCNC(=O)CCCCCNC(=O)CN(CC(=O)NCCCCCNC(=O)CN(CC(=O)NCCCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)ON2C(=O)CCC2=O)CC(=O)NCCCCCC(=O)NCCOC2O[C@@H](C)[C@@H](O)[C@@H](O)[C@@H]2O)C(=O)NCCO[C@@H]2O[C@@H](C)[C@@H](O)[C@@H](O)[C@@H]2O)[C@@H](O)[C@H](O)[C@@H]1O]

### Step 1: Benzyl (6-(((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)amino)-6-oxohexyl)carbamate

The title compound was prepared using procedures analogous to those described for Intermediate 4, Step 1, substituting 2,2′-(((S)-6-amino-1-oxo-1-((2-(((2R,3S,4R,5S,6S)-3,4,5-trihydroxy-6-methyltetrahydro-2H-pyran-2-yl)oxy)ethyl)amino)hexan-2-yl)azanediyl) bis(N-(2-(((2R,3S,4R,5S,6S)-3,4,5-trihydroxy-6-methyltetrahydro-2H-pyran-2-yl)oxy)ethyl)acetamide) for AEM to isolate title compound. UPLC-MS Method A: m/z=1077.6 (z=1); t_{R}=4.12 min.

### Step 2: 2, 2′-(((S)-6-(6-Aminohexanamido)-1-oxo-1-((2-(((2R,3S,4R,5S,6S)-3,4,5-trihydroxy-6-methyltetrahydro-2H-pyran-2-yl)oxy)ethyl)amino)hexan-2-yl)azanediyl)bis(N-(2-(((2R,3S,4R,5S,6S)-3,4,5-trihydroxy-6-methyltetrahydro-2H-pyran-2-yl)oxy)ethyl)acetamide)

The title compound was prepared using procedures analogous to those described for Intermediate 5, Step 2, substituting benzyl (6-(((S)-5-(bis(2-oxo-2-((2-(((2R,3S,4R,5S,6S)-3,4,5-trihydroxy-6-methyltetrahydro-2H-pyran-2-yl)oxy)ethyl) amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)amino)-6-oxohexyl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy) ethyl)amino)-6-oxohexyl)carbamate to isolate title compound. UPLC-MS Method A: m/z=943.5 (z=1); t_{R}=4.00 min.

### Step 3: Benzyl ((S)-4,13,20,24-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-22-(2-oxo-2-((6-oxo-6-((2-(((2R,3S,4R,5S,6)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,19,22,25-hexaazatriacontan-30-yl)carbamate

The title compound was prepared using procedures analogous to those described for Intermediate 12, Step 1, substituting 2,2′-(((S)-6-(6-aminohexanamido)-1-oxo-1-((2-(((2R,3S,4R,5S,6S)-3,4,5-trihydroxy-6-methyltetrahydro-2H-pyran-2-yl)oxy)ethyl)amino)hexan-2-yl) azanediyl)bis(N-(2-(((2R,3S,4R,5S,6S)-3,4,5-trihydroxy-6-methyltetrahydro-2H-pyran-2-yl)oxy) ethyl)acetamide) for AETM to isolate the title compound. UPLC-MS Method A: m/z=1637.8 (z=1); t_{R}=4.34 min.

### Step 4: 2, 2′-(((S)-13-(2-((5-Aminopentyl)amino)-2-oxoethyl)-4,11,15,22,29-pentaoxo-1,32-bis(((2R,3S,4R,5S,6S)-3,4,5-trihydroxy-6-methyltetrahydro-2H-pyran-2-yl)oxy)-3,10,13,16,23,30-hexaazadotriacontan-28-yl)azanediyl)bis(N-(2-(((2R,3S,4R,5S,6S)-3,4,5-trihydroxy-6-methyltetrahydro-2H-pyran-2-yl)oxy)ethyl)acetamide)

The title compound was prepared using procedures analogous to those described for Intermediate 5, Step 2, substituting benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate for benzyl ((S)-4,13,20,24-tetraoxo-6-(2-oxo-2-((2-(((2R,3S,4R,5S,6S)-3,4,5-trihydroxy-6-methyltetrahydro-2H-pyran-2-yl)oxy)ethyl) amino)ethyl)-22-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,19,22,25-hexaazatriacontan-30-yl)carbamate to isolate title compound. UPLC-MS Method A: m/z=1503.8 (z=1); t_{R}=4.15 min.

### Step 5: Benzyl (S)-34-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,20,24,32,36-hexaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-22-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,19,22,25,31,34,37-nonaazatritetracontan-43-oate

The title compound was prepared using procedures analogous to those described for Example 1, Step 4, substituting 2,2′-(((S)-13-(2-((5-aminopentyl)amino)-2-oxoethyl)-4,11,15,22,29-pentaoxo-1,32-bis(((2R,3S,4R,5S,6S)-3,4,5-trihydroxy-6-methyltetrahydro-2H-pyran-2-yl)oxy)-3,10,13,16,23,30-hexaazadotriacontan-28-yl)azanediyl)bis(N-(2-(((2R,3S,4R,5S,6S)-3,4,5-trihydroxy-6-methyltetrahydro-2H-pyran-2-yl)oxy)ethyl)acetamide) for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl) azanediyl)bis(N-(2-(((2R,3S,4R,5S,6S)-3,4,5-trihydroxy-6-methyltetrahydro-2H-pyran-2-yl)oxy) ethyl)acetamide) to isolate the title compound. UPLC-MS Method A: m/z=1261.7 (z=2); t_{R}=4.37.

### Step 6: (S)-34-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,20,24,32,36-hexaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-22-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl) amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,19,22,25,31,34,37-nonaazatritetracontan-43-oic acid

The title compound was prepared using procedures analogous to those described for Example 1, Step 5, substituting benzyl (S)-34-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,20,24,32,36-hexaoxo-6-(2-oxo-2-((2-(((2R,3S,4R,5S,6S)-3,4,5-trihydroxy-6-methyltetrahydro-2H-pyran-2-yl)oxy)ethyl) amino)ethyl)-22-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,19,22,25,31,34,37-nonaazatritetracontan-43-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1216.6 (z=2); t_{R}=4.19 min.

### Step 7: 2,5-Dioxopyrrolidin-1-yl (S)-34-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,20,24,32,36-hexaoxo-6-(2-oxo-2-((2-(((2R,3S,4R,5S,6S)-3,4,5-trihydroxy-6-methyltetrahydro-2H-pyran-2-yl)oxy)ethyl) amino)ethyl)-22-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,19,22,25,31,34,37-nonaazatritetracontan-43-oate

The title compound was prepared using procedures analogous to those described for Example 1, Step 6, substituting (S)-34-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)amino)-2-oxoethyl)-4,13,20, 24,32,36-hexaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-22-(2-oxo-2-((6-oxo-6-((2-(((2R,3S,4R,5S,6S)-3,4,5-trihydroxy-6-methyltetrahydro-2H-pyran-2-yl) oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,19,22,25,31,34,37-nonaazatritetracontan-43-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oic acid to isolate title compound. UPLC-MS Method A: m/z=1265.1 (z=2); t_{R}=4.22 min.

### Example 13: 2,5-Dioxopyrrolidin-1-yl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29,36-hexaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-40,43,46,49,52,55-hexaoxa-3,6,12,15,18,24,27,30,37-nonaazaoctapentacontan-58-oate (ML-13)

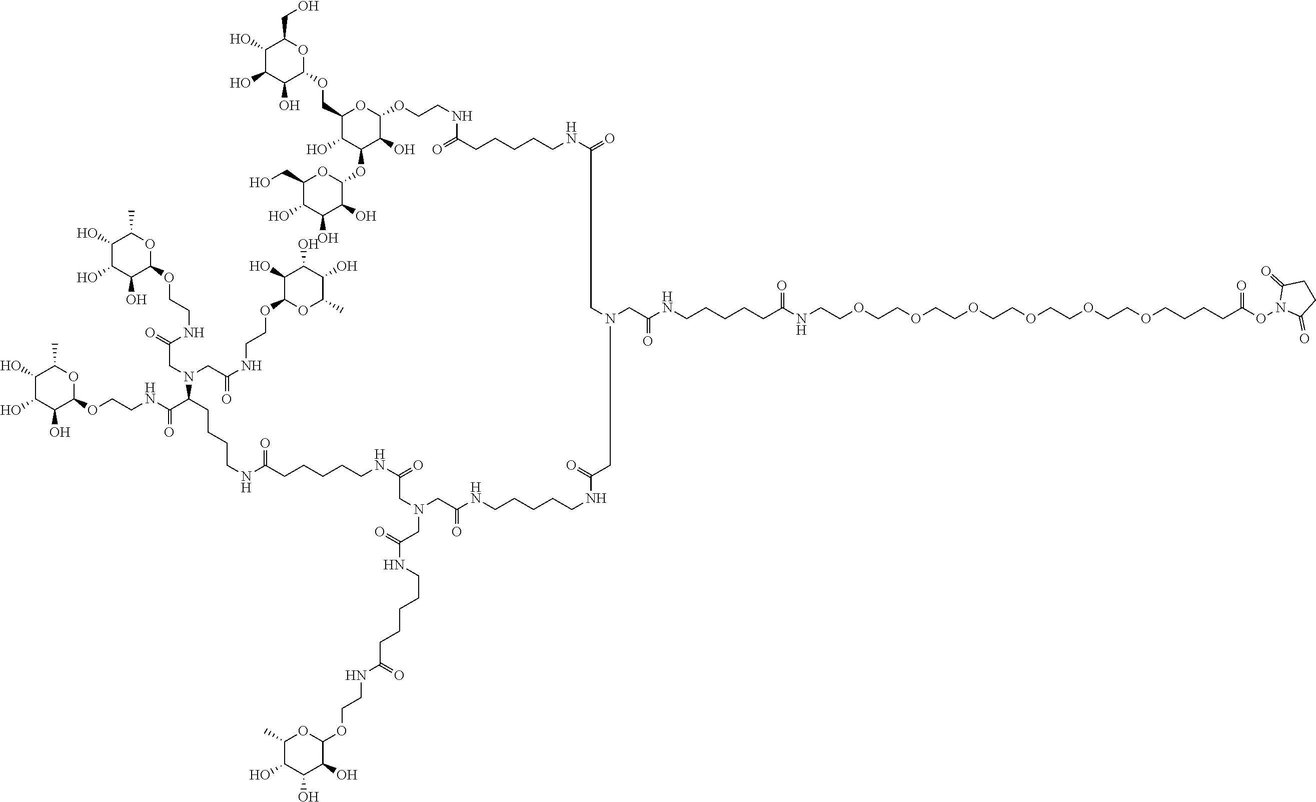 [SMILES: C[C@@H]1O[C@@H](OCCNC(=O)CN(CC(=O)NCCO[C@@H]2O[C@@H](C)[C@@H](O)[C@@H](O)[C@@H]2O)[C@@H](CCCCNC(=O)CCCCCNC(=O)CN(CC(=O)NCCCCCNC(=O)CN(CC(=O)NCCCCCC(=O)NCCOCCOCCOCCOCCOCCOCCCCC(=O)ON2C(=O)CCC2=O)CC(=O)NCCCCCC(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC(=O)NCCCCCC(=O)NCCOC2O[C@@H](C)[C@@H](O)[C@@H](O)[C@@H]2O)C(=O)NCCO[C@@H]2O[C@@H](C)[C@@H](O)[C@@H](O)[C@@H]2O)[C@@H](O)[C@H](O)[C@@H]1O]

### Step 1: (S)-27-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17, 25, 29, 36-hexaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl) amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-40, 43, 46, 49, 52,55-hexaoxa-3, 6,12,15,18, 24, 27, 30, 37-nonaazaoctapentacontan-58-oic acid

To a solution of 2,5-dioxopyrrolidin-1-yl (S)-27-(2-((6-((2-(((2S,3S,4S,5R,6R)-3,5-dihydroxy-4-(((2R,3 S,4S,5 S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3 S,4R,5 S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3 S,4R,5 S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate (78.4 mg, 0.032 mmol) in DMF (2 mL) and DIPEA (0.012 mL, 0.071 mmol) was added to the solution. To the solution, NH₂-PEG(6)-CO₂H (13.76 mg, 0.039 mmol) was added and aged for 16 h. The solution was triturated from ACN and was decanted, and the residue was taken into water and freeze dried to isolate the title compound. UPLC-MS Method A: m/z=1327.5 (z=2); t_{R}=1.99 min.

### Step 2: 2,5-Dioxopyrrolidin-1-yl (S)-27-(2-((6-((2-(((2S,3S,4S,5R,66R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29,36-hexaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-40,43,46,49,52,55-hexaoxa-3,6,12,15,18,24,27,30,37-nonaazaoctapentacontan-58-oate

The title compound was prepared using procedures analogous to those described for Example 1, Step 6, substituting (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29,36-hexaoxo-6-(2-oxo-2-((2-(((2R,3S,4R,5S,6S)-3,4,5-trihydroxy-6-methyltetrahydro-2H-pyran-2-yl)oxy)ethyl) amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-40,43,46,49,52,55-hexaoxa-3,6,12,15,18,24,27,30,37-nonaazaoctapentacontan-58-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,15,18,24,27,30-octaazahexatriacontan-36-oic acid to isolate title compound. UPLC-MS Method A: m/z=1376.1 (z=2); t_{R}=2.19 min.

### Example 14: 2,5-Dioxopyrrolidin-1-yl 13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-25-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25,28-heptaazatetratriacontan-34-oate (ML-14)

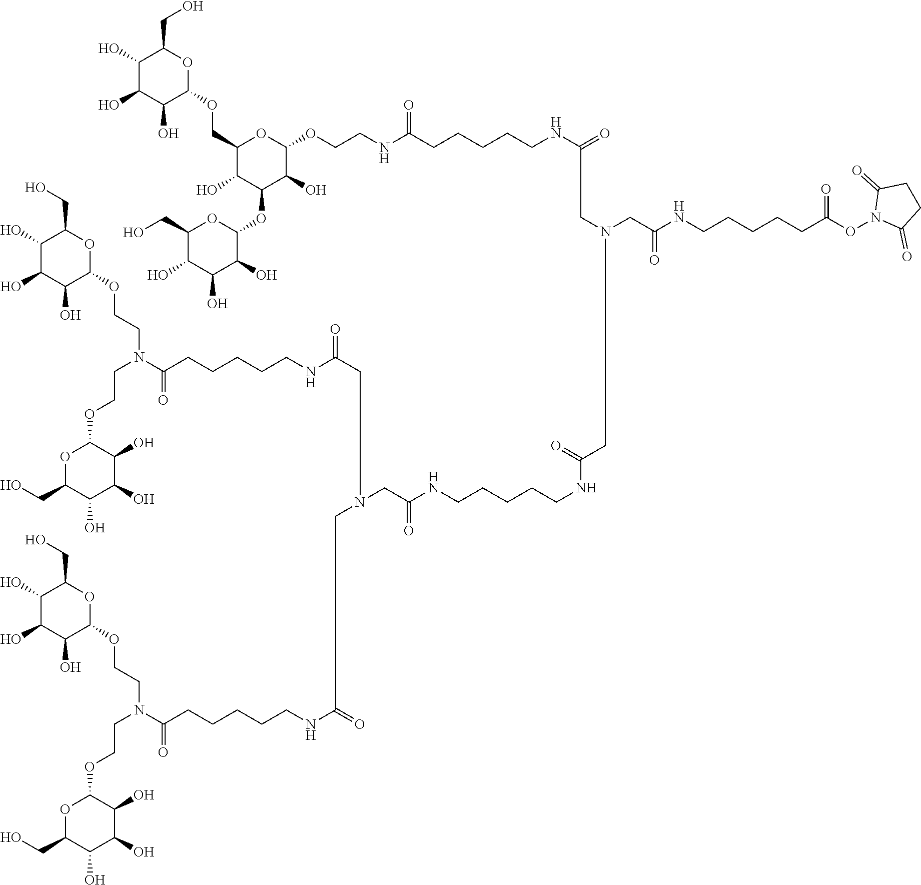 [SMILES: O=C(CCCCCNC(=O)CN(CC(=O)NCCCCCNC(=O)CN(CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)NCCCCCC(=O)ON1C(=O)CCC1=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl (6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamate

To a solution of 2,5-dioxopyrrolidin-1-yl 6-(((benzyloxy)carbonyl)amino) hexanoate (1200 mg, 3.31 mmol) in DMF (60 mL) was added DIPEA (0.694 mL, 3.97 mmol), followed by bis{2-[(α-D-mannopyranosyl)oxy]ethyl}amine (WO2018/175272 A1) (2789 mg, 3.64 mmol), and the reaction was stirred for 16 h. The solution was added to 50 mL of water, and the aqueous portion was extracted 3× with EtOAc. The organic was then washed with citric acid (2×15 mL), followed by brine (25 mL), dried over MgSO₄, and concentrated under reduced pressure. The resulting residue was taken up in MeOH (60 mL), where a solution of 30% NaOMe in MeOH (1 mL, 5.38 mmol) was added and stirred for 1 h. The solution was concentrated under reduced pressure and purified by C18 reverse phase chromatography. UPLC-MS Method A: m/z=677.3 (z=1); t_{R}=2.80 min.

### Step 2: 6-Amino-N,N-bis(2-(((2S,3S,4S,5S,6R)-3,4,5-trihydroxy-6-(hydroxymethyl)tetrahydro-2H-pyran-2-yl)oxy)ethyl)hexanamide

The title compound was prepared using procedures analogous to those described for Intermediate 5, Step 2, substituting benzyl (6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) carbamate to isolate title compound. UPLC-MS Method A: m/z=543.2 (z=1); t_{R}=1.36 min.

### Step 3: Benzyl (13-(2-((6-(bis(2-(((2S,3S,4S,5S,6R)-3,4,5-trihydroxy-6-(hydroxymethyl) tetrahydro-2H-pyran-2-yl)oxy)ethyl)amino)-6-oxohexyl)amino)-2-oxoethyl)-4,11,15-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16-tetraazahenicosan-21-yl)carbamate

To a solution of 13-(carboxymethyl)-3,11-dioxo-1-phenyl-2-oxa-4,10,13-triazapentadecan-15-oic acid (400 mg, 0.977 mmol) in DMF (10 mL) at rt was added EDC (468 mg, 2.442 mmol) and HOBT (15.0 mg, 0.098 mmol). After stirring 30 min, to the reaction mixture was added a solution of 6-amino-N,N-bis(2-(((2S,3S,4S,5S,6R)-3,4,5-trihydroxy-6-(hydroxymethyl)tetrahydro-2H-pyran-2-yl)oxy)ethyl)hexanamide) (1157 mg, 2.132 mmol) in DMF (10 mL) dropwise. After stirring for 16 h, the mixture was concentrated, and the residue was purified by C18 reverse phase chromatography to provide the title compound. UPLC-MS Method A: m/z=1459.8 (z=1); t_{R}=2.69 min.

### Step 4: 6,6′-((2,2′-((2-((5-Aminopentyl)amino)-2-oxoethyl)azanediyl)bis(acetyl))bis(azanediyl)) bis(N,N-bis(2-(((2S,3S,4S,5S,6R)-3,4,5-trihydroxy-6-(hydroxymethyl)tetrahydro-2H-pyran-2-yl)oxy)ethyl)hexanamide)

The title compound was prepared using procedures analogous to those described for Intermediate 5, Step 2, substituting benzyl (13-(2-((6-(bis(2-(((2S,3S,4S,5S,6R)-3,4,5-trihydroxy-6-(hydroxymethyl)tetrahydro-2H-pyran-2-yl)oxy)ethyl) amino)-6-oxohexyl)amino)-2-oxoethyl)-4,11,15-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16-tetraazahenicosan-21-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl) carbamate to isolate title compound. UPLC-MS Method A: m/z=1324.7 (z=1); t_{R}=1.70 min.

### Step 5: Benzyl 13-(2-((6-(bis(2-(((2S,38.4S, S, 6R)-3,4,5-trihydroxy-6-(hydroxymethyl) tetrahydro-2H-pyran-2-yl)oxy)ethyl)amino)-6-oxohexyl)amino)-2-oxoethyl)-25-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl)amino)-2-oxoethyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25,28-heptaazatetratriacontan-34-oate

The title compound was prepared using procedures analogous to those described for Example 1, Step 4, substituting 6,6′-((2,2′-((2-((5-aminopentyl)amino)-2-oxoethyl)azanediyl) bis(acetyl))bis(azanediyl))bis(N,N-bis(2-(((2S,3S,4S,5S,6R)-3,4,5-trihydroxy-6-(hydroxymethyl) tetrahydro-2H-pyran-2-yl)oxy)ethyl) hexanamide) for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) to isolate the title compound. UPLC-MS Method A: m/z=1172.6 (z=2); t_{R}=2.73.

### Step 6: 13-(2-((6-(Bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-25-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25,28-heptaazatetratriacontan-34-oic acid

The title compound was prepared using procedures analogous to those described for Example 1, Step 5, substituting benzyl 13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-25-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25,28-heptaazatetratriacontan-34-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1127.6 (z=2); t_{R}=2.02 min.

### Step 7: 2,5-Dioxopyrrolidin-1-yl 13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-25-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25,28-heptaazatetratriacontan-34-oate

The title compound was prepared using procedures analogous to those described for Example 1, Step 6, substituting 13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-25-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25,28-heptaazatetratriacontan-34-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1176.5 (z=2); t_{R}=2.12 min.

### Example 15: (S)-2,5-Dioxopyrrolidin-1-yl 15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oate (ML-15)

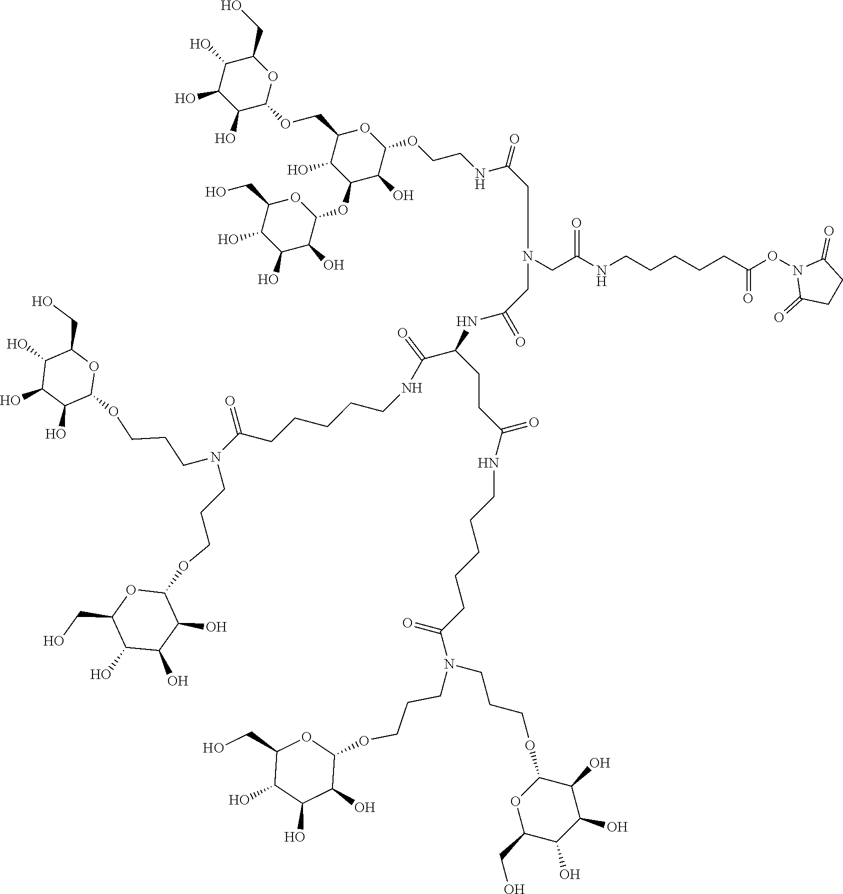 [SMILES: O=C(CC[C@H](NC(=O)CN(CC(=O)NCCCCCC(=O)ON1C(=O)CCC1=O)CC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)C(=O)NCCCCCC(=O)N(CCCO[C@H]1O[C@H](CO)[C@@H](O)[C@H](O)[C@@H]1O)CCCO[C@H]1O[C@H](CO)[C@@H](O)[C@H](O)[C@@H]1O)NCCCCCC(=O)N(CCCO[C@H]1O[C@H](CO)[C@@H](O)[C@H](O)[C@@H]1O)CCCO[C@H]1O[C@H](CO)[C@@H](O)[C@H](O)[C@@H]1O]

### Step 1: N-Benzyl-3-(((2S,3S,4S,5R,6R)-3,4,5-tris(benzyloxy)-6-((benzyloxy)methyl)tetrahydro-2H-pyran-2-yl)oxy)-N-(3-(((2S,3S,4S,5R,6R)-3,4,5-tris(benzyloxy)-6-((benzyloxy)methyl) tetrahydro-2H-pyran-2-yl)oxy)propyl)propan-1-amine

To a solution of 3-(((2S,3S,4S,5R,6R)-3,4,5-tris(benzyloxy)-6-((benzyloxy)methyl) tetrahydro-2H-pyran-2-yl)oxy)propanal (16 g, 26.7 mmol) and benzylamine (1.3 g, 12.13 mmol) in DCM was added TFA (0.935 mL, 12.13 mmol). To this mixture was added, portion Wise, sodium triacetoxyborohydride (7.71 g, 36.4 mmol), and the resulting mixture was stirred at rt for 3 d. The mixture was evaporated and partitioned between EtOAc (200 mL) and sat. NaHCO₃ (200 mL). The organic layer washed with sat. NaCl (100 mL), dried over Na₂SO₄, filtered and evaporated. The residue was purified by silica gel column chromatography to isolate title compound. UPLC-MS Method A: m/z=1268.5 (z=1); t_{R}=4.21 min.

### Step 2: (2R,2′R, 3S,3′S,4S,4′S,5S,5′S,6S,6′S)-6,6′-((Azanediylbis(propane-3,1-diyl))bis(oxy))bis(2-(hydroxymethyl)tetrahydro-2H-pyran-3,4,5-triol) hydrochloride

To a solution of N-benzyl-3-(((2S,3S,4S,5R,6R)-3,4,5-tris(benzyloxy)-6-((benzyloxy) methyl)tetrahydro-2H-pyran-2-yl)oxy)-N-(3-(((2S,3S,4S,5R,6R)-3,4,5-tris(benzyloxy)-6-((benzyloxy)methyl)tetrahydro-2H-pyran-2-yl)oxy)propyl)propan-1-amine (6.69 g, 5.27 mmol) in a mixture of MeOH (100 mL) and water (30 mL) was added 1M HCl (10.55 mL, 10.55 mmol). The mixture was flushed with N_{2(g)}, and 10% Pd/C (560 mg) was added. The resulting mixture was stirred under a balloon of H_{2(g)} overnight. The mixture was filtered through CELITE™ diatomaceous earth, and the filtrate was evaporated to remove the MeOH. The remaining aqueous portion was freeze dried to isolate title compound. UPLC-MS Method A: m/z=458.2 (z=1); t_{R}=0.86 min.

### Step 3: Benzyl (6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamate

To a mixture of (2R,2′R,3S,3′S,4S,4′S,5S,5′S,6S,6′S)-6,6′-((azanediylbis(propane-3,1-diyl))bis(oxy))bis(2-(hydroxymethyl)tetrahydro-2H-pyran-3,4,5-triol) hydrochloride (2.6 g, 5.26 mmol) and 2,5-dioxopyrrolidin-1-yl 6-(((benzyloxy)carbonyl)amino)hexanoate (2.29 g, 6.32 mmol) in anhydrous DMF (50 mL) was added DIPEA (2.21 mL, 12.63 mmol), and the mixture was stirred at rt overnight. The mixture was evaporated, and the residue purified by C18 reverse phase chromatography to isolate the title compound. UPLC-MS Method A: m/z=705.3 (z=1); t_{R}=2.70 min.

### Step 4: 6-Amino-N,N-bis(3-(((2S,3S,4S,5S,6R)-3,4,5-trihydroxy-6-(hydroxymethyl)tetrahydro-2H-pyran-2-yl)oxy)propyl)hexanamide

The title compound was prepared using procedures analogous to those described for Intermediate 5, Step 2, substituting benzyl (6-(bis(3-(((2S,3S,4S,5S,6R)-3,4,5-trihydroxy-6-(hydroxymethyl)tetrahydro-2H-pyran-2-yl)oxy)propyl) amino)-6-oxohexyl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl) carbamate to isolate title compound. UPLC-MS Method A: m/z=571.3 (z=1); t_{R}=1.11 min.

### Step 5: Benzyl ((S)-5,12,16,23-tetraoxo-1,27-bis(((2S,3S,4S,5S,6R)-3,4,5-trihydroxy-6-(hydroxymethyl)tetrahydro-2H-pyran-2-yl)oxy)-4,24-bis(3-(((2S,3S,4S,5S,6R)-3,4,5-trihydroxy-6-(hydroxymethyl)tetrahydro-2H-pyran-2-yl)oxy)propyl)-4,11,17,24-tetraazaheptacosan-13-yl)carbamate

To a solution of (S)-2-(((benzyloxy)carbonyl)amino)pentanedioic acid (145 mg, 0.516 mmol) and 6-amino-N,N-bis(3-(((2S,3S,4S,5S,6R)-3,4,5-trihydroxy-6-(hydroxymethyl) tetrahydro-2H-pyran-2-yl)oxy)propyl)hexanamide (706 mg, 1.237 mmol) in anhydrous DMF (4 mL) was added HOBt (174 mg, 1.134 mmol) followed by EDC (296 mg, 1.547 mmol), and the resulting mixture was stirred at rt for 1 h. The mixture purified by C18 reverse phase chromatography to isolate the title compound. UPLC-MS Method A: m/z=1386.6 (z=1); t_{R}=2.16 min.

### Step 6: (S)-2-Amino-N1,N5-bis(6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)pentanediamide

The title compound was prepared using procedures analogous to those described for Intermediate 5, Step 2, substituting benzyl ((S)-5,12,16,23-tetraoxo-1,27-bis(((2S,3S,4S,5S,6R)-3,4,5-trihydroxy-6-(hydroxymethyl)tetrahydro-2H-pyran-2-yl)oxy)-4,24-bis(3-(((2S,3S,4S,5S,6R)-3,4,5-trihydroxy-6-(hydroxymethyl)tetrahydro-2H-pyran-2-yl)oxy)propyl)-4,11,17,24-tetraazaheptacosan-13-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)carbamate to isolate title compound. UPLC-MS Method A: m/z=1252.6 (z=1); t_{R}=1.62 min.

### Step 7: Benzyl (S)-15-((6-(bis(3-(((2S,3S,4S,5S,6R)-3,4,5-trihydroxy-6-(hydroxymethyl) tetrahydro-2H-pyran-2-yl)oxy)propyl)amino)-6-oxohexyl)carbamoyl)-19-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oate

To a mixture of N-(2-((6-(benzyloxy)-6-oxohexyl)amino)-2-oxoethyl)-N-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl) glycine (100 mg, 0.108 mmol) and (S)-2-amino-N1,N5-bis(6-(bis(3-(((2S,3S,4S,5S,6R)-3,4,5-trihydroxy-6-(hydroxymethyl) tetrahydro-2H-pyran-2-yl)oxy)propyl)amino)-6-oxohexyl) pentanediamide (152 mg, 0.13 mmol) in anhydrous DMF (1.5 mL) was added HOBt (20 mg, 0.13 mmol) and EDC (42 mg, 0.216 mmol), and the resulting mixture was stirred at rt for 90 min. The mixture was added dropwise to ACN (40 mL) to form a precipitate. The precipitated mixture was centrifuged at 3500 rpm for 25 min. The supernatant was decanted, and the pellet was dissolved in water (4 mL) and purified by C18 reverse phase to isolate the title compound. UPLC-MS Method A: m/z=1080.1 (z=2); t_{R}=2.33 min.

### Step 8: (S)-15-((6-(Bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oic acid

The title compound was prepared using procedures analogous to those described for Example 1, Step 5, substituting benzyl (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl) carbamoyl)-19-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1034.6 (z=2); t_{R}=1.68 min.

### Step 9: (S)-2,5-Dioxopyrrolidin-1-yl 15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oate

The title compound was prepared using procedures analogous to those described for Example 1, Step 6, substituting (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1083.6 (z=2); t_{R}=1.77 min.

### Example 16: 2,5-Dioxopyrrolidin-1-yl (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2R,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl) amino)-6-oxohexyl) amino)-2-oxoethyl)-5,12,17,21-tetraoxo-1-(((2S,3S,4S,5S,6R)-3,4,5-trihydroxy-6-(hydroxymethyl) tetrahydro-2H-pyran-2-yl)oxy)-4-(3-(((2S,3S,4S,5S,6R)-3,4,5-trihydroxy-6-(hydroxymethyl) tetrahydro-2H-pyran-2-yl)oxy)propyl)-4,11,16,19,22-pentaazaoctacosan-28-oate (ML-16)

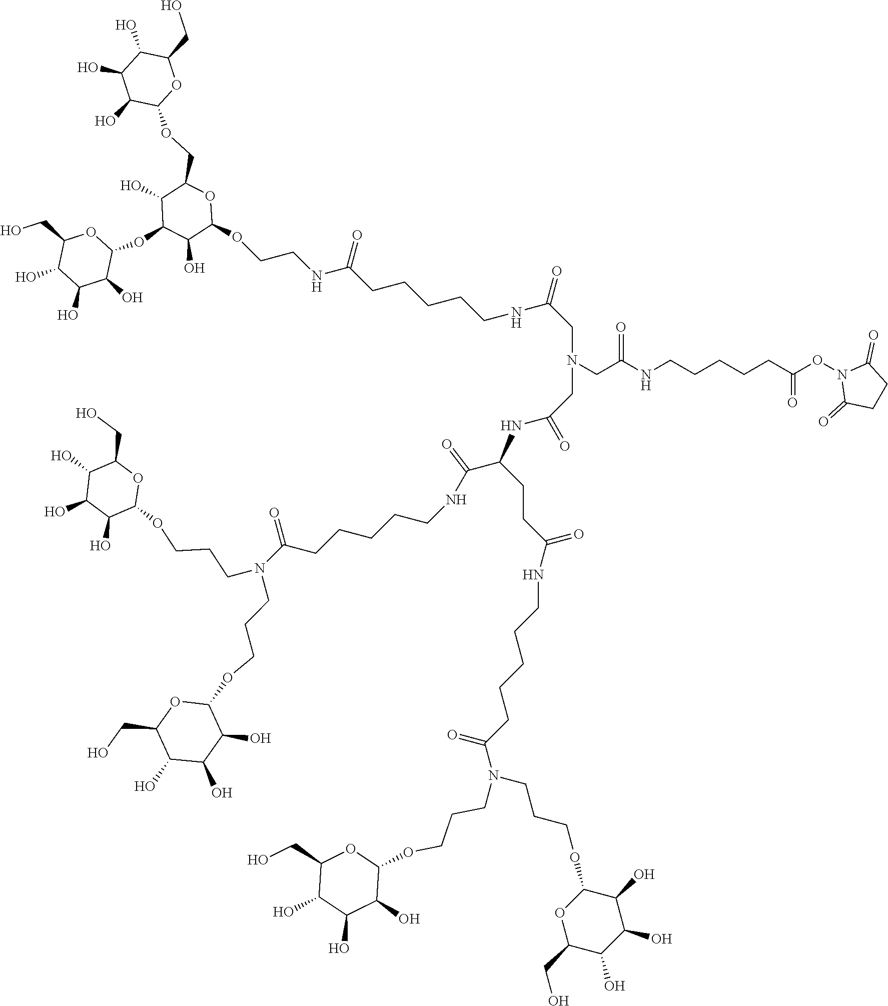 [SMILES: O=C(CCCCCNC(=O)CN(CC(=O)NCCCCCC(=O)ON1C(=O)CCC1=O)CC(=O)N[C@@H](CCC(=O)NCCCCCC(=O)N(CCCO[C@H]1O[C@H](CO)[C@@H](O)[C@H](O)[C@@H]1O)CCCO[C@H]1O[C@H](CO)[C@@H](O)[C@H](O)[C@@H]1O)C(=O)NCCCCCC(=O)N(CCCO[C@H]1O[C@H](CO)[C@@H](O)[C@H](O)[C@@H]1O)CCCO[C@H]1O[C@H](CO)[C@@H](O)[C@H](O)[C@@H]1O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl (S)-15-((6-(bis(3-(((2S,3S,4,5S,6R)-3,4,5-trihydroxy-6-(hydroxymethyl) tetrahydro-2H-pyran-2-yl)oxy)propyl)amino)-6-oxohexyl)carbamoyl)-19-(2-((6-((2-(((2R,3S,4S,5R,6R)-3,5-dihydroxy-4-(((2R,3S,4S,5S,6R)-3,4,5-trihydroxy)-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl) amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oate

The title compound was prepared using procedures analogous to those described for Example 1, Step 4, substituting (S)-2-amino-N1,N5-bis(6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl) pentanediamide for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide to isolate title compound. UPLC-MS Method A: m/z=1136.7 (z=2); t_{R}=2.38 min.

### Step 2: (S)-15-((6-(Bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oic acid

The title compound was prepared using procedures analogous to those described for Example 1, Step 5, substituting benzyl (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1091.6 (z=2); t_{R}=1.72 min.

### Step 3: 2,5-Dioxopyrrolidin-1-yl (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2R,3S,4S5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-5,12,17,21-tetraoxo-1-(((2S,3S,4,5S,6R)-3,4,5-trihydroxy-6-(hydroxymethyl) tetrahydro-2H-pyran-2-yl)oxy)-4-(3-(((2S,3S,4S,5S,6R)-3,4,5-trihydroxy-6-(hydroxymethyl) tetrahydro-2H-pyran-2-yl)oxy)propyl)-4,11,16,19,22-pentaazaoctacosan-28-oate

The title compound was prepared using procedures analogous to those described for Example 1, Step 6, substituting (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-5,12,17,21-tetraoxo-1-(((2S,3S,4S,5S,6R)-3,4,5-trihydroxy-6-(hydroxymethyl) tetrahydro-2H-pyran-2-yl)oxy)-4-(3-(((2S,3S,4S,5S,6R)-3,4,5-trihydroxy-6-(hydroxymethyl) tetrahydro-2H-pyran-2-yl)oxy)propyl)-4,11,16,19,22-pentaazaoctacosan-28-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1140.2 (z=2); t_{R}=1.85 min.

### Example 17: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate (ML-17)

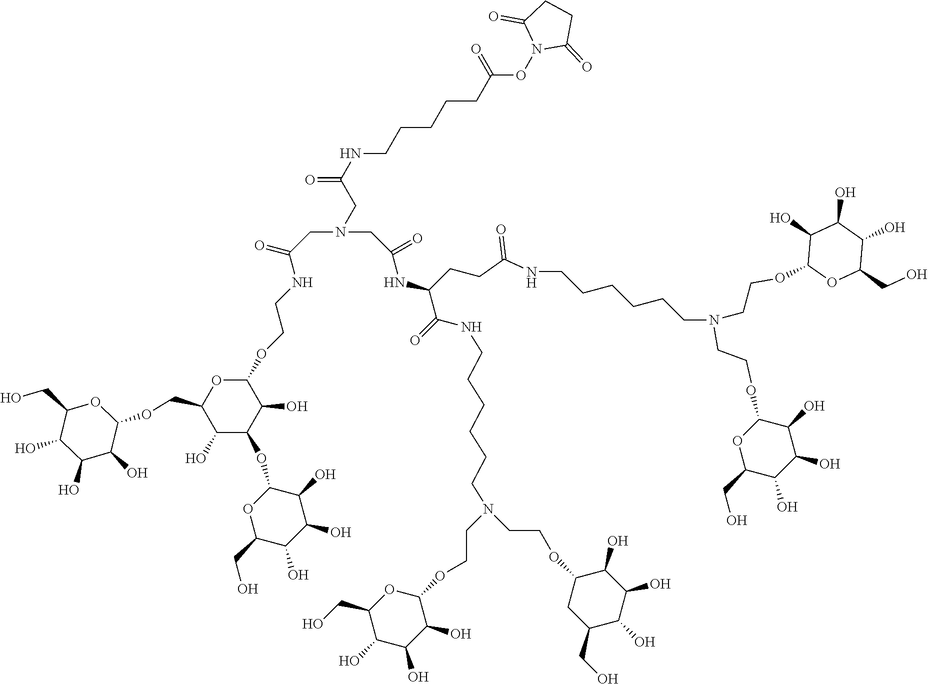 [SMILES: O=C(CC[C@H](NC(=O)CN(CC(=O)NCCCCCC(=O)ON1C(=O)CCC1=O)CC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)C(=O)NCCCCCCN(CCO[C@H]1O[C@H](CO)[C@@H](O)[C@H](O)[C@@H]1O)CCO[C@H]1C[C@H](CO)[C@@H](O)[C@H](O)[C@@H]1O)NCCCCCCN(CCO[C@H]1O[C@H](CO)[C@@H](O)[C@H](O)[C@@H]1O)CCO[C@H]1O[C@H](CO)[C@@H](O)[C@H](O)[C@@H]1O]

### Step 1: Benzyl (6-hydroxyhexyl)carbamate

6-Aminohexan-1-ol (5 g, 42.7 mmol) was dissolved in water (100 mL), and 5M aqueous NaOH (17 mL, 85 mmol) was added. The mixture was cooled to 0° C. under N₂. CBZ—Cl (7.31 mL, 51.2 mmol) dissolved in anhydrous THF (50 mL) was added dropwise over 20 min. The reaction mixture was stirred at 0° C. for 15 min after addition was complete; the mixture was then warmed to RT and stirred overnight. 1M HCl was added until the pH reached approximately 3. The reaction mixture was then partitioned twice with EtOAc; the organics were combined and rinsed with water, brine, dry (MgSO₄), filtered and concentrated in vacuo to give a slurry before completely dry. Approximately 300 mL hexanes were added. The reaction mixture was stirred vigorously for 15 min. The reaction mixture was then filtered, rinsed with hexanes, and dried under high vacuum to isolate title compound. ¹H NMR δ (ppm)(CHCl₃-d): 1.38 (5H, d, J=10.83 Hz), 1.41 (1H, s), 1.45 (1H, s), 3.22 (2H, q, J=6.73 Hz), 3.65 (3H, q, J=5.85 Hz), 4.79 (1H, s), 5.12 (2H, s), 7.39 (4H, d, J=4.36 Hz).

### Step 2: Benzyl (6-oxohexyl)carbamate

To a solution of benzyl (6-hydroxyhexyl)carbamate (6 g, 23.87 mmol) in DCM (200 mL) was added Dess Martin Periodinane (13.16 g, 31 mmol), and the resulting mixture was stirred at rt for 45 min. The mixture was washed with sat. NaHCO₃ (2×200 mL) and sat. NaCl (100 mL), then dried over Na₂SO₄, filtered, and evaporated. The residue was purified by normal phase silica gel column chromatography to isolate title compound. ¹H NMR δ (ppm)(CHCl₃-d): 1.38 (3H, t, J=8.43 Hz), 2.46 (2H, t, J=7.38 Hz), 3.23 (2H, q, J=6.76 Hz), 4.80 (1H, s), 5.12 (2H, s), 7.38 (5H, d, J=4.29 Hz), 9.78 (1H, s).

### Step 3: Benzyl (6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamate

To a mixture of bis{2-[(2,3,4,6-tetra-O-acetyl-α-D-mannopyranosyl)oxy]ethyl}amine (WO2020247297A1) (8.7 g, 11.36 mmol) and benzyl (6-oxohexyl) carbamate (3.4 g, 13.63 mmol) in DCM (100 mL) was added AcOH (0.65 mL, 11.36 mmol), followed by sodium triacetoxyboro-hydride (3.61 g, 17.04 mmol), and the resulting mixture was stirred at rt overnight. The liquid was evaporated, and the residue was partitioned between EtOAc (150 mL) and sat. NaHCO₃ (200 mL). The organic layer was separated and washed with sat. NaCl (100 mL), dried over Na₂SO4, filtered and evaporated. The residue was taken up in anhydrous MeOH (100 mL) and treated with NaOMe (1.06 mL, 30% solution in MeOH, 5.68 mmol), and the mixture was stirred at rt for 3 h. The mixture was evaporated and purified by C18 reverse phase chromatography to isolate title compound. UPLC-MS Method A: m/z=663.3 (z=1); t_{R}=2.48 min.

### Step 4: (2R,2′R, 3S,3′S,4S,4′S,5S,5′S,6S,6′S)-6,6′-((((6-Aminohexyl)azanediyl)bis(ethane-2,1-diyl))bis(oxy))bis(2-(hydroxymethyl)tetrahydro-2H-pyran-3,4,5-triol)

The title compound was prepared using procedures analogous to those described for Intermediate 5, Step 2, substituting benzyl (6-(bis(2-(((2S,3S,4S,5S,6R)-3,4,5-trihydroxy-6-(hydroxymethyl)tetrahydro-2H-pyran-2-yl)oxy)ethyl) amino)hexyl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl) carbamate to isolate title compound. UPLC-MS Method A: m/z=529.3 (z=1); t_{R}=0.87 min.

### Step 5: Benzyl ((S)-11,15-dioxo-1,25-bis(((2S,3S,4S,5S,6R)-3,4,5-trihydroxy-6-(hydroxymethyl) tetrahydro-2H-pyran-2-yl)oxy)-3,23-bis(2-(((2S,3S,4S,5S,6R)-3,4,5-trihydroxy-6-(hydroxymethyl)tetrahydro-2H-pyran-2-yl)oxy)ethyl)-3,10,16,23-tetraazapentacosan-12-yl)carbamate

To a solution of (S)-2-(((benzyloxy)carbonyl)amino)pentanedioic acid (500 mg, 1.78 mmol) and (2R,2′R,3S,3′S,4S,4′S,5S,5′S,6S,6′S)-6,6′-((((6-aminohexyl)azanediyl)bis(ethane-2,1-diyl))bis(oxy))bis(2-(hydroxymethyl)tetrahydro-2H-pyran-3,4,5-triol) (2.25 g, 4.27 mmol) in anhydrous DMF (10 mL) was added HOBt (599 mg, 3.91 mmol) followed by EDC (1.02 g, 5.33 mmol), and the mixture stirred at rt for 1 h. The mixture was purified by C18 reverse chromatography to isolate the title compound. UPLC-MS Method A: m/z=1302.7 (z=1); t_{R}=1.94 min

### Step 6: (S)-2-Amino-N1,N5-bis(6-(bis(2-(((2S,3S,4S,5S,6R)-3,4,5-trihydroxy-6-(hydroxymethyl)tetrahydro-2H-pyran-2-yl)oxy)ethyl)amino)hexyl)pentanediamide

The title compound was prepared using procedures analogous to those described for Intermediate 5, Step 2, substituting benzyl ((S)-11,15-dioxo-1,25-bis(((2S,3S,4S,5S,6R)-3,4,5-trihydroxy-6-(hydroxymethyl)tetrahydro-2H-pyran-2-yl)oxy)-3,23-bis(2-(((2S,3S,4S,5S,6R)-3,4,5-trihydroxy-6-(hydroxymethyl)tetrahydro-2H-pyran-2-yl)oxy)ethyl)-3,10,16,23-tetraazapentacosan-12-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)carbamate to isolate title compound. UPLC-MS Method A: m/z=1168.6 (z=1); t_{R}=0.87 min.

### Step 7: Benzyl (S)-14-((6-(bis(2-(((2S,3S,4,5S,6R)-3,4,5-trihydroxy-6-(hydroxymethyl) tetrahydro-2H-pyran-2-yl)oxy)ethyl)amino)hexyl)carbamoyl)-18-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

To a mixture of N-(2-((6-(benzyloxy)-6-oxohexyl)amino)-2-oxoethyl)-N-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)glycine (100 mg, 0.108 mmol) and (S)-2-amino-N1,N5-bis(6-(bis(2-(((2S,3S,4S,5S,6R)-3,4,5-trihydroxy-6-(hydroxymethyl) tetrahydro-2H-pyran-2-yl)oxy)ethyl)amino)hexyl) pentanediamide (152 mg, 0.13 mmol) in anhydrous DMF (1.5 mL) was added HOBt (20 mg, 0.13 mmol) and EDC (42 mg, 0.216 mmol), and the resulting mixture stirred at rt for 2 h. The mixture added dropwise to ACN to form a precipitate, which was centrifuged at 3500 rpm for 25 min. The supernatant was decanted, and the remaining pellet dissolved in water and purified by C18 reverse phase chromatography to isolate the title compound. UPLC-MS Method A: m/z=1037.6 (z=2); t_{R}=2.21 min.

### Step 8: (S)-14-((6-(Bis(2-(((2S,3S,4S,5R,6R)-3,4,5-trihydroxy-6-(hydroxymethyl)tetrahydro-2H-pyran-2-yl)oxy)ethyl)amino)hexyl)carbamoyl)-18-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid

The title compound was prepared using procedures analogous to those described for Example 1, Step 5, substituting benzyl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)-11,16,20-trioxo-1-(((2S,3 S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,15,18,24,27,30-octaazahexatriacontan-36-oate to isolate title compound. UPLC-MS Method A: m/z=992.5 (z=2); t_{R}=1.22 min.

### Step 9: 2,5-Dioxopyrrolidin-1-yl (S)-14-((6-(bis(2-(((2S,3S,4,5S,6R)-3,4,5-trihydroxy-6-(hydroxymethyl)tetrahydro-2H-pyran-2-yl)oxy)ethyl)amino)hexyl)carbamoyl)-18-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 6, substituting (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1041.0 (z=2); t_{R}=1.58 min.

### Example 18: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2R,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-11,16,20-trioxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate (ML-18)

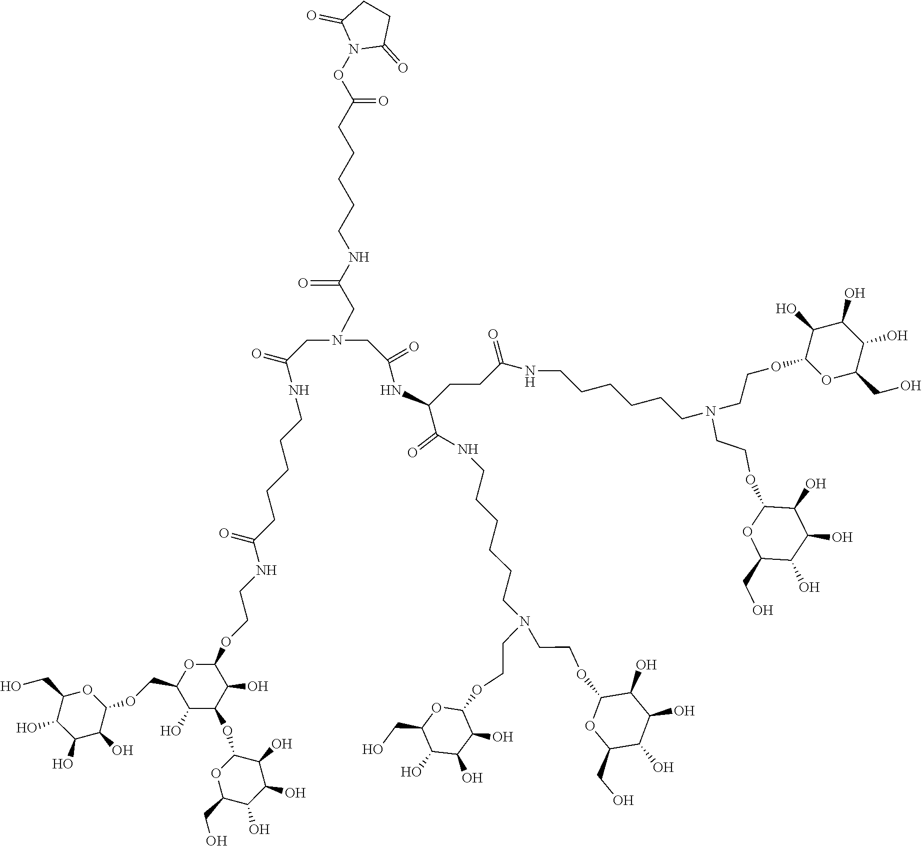 [SMILES: O=C(CCCCCNC(=O)CN(CC(=O)NCCCCCC(=O)ON1C(=O)CCC1=O)CC(=O)N[C@@H](CCC(=O)NCCCCCCN(CCO[C@H]1O[C@H](CO)[C@@H](O)[C@H](O)[C@@H]1O)CCO[C@H]1O[C@H](CO)[C@@H](O)[C@H](O)[C@@H]1O)C(=O)NCCCCCCN(CCO[C@H]1O[C@H](CO)[C@@H](O)[C@H](O)[C@@H]1O)CCO[C@H]1O[C@H](CO)[C@@H](O)[C@H](O)[C@@H]1O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: N-(2-((6-(Benzyloxy)-6-oxohexyl)amino)-2-oxoethyl)-N-(2-((6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)glycine

To a solution of 2,2′-((2-((6-(benzyloxy)-6-oxohexyl)amino)-2-oxoethyl)azanediyl) diacetic acid (650 mg, 1.65 mmol) in anhydrous DCM (12 mL) cooled in an ice bath was added trifluoroacetic anhydride (0.279 mL, 1.98 mmol). The resulting mixture was stirred at ice bath temperature for 3 h. The mixture was cooled to −15° C., and a solution of Et₃N (0.551 mL, 3.96 mmol) in DMF (6 mL) was added slowly over approximately 20 min. After complete addition, the mixture was stirred at −15° C. for a further 30 min, then the mixture was warmed to rt and a solution of 6-amino-N-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl) oxy)ethyl)hexanamide (1.3 g, 1.98 mmol) in DMF (12 mL) added. The mixture was stirred for 1 h. The mixture was evaporated, and the solution was added to ACN (130 mL). The resulting suspension was centrifuged at 3500 rpm for 20 min. The supernatant was decanted, and the pellet dissolved in water and purified by C18 reverse phase chromatography. UPLC-MS Method A: m/z=1037.6 (z=2); t_{R}=2.85 min.

### Step 2: Benzyl (S)-14-((6-(Bis(2-(((2S,3S,4S,5S,6R)-3,4,5-trihydroxy-6-(hydroxymethyl) tetrahydro-2H-pyran-2-yl)oxy)ethyl)amino)hexyl)carbamoyl)-18-(2-((6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

To a mixture of N-(2-((6-(benzyloxy)-6-oxohexyl)amino)-2-oxoethyl)-N-(2-((6-((2-(((2R,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)glycine (100 mg, 0.096 mmol) and (S)-2-amino-N1,N5-bis(6-(bis(2-(((2S,3S,4S,5S,6R)-3,4,5-trihydroxy-6-(hydroxymethyl)tetrahydro-2H-pyran-2-yl)oxy)ethyl)amino) hexyl)pentane diamide (135 mg, 0.116 mmol) in anhydrous DMF (1.5 mL) was added HOBt (18 mg, 0.116 mmol) and EDC (37 mg, 0.193 mmol), and the resulting mixture stirred at rt for 2 h. The mixture was added dropwise to ACN (40 mL) to form a precipitate, which was centrifuged at 3500 rpm for 25 min. The supernatant was decanted, and the remaining pellet was dissolved in water and purified by C18 reverse phase chromatography to isolate title compound. UPLC-MS Method A: m/z=1136.7 (z=2); t_{R}=2.38 mm.

### Step 3: (S)-14-((6-(Bis(2-(((2S,3S,4S,5S6R)-3,4,5-trihydroxy-6-(hydroxymethyl)tetrahydro-2H-pyran-2-yl)oxy)ethyl)amino)hexyl)carbamoyl)-18-(2-((6-((2-(((2R,3S,4S,5R,6R)-3,5-dihydroxy-4-(((2R,3S,4,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid

The title compound was prepared using procedures analogous to those described for Example 1, Step 5, substituting benzyl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2R,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-11,16,20-trioxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1091.6 (z=2); t_{R}=1.72 min.

### Step 4: 2,5-Dioxopyrrolidin-1-yl (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2R,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl)amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oate

The title compound was prepared using procedures analogous to those described for Example 1, Step 6, substituting (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2R,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-11,16,20-trioxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oic acid for (S)-27-(2-((6-((2-(((2S,3 S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1140.2 (z=2); t_{R}=1.85 min.

### Example 19: 2,5-Dioxopyrrolidin-1-yl (R)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-4,11,16,20-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate (ML-19)

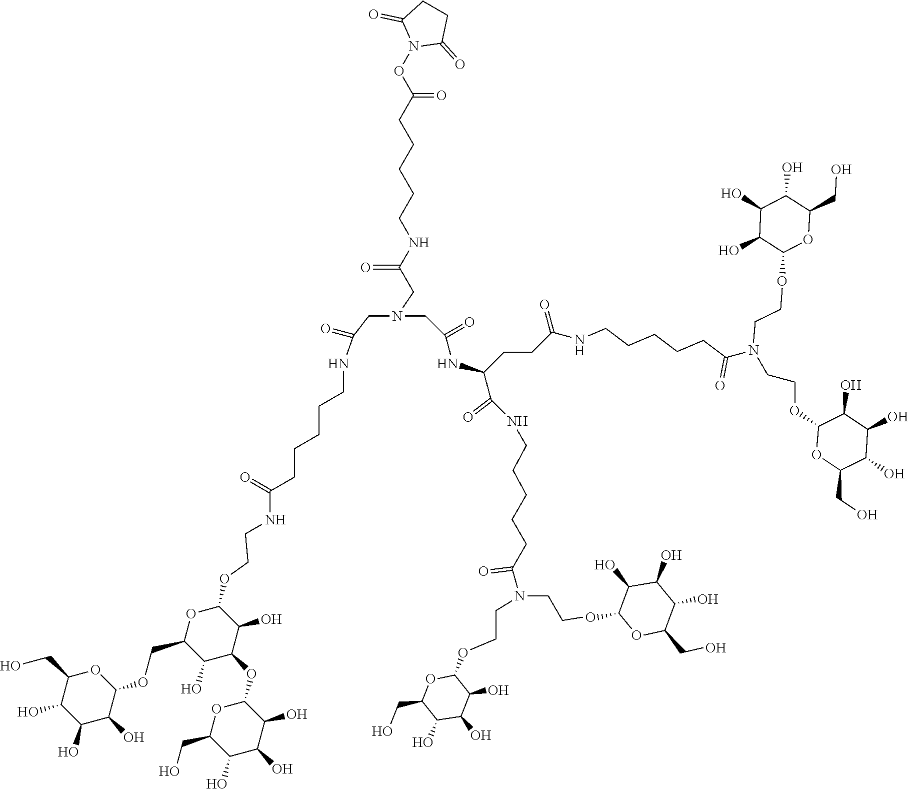 [SMILES: O=C(CCCCCNC(=O)CN(CC(=O)NCCCCCC(=O)ON1C(=O)CCC1=O)CC(=O)N[C@@H](CCC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)C(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl ((R)-4,11,15,22-tetraoxo-1,25-bis(((2S,3S,4S,5S,6R)-3,4,5-trihydroxy-6-(hydroxymethyl)tetrahydro-2H-pyran-2-yl)oxy)-3,23-bis(2-(((2S,3S,4S,5S,6R)-3,4,5-trihydroxy-6-(hydroxymethyl)tetrahydro-2H-pyran-2-yl)oxy)ethyl)-3,10,16,23-tetraazapentacosan-12-yl)carbamate

To a mixture of acid N-Cbz-D-glutamic acid (200 mg, 0.711 mmol) and 6-amino-N,N-bis(2-(((2S,3S,4S,5S,6R)-3,4,5-trihydroxy-6-(hydroxymethyl)tetrahydro-2H-pyran-2-yl)oxy) ethyl) hexanamide (965 mg, 1.778 mmol) in anhydrous DMF (4 mL) was added EDC (545 mg, 2.84 mmol) and HOBt (272 mg, 1.778 mmol), and the resulting mixture stirred at rt for 3 h. The mixture was purified by C18 reverse phase chromatography to isolate title compound. UPLC-MS Method A: m/z=1330.7 (z=1); t_{R}=4.58 min.

### Step 2: (R)-2-Amino-N1,N5-bis(6-(bis(2-(((2S,3S,4S,5S,6R)-3,4,5-trihydroxy-6-(hydroxymethyl) tetrahydro-2H-pyran-2-yl)oxy)ethyl)amino)-6-oxohexyl)pentanediamide

The title compound was prepared using procedures analogous to those described for Intermediate 5, Step 2, substituting benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate for benzyl ((R)-4,11,15,22-tetraoxo-1,25-bis(((2S,3S,4S,5S,6R)-3,4,5-trihydroxy-6-(hydroxymethyl)tetrahydro-2H-pyran-2-yl)oxy)-3,23-bis(2-(((2S,3S,4S,5S,6R)-3,4,5-trihydroxy-6-(hydroxymethyl) tetrahydro-2H-pyran-2-yl)oxy)ethyl)-3,10,16,23-tetraazapentacosan-12-yl)carbamateto isolate title compound. UPLC-MS Method A: m/z=1168.6 (z=1); t_{R}=0.87 min.

### Step 3: Benzyl (R)-14-((6-(bis(2-(((2S,3S,4S,5S,6R)-3,4,5-trihydroxy-6-(hydroxymethyl) tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,34S,5S6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 4, substituting (R)-2-amino-N1,N5-bis(6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl) pentanediamide for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide to isolate title compound. UPLC-MS Method A: m/z=1108.6 (z=2); t_{R}=4.43 min.

### Step 4: (R)-14-((6-(Bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid

The title compound was prepared using procedures analogous to those described for Example 1, Step 5, substituting benzyl (R)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1063.1 (z=2); t_{R}=4.50 min.

### Step 5: 2,5-Dioxopyrrolidin-1-yl (R)-14-((6-(bis(2-(((2S,3 S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 6, substituting (R)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1112.1 (z=2); t_{R}=4.35 min.

### Example 20: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-4,11,16,20-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate (ML-20)

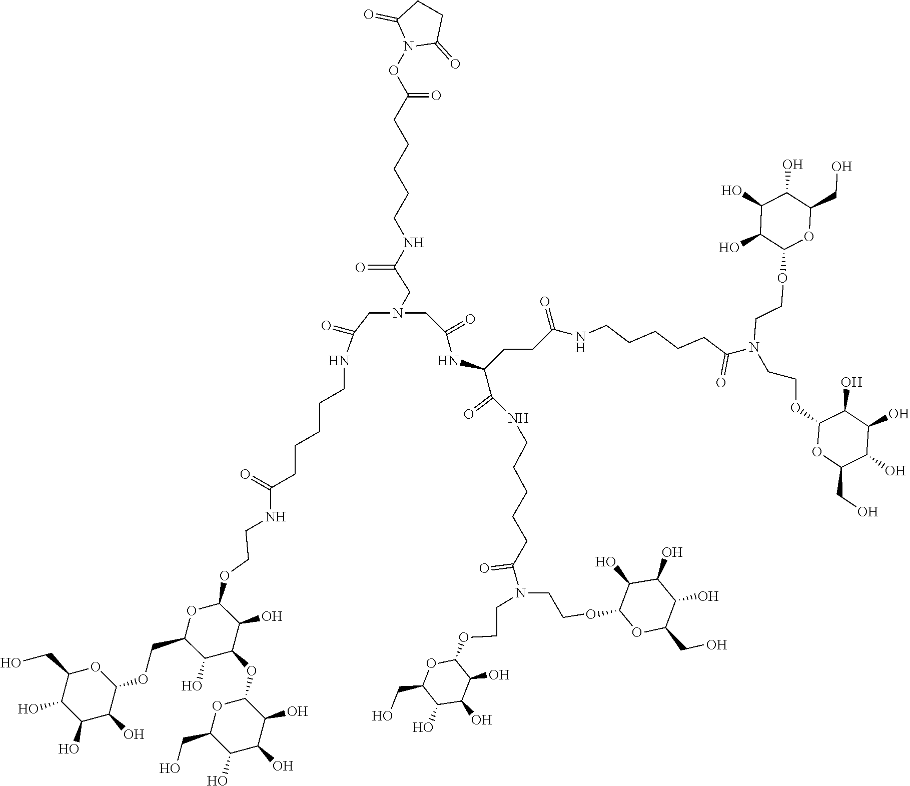 [SMILES: O=C(CCCCCNC(=O)CN(CC(=O)NCCCCCC(=O)ON1C(=O)CCC1=O)CC(=O)N[C@@H](CCC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)C(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: (S)-3-(2-((6-(Benzyloxy)-6-oxohexyl)amino)-2-oxoethyl)-7-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl) carbamoyl)-5,10,17-trioxo-20-(((2S,3S,4S,5S,6R)-3,4,5-trihydroxy-6-(hydroxymethyl) tetrahydro-2H-pyran-2-yl)oxy)-18-(2-(((2S,3S,4S,5S,6R)-3,4,5-trihydroxy-6-(hydroxymethyl) tetrahydro-2H-pyran-2-yl)oxy)ethyl)-3,6,11,18-tetraazaicosanoic acid

To a solution of 2,2′-((2-((6-(benzyloxy)-6-oxohexyl)amino)-2-oxoethyl)azanediyl) diacetic acid (200 mg, 0.507 mmol) in anhydrous DCM (2 mL), cooled in an ice bath, was added trifluoroacetic anhydride (0.09 mL, 0.634 mmol), and the mixture was stirred at ice bath temperature for 2 h. The mixture was cooled to −15° C., and a solution of TEA (0.17 mL, 1.22 mmol) in DMF (1 mL) added slowly over 20 min. After complete addition, the mixture was stirred at −15° C. for a further 30 min, then a solution of (S)-2-amino-N1,N5-bis(6-(bis(2-(((2S,3S,4S,5S,6R)-3,4,5-trihydroxy-6-(hydroxymethyl)tetrahydro-2H-pyran-2-yl)oxy)ethyl) amino)-6-oxohexyl) pentane diamide (728 mg, 0.608 mmol) in DMF (2 mL) was added. The mixture was allowed to warm to rt and stirred for 2 h. The mixture was evaporated and purified by C18 reverse phase chromatography to isolate title compound. UPLC-MS Method A: m/z=1572.8 (z=1); t_{R}=2.62 min.

### Step 2: Benzyl (S)-14-((6-(bis(2-(((2S,3S,4S,5S,6R)-3,4,5-trihydroxy-6-(hydroxymethyl) tetrahydro-2H-pyran-2-yl)oxy)ethyl)amino)-6-oxohexyl)carbamoyl)-18-(2-((6-((2-(((2R,3S,4S,5R,6R)-3,5-dihydroxy-4-(((2R,3S,4S,5S,66R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,11,16,20-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate

To a mixture of (S)-3-(2-((6-(benzyloxy)-6-oxohexyl)amino)-2-oxoethyl)-7-((6-(bis(2-(((2S,3S,4S,5S,6R)-3,4,5-trihydroxy-6-(hydroxymethyl)tetrahydro-2H-pyran-2-yl)oxy)ethyl) amino)-6-oxohexyl)carbamoyl)-5,10,17-trioxo-20-(((2S,3S,4S,5S,6R)-3,4,5-trihydroxy-6-(hydroxymethyl)tetrahydro-2H-pyran-2-yl)oxy)-18-(2-(((2S,3S,4S,5S,6R)-3,4,5-trihydroxy-6-(hydroxymethyl)tetrahydro-2H-pyran-2-yl)oxy)ethyl)-3,6,11,18-tetraazaicosanoic acid (57 mg, 0.036 mmol) and 6-amino-N-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)hexanamide (31 mg, 0.047 mmol) in anhydrous DMF (2 mL) was added EDC (14 mg, 0.072 mmol) and HOBT (6 mg, 0.036 mmol), and the resulting mixture was stirred at rt for 4 h. The mixture was purified by B18 reverse phase chromatography to isolate title compound. UPLC-MS Method A: m/z=1108.6 (z=2); t_{R}=4.09 min.

### Step 3: (S)-14-((6-(Bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,11,16,20-tetraoxo-1-(((2S,3S,4,5S,6R)-3,4,5-trihydroxy-6-(hydroxymethyl)tetrahydro-2H-pyran-2-yl)oxy)-3-(2-(((2S,3S,4,5S,6R)-3,4,5-trihydroxy-6-(hydroxymethyl)tetrahydro-2H-pyran-2-yl)oxy)ethyl)-3,10,15,18,21-pentaazaheptacosan-27-oic acid

The title compound was prepared using procedures analogous to those described for Example 1, Step 5, substituting benzyl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1063.1 (z=2); t_{R}=4.30 min.

### Step 4: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2R 3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 6, substituting (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl) amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1112.1 (z=2); t_{R}=4.18 min.

### Example 21: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate (ML-21)

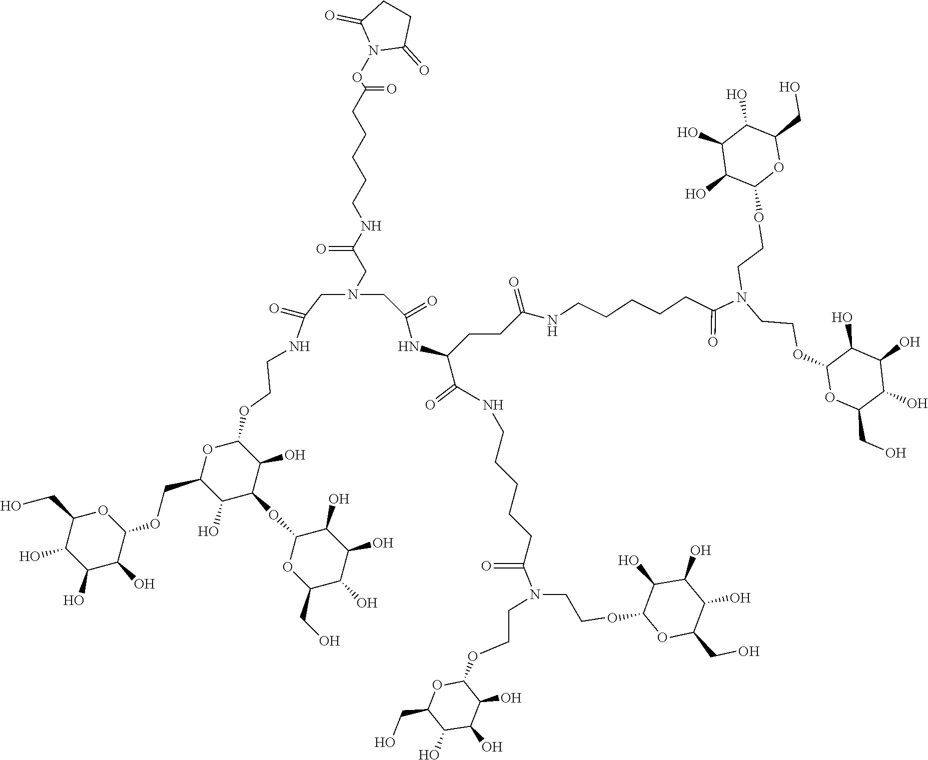 [SMILES: O=C(CC[C@H](NC(=O)CN(CC(=O)NCCCCCC(=O)ON1C(=O)CCC1=O)CC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)C(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O]

### Step 1: Benzyl (S)-14-((6-(bis(2-(((2S,3S,4,5S,6R)-3,4,5-trihydroxy-6-(hydroxymethyl) tetrahydro-2H-pyran-2-yl)oxy)ethyl)amino)-6-oxohexyl)carbamoyl)-18-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,354S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 18, Step 2, substituting (S)-2-amino-N1,N5-bis(6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl) pentanediamide for (S)-2-amino-N1,N5-bis(6-(bis(2-(((2S,3S,4S,5S,6R)-3,4,5-trihydroxy-6-(hydroxymethyl)tetrahydro-2H-pyran-2-yl)oxy)ethyl)amino)hexyl)pentanediamide to isolate title compound. UPLC-MS Method A: m/z=1052.1 (z=2); t_{R}=2.45 min.

### Step 2: (S)-14-((6-(Bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((2-(((2S,3S,4S,5R,6R)-3,5-dihydroxy-4-(((2R,3S,4,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid

The title compound was prepared using procedures analogous to those described for Example 1, Step 5, substituting benzyl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1006.5 (z=2); t_{R}=1.85 min.

### Step 3: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((2-(((2S,3S,4S,5R,6R)-3,5-dihydroxy-4-(((2R,3S,4,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 6, substituting (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,15,18,24,27,30-octaazahexatriacontan-36-oic acid to isolate title compound. UPLC-MS Method A: t_{R}=1.86 min; m/z=1055.5 (z=2).

### Example 22: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-11,16,20-trioxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate (ML-22)

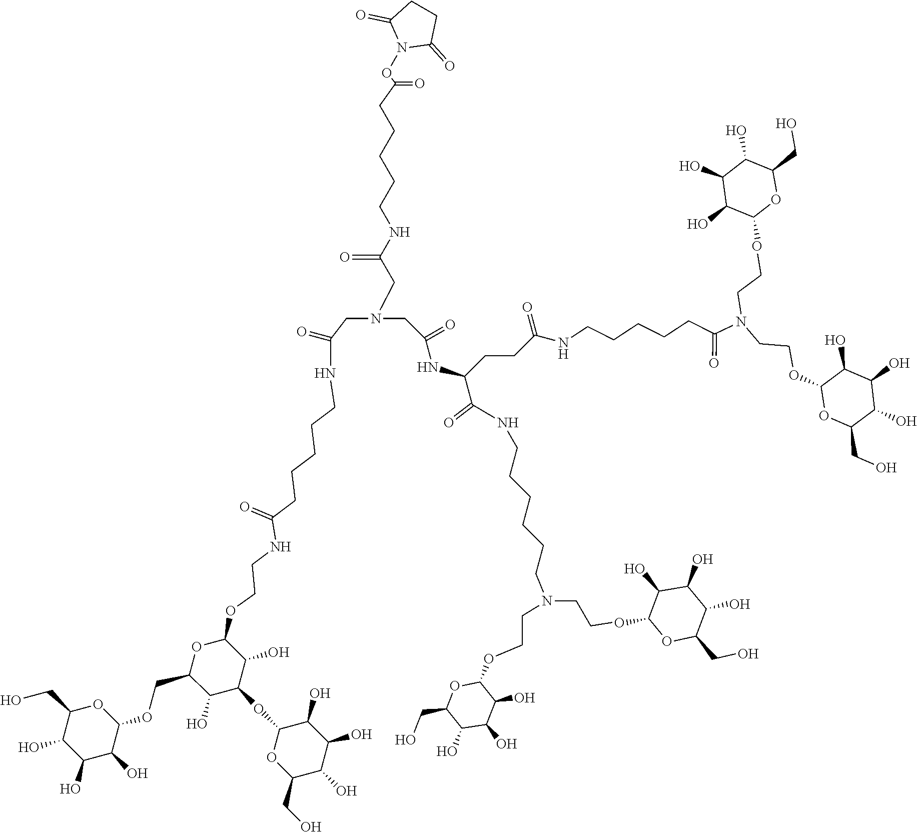 [SMILES: O=C(CCCCCNC(=O)CN(CC(=O)NCCCCCC(=O)ON1C(=O)CCC1=O)CC(=O)N[C@@H](CCC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)C(=O)NCCCCCCN(CCO[C@H]1O[C@H](CO)[C@@H](O)[C@H](O)[C@@H]1O)CCO[C@H]1O[C@H](CO)[C@@H](O)[C@H](O)[C@@H]1O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@H]1O]

### Step 1: Benzyl (S)-14-((6-(bis(2-(((2S,3S,4S,5S,6R)-3,4,5-trihydroxy-6-(hydroxymethyl) tetrahydro-2H-pyran-2-yl)oxy)ethyl)amino)hexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 4, substituting (S)-2-amino-N1,N5-bis(6-(bis(2-(((2S,3S,4S,5S,6R)-3,4,5-trihydroxy-6-(hydroxymethyl)tetrahydro-2H-pyran-2-yl)oxy)ethyl)amino)hexyl)pentanediamide for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy) ethyl)acetamide to isolate title compound. UPLC-MS Method A: m/z=1094.6 (z=2); t_{R}=2.47 min.

### Step 2: (S)-14-((6-(Bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid

The title compound was prepared using procedures analogous to those described for Example 1, Step 5, substituting benzyl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-11,16,20-trioxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1049.1 (z=2); t_{R}=1.74 min.

### Step 3: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 6, substituting (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-11,16,20-trioxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: t_{R}=1.91 min; m/z=1097.2 (z=2).

### Example 23: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl) amino)-2-oxoethyl)-4,11,16,20-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate (ML-23)

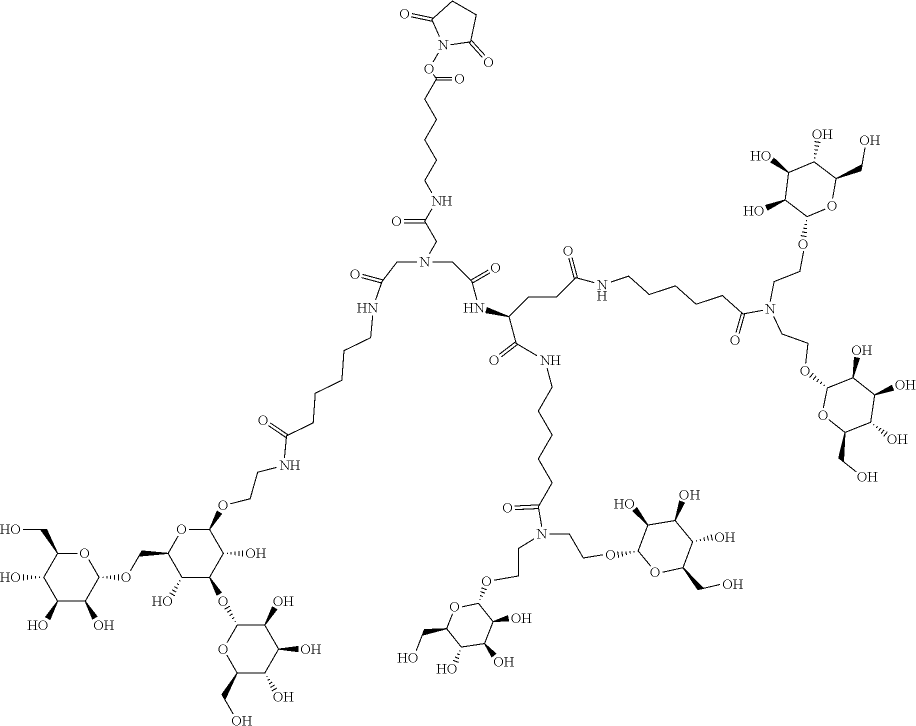 [SMILES: O=C(CCCCCNC(=O)CN(CC(=O)NCCCCCC(=O)ON1C(=O)CCC1=O)CC(=O)N[C@@H](CCC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)C(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@H]1O]

### Step 1: (2S,3S,4S,5S,6R)-2-(((2R,3R,4S,5R,6R)-6-(2-Aminoethoxy)-3,5-dihydroxy-4-(((2R,3S,4S,5S,6R)-3,4,5-trihydroxy-6-(hydroxymethyl)tetrahydro-2H-pyran-2-yl)oxy)tetrahydro-2H-pyran-2-yl)methoxy)-6-(hydroxymethyl)tetrahydro-2H-pyran-3,4,5-triol hydrochloride

To a solution of benzyl (2-{[α-D-mannopyranosyl-(1→3)-[α-D-mannopyranosyl-(1→6)]-4-O-benzyl-β-D-glucopyranosyl]oxy}ethyl)carbamate (WO2016164288A1) (3 g, 3.89 mmol) in water (30 mL) was added 10% Pd/C (414 mg), and the resulting mixture was stirred under H_{2(g)} overnight. The mixture was filtered through CELITE™ diatomaceous earth, and the filtrate treated with conc. HCl (0.64 mL, 7.77 mL) and 10% Pd/C (414 mg), and the resulting mixture stirred under H_{2(g)} overnight. The mixture was filtered through CELITE™ diatomaceous earth, and the filtrate was freeze dried to isolate title compound. MS Method A: m/z=548.2 (z=1); t_{R}=1.04 min.

### Step 2: Benzyl (6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate

The title compound was prepared using procedures analogous to those described for Intermediate 4, Step 1, substituting (2S,3S,4S,5S,6R)-2-(((2R,3R,4S,5R,6R)-6-(2-aminoethoxy)-3,5-dihydroxy-4-(((2R,3S,4S,5S,6R)-3,4,5-trihydroxy-6-(hydroxymethyl) tetrahydro-2H-pyran-2-yl)oxy)tetrahydro-2H-pyran-2-yl)methoxy)-6-(hydroxymethyl) tetrahydro-2H-pyran-3,4,5-triol hydrochloride for AEM to isolate title compound. UPLC-MS Method A: m/z=795.3 (z=1); t_{R}=2.78 min.

### Step 3: 6-Amino-N-(2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) hexanamide

The title compound was prepared using procedures analogous to those described for Intermediate 5, Step 2, substituting benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate for benzyl (6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)carbamate to isolate title compound. UPLC-MS Method A: m/z=661.3 (z=1); t_{R}=0.92 min.

### Step 4: N-(2-((6-(Benzyloxy)-6-oxohexyl)amino)-2-oxoethyl)-N-(2-((6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)glycine

To a solution of 2,2′-((2-((6-(benzyloxy)-6-oxohexyl)amino)-2-oxoethyl)azanediyl) diacetic acid (300 mg, 0.761 mmol) in anhydrous DCM (3 mL) cooled in an ice bath was added TFAA (0.134 mL, 0.951 mmol), and the resulting mixture was stirred at ice bath temperature for 3 h. The mixture was cooled to −15° C., and a solution of TEA (0.254 mL, 1.83 mmol) in DMF (1.5 mL) was added slowly over approximately 20 min. After complete addition, the mixture was stirred at −15° C. for a further 30 min, then a solution of 6-amino-N-(2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)hexanamide (553 mg, 0.837 mmol) in DMF (3 mL) was added and allowed to warm to rt and stirred for 2 h. The mixture was evaporated and purified by C18 reverse phase chromatography, and the material was freeze dried to isolate title compound. UPLC-MS Method A: m/z=1037.5 (z=1); t_{R}=2.93 min.

### Step 5: Benzyl (S)-14-((6-(bis(2-(((2S,3S,4S,5S,6R)-3,4,5-trihydroxy-6-(hydroxymethyl) tetrahydro-2H-pyran-2-yl)oxy)ethyl)amino)-6-oxohexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

To a mixture of N-(2-((6-(benzyloxy)-6-oxohexyl)amino)-2-oxoethyl)-N-(2-((6-((2-(((2R,3R,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl) amino)-6-oxohexyl) amino)-2-oxoethyl)glycine (200 mg, 0.193 mmol) and (S)-2-amino-N1,N5-bis(6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)pentanediamide (300 mg, 0.251 mmol) in anhydrous DMF (2 mL) was added EDC (74 mg, 0.386 mmol) and DMAP (35 mg, 0.289 mmol), and the resulting mixture was stirred at rt overnight. The mixture was purified by C18 reverse phase chromatography to isolate title compound. UPLC-MS Method A: m/z=1108.6 (z=2); t_{R}=2.61 min.

### Step 6: (S)-14-((6-(Bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid

The title compound was prepared using procedures analogous to those described for Example 1, Step 5, substituting benzyl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-4,11,16,20-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1063.1 (z=2); t_{R}=1.74 min.

### Step 7: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 6, substituting (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oic acid to isolate title compound. UPLC-MS Method A: m/z=1112.1 (z=2); t_{R}=1.87 min.

### Example 24: 2,5-Dioxopyrrolidin-1-yl (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oate (ML-24)

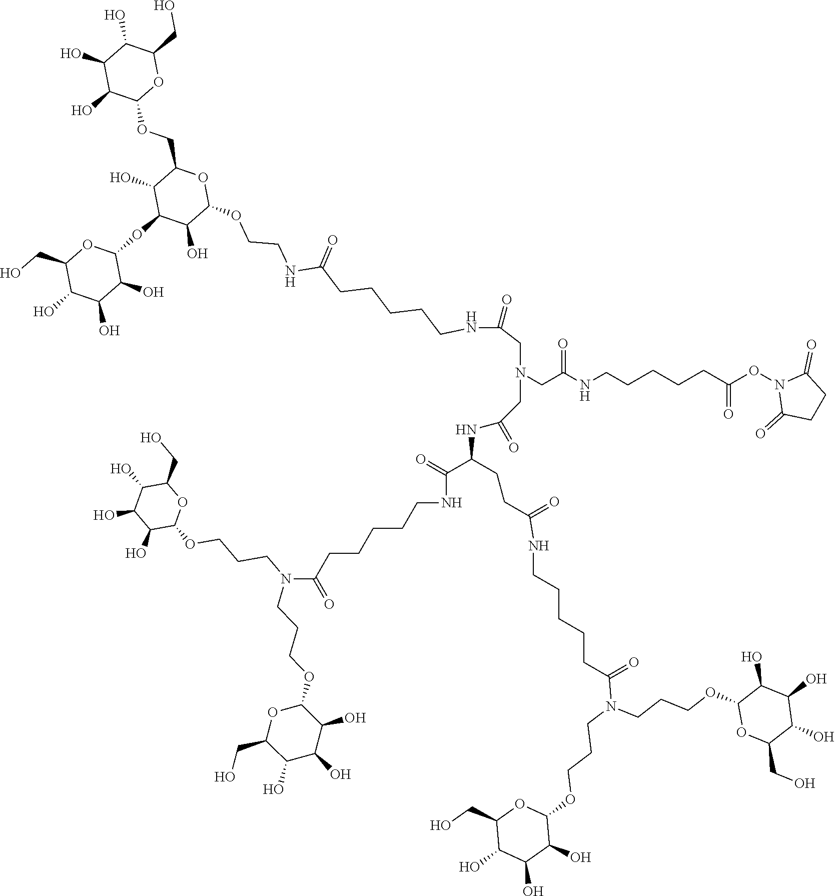 [SMILES: O=C(CCCCCNC(=O)CN(CC(=O)NCCCCCC(=O)ON1C(=O)CCC1=O)CC(=O)N[C@@H](CCC(=O)NCCCCCC(=O)N(CCCO[C@H]1O[C@H](CO)[C@@H](O)[C@H](O)[C@@H]1O)CCCO[C@H]1O[C@H](CO)[C@@H](O)[C@H](O)[C@@H]1O)C(=O)NCCCCCC(=O)N(CCCO[C@H]1O[C@H](CO)[C@@H](O)[C@H](O)[C@@H]1O)CCCO[C@H]1O[C@H](CO)[C@@H](O)[C@H](O)[C@@H]1O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl (S)-15-((6-(bis(3-(((2S,3S,4S,5S,6R)-3,4,5-trihydroxy-6-(hydroxymethyl) tetrahydro-2H-pyran-2-yl)oxy)propyl)amino)-6-oxohexyl)carbamoyl)-19-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl)amino)-2-oxoethyl)-5,12,17,21-tetraoxo-1-(((2S,3S,4S,5S,6R)-3,4,5-trihydroxy-6-(hydroxymethyl) tetrahydro-2H-pyran-2-yl)oxy)-4-(3-(((2S,3S,4S,5S,6R)-3,4,5-trihydroxy-6-(hydroxymethyl) tetrahydro-2H-pyran-2-yl)oxy)propyl)-4,11,16,19,22-pentaazaoctacosan-28-oate

The title compound was prepared using procedures analogous to those described for Example 1, Step 4, substituting (S)-2-amino-N1,N5-bis(6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl) pentanediamide for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) to isolate the title compound. UPLC-MS Method A: m/z=1136.5 (z=2); t_{R}=2.43.

### Step 2: (S)-15-((6-(Bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oic acid

The title compound was prepared using procedures analogous to those described for Example 1, Step 5, substituting benzyl (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-5,12,17,21-tetraoxo-1-(((2S,3S,4S,5S,6R)-3,4,5-trihydroxy-6-(hydroxymethyl) tetrahydro-2H-pyran-2-yl)oxy)-4-(3-(((2S,3S,4S,5S,6R)-3,4,5-trihydroxy-6-(hydroxymethyl) tetrahydro-2H-pyran-2-yl)oxy)propyl)-4,11,16,19,22-pentaazaoctacosan-28-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1063.1 (z=2); t_{R}=1.74 min.

### Step 3: 2,5-Dioxopyrrolidin-1-yl (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-5,12,17,21-tetraoxo-1-(((2S,3S,4S,5S,6R)-3,4,5-trihydroxy-6-(hydroxymethyl)tetrahydro-2H-pyran-2-yl)oxy)-4-(3-(((2S,3S,4S,5S,6R)-3,4,5-trihydroxy-6-(hydroxymethyl)tetrahydro-2H-pyran-2-yl)oxy)propyl)-4,11,16,19,22-pentaazaoctacosan-28-oate

The title compound was prepared using procedures analogous to those described for Example 1, Step 6, substituting (S)-15-((6-(bis(3-(((2S,3S,4S,5S,6R)-3,4,5-trihydroxy-6-(hydroxymethyl)tetrahydro-2H-pyran-2-yl)oxy)propyl)amino)-6-oxohexyl)carbamoyl)-19-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-5,12,17,21-tetraoxo-1-(((2S,3S,4S,5S,6R)-3,4,5-trihydroxy-6-(hydroxymethyl) tetrahydro-2H-pyran-2-yl)oxy)-4-(3-(((2S,3S,4S,5S,6R)-3,4,5-trihydroxy-6-(hydroxymethyl) tetrahydro-2H-pyran-2-yl)oxy)propyl)-4,11,16,19,22-pentaazaoctacosan-28-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: t_{R}=1.88 min; m/z=1140.1 (z=2).

### Example 25: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-11,16,20-trioxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate (ML-25)

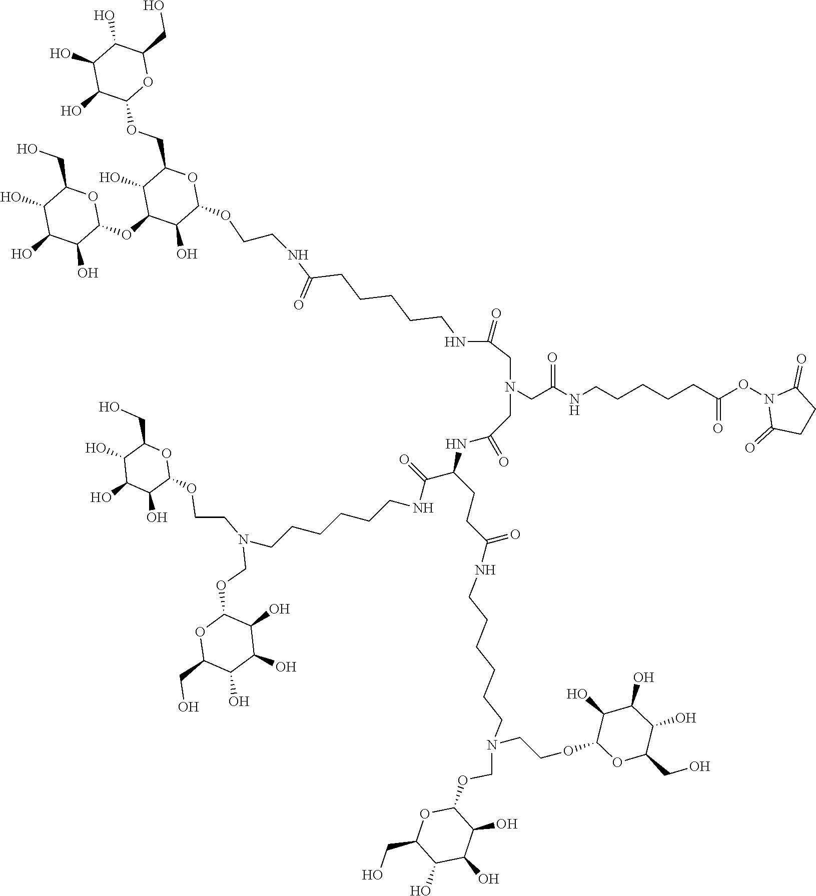 [SMILES: O=C(CCCCCNC(=O)CN(CC(=O)NCCCCCC(=O)ON1C(=O)CCC1=O)CC(=O)N[C@@H](CCC(=O)NCCCCCCN(CCO[C@H]1O[C@H](CO)[C@@H](O)[C@H](O)[C@@H]1O)CO[C@H]1O[C@H](CO)[C@@H](O)[C@H](O)[C@@H]1O)C(=O)NCCCCCCN(CCO[C@H]1O[C@H](CO)[C@@H](O)[C@H](O)[C@@H]1O)CO[C@H]1O[C@H](CO)[C@@H](O)[C@H](O)[C@@H]1O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl (S)-14-((6-(bis(2-(((2S,3S,4S,5S,6R)-3,4,5-trihydroxy-6-(hydroxymethyl) tetrahydro-2H-pyran-2-yl)oxy)ethyl)amino)hexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-11,16,20-trioxo-J-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 4, substituting (S)-2-amino-N1,N5-bis(6-(bis(2-(((2S,3S,4S,5S,6R)-3,4,5-trihydroxy-6-(hydroxymethyl)tetrahydro-2H-pyran-2-yl)oxy)ethyl)amino)hexyl)pentanediamide for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy) ethyl)acetamide) to isolate the title compound. UPLC-MS Method A: m/z=1094.6 (z=2); t_{R}=4.30.

### Step 2: (S)-14-((6-(Bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid

The title compound was prepared using procedures analogous to those described for Example 1, Step 5, substituting benzyl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-11,16,20-trioxo-1-(((2S,3 S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1049.6 (z=2); t_{R}=4.15 min.

### Step 3: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl)amino)-2-oxoethyl)-11,16,20-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 6, substituting (S)-14-((6-(bis(2-(((2S,3S,4S,5S,6R)-3,4,5-trihydroxy-6-(hydroxymethyl)tetrahydro-2H-pyran-2-yl)oxy)ethyl)amino)hexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-11,16,20-trioxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: t_{R}=4.23 min; m/z=1098.1 (z=2).

### Example 26: 2,5-Dioxopyrrolidin-1-yl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-16-((6-oxo-6-((2-(((2S,3S,4S,5S,6R)-3,4,5-trihydroxy-6-(hydroxymethyl) tetrahydro-2H-pyran-2-yl)oxy)ethyl)amino)hexyl)carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,17,24,27,30-heptaazahexatriacontan-36-oate (ML-26)

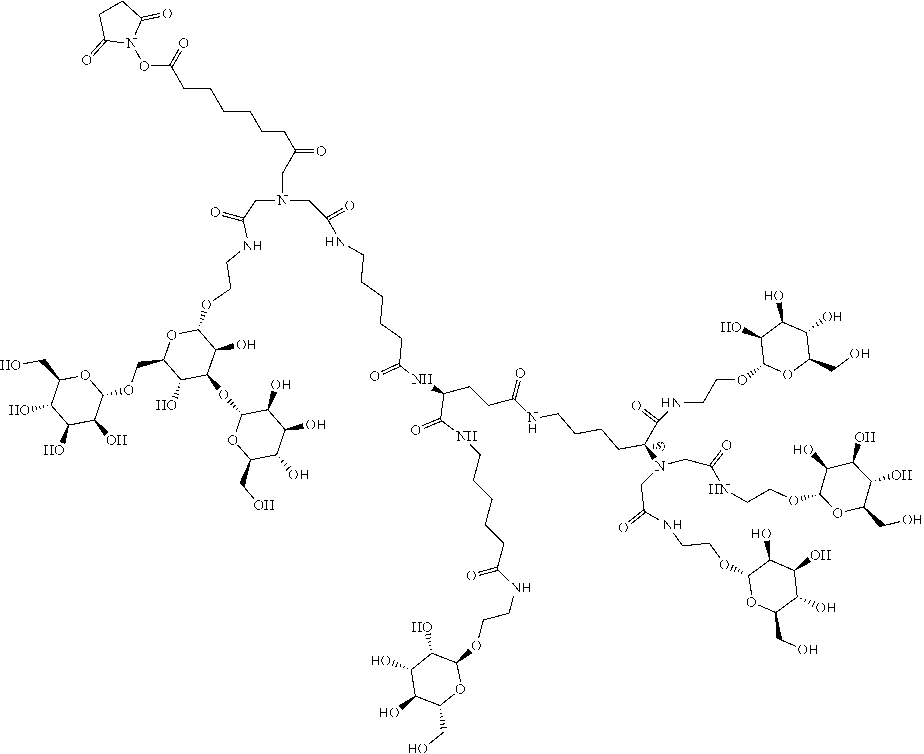

### Step 1: Benzyl ((7S,16S)-4,13,17,24-tetraoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-1,27-bis(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,18,25-pentaazaheptacosan-16-yl)carbamate

To a solution of N²-((benzyloxy)carbonyl)-N⁵—((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl) amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)-L-glutamine (850 mg, 0.745 mmol) and 6-amino-N-(2-(((2S,3S,4S,5S,6R)-3,4,5-trihydroxy-6-(hydroxymethyl)tetrahydro-2H-pyran-2-yl)oxy)ethyl) hexanamide (301 g, 0.894 mmol) in anhydrous DMF (5 mL) was added EDC (286 mg, 1.49 mmol) and DMAP (45 mg, 0.372 mmol), and the resulting mixture was stirred at rt overnight. The mixture was evaporated, and the residue purified by C18 reverse phase chromatography to isolate title compound. UPLC-MS Method A: m/z=1460.2 (z=1); t_{R}=3.53 min.

### Step 2: (S)-2-Amino-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)-N1-(6-oxo-6-((2-(((2S,3S,4S,5S,6R)-3,4,5-trihydroxy-6-(hydroxymethyl)tetrahydro-2H-pyran-2-yl)oxy) ethyl)amino)hexyl)pentanediamide

The title compound was prepared using procedures analogous to those described for Intermediate 5, Step 2, substituting benzyl ((7S,16S)-4,13,17,24-tetraoxo-6-(2-oxo-2-((2-(((2S,3S,4S,5S,6R)-3,4,5-trihydroxy-6-(hydroxymethyl) tetrahydro-2H-pyran-2-yl)oxy)ethyl) amino)ethyl)-1,27-bis(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,18,25-pentaazaheptacosan-16-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl) tetrahydro-2H-pyran-2-yl)oxy)ethyl) amino)-6-oxohexyl)carbamate to isolate title compound. UPLC-MS Method A: m/z=1325.6 (z=1); t_{R}=3.43 min.

### Step 3: Benzyl ((7S,16S)-4,13,18-trioxo-6-(2-oxo-2-((2-(((2S,3S,4,5S,6R)-3,4,5-trihydroxy-6-(hydroxymethyl)tetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl) carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,17-tetraazatricosan-23-yl)carbamate

To a mixture of (S)-2-amino-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)-N1-(6-oxo-6-((2-(((2S,3S,4S,5S,6R)-3,4,5-trihydroxy-6-(hydroxymethyl) tetrahydro-2H-pyran-2-yl)oxy)ethyl)amino)hexyl)pentane diamide (140 mg, 0.106 mmol) and 2,5-dioxopyrrolidin-1-yl 6-(((benzyloxy)carbonyl)amino)hexanoate (50 mg, 0.137 mmol) in anhydrous DMF (3 mL) was added DIPEA (0.026 mL, 0.148 mmol), and the resulting mixture was stirred at rt for 3 h. TFA (0.012 mL, 0.158 mmol) was added to the mixture, and stirring was continued for 10 min. The mixture was then evaporated. The mixture was then added dropwise into anhydrous ACN. The mixture was spun in a centrifuge at 3500 rpm for 15 min. The supernatant was decanted, and the pellet was re-suspended in ACN and spun in a centrifuge at 3500 rpm for a further 15 min. The supernatant was decanted, and the pellet dissolved in water (4 mL) and freeze dried to isolate title compound. UPLC-MS Method A: m/z=1572.7 (z=1); t_{R}=3.50 min.

### Step 4: (S)-2-(6-Aminohexanamido)-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)-N1-(6-oxo-6-((2-(((2S,3S,4S,5S,6R)-3,4,5-trihydroxy-6-(hydroxymethyl)tetrahydro-2H-pyran-2-yl)oxy) ethyl)amino)hexyl)pentanediamide

The title compound was prepared using procedures analogous to those described for Intermediate 5, Step 2, substituting benzyl ((7S,16S)-4,13,18-trioxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,17-tetraazatricosan-23-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)carbamate to isolate title compound. UPLC-MS Method A: m/z=1439.7 (z=1); t_{R}=1.02 min.

### Step 5: Benzyl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy) ethyl)amino)hexyl) carbamoyl)-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl) tetrahydro-2H-pyran-2-yl)oxy)ethyl)carbamoyl)-3,6,12,17,24,27,30-heptaazahexatriacontan-36-oate

To a solution of N-(2-((6-(benzyloxy)-6-oxohexyl)amino)-2-oxoethyl)-N-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl) glycine (80 mg, 0.087 mmol) and (S)-2-(6-aminohexanamido)-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)amino)-6-oxo-6-((2-(((2S,3S,4S,5S,6R)-3,4,5-trihydroxy-6-(hydroxymethyl) tetrahydro-2H-pyran-2-yl)oxy)ethyl)amino) hexyl)-N1-(6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)pentane diamide (125 mg, 0.087 mmol) in DMF (2 mL) was added EDC (33 mg, 0.173 mmol) and DMAP (11 mg, 0.087 mmol), and the resulting mixture stirred at rt overnight. The reaction was evaporated, and the residue was purified by C18 reverse phase chromatography to isolate title compound. UPLC-MS Method A: m/z=1173.0 (z=2); t_{R}=2.35 min.

### Step 6: (7S,16S)-27-(2-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy) ethyl)amino)hexyl) carbamoyl)-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl) tetrahydro-2H-pyran-2-yl)oxy)ethyl)carbamoyl)-3,6,12,17,24,27,30-heptaazahexatriacontan-36-oic acid

The title compound was prepared using procedures analogous to those described for Example 1, Step 5, substituting benzyl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-16-((6-oxo-6-((2-(((2S,3S,4S,5S,6R)-3,4,5-trihydroxy-6-(hydroxymethyl) tetrahydro-2H-pyran-2-yl)oxy)ethyl)amino)hexyl)carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,17,24,27,30-heptaazahexatriacontan-36-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1128.1 (z=2); t_{R}=1.55 min.

### Step 7: 2,5-Dioxopyrrolidin-1-yl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)carbamoyl)-1-(((2,3S4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,17,24,27,30-heptaazahexatriacontan-36-oate

The title compound was prepared using procedures analogous to those described for Example 1, Step 6, substituting (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino) ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,17,24,27,30-heptaazahexatriacontan-36-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: t_{R}=1.70 min; m/z=1176.6 (z=2).

### Example 27: 2,5-Dioxopyrrolidin-1-yl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy) ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl)carbamoyl)-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl) tetrahydro-2H-pyran-2-yl)oxy)ethyl)carbamoyl)-3,6,12,17,24,27,30-heptaazahexatriacontan-36-oate (ML-27)

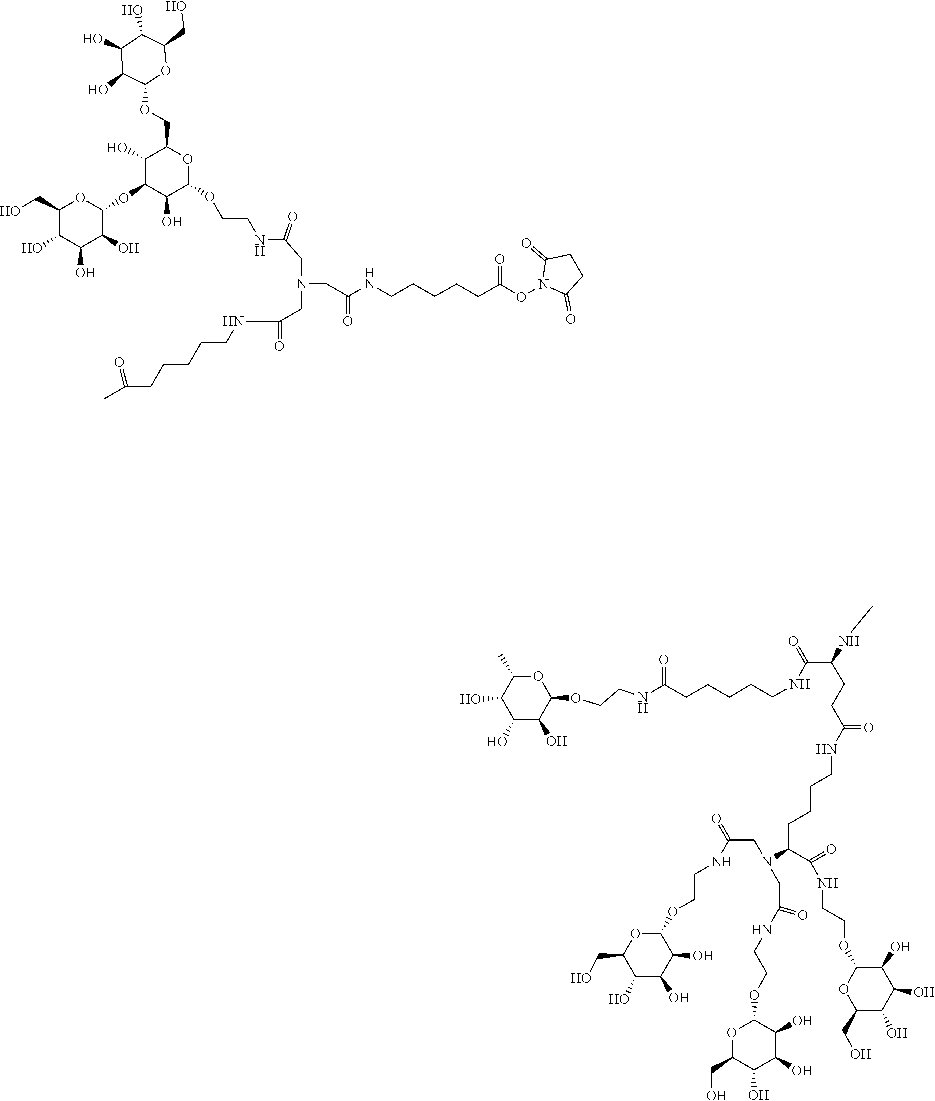 [SMILES: CC(=O)CCCCCNC(=O)CN(CC(=O)NCCCCCC(=O)ON1C(=O)CCC1=O)CC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O ; CN[C@@H](CCC(=O)NCCCC[C@@H](C(=O)NCCO[C@H]1O[C@H](CO)[C@@H](O)[C@H](O)[C@@H]1O)N(CC(=O)NCCO[C@H]1O[C@H](CO)[C@@H](O)[C@H](O)[C@@H]1O)CC(=O)NCCO[C@H]1O[C@H](CO)[C@@H](O)[C@H](O)[C@@H]1O)C(=O)NCCCCCC(=O)NCCO[C@@H]1O[C@@H](C)[C@@H](O)[C@@H](O)[C@@H]1O]

### Step 1: Benzyl ((7S,16S)-4,13,17,24-tetraoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-27-(((2R,3S,4R,5S,6S)-3,4,5-trihydroxy-6-methyltetrahydro-2H-pyran-2-yl)oxy)-3,6,12,18,25-pentaazaheptacosan-16-yl)carbamate

The title compound was prepared using procedures analogous to those described for Example 26, Step 1, substituting 6-amino-N-(2-(((2R,3S,4R,5S,6S)-3,4,5-trihydroxy-6-methyltetrahydro-2H-pyran-2-yl)oxy)ethyl)hexanamide for 6-amino-N-(2-(((2S,3S,4S,5S,6R)-3,4,5-trihydroxy-6-(hydroxymethyl)tetrahydro-2H-pyran-2-yl)oxy)ethyl)hexanamide to isolate title compound. UPLC-MS Method A: m/z=1443.7 (z=1); t_{R}=3.49 min.

### Step 2: (S)-2-Amino-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)-N1-(6-oxo-6-((2-(((2R,3S,4R,5S,6S)-3,4,5-trihydroxy-6-methyltetrahydro-2H-pyran-2-yl)oxy)ethyl)amino)hexyl)pentanediamide

The title compound was prepared using procedures analogous to those described for Intermediate 5, Step 2, substituting benzyl ((7S,16S)-4,13,17,24-tetraoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino) ethyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy) ethyl)carbamoyl)-27-(((2R,3S,4R,5S,6S)-3,4,5-trihydroxy-6-methyltetrahydro-2H-pyran-2-yl)oxy)-3,6,12,18,25-pentaazaheptacosan-16-yl) carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)carbamate to isolate title compound. UPLC-MS Method A: m/z=1309.5 (z=1); t_{R}=3.73 min.

### Step 3: Benzyl ((7S,16S)-4,13,18-trioxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl) carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl) carbamoyl)-3,6,12,17-tetraazatricosan-23-yl)carbamate

To a mixture of (S)-2-amino-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy) ethyl)amino)hexyl)-N1-(6-oxo-6-((2-(((2R,3S,4R,5S,6S)-3,4,5-trihydroxy-6-methyltetrahydro-2H-pyran-2-yl)oxy)ethyl)amino)hexyl)pentanediamide (357 mg, 0.273 mmol) and 2,5-dioxopyrrolidin-1-yl 6-(((benzyloxy)carbonyl)amino)hexanoate (128 mg, 0.354 mmol) in anhydrous DMF (4 mL) was added DIPEA (0.067 mL, 0.382 mmol), and the resulting mixture stirred at rt for 4 h. TFA (0.032 mL, 0.409 mmol) was added, and stirring was continued for 10 min. The solution was added dropwise into anhydrous ACN. The mixture was spun in a centrifuge at 3500 rpm for 15 min. The supernatant was decanted, and the pellet re-suspended in ACN and spun in a centrifuge at 3500 rpm for a further 15 min. The supernatant was decanted, and the pellet was dissolved in water and freeze dried to isolate title compound. UPLC-MS Method A: m/z=1557.8 (z=1); t_{R}=3.68 min.

### Step 4: (S)-2-(6-Aminohexanamido)-N5-((S)-5-(bis(2-oxo-2-((2-(((2S,3S,4S,5.6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)-N1-(6-oxo-6-((2-(((2R,3S,4R,5S,6S)-3,4,5-trihydroxy-6-methyltetrahydro-2H-pyran-2-yl)oxy)ethyl)amino)hexyl)pentanediamide

The title compound was prepared using procedures analogous to those described for Intermediate 5, Step 2, substituting benzyl ((7S,16S)-4,13,18-trioxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy) ethyl)amino)hexyl)carbamoyl)-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl) tetrahydro-2H-pyran-2-yl)oxy)ethyl)carbamoyl)-3,6,12,17-tetraazatricosan-23-yl)carbamate for benzyl (6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)carbamate to isolate title compound. UPLC-MS Method A: m/z=1422.6 (z=1); t_{R}=3.82 min.

### Step 5: Benzyl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino) hexyl)carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl) carbamoyl)-3,6,12,17,24,27,30-heptaazahexatriacontan-36-oate

To a solution of N-(2-((6-(benzyloxy)-6-oxohexyl)amino)-2-oxoethyl)-N-(2-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-2-oxoethyl) glycine (100 mg, 0.108 mmol) and (S)-2-(6-aminohexanamido)-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)amino)-6-oxo-6-((2-(((2S,3S,4S,5S,6R)-3,4,5-trihydroxy-6-(hydroxymethyl) tetrahydro-2H-pyran-2-yl)oxy)ethyl)amino)hexyl)-N1-(6-oxo-6-((2-(((2R,3S,4R,5S,6S)-3,4,5-trihydroxy-6-methyltetrahydro-2H-pyran-2-yl)oxy)ethyl) amino)hexyl)pentane diamide (185 mg, 0.130 mmol) in DMF (2 mL) was added EDC (41 mg, 0.216 mmol) and DMAP (13 mg, 0.108 mmol), and the resulting mixture stirred at rt overnight. The mixture was evaporated, and the residue was purified by C18 reverse phase chromatography to isolate title compound. UPLC-MS Method A: m/z=1165.1 (z=2); t_{R}=2.40 min.

### Step 6: (7S,16S)-27-(2-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino) hexyl)carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl) carbamoyl)-3,6,12,17,24,27,30-heptaazahexatriacontan-36-oic acid

The title compound was prepared using procedures analogous to those described for Example 1, Step 5, substituting benzyl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3 S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl)carbamoyl)-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl) tetrahydro-2H-pyran-2-yl)oxy)ethyl)carbamoyl)-3,6,12,17,24,27,30-heptaazahexatriacontan-36-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl) oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1128.1 (z=2); t_{R}=1.55 min.

### Step 7: 2,5-Dioxopyrrolidin-1-yl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl) carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,17,24,27,30-heptaazahexatriacontan-36-oate

The title compound was prepared using procedures analogous to those described for Example 1, Step 6, substituting (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,25,29-pentaoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino) ethyl)-16-((6-oxo-6-((2-(((2R,3S,4R,5S,6S)-3,4,5-trihydroxy-6-methyltetrahydro-2H-pyran-2-yl)oxy)ethyl) amino)hexyl)carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,17,24,27,30-heptaazahexatriacontan-36-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R, S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1168.6 (z=2); t_{R}=1.75 min.

### Example 28: 2,5-Dioxopyrrolidin-1-yl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-16-((6-oxo-6-((2-(((2R,3S,4R,5S,6S)-3,4,5-trihydroxy-6-methyltetrahydro-2H-pyran-2-yl)oxy) ethyl)amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,17,24,27,30-heptaazakexatriacontan-36-oate (ML-28)

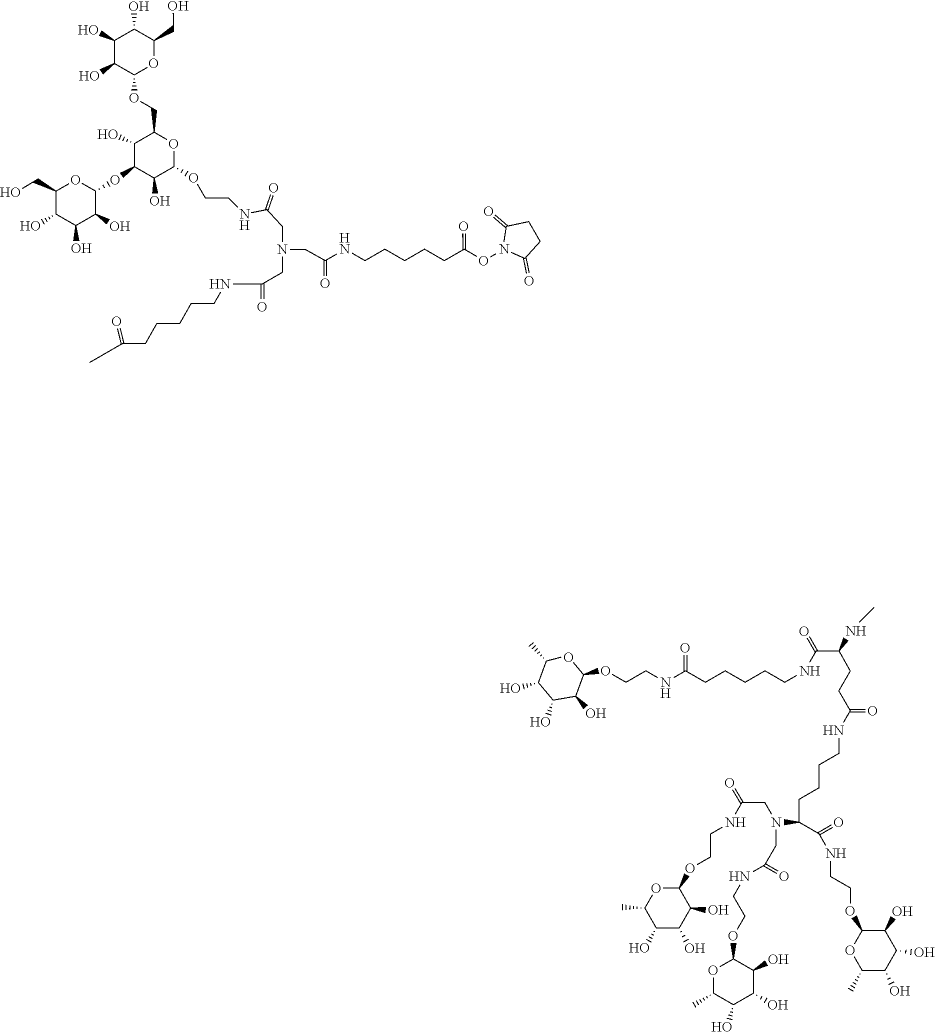 [SMILES: CC(=O)CCCCCNC(=O)CN(CC(=O)NCCCCCC(=O)ON1C(=O)CCC1=O)CC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O ; CN[C@@H](CCC(=O)NCCCC[C@@H](C(=O)NCCO[C@@H]1O[C@@H](C)[C@@H](O)[C@@H](O)[C@@H]1O)N(CC(=O)NCCO[C@@H]1O[C@@H](C)[C@@H](O)[C@@H](O)[C@@H]1O)CC(=O)NCCO[C@@H]1O[C@@H](C)[C@@H](O)[C@@H](O)[C@@H]1O)C(=O)NCCCCCC(=O)NCCO[C@@H]1O[C@@H](C)[C@@H](O)[C@@H](O)[C@@H]1O]

### Step 1: Benzyl ((7S,16S)-4,13,17,24-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-1,27-bis(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,18,25-pentaazaheptacosan-16-yl)carbamate

To a solution of N²-((benzyloxy)carbonyl)-N⁵—((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl) amino)-6-oxo-6-((2-(((2R,3S,4R,5S,6S)-3,4,5-trihydroxy-6-methyltetrahydro-2H-pyran-2-yl)oxy)ethyl)amino) hexyl)-L-glutamine (550 mg, 0.503 mmol) and 6-amino-N-(2-(((2R,3S,4R,5S,6S)-3,4,5-trihydroxy-6-methyltetrahydro-2H-pyran-2-yl)oxy)ethyl)hexanamide (193 mg, 0.604 mmol) in anhydrous DMF (3 mL) was added EDC (193 mg, 1 mmol) and DMAP (30 mg, 0.252 mmol), and the mixture was stirred at rt overnight. The mixture was evaporated, and the residue purified by C18 reverse phase chromatography to isolate title compound. UPLC-MS Method A: m/z=1395.6 (z=1); t_{R}=3.77 min.

### Step 2: (S)-2-Amino-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)-N-(6-oxo-6-((2-(((2R,3S,4R,5S,6S)-3,4,5-trihydroxy-6-methyltetrahydro-2H-pyran-2-yl)oxy)ethyl)amino) hexyl)pentane diamide

The title compound was prepared using procedures analogous to those described for Example 1, Step 5, substituting benzyl ((7S,16S)-4,13,17,24-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-1,27-bis(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,18,25-pentaazaheptacosan-16-yl)carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl) oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1261.7 (z=1); t_{R}=3.48 min.

### Step 3: Benzyl ((7S,16S)-4,13,18-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,17-tetraazatricosan-23-yl)carbamate

The title compound was prepared using procedures analogous to those described for Example 27, Step 3, substituting (S)-2-amino-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)-N1-(6-oxo-6-((2-(((2R,3S,4R,5S,6S)-3,4,5-trihydroxy-6-methyltetrahydro-2H-pyran-2-yl)oxy)ethyl)amino) hexyl)pentane diamide for (S)-2-amino-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino) ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)-N1-(6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)pentanediamide to isolate title compound. UPLC-MS Method A: m/z=1509.8 (z=2); t_{R}=3.51 min.

### Step 4: (S)-2-(6-Aminohexanamido)-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)-N1-(6-oxo-6-((2-(((2R,3S,4R,5S,6S)-3,4,5-trihydroxy-6-methyltetrahydro-2H-pyran-2-yl)oxy)ethyl)amino)hexyl) pentane diamide

The title compound was prepared using procedures analogous to those described for Example 1, Step 5, substituting benzyl ((7S,16S)-4,13,18-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino) hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,17-tetraazatricosan-23-yl)carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy) methyl)tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl) oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,15,18,24,27,30-octaazahexatriacontan-36-oate to isolate title compound. UPLC-MS Method A: m/z=1374.8 (z=2); t_{R}=3.54 min.

### Step 5: Benzyl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl) carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,17,24,27,30-heptaazahexatriaeontan-36-oate

The title compound was prepared using procedures analogous to those described for Example 27, Step 5, substituting (S)-2-(6-aminohexanamido)-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino) ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)-N1-(6-oxo-6-((2-(((2R,3S,4R,5S,6S)-3,4,5-trihydroxy-6-methyltetrahydro-2H-pyran-2-yl)oxy)ethyl)amino)hexyl)pentanediamide for (S)-2-(6-aminohexanamido)-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)-N1-(6-oxo-6-((2-(((2R,3S,4R,5S,6S)-3,4,5-trihydroxy-6-methyltetrahydro-2H-pyran-2-yl)oxy)ethyl)amino) hexyl)pentane diamide to isolate title compound. UPLC-MS Method A: m/z=1141.0 (z=2); t_{R}=2.48 min.

### Step 6: (7S,16S)-27-(2-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl) carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,17,24,27,30-heptaazahexatriacontan-36-oic acid

The title compound was prepared using procedures analogous to those described for Example 1, Step 5, substituting benzyl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy) ethyl)amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,17,24,27,30-heptaazahexatriacontan-36-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1095.5 (z=2); t_{R}=1.73 min.

### Step 7: 2,5-Dioxopyrrolidin-1-yl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino) hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,17,24,27,30-heptaazahexatriacontan-36-oate

The title compound was prepared using procedures analogous to those described for Example 1, Step 6, substituting (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy) ethyl)amino)hexyl) carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,17,24,27,30-heptaazahexatriacontan-36-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl) amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oic acid to isolate title compound. UPLC-MS Method A: m/z=1144.6 (z=2); t_{R}=1.88 min.

### Example 29: 2,5-dioxopyrrolidin-1-yl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,17,24,27,30-heptaazahexatriacontan-36-oate (ML-29)

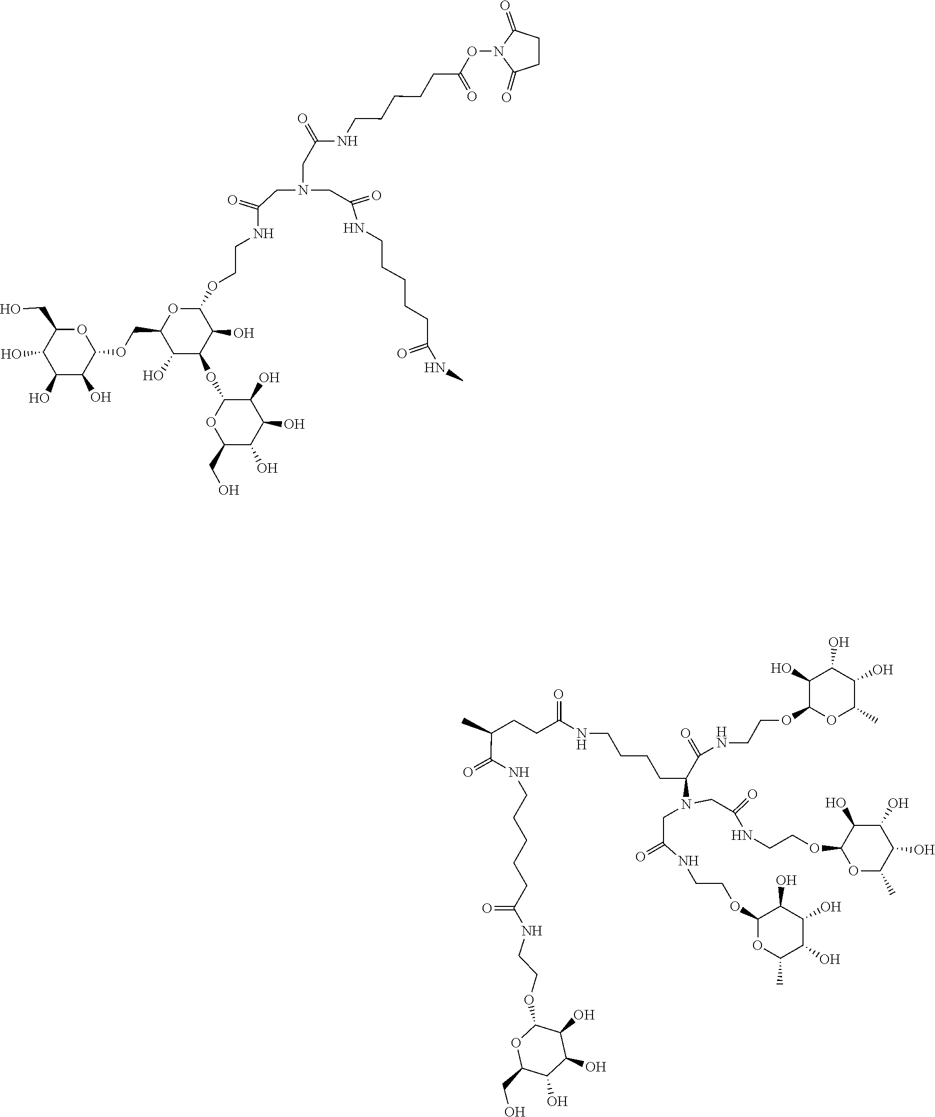

### Step 1: Benzyl ((7S,16S)-4,13,17,24-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-1,27-bis(((2R,3S,4R,5S,6S)-3,4,5-trihydroxy-6-methyltetrahydro-2H-pyran-2-yl)oxy)-7-((2-(((2R,3S,4R,8S,6S)-3,4,5-trihydroxy-6-methyltetrahydro-2H-pyran-2-yl)oxy)ethyl)carbamoyl)-3,6,12,18,25-pentaazaheptacosan-16-yl)carbamate

The title compound was prepared using procedures analogous to those described for Example 26, Step 1, substituting N2-((benzyloxy)carbonyl)-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino) ethyl)amino)-6-oxo-6-((2-(((2R,3S,4R,5S,6S)-3,4,5-trihydroxy-6-methyltetrahydro-2H-pyran-2-yl)oxy)ethyl) amino)hexyl)-L-glutamine for N2-((benzyloxy)carbonyl)-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl) amino)-6-oxo-6-((2-(((2S,3S,4S,5S,6R)-3,4,5-trihydroxy-6-(hydroxymethyl) tetrahydro-2H-pyran-2-yl)oxy)ethyl)amino)hexyl)-L-glutamine and 6-amino-N-(2-(((2S,3S,4S,5S,6R)-3,4,5-trihydroxy-6-(hydroxymethyl)tetrahydro-2H-pyran-2-yl)oxy)ethyl) hexanamide for 6-amino-N-(2-(((2R,3S,4R,5S,6S)-3,4,5-trihydroxy-6-methyltetrahydro-2H-pyran-2-yl)oxy)ethyl)hexanamide to isolate title compound. UPLC-MS Method A: m/z=1411.7 (z=1); t_{R}=3.51 min.

### Step 2: (S)-2-Amino-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)-N1-(6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)pentane diamide

The title compound was prepared using procedures analogous to those described for Example 1, Step 5, substituting benzyl ((7S,16S)-4,13,17,24-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-27-(((2S,3S,4S,5S,6R)-3,4,5-trihydroxy-6-(hydroxymethyl)tetrahydro-2H-pyran-2-yl)oxy)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,18,25-pentaazaheptacosan-16-yl)carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1277.7 (z=1); t_{R}=3.63 min.

### Step 3: Benzyl ((7S,16S)-4,13,18-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl) carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,17-tetraazatricosan-23-yl)carbamate

The title compound was prepared using procedures analogous to those described for Example 27, Step 3, substituting (S)-2-amino-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)-N1-(6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)pentanediamide for (S)-2-amino-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)amino)-6-oxo-6-((2-(((2S,3S,4S,5S,6R)-3,4,5-trihydroxy-6-(hydroxymethyl) tetrahydro-2H-pyran-2-yl)oxy)ethyl)amino)hexyl)-N1-(6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)pentanediamide to isolate title compound. UPLC-MS Method A: m/z=15025.8 (z=2); t_{R}=3.39 min.

### Step 4: (S)-2-(6-Aminohexanamido)-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino)hexyl)-N1-(6-oxo-6-((2-(((2S,3S,4S,5S,6R)-3,4,5-trihydroxy-6-(hydroxymethyl)tetrahydro-2H-pyran-2-yl)oxy)ethyl)amino) hexyl)pentanediamide

The title compound was prepared using procedures analogous to those described for Example 1, Step 5, substituting benzyl ((7S,16S)-4,13,18-trioxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,17-tetraazatricosan-23-yl)carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy) methyl)tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,15,18,24,27,30-octaazahexatriacontan-36-oate to isolate title compound. UPLC-MS Method A: m/z=1391.7 (z=2); t_{R}=3.53 min.

### Step 5: Benzyl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,17,24,27,30-heptaazahexatriacontan-36-oate

The title compound was prepared using procedures analogous to those described for Example 27, Step 5, substituting (S)-2-(6-aminohexanamido)-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl) amino)-6-oxo-6-((2-(((2R,3S,4R,5S,6S)-3,4,5-trihydroxy-6-methyltetrahydro-2H-pyran-2-yl)oxy)ethyl)amino)hexyl)-N1-(6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)pentane diamide for (S)-2-amino-N5-((S)-5-(bis(2-oxo-2-((2-(((2S,3S,4S,5S,6R)-3,4,5-trihydroxy-6-(hydroxymethyl) tetrahydro-2H-pyran-2-yl)oxy)ethyl)amino) ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)-N1-(6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)pentane diamide to isolate title compound. UPLC-MS Method A: m/z=1149.1 (z=2); t_{R}=2.30 min.

### Step 6: (7S,16S)-27-(2-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,17,24,27,30-heptaazahexatriacontan-36-oic acid

The title compound was prepared using procedures analogous to those described for Example 1, Step 5, substituting benzyl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,17,24,27,30-heptaazahexatriacontan-36-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1103.5 (z=2); t_{R}=1.69 min.

### Step 7: 2,5-Dioxopyrrolidin-1-yl (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl) amino)hexyl) carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,17,24,27,30-heptaazahexatriacontan-36-oate

The title compound was prepared using procedures analogous to those described for Example 1, Step 6, substituting (7S,16S)-27-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl) oxy)ethyl)amino) hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,17,24,27,30-heptaazahexatriacontan-36-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1144.6 (z=2); t_{R}=1.88 mm.

### Example 30: 2,5-Dioxopyrrolidin-1-yl (7S,16S)-20-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,22-tetraoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-16-((6-oxo-6-((2-(((2R,3S,4R,5S,6S)-3,4,5-trihydroxy-6-methyltetrahydro-2H-pyran-2-yl) oxy)ethyl)amino)hexyl)carbamoyl)-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl) tetrahydro-2H-pyran-2-yl)oxy)ethyl)carbamoyl)-3,6,12,17,20,23-hexaazanonacosan-29-oate (ML-30)

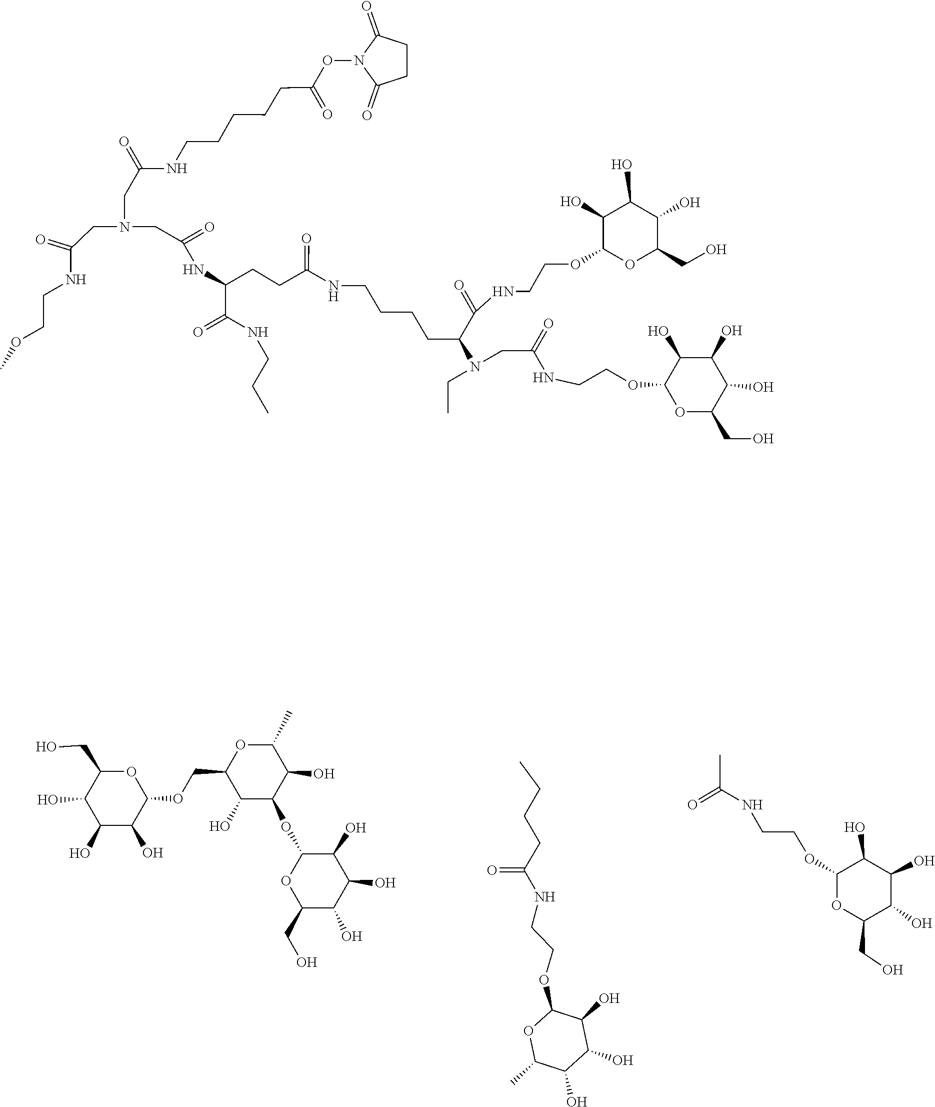

### Step 1: Benzyl (7S,16S)-20-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-4,13,18,22-tetraoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl) carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl) carbamoyl)-3,6,12,17,20,23-hexaazanonacosan-29-oate

The title compound was prepared using procedures analogous to those described for Example 27, Step 5, substituting (S)-2-amino-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl) oxy)ethyl)amino) hexyl)-N1-(6-oxo-6-((2-(((2R,3S,4R,5S,6S)-3,4,5-trihydroxy-6-methyltetrahydro-2H-pyran-2-yl)oxy)ethyl)amino)hexyl)pentanediamide for (S)-2-(6-aminohexanamido)-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)amino)-6-oxo-6-((2-(((2S,3S,4S,5S,6R)-3,4,5-trihydroxy-6-(hydroxymethyl) tetrahydro-2H-pyran-2-yl)oxy)ethyl) amino)hexyl)-N1-(6-oxo-6-((2-(((2S,3S,4S,5S,6R)-3,4,5-trihydroxy-6-(hydroxymethyl) tetrahydro-2H-pyran-2-yl)oxy)ethyl)amino)hexyl)pentane diamide to isolate title compound. UPLC-MS Method A: m/z=1108.5 (z=2); t_{R}=3.58 min.

### Step 2: (7S,16S)-20-(2-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,22-tetraoxo-6-(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl) carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl) carbamoyl)-3,6,12,17,20,23-hexaazanonacosan-29-oic acid

The title compound was prepared using procedures analogous to those described for Example 1, Step 5, substituting benzyl (7S,16S)-20-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,22-tetraoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-16-((6-oxo-6-((2-(((2R,3S,4R,5S,6S)-3,4,5-trihydroxy-6-methyltetrahydro-2H-pyran-2-yl)oxy)ethyl) amino)hexyl)carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,17,20,23-hexaazanonacosan-29-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1063.0 (z=2); t_{R}=3.48 min.

### Step 3: 2,5-Dioxopyrrolidin-1-yl (7S,16S)-20-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,22-tetraoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl)carbamoyl)-1-(((2S,3S,4S,5S,6R)-3,4,5-trihydroxy-6-(hydroxymethyl)tetrahydro-2H-pyran-2-yl) oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,6,12,17,20,23-hexaazanonacosan-29-oate

The title compound was prepared using procedures analogous to those described for Example 1, Step 6, substituting (7S,16S)-20-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,22-tetraoxo-6-(2-oxo-2-((2-(((2S,3S,4S,5S,6R)-3,4,5-trihydroxy-6-(hydroxymethyl)tetrahydro-2H-pyran-2-yl)oxy)ethyl) amino)ethyl)-16-((6-oxo-6-((2-(((2R,3S,4R,5S,6S)-3,4,5-trihydroxy-6-methyltetrahydro-2H-pyran-2-yl)oxy)ethyl)amino)hexyl)carbamoyl)-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl) oxy)ethyl)carbamoyl)-3,6,12,17,20,23-hexaazanonacosan-29-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1122.5 (z=2); t_{R}=3.53 min.

### Example 31: 2,5-Dioxopyrrolidin-1-yl (7S,16S)-20-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,22-tetraoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,17,20,23-hexaazanonacosan-29-oate (ML-31)

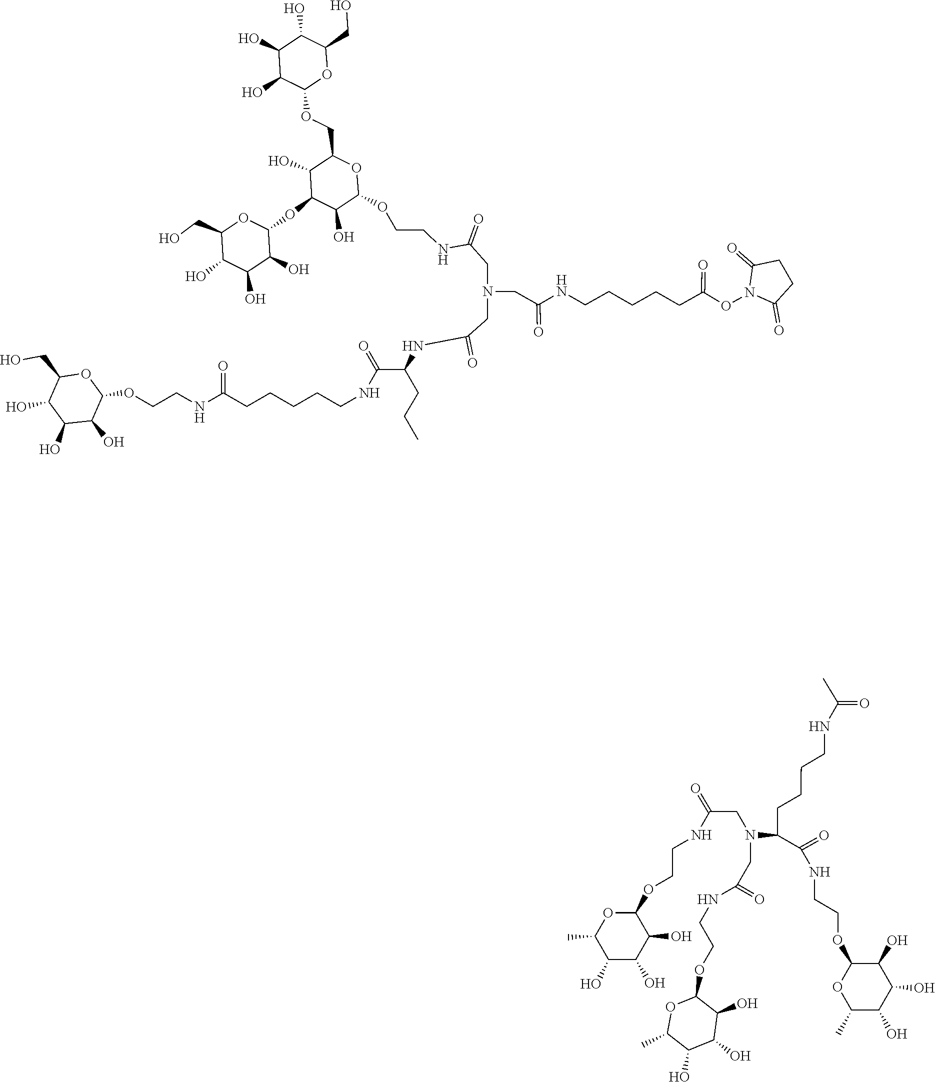 [SMILES: CCC[C@H](NC(=O)CN(CC(=O)NCCCCCC(=O)ON1C(=O)CCC1=O)CC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)C(=O)NCCCCCC(=O)NCCO[C@H]1O[C@H](CO)[C@@H](O)[C@H](O)[C@@H]1O ; CC(=O)NCCCC[C@@H](C(=O)NCCO[C@@H]1O[C@@H](C)[C@@H](O)[C@@H](O)[C@@H]1O)N(CC(=O)NCCO[C@@H]1O[C@@H](C)[C@@H](O)[C@@H](O)[C@@H]1O)CC(=O)NCCO[C@@H]1O[C@@H](C)[C@@H](O)[C@@H](O)[C@@H]1O]

### Step 1: Benzyl (7S,16S)-20-(2-((2-(((2S,3S,4S,5R,66R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-2-oxoethyl)-4,13,18,22-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl) amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,17,20,23-hexaazanonacosan-29-oate

The title compound was prepared using procedures analogous to those described for Example 27, Step 5, substituting (S)-2-amino-N5-((S)-5-(bis(2-oxo-2-((2-(((2R,3S,4R,5S,6S)-3,4,5-trihydroxy-6-methyltetrahydro-2H-pyran-2-yl)oxy)ethyl)amino)ethyl)amino)-6-oxo-6-((2-(((2R,3S,4R,5S,6S)-3,4,5-trihydroxy-6-methyltetrahydro-2H-pyran-2-yl)oxy)ethyl)amino) hexyl)-N1-(6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)pentane diamide for (S)-2-(6-aminohexanamido)-N5-((S)-5-(bis(2-oxo-2-((2-(((2S,3S,4S,5S,6R)-3,4,5-trihydroxy-6-(hydroxymethyl)tetrahydro-2H-pyran-2-yl)oxy)ethyl)amino)ethyl)amino)-6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)-N1-(6-oxo-6-((2-(((2S,3S,4S,5S,6R)-3,4,5-trihydroxy-6-(hydroxymethyl)tetrahydro-2H-pyran-2-yl)oxy)ethyl)amino) hexyl) pentane diamide to isolate title compound. UPLC-MS Method A: m/z=1092.0 (z=2); t_{R}=3.47 min.

### Step 2: (7S,16S)-20-(2-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,22-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl) carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,17,20,23-hexaazanonacosan-29-oic acid

The title compound was prepared using procedures analogous to those described for Example 1, Step 5, substituting benzyl (7S,16S)-20-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,22-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,17,20,23-hexaazanonacosan-29-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1047.0 (z=2); t_{R}=3.54 min.

### Step 3: 2,5-Dioxopyrrolidin-1-yl (7S,16S)-20-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-2-oxoethyl)-4,13,18,22-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy)ethyl)amino)hexyl) carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,17,20,23-hexaazanonacosan-29-oate

The title compound was prepared using procedures analogous to those described for Example 1, Step 6, substituting (7S,16S)-20-(2-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-2-oxoethyl)-4,13,18,22-tetraoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-16-((6-oxo-6-((2-(((2S,3S,4S,5S,6R)-3,4,5-trihydroxy-6-(hydroxymethyl)tetrahydro-2H-pyran-2-yl)oxy) ethyl)amino)hexyl)carbamoyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,17,20,23-hexaazanonacosan-29-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy) methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl) oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino) hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1095.5 (z=2); t_{R}=3.51 min.

### Example 32: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-4,11,16,20-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,15,18,21-pentaazaheptacosan-27-oate (ML-32)

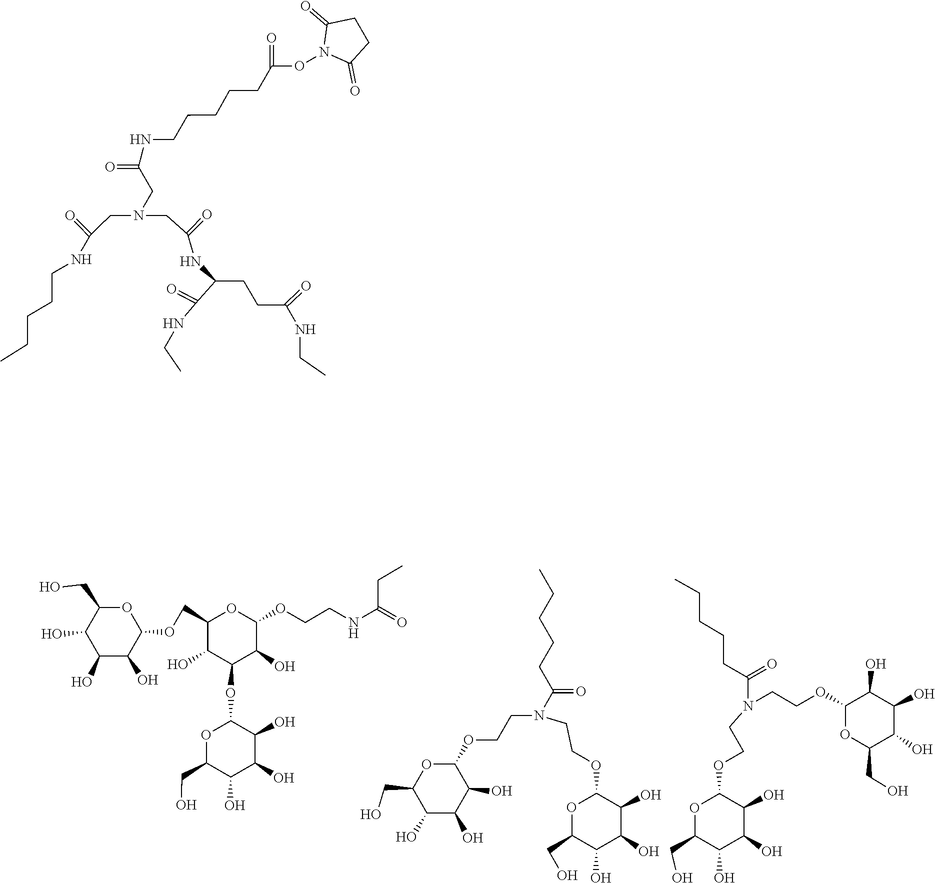

### Step 1: Benzyl (S)-14-((6-(bis(2-(((2S,3S,4S,5S,6R)-3,4,5-trihydroxy-6-(hydroxymethyl) tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl) tetrahydro-2H-pyran-2-yl)oxy)ethyl)amino)-6-oxohexyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 4, substituting (S)-2-amino-N1,N5-bis(6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl) pentanediamide for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) to isolate the title compound. UPLC-MS Method A: m/z=1108.5 (z=2); t_{R}=2.58.

### Step 2: (S)-14-((6-(Bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,11,16,20-tetraoxo-1-(((2S,3S,4,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid

The title compound was prepared using procedures analogous to those described for Example 1, Step 5, substituting benzyl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3 S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1063.5 (z=2); t_{R}=1.89 min.

### Step 3: 2,5-Dioxopyrrolidin-1-yl (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,354S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oate

The title compound was prepared using procedures analogous to those described for Example 1, Step 6, substituting (S)-14-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl) amino)-2-oxoethyl)-4,11,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,15,18,21-pentaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oic acid to isolate title compound. UPLC-MS Method A: m/z=1111.5 (z=2); t_{R}=2.02 min.

### Example 33: 2,5-Dioxopyrrolidin-1-yl (S)-7-(bis(2-(((2S,3S,4S,5S,6R)-3,4,5-trihydroxy-6-(hydroxymethyl)tetrahydro-2H-pyran-2-yl)oxy)ethyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,9,16,20-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,8,15,18,21-pentaazaheptacosan-27-oate (ML-33)

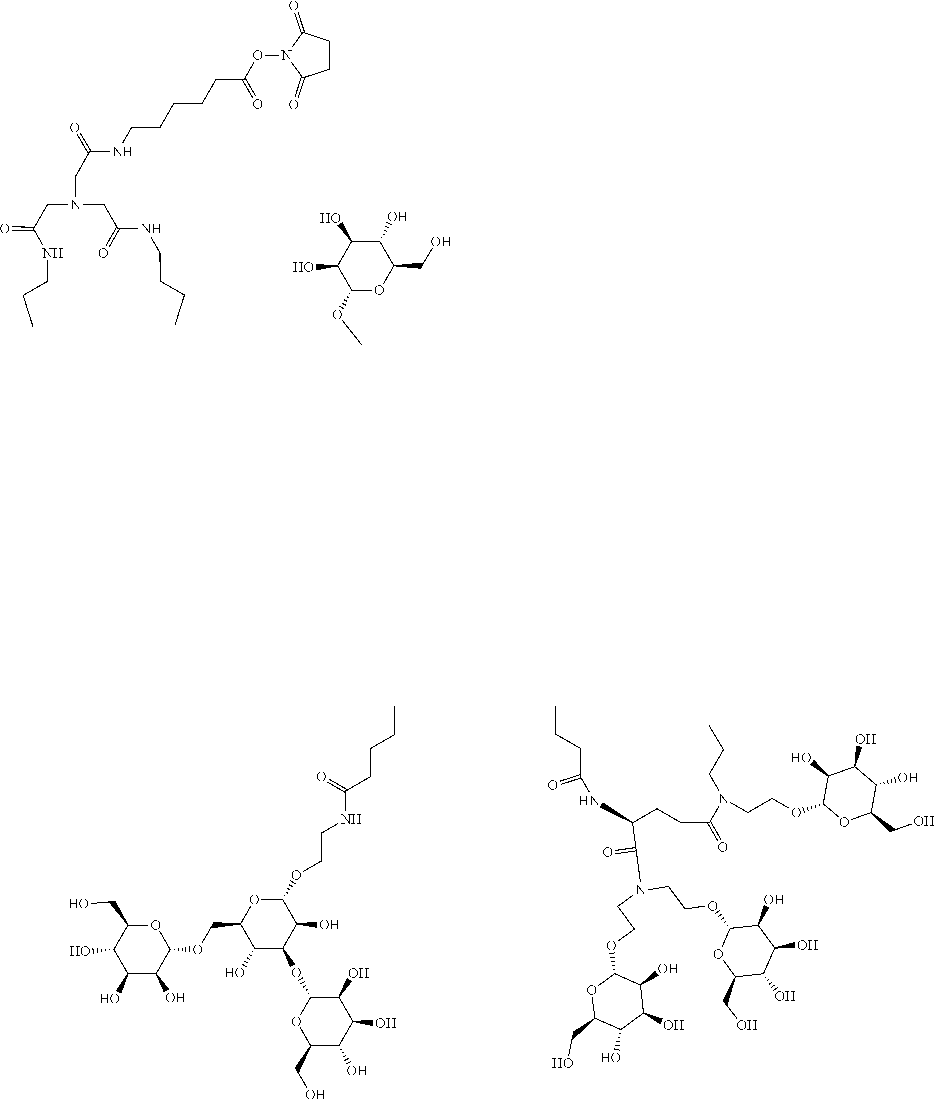 [SMILES: CCCCNC(=O)CN(CC(=O)NCCC)CC(=O)NCCCCCC(=O)ON1C(=O)CCC1=O ; CO[C@H]1O[C@H](CO)[C@@H](O)[C@H](O)[C@@H]1O ; CCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O ; CCCC(=O)N[C@@H](CCC(=O)N(CCC)CCO[C@H]1O[C@H](CO)[C@@H](O)[C@H](O)[C@@H]1O)C(=O)N(CCO[C@H]1O[C@H](CO)[C@@H](O)[C@H](O)[C@@H]1O)CCO[C@H]1O[C@H](CO)[C@@H](O)[C@H](O)[C@@H]1O]

### Step 1: Benzyl ((S)-1,5-bis(bis(2-(((2S,3S,4S,5S,6R)-3,4,5-trihydroxy-6-(hydroxymethyl) tetrahydro-2H-pyran-2-yl)oxy)ethyl)amino)-1,5-dioxopentan-2-yl)carbamate

To a mixture of (S)-2-(((benzyloxy)carbonyl)amino)pentanedioic acid (500 mg, 1.778 mmol) and bis{2-[(2,3,4,6-tetra-O-acetyl-α-D-mannopyranosyl)oxy]ethyl}amine (WO2020247297A1) (3.4 g, 4.44 mmol) in DMF (5 mL) was added EDC (1.02 g, 5.33 mmol), HOBt (27 mg, 0.178 mmol) and Et₃N (0.025 mL, 0.178 mmol), and the mixture was stirred at rt overnight. The mixture was evaporated, and the residue was partitioned between EtOAc (100 mL) and water (100 mL) and extracted with EtOAc (2×100 mL). The combined EtOAc layers were washed with 1N HCl (3×75 mL) and sat. NaCl (50 mL), then dried over Na₂SO₄, filtered and evaporated. The residue was purified by normal phase silica gel column chromatography. The resulting material was taken into MeOH (10 mL) and was added to NaOMe solution (0.03 mL of a 4.37M solution in MeOH, 0.129 mmol), and the resulting mixture stirred at rt. After 30 min, MeOH (10 mL) was added to return all material to solution, and stirring was continued for 2 h. The mixture was quenched by the addition of DOWEX™ ion exchange resin (50 W), which had been pre-washed with MeOH (˜2 g). The mixture was stirred for 10 min, then filtered, and the filtrate was evaporated. The residue was taken up in water and freeze dried to isolate title compound. UPLC-MS Method A: m/z=1104.5 (z=1); t_{R}=3.75 min.

### Step 2: (S)-2-Amino-N1,N1,N5,N5-tetrakis(2-(((2S,3S,4S,5S,6R)-3,4,5-trihydroxy-6-(hydroxymethyl)tetrahydro-2H-pyran-2-yl)oxy)ethyl)pentanediamide

The title compound was prepared using procedures analogous to those described for Example 1, Step 5, substituting benzyl ((S)-1,5-bis(bis(2-(((2S,3S,4S,5S,6R)-3,4,5-trihydroxy-6-(hydroxymethyl)tetrahydro-2H-pyran-2-yl)oxy)ethyl)amino)-1,5-dioxopentan-2-yl)carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=970.5 (z=1); t_{R}=1.24 min.

### Step 3: Benzyl (6-(((S)-1,5-bis(bis(2-(((2S,3S,4S,5S,6R)-3,4,5-trihydroxy-6-(hydroxymethyl) tetrahydro-2H-pyran-2-yl)oxy)ethyl)amino)-1,5-dioxopentan-2-yl)amino)-6-oxohexyl)carbamate

To a solution of (S)-2-amino-N1,N1,N5,N5-tetrakis(2-(((2S,3S,4S,5S,6R)-3,4,5-trihydroxy-6-(hydroxymethyl)tetrahydro-2H-pyran-2-yl)oxy)ethyl)pentanediamide (695 mg, 0.72 mmol) in anhydrous DMF (3 mL) was added 2,5-dioxopyrrolidin-1-yl 6-(((benzyloxy) carbonyl)amino) hexanoate (28 mg, 0.79 mmol) and DIPEA (0.15 mL, 0.86 mmol), and the resulting mixture was stirred at rt overnight. The mixture was added dropwise to ACN (60 mL), and the mixture was spun in a centrifuge (30 min at 3500 rpm). The supernatant was discarded, and the pellet was re-suspended in ACN and spun in a centrifuge (15 mins at 3500 rpm). The supernatant was discarded. The pellet was then purified by C18 reverse phase chromatography to isolate title compound. UPLC-MS Method A: m/z=1217.5 (z=1); t_{R}=3.89 min.

### Step 4: (S)-2-(6-Aminohexanamido)-N1,N1,N5,N5-tetrakis(2-(((2S,3S4S,5S,6R)-3,4,5-trihydroxy-6-(hydroxymethyl)tetrahydro-2H-pyran-2-yl)oxy)ethyl)pentanediamide

The title compound was prepared using procedures analogous to those described for Example 1, Step 5, substituting benzyl (6-(((S)-1,5-bis(bis(2-(((2S,3S,4S,5S,6R)-3,4,5-trihydroxy-6-(hydroxymethyl)tetrahydro-2H-pyran-2-yl)oxy)ethyl)amino)-1,5-dioxopentan-2-yl)amino)-6-oxohexyl)carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy) ethyl)carbamoyl)-3,6,12,15,18,24,27,30-octaazahexatriacontan-36-oate to isolate title compound. UPLC-MS Method A: m/z=1083.5 (z=1); t_{R}=1.30 min.

### Step 5: Benzyl (S)-7-(bis(2-(((2S,3S,4S,5S,6R)-3,4,5-trihydroxy-6-(hydroxymethyl)tetrahydro-2H-pyran-2-yl)oxy)ethyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy) ethyl)amino)-6-oxohexyl)amino)-2-oxoethyl)-4,9,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,8,15,18,21-pentaazaheptacosan-27-oate

The title compound was prepared using procedures analogous to those described for Example 1, Step 4, substituting (S)-2-(6-aminohexanamido)-N1,N1,N5,N5-tetrakis(2-(((2S,3S,4S,5S,6R)-3,4,5-trihydroxy-6-(hydroxymethyl)tetrahydro-2H-pyran-2-yl)oxy)ethyl) pentanediamide for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) to isolate the title compound. UPLC-MS Method A: m/z=1052.0 (z=2); t_{R}=4.15 min.

### Step 6: (S)-7-(Bis(2-(((2S,3S,4S,5S,6R)-3,4,5-trihydroxy-6-(hydroxymethyl)tetrahydro-2H-pyran-2-yl)oxy)ethyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-4,9,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,8,15,18,21-pentaazaheptacosan-27-oic acid

The title compound was prepared using procedures analogous to those described for Example 1, Step 5, substituting benzyl (S)-7-(bis(2-(((2S,3S,4S,5S,6R)-3,4,5-trihydroxy-6-(hydroxymethyl)tetrahydro-2H-pyran-2-yl)oxy)ethyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)amino)-2-oxoethyl)-4,9,16,20-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,8,15,18,21-pentaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1006.5 (z=2); t_{R}=3.86 min.

### Step 7: 2,5-Dioxopyrrolidin-1-yl (S)-7-(bis(2-(((2S,3S,4S,5S,6R)-3,4,5-trihydroxy-6-(hydroxymethyl)tetrahydro-2H-pyran-2-yl)oxy)ethyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,9,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,8,15,18,21-pentaazaheptacosan-27-oate

The title compound was prepared using procedures analogous to those described for Example 1, Step 6, substituting (S)-7-(bis(2-(((2S,3S,4S,5S,6R)-3,4,5-trihydroxy-6-(hydroxymethyl)tetrahydro-2H-pyran-2-yl)oxy)ethyl)carbamoyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)amino)-2-oxoethyl)-4,9,16,20-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,8,15,18,21-pentaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1055.5 (z=2); t_{R}=3.8 min.

### Example 34: 2,5-Dioxopyrrolidin-1-yl 6-(2-(bis(2-(((2S,3S,4S,5S,6R)-3,4,5-trihydroxy-6-(hydroxymethyl)tetrahydro-2H-pyran-2-yl)oxy)ethyl)amino)-2-oxoethyl)-18-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl)amino)-2-oxoethyl)-4,8,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,6,9,15,18,21-hexaazaheptacosan-27-oate (ML-34)

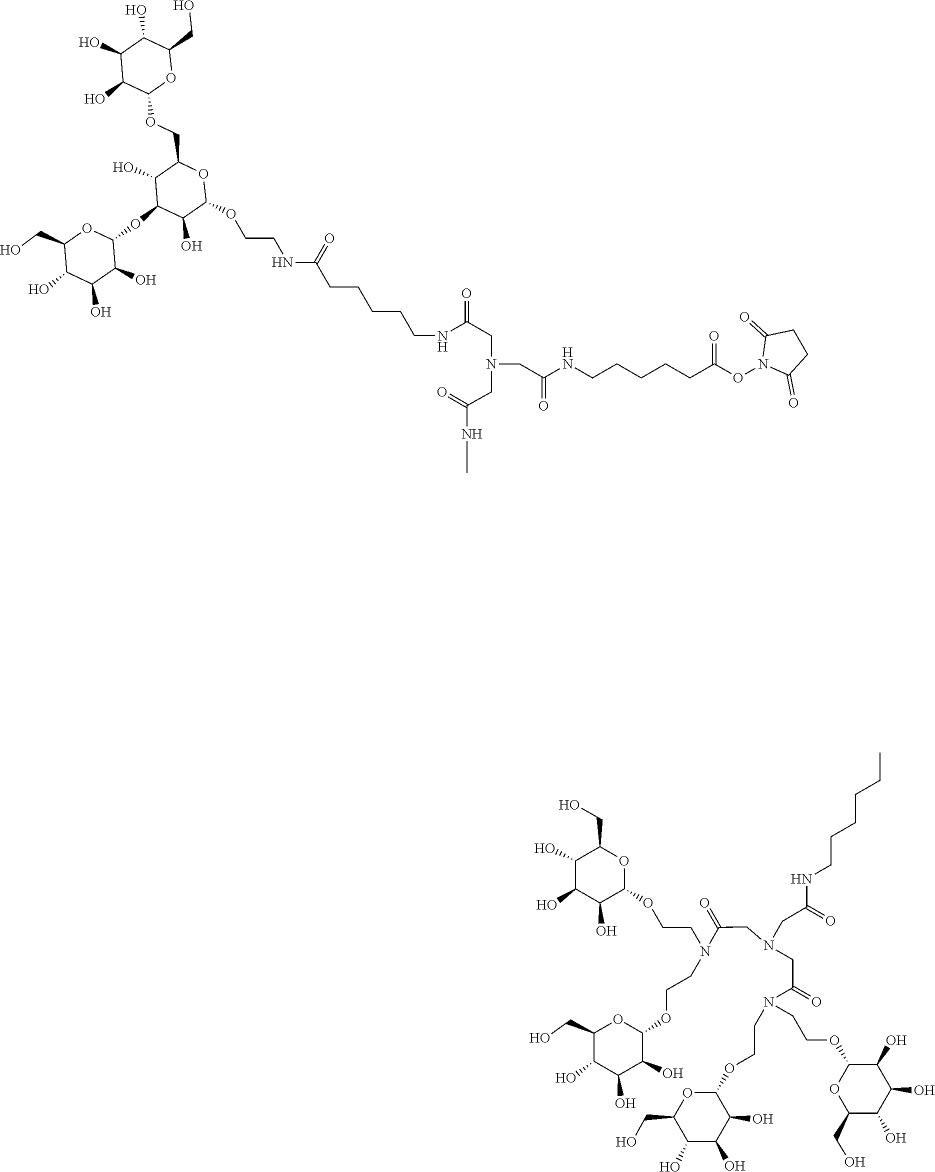 [SMILES: CNC(=O)CN(CC(=O)NCCCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CC(=O)NCCCCCC(=O)ON1C(=O)CCC1=O ; CCCCCCNC(=O)CN(CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O]

### Step 1: Benzyl (5-(2-(bis(2-(bis(2-(((2S,3S,4S,5S,6R)-3,4,5-trihydroxy-6-(hydroxymethyl) tetrahydro-2H-pyran-2-yl)oxy)ethyl)amino)-2-oxoethyl)amino)acetamido)pentyl)carbamate

The title compound was prepared using procedures analogous to those described for Example 33, Step 1, substituting (S)-2-(((benzyloxy)carbonyl)amino)pentane dioic acid for 13-(carboxymethyl)-3,11-dioxo-1-phenyl-2-oxa-4,10,13-triazapentadecan-15-oic acid to isolate title compound. UPLC-MS Method A: m/z=1232.6 (z=1); t_{R}=3.95 min.

### Step 2: 2,2′-((2-((5-Aminopentyl)amino)-2-oxoethyl)azanediyl)bis(N,N-bis(2-(((2S,3S,4S,5S,6R)-3,4,5-trihydroxy-6-(hydroxymethyl)tetrahydro-2H-pyran-2-yl)oxy)ethyl)acetamide)

The title compound was prepared using procedures analogous to those described for Example 1, Step 5, substituting benzyl (5-(2-(bis(2-(bis(2-(((2S,3S,4S,5S,6R)-3,4,5-trihydroxy-6-(hydroxymethyl)tetrahydro-2H-pyran-2-yl)oxy)ethyl)amino)-2-oxoethyl)amino)acetamido) pentyl)carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1014.0 (z=1); t_{R}=1.25 min.

### Step 3: Benzyl 6-(2-(bis(2-(((2S,3S,4S,5S,6R)-3,4,5-trihydroxy-6-(hydroxymethyl)tetrahydro-2H-pyran-2-yl)oxy)ethyl)amino)-2-oxoethyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,8,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,6,9,15,18,21-hexaazaheptacosan-27-oate

The title compound was prepared using procedures analogous to those described for Example 1, Step 4, substituting 2,2′-((2-((5-aminopentyl)amino)-2-oxoethyl)azanediyl)bis(N,N-bis(2-(((2S,3S,4S,5S,6R)-3,4,5-trihydroxy-6-(hydroxymethyl)tetrahydro-2H-pyran-2-yl)oxy) ethyl)acetamide) for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) to isolate the title compound. UPLC-MS Method A: m/z=1059.5 (z=2); t_{R}=4.00 mm.

### Step 4: 6-(2-(Bis(2-(((2S,3S,4S,5S,6R)-3,4,5-trihydroxy-6-(hydroxymethyl)tetrahydro-2H-pyran-2-yl)oxy)ethyl)amino)-2-oxoethyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,8,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,6,9,15,18,21-hexaazaheptacosan-27-oic acid

The title compound was prepared using procedures analogous to those described for Example 1, Step 5, substituting benzyl 6-(2-(bis(2-(((2S,3S,4S,5S,6R)-3,4,5-trihydroxy-6-(hydroxymethyl)tetrahydro-2H-pyran-2-yl)oxy)ethyl)amino)-2-oxoethyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,8,16,20-tetraoxo-1-(((2S,3S,4S,5S,6R)-3,4,5-trihydroxy-6-(hydroxymethyl) tetrahydro-2H-pyran-2-yl) oxy)-3-(2-(((2S,3S,4S,5S,6R)-3,4,5-trihydroxy-6-(hydroxymethyl) tetrahydro-2H-pyran-2-yl)oxy) ethyl)-3,6,9,15,18,21-hexaazaheptacosan-27-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1014.0 (z=2); t_{R}=3.87 min.

### Step 5: 2,5-Dioxopyrrolidin-1-yl 6-(2-(bis(2-(((2S,3S,4S,5S,6R)-3,4,5-trihydroxy-6-(hydroxymethyl)tetrahydro-2H-pyran-2-yl)oxy)ethyl)amino)-2-oxoethyl)-18-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl)amino)-2-oxoethyl)-4,8,16,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,6,9,15,18,21-hexaazaheptacosan-27-oate

The title compound was prepared using procedures analogous to those described for Example 1, Step 6, substituting 6-(2-(bis(2-(((2S,3S,4S,5S,6R)-3,4,5-trihydroxy-6-(hydroxymethyl)tetrahydro-2H-pyran-2-yl)oxy)ethyl)amino)-2-oxoethyl)-18-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,8,16,20-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,6,9,15,18,21-hexaazaheptacosan-27-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl) amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy) ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1063.0 (z=2); t_{R}=3.88 min.

### Example 35: 2,5-Dioxopyrrolidin-1-yl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-11-(2-(((S)-1,5-dioxo-1,5-bis((2-(((2S,3S,4S,5S,6R)-3,4,5-trihydroxy-6-(hydroxymethyl)tetrahydro-2H-pyran-2-yl)oxy) ethyl)amino)pentan-2-yl)amino)-2-oxoethyl)-4,9,13,21,25-pentaoxo-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,8,11,14,20,23,26-heptaazadotriacontan-32-oate (ML-35)

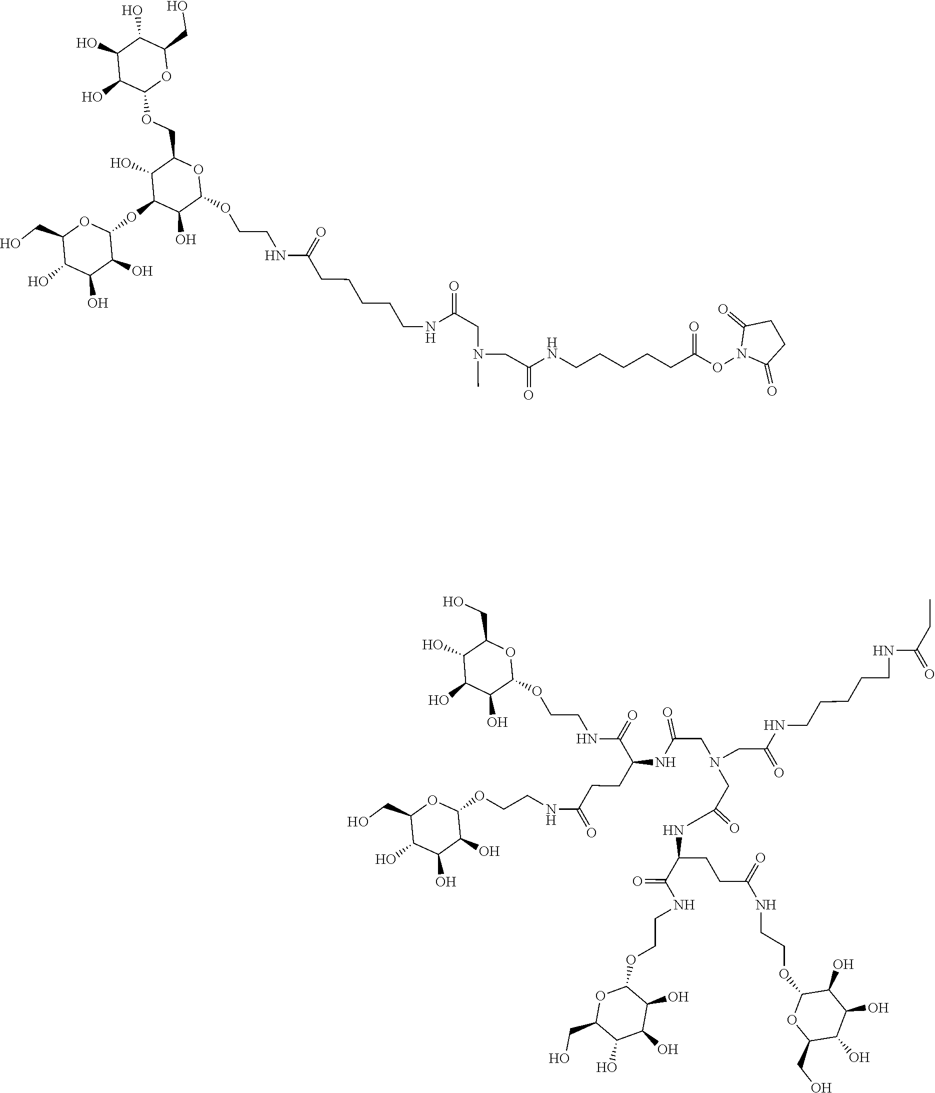 [SMILES: CN(CC(=O)NCCCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)CC(=O)NCCCCCC(=O)ON1C(=O)CCC1=O ; CCC(=O)NCCCCCNC(=O)CN(CC(=O)N[C@@H](CCC(=O)NCCO[C@H]1O[C@H](CO)[C@@H](O)[C@H](O)[C@@H]1O)C(=O)NCCO[C@H]1O[C@H](CO)[C@@H](O)[C@H](O)[C@@H]1O)CC(=O)N[C@@H](CCC(=O)NCCO[C@H]1O[C@H](CO)[C@@H](O)[C@H](O)[C@@H]1O)C(=O)NCCO[C@H]1O[C@H](CO)[C@@H](O)[C@H](O)[C@@H]1O]

### Step 1: Benzyl ((S)-1,5-dioxo-1,5-bis((2-(((2S,3S,4S,5S,6R)-3,4,5-trihydroxy-6-(hydroxymethyl)tetrahydro-2H-pyran-2-yl)oxy)ethyl)amino)pentan-2-yl)carbamate

To a solution of ((benzyloxy)carbonyl)-L-glutamic acid (1 g, 3.56 mmol) and (2S,3S,4S,5S,6R)-2-(2-aminoethoxy)-6-(hydroxymethyl)tetrahydro-2H-pyran-3,4,5-triol (2.38 g, 10.7 mmol) in anhydrous DMF (15 mL) was added EDC (2.73 g, 14.2 mmol), HOBt (54 mg, 0.36 mmol), and TEA (0.05 mL, 0.36 mmol), and the resulting mixture stirred at rt overnight. The mixture was evaporated, and the residue was purified by reverse phase C18 chromatography to isolate title compound. UPLC-MS Method A: m/z=692.1 (z=1); t_{R}=3.86 min.

### Step 2: (S)-2-Amino-N1,N5-bis(2-(((2S,3S,4S,5S,6R)-3,4,5-trihydroxy-6-(hydroxymethyl) tetrahydro-2H-pyran-2-yl)oxy)ethyl)pentanediamide

The title compound was prepared using procedures analogous to those described for Example 1, Step 5, substituting benzyl ((S)-1,5-dioxo-1,5-bis((2-(((2S,3S,4S,5S,6R)-3,4,5-trihydroxy-6-(hydroxymethyl)tetrahydro-2H-pyran-2-yl)oxy)ethyl)amino)pentan-2-yl)carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=558.1 (z=1); t_{R}=1.26 min.

### Step 3: Benzyl ((S)-11-(2-(((S)-1,5-dioxo-1,5-bis((2-(((2S,3S,4S,5S,6R)-3,4,5-trihydroxy-6-(hydroxymethyl)tetrahydro-2H-pyran-2-yl)oxy)ethyl)amino)pentan-2-yl)amino)-2-oxoethyl)-4,9,13-trioxo-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,8,11,14-tetraazanonadecan-19-yl)carbamate

To a suspension of 13-(carboxymethyl)-3,11-dioxo-1-phenyl-2-oxa-4,10,13-triazapentadecan-15-oic acid (540 mg, 1.32 mmol) and (S)-2-amino-N1,N5-bis(2-(((2S,3S,4S,5S,6R)-3,4,5-trihydroxy-6-(hydroxymethyl)tetrahydro-2H-pyran-2-yl)oxy)ethyl)pentanediamide (1.8 g, 3.3 mmol) in anhydrous DMF (0 mL) was added EDC (885 mg, 4.62 mmol), and DIPEA (0.46 mL, 2.64 mmol), and the resulting mixture stirred at rt overnight. The mixture was evaporated, and the residue was purified by C18 reverse phase chromatography to isolate title compound. UPLC-MS Method A: m/z=1489.1 (z=1); t_{R}=4.48 min.

### Step 4: (2S,2′S)-2,2′-((2,2′-((2-((5-Aminopentyl)amino)-2-oxoethyl)azanediyl)bis(acetyl))bis(azanediyl))bis(N1,N5-bis(2-(((2S,3S,4S,5S,6R)-3,4,5-trihydroxy-6-(hydroxymethyl) tetrahydro-2H-pyran-2-yl)oxy)ethyl)pentanediamide)

The title compound was prepared using procedures analogous to those described for Example 1, Step 5, substituting benzyl ((S)-11-(2-(((S)-1,5-dioxo-1,5-bis((2-(((2S,3S,4S,5S,6R)-3,4,5-trihydroxy-6-(hydroxymethyl)tetrahydro-2H-pyran-2-yl)oxy)ethyl)amino)pentan-2-yl)amino)-2-oxoethyl)-4,9,13-trioxo-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl) tetrahydro-2H-pyran-2-yl)oxy)ethyl)carbamoyl)-3,8,11,14-tetraazanonadecan-19-yl)carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1355.1 (z=1); t_{R}=3.99 min.

### Step 5: Benzyl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-11-(2-(((S)-1,5-dioxo-1,5-bis((2-(((2S,3S,4S,5S,6R)-3,4,5-trihydroxy-6-(hydroxymethyl)tetrahydro-2H-pyran-2-yl)oxy)ethyl) amino)pentan-2-yl)amino)-2-oxoethyl)-4,9,13,21,25-pentaoxo-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl) tetrahydro-2H-pyran-2-yl)oxy)ethyl)carbamoyl)-3,8,11,14,20,23,26-heptaazadotriacontan-32-oate

The title compound was prepared using procedures analogous to those described for Example 1, Step 4, substituting 2S,2′S)-2,2′-((2,2′-((2-((5-aminopentyl)amino)-2-oxoethyl) azanediyl)bis(acetyl))bis(azanediyl))bis(N1,N5-bis(2-(((2S,3S,4S,5S,6R)-3,4,5-trihydroxy-6-(hydroxymethyl)tetrahydro-2H-pyran-2-yl)oxy)ethyl)pentanediamide) for 2,2′-(((S)-13-(2-((5-aminopentyl)amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) to isolate the title compound. UPLC-MS Method A: m/z=1187.6 (z=2); t_{R}=4.42 min.

### Step 6: (S)-23-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-11-(2-(((S)-1,5-dioxo-1,5-bis((2-(((2S,3S,4S,5S,6R)-3,4,5-trihydroxy-6-(hydroxymethyl)tetrahydro-2H-pyran-2-yl)oxy)ethyl)amino)pentan-2-yl)amino)-2-oxoethyl)-4,9,13,21,25-pentaoxo-1-(((2S,3S,4S,5S,6R)-3,4,5-trihydroxy-6-(hydroxymethyl) tetrahydro-2H-pyran-2-yl)oxy)-7-((2-(((2S,3S,4S,5S,6R)-3,4,5-trihydroxy-6-(hydroxymethyl) tetrahydro-2H-pyran-2-yl)oxy)ethyl)carbamoyl)-3,8,11,14,20,23,26-heptaazadotriacontan-32-oic acid

The title compound was prepared using procedures analogous to those described for Example 1, Step 5, substituting benzyl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-11-(2-(((S)-1,5-dioxo-1,5-bis((2-(((2S,3S,4S,5S,6R)-3,4,5-trihydroxy-6-(hydroxymethyl)tetrahydro-2H-pyran-2-yl)oxy)ethyl)amino)pentan-2-yl)amino)-2-oxoethyl)-4,9,13,21,25-pentaoxo-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,8,11,14,20,23,26-heptaazadotriacontan-32-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1142.1 (z=2); t_{R}=3.78 mm.

### Step 7: 2,5-Dioxopyrrolidin-1-yl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-11-(2-(((S)-1,5-dioxo-1,5-bis((2-(((2S,3S,4S,5S,6R)-3,4,5-trihydroxy-6-(hydroxymethyl)tetrahydro-2H-pyran-2-yl)oxy)ethyl) amino)pentan-2-yl)amino)-2-oxoethyl)-4,9,13,21,25-pentaoxo-J-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,8,11,14,20,23,26-heptaazadotriacontan-32-oate

The title compound was prepared using procedures analogous to those described for Example 1, Step 6, substituting (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-11-(2-(((S)-1,5-dioxo-1,5-bis((2-(((2S,3S,4S,5S,6R)-3,4,5-trihydroxy-6-(hydroxymethyl)tetrahydro-2H-pyran-2-yl)oxy) ethyl)amino)pentan-2-yl)amino)-2-oxoethyl)-4,9,13,21,25-pentaoxo-1-(((2S,3S,4S,5S,6R)-3,4,5-trihydroxy-6-(hydroxymethyl)tetrahydro-2H-pyran-2-yl)oxy)-7-((2-(((2S,3S,4S,5S,6R)-3,4,5-trihydroxy-6-(hydroxymethyl)tetrahydro-2H-pyran-2-yl)oxy)ethyl)carbamoyl)-3,8,11,14,20,23,26-heptaazadotriacontan-32-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino) ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1191.1 (z=2); t_{R}=4.09 min.

### Example 36: 2,5-Dioxopyrrolidin-1-yl (7S,17S)-21-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-17-((4-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-4-oxobutyl)carbamoyl)-4,9,14,19,23-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,13,18,21,24-hexaazatriacontan-30-oate (ML-36)

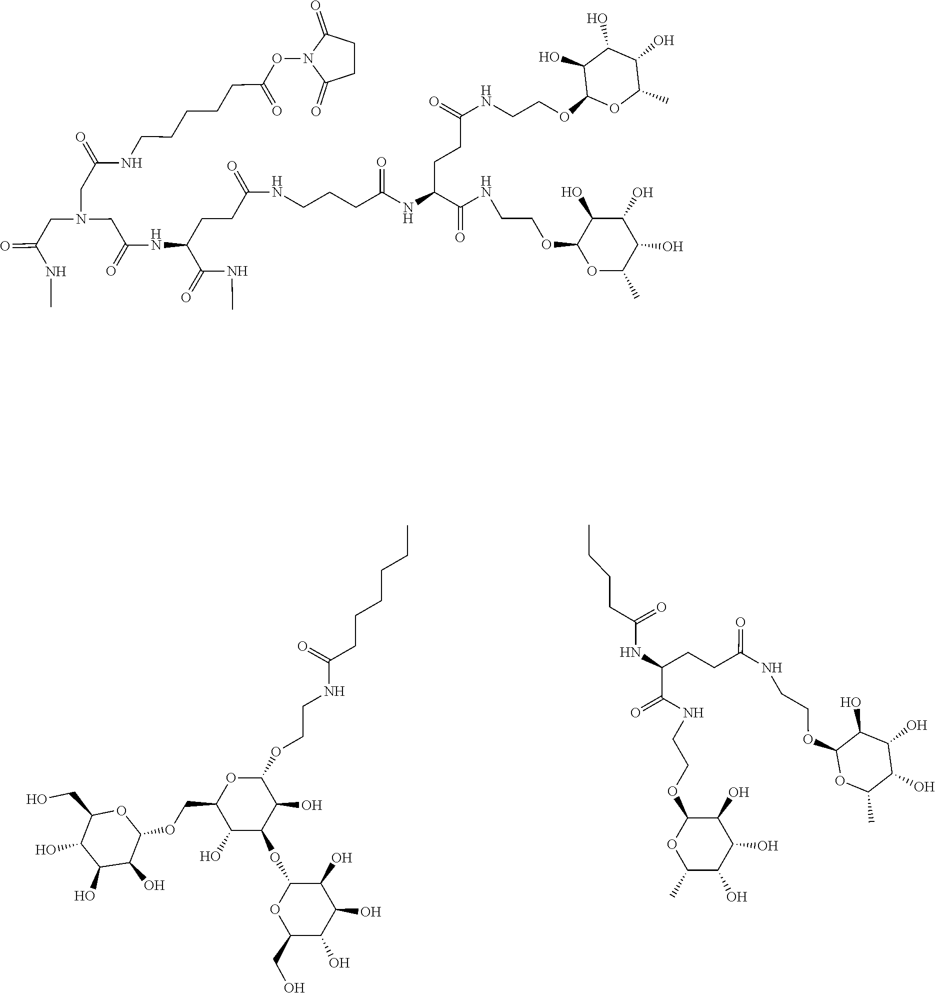 [SMILES: CNC(=O)CN(CC(=O)NCCCCCC(=O)ON1C(=O)CCC1=O)CC(=O)N[C@@H](CCC(=O)NCCCC(=O)N[C@@H](CCC(=O)NCCO[C@@H]1O[C@@H](C)[C@@H](O)[C@@H](O)[C@@H]1O)C(=O)NCCO[C@@H]1O[C@@H](C)[C@@H](O)[C@@H](O)[C@@H]1O)C(=O)NC ; CCCCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O ; CCCCC(=O)N[C@@H](CCC(=O)NCCO[C@@H]1O[C@@H](C)[C@@H](O)[C@@H](O)[C@@H]1O)C(=O)NCCO[C@@H]1O[C@@H](C)[C@@H](O)[C@@H](O)[C@@H]1O]

### Step 1: 2,5-dioxopyrrolidin-1-yl 4-(((benzyloxy)carbonyl)amino)butanoate

A mixture of N—CBz-4-amionobutyric acid (1 g, 4.21 mmol), dipyrrolidino(N-succinimidyloxy)carbenium hexafluorophosphate (2.25 g, 5.48 mmol) and DIPEA (0.96 mL, 5.48 mmol) in DMF (20 mL) was stirred at rt overnight. The mixture was evaporated, and the residue purified by silica gel column chromatography to isolate title compound.

### Step 2: Benzyl (4-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-4-oxobutyl)carbamate

To a solution of (S)-2-amino-N1,N5-bis(2-(((2R,3S,4R,5S,6S)-3,4,5-trihydroxy-6-methyltetrahydro-2H-pyran-2-yl)oxy)ethyl)pentanediamide (450 mg, 0.86 mmol) and 2,5-dioxopyrrolidin-1-yl 4-(((benzyloxy)carbonyl)amino)butanoate (315 mg, 0.94 mmol) in anhydrous DMF (4 mL) was added DIPEA (0.15 mL, 0.86 mmol) and the resulting mixture was stirred at rt for 2 h. The mixture was evaporated, and the residue was purified by C18 reverse phase chromatography to isolate title compound. UPLC-MS Method A: m/z=745.2 (z=1); t_{R}=4.13 min.

### Step 3: (S)-2-(4-Aminobutanamido)-N1,N5-bis(2-(((2R,3S,4R,5S,6S)-3,4,5-trihydroxy-6-methyltetrahydro-2H-pyran-2-yl)oxy)ethyl)pentanediamide

The title compound was prepared using procedures analogous to those described for Example 1, Step 5, substituting benzyl (4-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-4-oxobutyl) carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl) oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=611.2 (z=1); t_{R}=3.43 min.

### Step 4: Benzyl ((7S,15S,25S)-4,9,14,18,23,28-hexaoxo-1,31-bis(((2R,3S,4R,5S,6S)-3,4,5-trihydroxy-6-methyltetrahydro-2H-pyran-2-yl)oxy)-7,25-bis((2-(((2R,3S,4R,5S,6S)-3,4,5-trihydroxy-6-methyltetrahydro-2H-pyran-2-yl)oxy)ethyl)carbamoyl)-3,8,13,19,24,29-hexaazahentriacontan-15-yl)carbamate

To a mixture of ((benzyloxy)carbonyl)-L-glutamic acid (75 mg, 0.267 mmol) and (S)-2-(4-aminobutanamido)-N1,N5-bis(2-(((2R,3S,4R,5S,6S)-3,4,5-trihydroxy-6-methyltetrahydro-2H-pyran-2-yl)oxy)ethyl)pentanediamide (358 g, 0.587 mmol) in DMF (2 mL) was added EDC (204 mg, 1.07 mol), HOBt (4 mg, 0.03 mmol) and TEA (0.003 mL, 0.03 mmol), and the resulting mixture was stirred at rt overnight. The mixture was evaporated, and the residue was purified by C18 reverse phase chromatography to isolate title compound. UPLC-MS Method A: m/z=1446.5 (z=1); t_{R}=3.40 min.

### Step 5: (2S,2′S)-2,2′-((4,4′-(((S)-2-aminopentanedioyl)bis(azanediyl))bis(butanoyl))bis(azanediyl)) bis(N1,N5-bis(2-(((2R,3S,4R,5S,6S)-3,4,5-trihydroxy-6-methyltetrahydro-2H-pyran-2-yl)oxy) ethyl)pentanediamide)

The title compound was prepared using procedures analogous to those described for Example 1, Step 5, substituting benzyl ((7S,15S,25S)-4,9,14,18,23,28-hexaoxo-1,31-bis(((2R,3S,4R,5S,6S)-3,4,5-trihydroxy-6-methyltetrahydro-2H-pyran-2-yl)oxy)-7,25-bis((2-(((2R,3S,4R,5S,6S)-3,4,5-trihydroxy-6-methyltetrahydro-2H-pyran-2-yl)oxy)ethyl)carbamoyl)-3,8,13,19,24,29-hexaazahentriacontan-15-yl)carbamate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino) ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1322.5 (z=1); t_{R}=2.69 min.

### Step 6: Benzyl (7S,17S)-21-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl) oxy)ethyl) amino)-6-oxohexyl)amino)-2-oxoethyl)-17-((4-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl) amino)-4-oxobutyl) carbamoyl)-4,9,14,19,23-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,13,18,21,24-hexaazatriacontan-30-oate

The title compound was prepared using procedures analogous to those described for Example 1, Step 4, substituting (2S,2′S)-2,2′-((4,4′-(((S)-2-aminopentanedioyl)bis(azanediyl)) bis(butanoyl))bis(azanediyl))bis(N1,N5-bis(2-(((2R,3S,4R,5S,6S)-3,4,5-trihydroxy-6-methyltetrahydro-2H-pyran-2-yl)oxy)ethyl)pentanediamide) for 2,2′-(((S)-13-(2-((5-aminopentyl) amino)-2-oxoethyl)-4,11,15,22-tetraoxo-1,25-bis(((2R,3S,4R,5S,6S)-3,4,5-trihydroxy-6-methyltetrahydro-2H-pyran-2-yl)oxy)-3,10,13,16,23-pentaazapentacosan-5-yl)azanediyl)bis(N-(2-(((2R,3S,4R,5S,6S)-3,4,5-trihydroxy-6-methyltetrahydro-2H-pyran-2-yl)oxy)ethyl)acetamide) to isolate the title compound. UPLC-MS Method A: m/z=1176.9 (z=2); t_{R}=²0.55 min.

### Step 7: (7S,17S)-21-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-17-((4-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl) amino)-4-oxobutyl) carbamoyl)-4,9,14,19,23-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,13,18,21,24-hexaazatriacontan-30-oic acid

The title compound was prepared using procedures analogous to those described for Example 1, Step 5, substituting benzyl (7S,17S)-21-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-17-((4-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-4-oxobutyl)carbamoyl)-4,9,14,19,23-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,13,18,21,24-hexaazatriacontan-30-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate to isolate title compound. UPLC-MS Method A: m/z=1131.3 (z=2); t_{R}=3.53 min.

### Step 8: 2,5-Dioxopyrrolidin-1-yl (7S,17S)-21-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-17-((4-(((S)-1,5-dioxo-1,5-bis((2-(((2R,3S,4R,5S,6S)-3,4,5-trihydroxy-6-methyltetrahydro-2H-pyran-2-yl)oxy) ethyl)amino)pentan-2-yl)amino)-4-oxobutyl)carbamoyl)-4,9,14,19,23-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,13,18,21,24-hexaazatriacontan-30-oate

The title compound was prepared using procedures analogous to those described for Example 1, Step 6, substituting (7S,17S)-21-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-17-((4-(((S)-1,5-dioxo-1,5-bis((2-(((2R,3S,4R,5S,6S)-3,4,5-trihydroxy-6-methyltetrahydro-2H-pyran-2-yl)oxy) ethyl) amino)pentan-2-yl)amino)-4-oxobutyl)carbamoyl)-4,9,14,19,23-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,13,18,21,24-hexaazatriacontan-30-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl) amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1180.1 (z=2); t_{R}=4.06 min.

### Example 37: 2,5-Dioxopyrrolidin-1-yl (S)-23-(2-((6-((2-(((2R,3R,4R,5S,6S)-3,5-dihydroxy-4-(((2S,3R,4R,5R,6S)-3,4,5-trihydroxy-6-(hydroxymethyl)tetrahydro-2H-pyran-2-yl)oxy)-6-((((2R,3R,4R,5R,6S)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)amino)-2-oxoethyl)-11-(2-(((S)-1,5-dioxo-1,5-bis((2-(((2S,3R,4S,5R,6R)-3,4,5-trihydroxy-6-methyltetrahydro-2H-pyran-2-yl)oxy)ethyl)amino)pentan-2-yl)amino)-2-oxoethyl)-4,9,13,21,25-pentaoxo-1-(((2S,3R,4S,5R,6R)-3,4,5-trihydroxy-6-methyltetrahydro-2H-pyran-2-yl)oxy)-7-((2-(((2S,3R,4S,5R,6R)-3,4,5-trihydroxy-6-methyltetrahydro-2H-pyran-2-yl)oxy)ethyl)carbamoyl)-3,8,11,14,20,23,26-heptaazanonacosan-29-oate (ML-37)

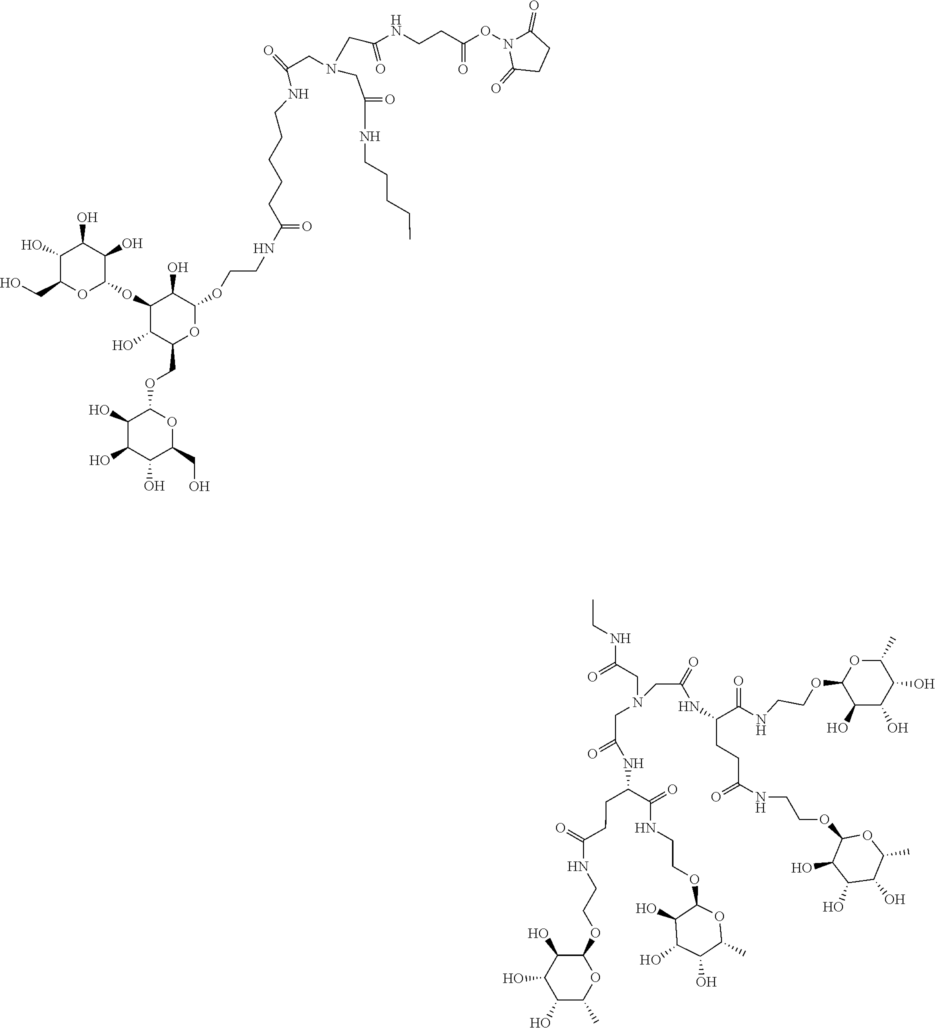 [SMILES: CCCCCNC(=O)CN(CC(=O)NCCCCCC(=O)NCCO[C@@H]1O[C@@H](CO[C@@H]2O[C@@H](CO)[C@H](O)[C@@H](O)[C@H]2O)[C@H](O)[C@@H](O[C@@H]2O[C@@H](CO)[C@H](O)[C@@H](O)[C@H]2O)[C@H]1O)CC(=O)NCCC(=O)ON1C(=O)CCC1=O ; CCNC(=O)CN(CC(=O)N[C@@H](CCC(=O)NCCO[C@H]1O[C@H](C)[C@H](O)[C@H](O)[C@H]1O)C(=O)NCCO[C@H]1O[C@H](C)[C@H](O)[C@H](O)[C@H]1O)CC(=O)N[C@@H](CCC(=O)NCCO[C@H]1O[C@H](C)[C@H](O)[C@H](O)[C@H]1O)C(=O)NCCO[C@H]1O[C@H](C)[C@H](O)[C@H](O)[C@H]1O]

### Step 1: N-(2-((3-(Benzyloxy)-3-oxopropyl)amino)-2-oxoethyl)-N-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)glycine

To a suspension of 2,2′-((2-((3-(benzyloxy)-3-oxopropyl)amino)-2-oxoethyl)azanediyl) diacetic acid (WO2016164288A1) (410 mg, 1.16 mmol) in anhydrous DCM (4 mL) cooled in an ice-bath was added trifluoroacetic anhydride (0.205 mL, 1.46 mol) and mixture stirred at ice-bath temperature for 3 h. The mixture was cooled to −30° C., and a solution of TEA (0.39 mL, 2.79 mol) in anhydrous DMF (2 mL) was added dropwise over 30 min. Stirring at −30° C. was continued for 30 min, then a mixture of 6-amino-N-(2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)hexanamide (769 mg, 1.16 mmol) in DMF (4 mL) was added. The resulting mixture was stirred at rt overnight. The mixture was evaporated, and the residue was purified by normal phase chromatography to isolate title compound. UPLC-MS Method A: m/z=996.0 (z=2); t_{R}=2.78 min.

### Step 2: Benzyl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl) amino)-2-oxoethyl)-4,9,13,21,25-pentaoxo-1-(((2R,3S,4R,5S,66S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,8,11,14,20,23,26-heptaazanonacosan-29-oate

To a solution of N-(2-((3-(benzyloxy)-3-oxopropyl)amino)-2-oxoethyl)-N-(2-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl) amino)-6-oxohexyl) amino)-2-oxoethyl)glycine (55 mg 0.055 mmol) and (2S,2′S)-2,2′-((2,2′-((2-((5-aminopentyl) amino)-2-oxoethyl)azanediyl)bis(acetyl))bis(azanediyl))bis(N1,N5-bis(2-(((2R,3S,4R,5S,6S)-3,4,5-trihydroxy-6-methyltetrahydro-2H-pyran-2-yl)oxy)ethyl)pentanediamide) (86 mg, 0.066 mmol) in anhydrous DMF (1 mL) was added EDC (21 mg, 0.11 mmol) and DMAP (7 mg, 0.055 mmol), and the mixture was stirred at rt overnight. The mixture was evaporated, and the residue was purified by C18 reverse phase chromatography to isolate title compound. UPLC-MS Method A: m/z=1134.4 (z=2); t_{R}=3.53 min.

### Step 3: (S)-23-(2-((6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl) oxy)ethyl)amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl)amino)pentan-2-yl)amino)-2-oxoethyl)-4,9,13,21,25-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,11,14,20,23,26-heptaazanonacosan-29-oic acid

The title compound was prepared using procedures analogous to those described for Example 1, Step 5, substituting benzyl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl) amino)pentan-2-yl)amino)-2-oxoethyl)-4,9,13,21,25-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,11,14,20,23,26-heptaazanonacosan-29-oate for benzyl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy) ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl) amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl) carbamoyl)-3,6,12,15,18,24,27,30-octaazahexatriacontan-36-oate to isolate title compound. UPLC-MS Method A: m/z=1089.4 (z=2); t_{R}=3.39 min.

### Step 4: 2,5-Dioxopyrrolidin-1-yl (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl)amino) pentan-2-yl)amino)-2-oxoethyl)-4,9,13,21,25-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,11,14,20,23,26-heptaazanonacosan-29-oate

The title compound was prepared using procedures analogous to those described for Example 1, Step 6, substituting (S)-23-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-2-oxoethyl)-11-(2-(((S)-1,5-dioxo-1,5-bis((2-(((2R,3S,4R,5S,6S)-3,4,5-trihydroxy-6-methyltetrahydro-2H-pyran-2-yl)oxy)ethyl) amino)pentan-2-yl)amino)-2-oxoethyl)-4,9,13,21,25-pentaoxo-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,8,11,14,20,23,26-heptaazanonacosan-29-oic acid for (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl) amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oic acid to isolate title compound. UPLC-MS Method A: m/z=1137.9 (z=2); t_{R}=2.88 min.

### Example 38: 2,5-Dioxopyrrolidin-1-yl (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-17-(4-(6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanamido)butyl)-4,11,15,18-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,19-pentaazapentacosan-25-oate (ML-38)

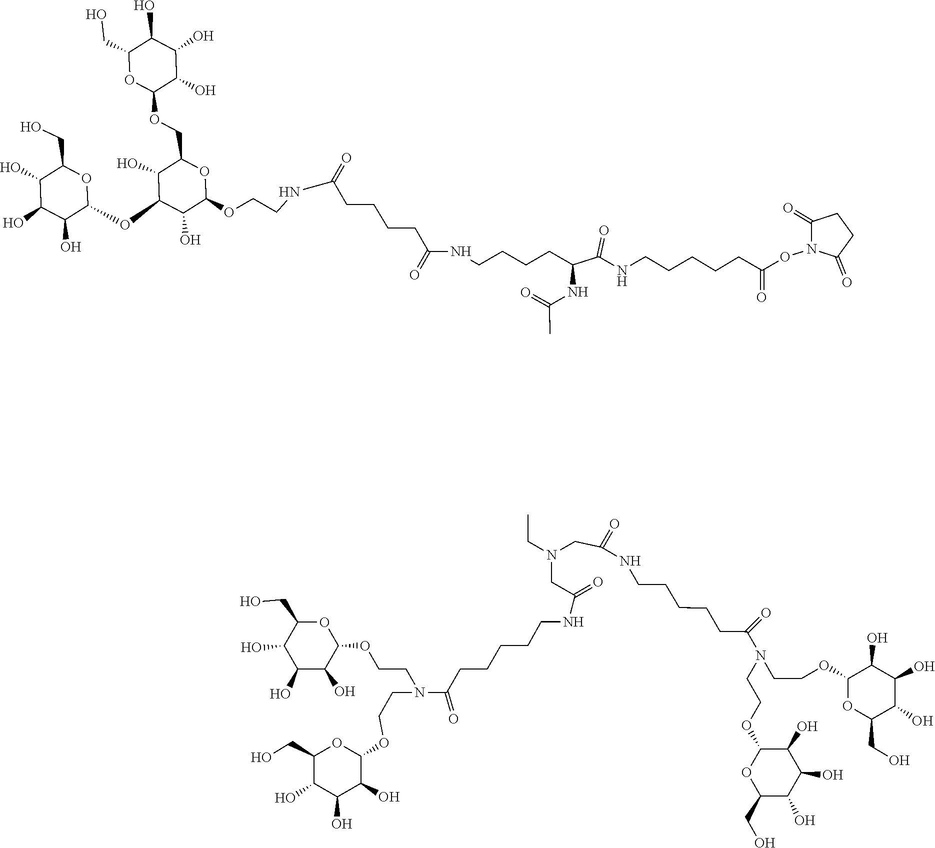 [SMILES: CC(=O)N[C@@H](CCCCNC(=O)CCCCC(=O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@H]1O)C(=O)NCCCCCC(=O)ON1C(=O)CCC1=O ; CCN(CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O]

### Step 1: Benzyl 6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanoate

A mixture of 2-aminoethyl O-α-D-mannopyranosyl-(1→3)-O-[α-D -mannopyranosyl-(1→6)]-β-D-glucopyranoside (WO2021021535) (756 mg, 1.38 mmol), adipic acid monobenzyl ester (424 mg, 1.795 mmol), HOBT (423 mg, 2.76 mmol), Hunig's base (723 μL, 4.14 mmol), and EDC (397 mg, 2.071 mmol) in DMF (6.90 mL) was stirred overnight. The mixture was concentrated and directly chromatographed on ISCO C18 column (130 g), flow 85 mL/min, water-ACN-0.05% TFA, grad 0-30% in 30 min, and the title compound was isolated by lyophilization of fractions. UPLC-MS Method A: m/z=766.44 (z=1); t_{R}=2.58 min.

### Step 2: 6-((2-(((2R,3R,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanoic acid

A solution of benzyl 6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoate (640 mg, 0.836 mmol) was hydrogenated and dissolved in water (8.3 mL) over Pearlman's catalyst (88 mg, 0.125 mmol) at 50 psi over 3 hours. The solvent was removed by lyophilization to furnish the title compound. UPLC-MS Method A: m/z=676.2 (z=1); t_{R}=1.11 min.

### Step 3: 2,5-Dioxopyrrolidin-1-yl 6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoate

6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanoic acid (533 mg, 0.774 mmol) was dissolved in DMF (7.74 mL) and cooled to 0° C. TSTU (280 mg, 0.929 mmol) was added, and the mixture was stirred for 5 min to dissolve. TEA (129 μL, 0.929 mmol) was added, and the mixture was stirred for 30 min. The reaction mixture was poured into 10× volumes of acetone, and the precipitate was collected by centrifugation. The precipitate was re-suspended in 10 mL of acetone, and centrifugation was repeated. The resulting pellet was pumped overnight to obtain the title material. UPLC-MS Method A: m/z=773.2 (z=1); t_{R}=1.37 min.

### Step 4: Benzyl (S)-6-(2-amino-6-(((benzyloxy)carbonyl) amino)hexanamido)hexanoate (TFA salt)

A mixture of (S)-6-(((benzyloxy)carbonyl)amino)-2-((tert-butoxycarbonyl)amino) hexanoic acid (1.0 g, 2.63 mmol), 6-amino-hexanoic acid benzyl ester, compound with toluene-4-sulfonic acid (1.345 g, 3.42 mmol), HOBT (604 mg, 3.94 mmol), Hünig's base (1.83 mL, 10.51 mmol), EDC (756 mg, 3.94 mmol) was stirred overnight, using DMF (13 mL) as the solvent. The reaction mixture was diluted with 100 mL of 1:1 mixture of EtOAc/Hex. The mixture was washed with 100 mL of 1M HCl, 100 mL of sat NaHCO₃, and 100 mL of brine. The organic phase was dried over Na₂SO₄ and concentrated. The product was re-dissolved in 10 mL of DCM, and 10 mL of TFA was added. The mixture was stirred for 3 h. allowing gasses to escape the flask to avoid pressure build-up. The mixture was then concentrated and pumped on high vacuum overnight to furnish the title material. UPLC-MS Method A: m/z=484.36 (z=1); t_{R}=4.00 min.

### Step 5: Benzyl (S)-17-(4-(((Benzyloxy)carbonyl)amino)butyl)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl) amino)-2-oxoethyl)-4,11,15,18-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,19-pentaazapentacosan-25-oate

To a solution of benzyl (S)-6-(2-amino-6-(((benzyloxy)carbonyl)amino)hexanamido) hexanoate (TFA salt) (1.12 g, 1.926 mmol) in DMF (19.26 mL) was added 2-(2,6-dioxomorpholino) acetic acid (0.333 g, 1.926 mmol), and the mixture was stirred for 5 min to dissolve. TEA (0.805 mL, 5.78 mmol) was added slowly, and the mixture was stirred for 3 h. 6-amino-N,N-bis[2-(α-D-mannopyranosyloxy)ethyl]hexanamide (WO2020247297) (2.090 g, 3.85 mmol), HOBT (0.885 g, 5.78 mmol), Hunig's base (1.009 mL, 5.78 mmol), and EDC (0.923 g, 4.81 mmol) were added, and the mixture was stirred overnight. The reaction mixture was concentrated and purified by reverse-phase HPLC on ISCO 100 g C18 column, 80 mL/min flow, ACN-water-0.05% TFA, grad 0-40% in 30 min followed by hold. The product was re-purified by reverse-phase chromatography on C-8 phase (Column Kromasil, C8 10 μM 100 A, size 250×50 mm; solvent A=water/0.05% TFA, solvent B=ACN/0.05% TFA), Flow=85 mL/min, gradient B in A 20-70% in 30 min. UPLC-MS Method B: m/z=1706.98 (z=1); t_{R}=3.33 min.

### Step 6: (S)-17-(4-Aminobutyl)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-4,11,15,18-tetraoxo-1-(((2S,3S,4,5S,6R)-3,4,5-trihydroxy-6-(hydroxymethyl)tetrahydro-2H-pyran-2-yl)oxy)-3-(2-(((2S,3S,4S,S,6R)-3,4,5-trihydroxy-6-(hydroxymethyl)tetrahydro-2H-pyran-2-yl)oxy)ethyl)-3,10,13,16,19-pentaazapentacosan-25-oic acid

Benzyl (S)-17-(4-(((benzyloxy)carbonyl)amino)butyl)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-4,11,15,18-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,19-pentaazapentacosan-25-oate (420 mg, 0.246 mmol) was hydrogenated over Pearlman's catalyst (25.9 mg, 0.037 mmol) in water (8.21 mL) at 50 psi over a period of 4 h. The catalyst was removed by filtration, and the water was removed by lyophilization to obtain the title material. UPLC-MS Method B: m/z=1428.49 (z=1); t_{R}=1.58 min.

### Step 7: (R)-13-(2-((6-(Bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-17-(4-(6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexanamido)butyl)-4,11,15,18-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,19-pentaazapentacosan-25-oic acid

To a solution of (S)-17-(4-aminobutyl)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-4,11,15,18-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,13,16,19-pentaazapentacosan-25-oic acid (70 mg, 0.047 mmol) in DMF (472 μL) was added triethylamine (132 μL, 0.945 mmol) and 2,5-dioxopyrrolidin-1-yl 6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl) oxy)ethyl)amino)-6-oxohexanoate (36.5 mg, 0.047 mmol). The reaction mixture was stirred overnight. The reaction mixture was poured into 10× volumes of acetone, and the product was collected by centrifugation. The product was re-suspended in 10 mL of acetone, and centrifugation was repeated. The product was dissolved in 2 mL of water and 0.2 mL of ACN and lyophilized to obtain the title material. UPLC-MS Method B: m/z=1070.09 (z=2); t_{R}=1.60 min.

### Step 8: 2,5-Dioxopyrrolidin-1-yl (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-17-(4-(6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanamido)butyl)-4,11,15,18-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,19-pentaazapentacosan-25-oate

To a solution of (R)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-17-(4-(6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanamido)butyl)-4,11,15,18-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,19-pentaazapentacosan-25-oic acid (80 mg, 0.037 mmol) and TSTU (13.51 mg, 0.045 mmol)) in DMF (374 μL)) was added TEA (7.82 μL, 0.056 mmol). After stirring for 1 h, the mixture was poured into 20 mL of 1:1 ether-acetone mixture, and the precipitate was collected by centrifugation. The precipitate was re-suspended in 10 mL of 1:1 ether-acetone, and centrifugation was repeated. The pellet was placed under high vacuum for 3 h to obtain the title product. UPLC-MS Method B: m/z=1119.15 (z=2); t_{R}=0.87 min.

### Example 39: 2,5-Dioxopyrrolidin-1-yl (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-21-(6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanamido)-4,11,15,22-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,23-pentaazanoinacosan-29-oate (ML-39)

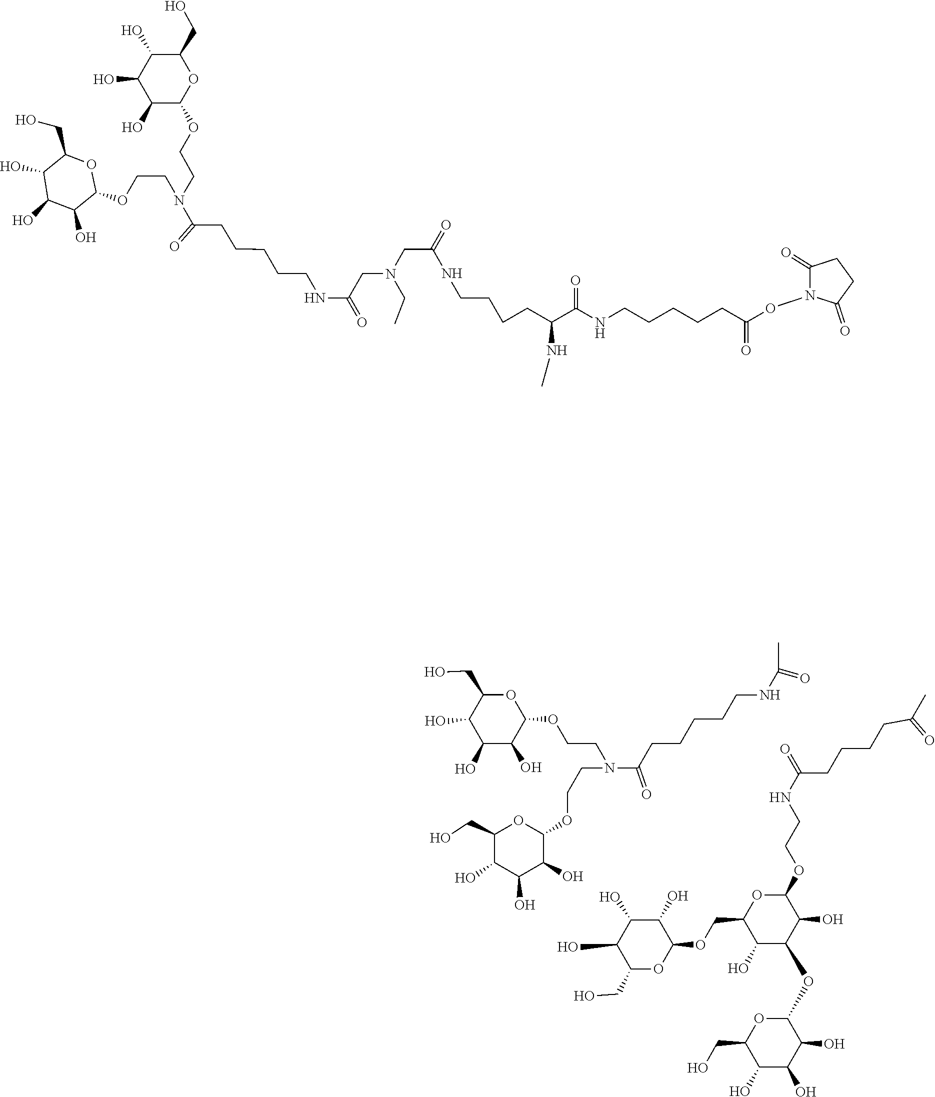 [SMILES: CCN(CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)NCCCC[C@H](NC)C(=O)NCCCCCC(=O)ON1C(=O)CCC1=O ; CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O ; CC(=O)CCCCC(=O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl 6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanoate

A mixture of 2-aminoethyl O-α-D-mannopyranosyl-(1→3)-O-[α-D-mannopyranosyl-(1→6)]-β-D-mannopyranoside (WO 2021021535) (200 mg, 0.365 mmol), adipic acid monobenzyl ester (112 mg, 0.475 mmol), HOBT (112 mg, 0.731 mmol), Hünig's base (191 μL, 1.096 mmol), and EDC (105 mg, 0.548 mmol)) was stirred, using DMF (3.65 mL) as the solvent. The mixture was concentrated and purified on 13 g ISCO C18 column using ACN-water-0.05% TFA, gradient 0-30% in 30 min, flow 13 mL/min to furnish the title material. UPLC-MS Method B: m/z=766.29 (z=1); t_{R}=2.57 min.

### Step 2: 6-((2-(((2R,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanoic acid

A solution of benzyl 6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoate (150 mg, 0.196 mmol) in water (6.53 mL) was hydrogenated over Pearlman's catalyst (27.5 mg, 0.039 mmol) at 50 psi over 3 h. The catalyst was removed by filtration, and the solvent was removed by lyophilization to furnish the title material. UPLC-MS Method B: m/z=676.27 (z=2); t_{R}=1.12 min.

### Step 3: 2,5-Dioxopyrrolidin-1-yl 6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoate 6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanoic acid (125 mg, 0.185 mmol) was dissolved in DMF (1.85 mL) and cooled to 0° C. TSTU (61.3 mg, 0.204 mmol) was added, and the mixture was stirred for 5 min to dissolve. TEA (33.5 μL, 0.241 mmol) was added, and the mixture was stirred for 30 min before being poured into 10× volumes of acetone. The precipitate was collected by centrifugation. The precipitate was re-suspended in 10 mL of 1:1 ether-acetone, and centrifugation was repeated. The pellet was placed under high vacuum overnight to obtain the title product. UPLC-MS Method B: m/z=773.41 (z=1); t_{R}=1.37 min.

### Step 4: Benzyl (S)-6-(6-amino-2-(((benzyloxy)carbonyl)amino)hexanamido)hexanoate (TFA salt)

A mixture of Z-LYS(BOC)—OH (1.0 g, 2.63 mmol), 6-amino-hexanoic acid benzyl ester, compound with toluene-4-sulfonic acid (1.345 g, 3.42 mmol), HOBT (604 mg, 3.94 mmol), Hünig's base (1.84 mL, 10.51 mmol), and EDC (756 mg, 3.94 mmol) were stirred overnight, using DMF (21 mL) as the solvent. The reaction mixture was diluted with 100 mL of 1:1 mixture of EtOAc/Hex. The mixture was washed with 100 mL of 1M HCl, 100 mL of sat NaHCO₃, and 100 mL of brine. The organic phase was dried over Na₂SO₄ and concentrated. The product was re-dissolved in 10 mL of DCM, and 10 mL of TFA was added. The mixture was stirred, allowing gasses to escape the flask to avoid pressure build-up. After 3 h, the mixture was concentrated and pumped on high vacuum overnight to furnish the title compound. UPLC-MS Method B: m/z=484.35 (z=1); t_{R}=3.89 min.

### Step 5: (S)-11-(((Benzyloxy)carbonyl)amino)-19-(carboxymethyl)-3,10,17-trioxo-1-phenyl-2-oxa-9,16,19-triazahenicosan-21-oic acid

To a solution of benzyl (S)-6-(6-amino-2-(((benzyloxy)carbonyl)amino)hexanamido) hexanoate (TFA salt) (1.2 g, 2.008 mmol) in DMF (10.04 mL) was added 2-(2,6-dioxomorpholino) acetic acid (0.348 g, 2.008 mmol), and the mixture was stirred for 5 min to dissolve. Then, Hünig's base (0.175 mL, 1.004 mmol) was added slowly (5 min), and the reaction mixture was stirred overnight. The mixture was concentrated on a rotary evaporator and purified on ISCO C18 column, solvent A=water—0.05% TFA, solvent B=ACN-0.05% TFA, gradient 0-55% in 40 min to obtain the title material. UPLC-MS Method B: m/z=657.42 (z=1); t_{R}=3.19 min.

### Step 6: Benzyl (S)-21-(((benzyloxy)carbonyl)amino)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-4,11,15,22-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,13,16,23-pentaazanonacosan-29-oate

A mixture of(S)-11-(((benzyloxy)carbonyl)amino)-19-(carboxymethyl)-3,10,17-trioxo-1-phenyl-2-oxa-9,16,19-triazahenicosan-21-oic acid (583 mg, 0.888 mmol), 6-amino-N,N-bis[2-(α-D-mannopyranosyloxy)ethyl]hexanamide (WO 2020247297A1) (1445 mg, 2.66 mmol). HOBt (408 mg, 2.66 mmol), EDC (511 mg, 2.66 mmol), Hünig's base (0.93 mL, 5.33 mmol) was stirred for 48 h. The mixture was concentrated and purified on a 100 g C18 ISCO column, flow 60 mL/min, ACN-Water-0.05% TFA, grad 0-40% over 30 min to obtain the title material. UPLC-MS Method B: m/z=1707.02 (z=1); t_{R}=3.30 min.

### Step 7: (R)-21-Amino-13-(2-((6-(bis(2-(((2S,3S,4S,5S,6R)-3,4,5-trihydroxy-6-(hydroxymethyl) tetrahydro-2H-pyran-2-yl)oxy)ethyl)amino)-6-oxohexyl)amino)-2-oxoethyl)-4,11,15,22-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oic acid

A solution of benzyl (S)-21-(((benzyloxy)carbonyl)amino)-13-(2-((6-(bis(2-(((2S,3S,4S,5S,6R)-3,4,5-trihydroxy-6-(hydroxymethyl)tetrahydro-2H-pyran-2-yl)oxy)ethyl) amino)-6-oxohexyl)amino)-2-oxoethyl)-4,11,15,22-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oate (1.241 g, 0.728 mmol) in water (14.55 mL) was hydrogenated over Pearlman's catalyst (0.077 g, 0.109 mmol) at 50 psi over 6 h. The catalyst was removed by filtration, and the solvent was removed by lyophilization to obtain the title material. UPLC-MS Method B: m/z=1482.94 (z=1); t_{R}=1.62 min.

### Step 8: (R)-21-Amino-13-(2-((6-(bis(2-(((2S,3S,4S,5S,6R)-3,4,5-trihydroxy-6-(hydroxymethyl) tetrahydro-2H-pyran-2-yl)oxy)ethyl)amino)-6-oxohexyl)amino)-2-oxoethyl)-4,11,15,22-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-v oxy)ethyl)-3,10,13,16,23-pentaazanonacosan-29-oic acid

A mixture of 2,5-dioxopyrrolidin-1-yl 6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexanoate (52.2 mg, 0.067 mmol), (R)-21-amino-13-(2-((6-(bis(2-(((2S,3S,4S,5S,6R)-3,4,5-trihydroxy-6-(hydroxymethyl)tetrahydro-2H-pyran-2-yl)oxy)ethyl) amino)-6-oxohexyl)amino)-2-oxoethyl)-4,11,15,22-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oic acid (100 mg, 0.067 mmol), TEA (188 μL, 1.350 mmol) was stirred, using DMF (675 L)) as the solvent, overnight. The reaction appeared to be complete on UPLC, and the reaction was quenched with ethanolamine (4.08 μL, 0.067 mmol) and stirred for an additional 1 h. The reaction mixture was concentrated and purified on 13 g ISCO C18 column, ACN-water-0.05% TFA, grad 0-30% in 30 min to obtain the title material after lyophilization. UPLC-MS Method B: m/z=1070.63 (z=1); t_{R}=1.59 min.

### Step 9: 2,5-Dioxopyrrolidin-1-yl (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-21-(6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)-4,11,15,22-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,13,16,23-pentaazanonacosan-29-oate

To a solution of (R)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-21-(6-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanamido)-4,11,15,22-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oic acid (97 mg, 0.045 mmol) and TSTU (16.38 mg, 0.054 mmol) in DMF (453 μL) was added TEA (9.48 μL, 0.068 mmol). The reaction mixture was stirred for 1 h, then poured into 20 mL of 1:1 ether-acetone mix. The precipitate was collected by centrifugation. The precipitate was re-suspended in 10 mL of 1:1 ether-acetone, and centrifugation was repeated. The pellet was placed under high vacuum overnight to obtain the title product. UPLC-MS Method B: m/z=1118.66 (z=2); t_{R}=1.74 min.

### Example 40: 2,5-Dioxopyrrolidin-1-yl (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-21-(6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)-4,11,15,22-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oate (ML-40)

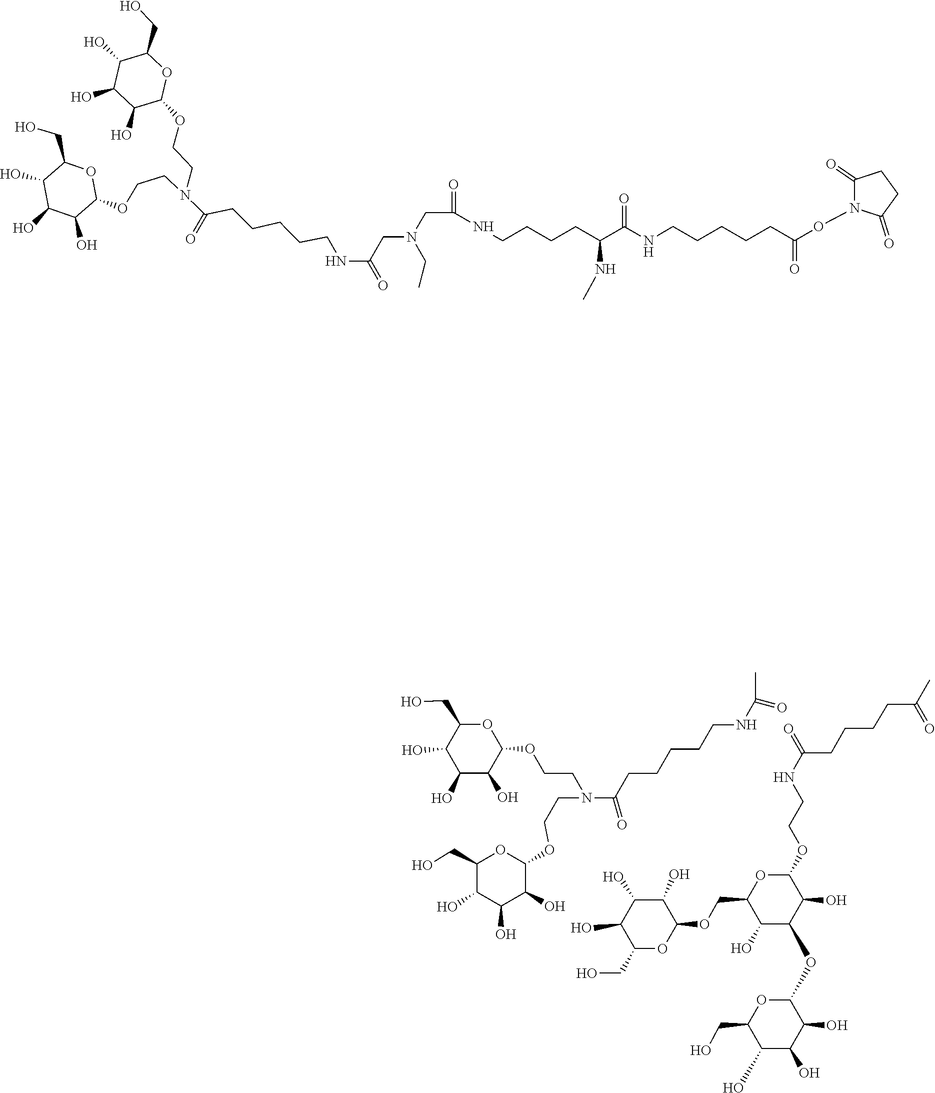

The title compound was synthesized analogously to Example 39, except that 2-aminoethyl O-α-D-mannopyranosyl-(1→3)-O-[α-D-mannopyranosyl-(1→6)]-α-D-mannopyranoside (WO2018175272A1) was used as the starting material in Step 1. UPLC-MS Method B: m/z=1119.18 (z=²); t_{R}=1.74 min.

### Example 41: 2,5-Dioxopyrrolidin-1-yl (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-21-(6-((2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)-4,11,15,22-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oate (ML-41)

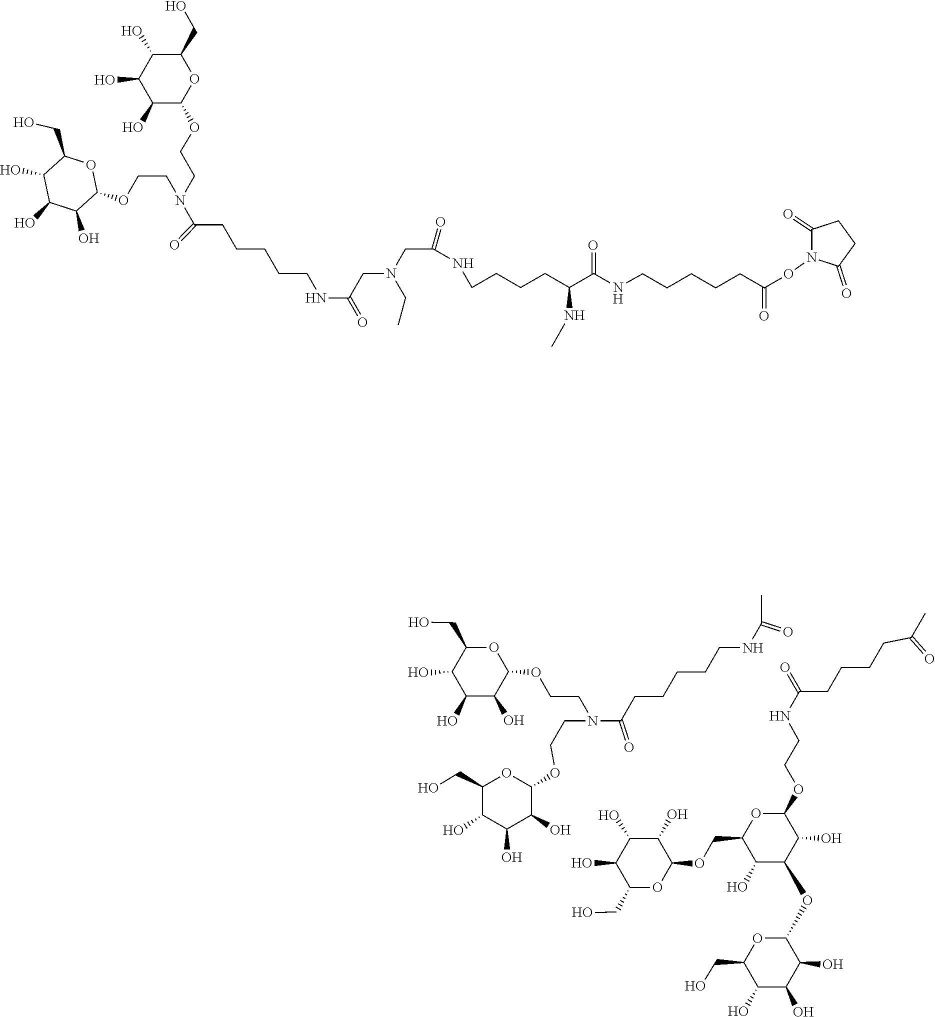

The title compound was synthesized analogously to Example 39, except that 2-aminoethyl O-α-D-mannopyranosyl-(1→3)-O-[α-D-mannopyranosyl-(1→6)]-β-D-glucopyranoside (WO2021021535) was used as the starting material in Step 1. UPLC-MS Method B: m/z=1119.16 (z=2); t_{R}=1.73 min.

### Example 42: 2,5-Dioxopyrrolidin-1-yl ('S)-13-(2-((6-(bis(2-(((2S,3S,4S,5S,6R)-3,4,5-trihydroxy-17-(4-(6-((2-(((2S,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-amino)-6-oxohexanamido)butyl)-4,11,15,18-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,19-pentaazapentacosan-25-oate (ML-42)

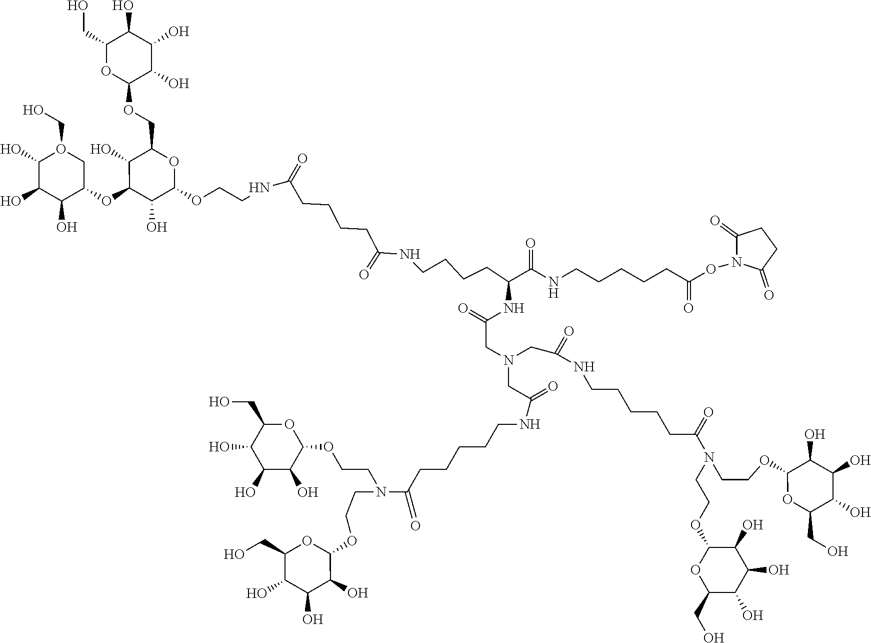

The title compound was synthesized analogously to Example 38, except that 2-Aminoethyl O-α-D-mannopyranosyl-(1→3)-O-[α-D-mannopyranosyl-(1→6)]-α-D-mannopyranoside (WO2021021535) was used as the starting material in Step 1. UPLC-MS Method B: m/z=1119.16 (z=2); t_{R}=0.87 min.

### Example 43: 2,5-Dioxopyrrolidin-1-yl (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-25-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)carbamoyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,26-hexaazadotriacontan-32-oate (ML-43)

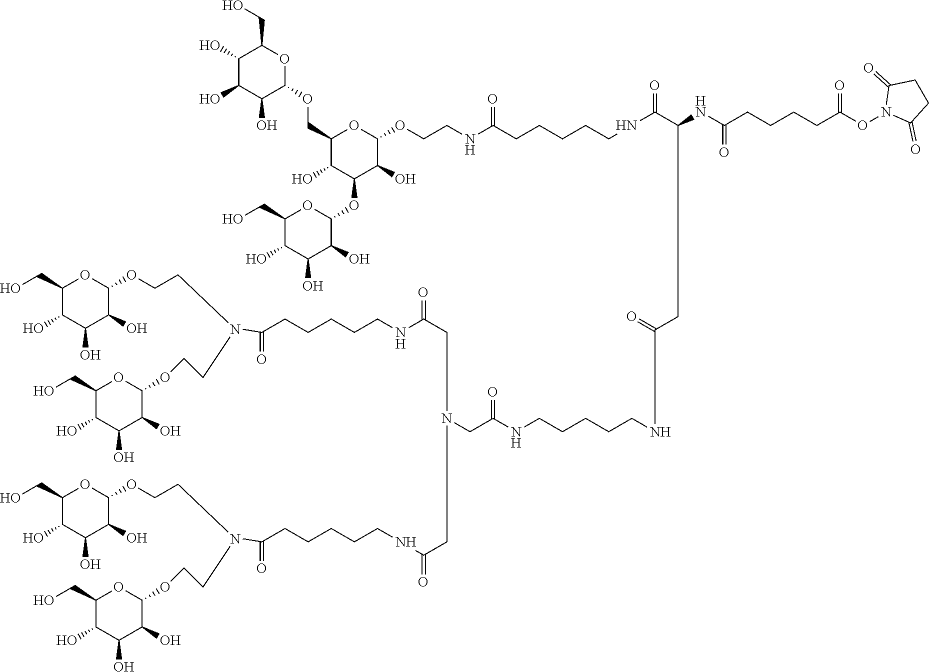 [SMILES: O=C(CCCCCNC(=O)[C@H](CC(=O)NCCCCCNC(=O)CN(CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)NC(=O)CCCCC(=O)ON1C(=O)CCC1=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: 6-(2-Phenoxyacetamido)-N-(2-(((2S,3S,4S,5R,6R)-3,4,5-tris(2-oxopropoxy)-6-((2-oxopropoxy)methyl)tetrahydro-2H-pyran-2-yl)oxy)ethyl)-N-(2-(((3S,4S,5R,6R)-3,4,5-tris(2-oxopropoxy)-6-((2-oxopropoxy)methyl)tetrahydro-2H-pyran-2-yl)oxy)ethyl)hexanamide

To the solution of bis{2-[(2,3,4,6-tetra-O-acetyl-α-D-mannopyranosyl)oxy]ethyl}amine (WO2020247297A1) (2.0 g, 2.61 mmol) in DMF (3 mL) at 0° C. was added 2,5-dioxopyrrolidin-1-yl 6-(((benzyloxy)carbonyl)amino)hexanoate (2.366 g, 6.53 mmol), followed by Hünig's Base (0.547 mL, 3.13 mmol). The mixture was gradually warmed to rt and stirred at rt for 24 h. The mixture was concentrated, and the residue was purified by flash chromatography on C18 reverse phase silica gel (120 g), eluting with 0-40% ACN in H₂O, to give the title compound. UPLC-MS Method B: m/z=1013.4 (z=2); t_{R}=4.28 min.

### Step 2: Benzyl (6-(bis(2-(((2S,3S,4S,5S,6R)-3,4,5-trihydroxy-6-(hydroxymethyl)tetrahydro-2H pyran-2-yl)oxy)ethyl)amino)-6-oxohexyl)carbamate

To the solution of 6-(2-phenoxyacetamido)-N-(2-(((2S,3S,4S,5R,6R)-3,4,5-tris(2-oxopropoxy)-6-((2-oxopropoxy)methyl)tetrahydro-2H-pyran-2-yl)oxy)ethyl)-N-(2-(((3S,4S,5R,6R)-3,4,5-tris(2-oxopropoxy)-6-((2-oxopropoxy)methyl)tetrahydro-2H-pyran-2-yl)oxy)ethyl) hexanamide (2.0 g, 1.974 mmol) in MeOH (10 mL), was added NaOMe 0.5M in MeOH (0.395 L, 0.197 mmol). The mixture was stirred at rt for 16 h. Pre-washed DOWEX™ 50WX-200(H) ion-exchange resin (5.7 g) was added to the reaction mixture. After gentle shaking, the resin was removed by filtration. The filtrate was concentrated, and the residue was purified by flash chromatography on C18 reverse phase silica gel (120 g), eluting with 0-15% ACN in H₂O, to give the title compound. UPLC-MS Method B: m/z=677.34 (z=1); t_{R}=2.11 min.

### Step 3: 6-Amino-N,N-bis(2-(((2S,3S,4S,5S,6R)-3,4,5-trihydroxy-6-(hydroxymethyl)tetrahydro 2H-pyran-2-yl)oxy)ethyl)hexanamide

To benzyl (6-(bis(2-(((2S,3S,4S,5S,6R)-3,4,5-trihydroxy-6-(hydroxymethyl) tetrahydro-2H pyran-2-yl)oxy)ethyl)amino)-6-oxohexyl)carbamate (1.0 g, 1.478 mmol) in water (10 mL) was added Pearlman's catalyst (79 mg, 0.074 mmol), and the reaction mixture was placed under H_{2(g)} and stirred at rt. After 16 h, the catalyst was removed by filtration, and the filtrate was lyophilized to provide the title compound. UPLC-MS Method B: m/z=543.29 (z=1); t_{R}=1.37 min.

### Step 4: Benzyl (13-(2-((6-(bis(2-(((2S,3S,4S,5S,6R)-3,4,5-trihydroxy-6-hydroxymethyl) tetrahydro-2H-pyran-2-yl)oxy)ethyl)amino)-6-oxohexyl)amino)-2-oxoethyl)-4,11,15-trioxo-1-(((2S,3S,4S,5S,6R)-3,4,5-trihydroxy-6-(hydroxymethyl)tetrahydro-2H-pyran-2yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16-tetraazahenicosan-21-yl)carbamate

To the solution of 13-(carboxymethyl)-3,11-dioxo-1-phenyl-2-oxa-4,10,13-triazapentadecan-15-oic acid (300 mg, 0.733 mmol) in DMF (3.0 mL) was added 6-amino-N,N-bis(2-(((2S,3S,4S,5S,6R)-3,4,5-trihydroxy-6-(hydroxymethyl)tetrahydro-2H-pyran-2-yl)oxy) ethyl)hexanamide (795 mg, 1.465 mmol), followed by H₂O (1.0 mL), then HOBT (281 mg, 1.832 mmol), and EDC (351 mg, 1.832 mmol) were added. The resulting mixture was stirred at rt overnight. The mixture was then purified by BIOTAGE SNAP ON 120 g C18 column, eluted with 0-20% ACN in water. The fractions were combined and lyophilized to yield the title compound. UPLC-MS Method B: m/z=1458.80 (z=1); t_{R}=2.64 min.

### Step 5: 6,6′-((2,2′-((2-((5-Aminopentyl)amino)-2-oxoethyl)azanediyl)bis(acetyl))bis(azanediyl)) bis(N,N-bis(2-(((2S,3S,4S,5S,6R)-3,4,5-trihydroxy-6-(hydroxymethyl)tetrahydro-2H-pyran-2-yl)oxy)ethyl)hexanamide)

To the solution of benzyl (13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-4,11,15-trioxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16-tetraazahenicosan-21-yl)carbamate (670 mg, 0.459 mmol) in water (10 mL) was added Pd/C (147 mg, 0.138 mmol), and the reaction mixture was placed under H_{2(g)} and stirred at rt overnight. The catalyst was removed by filtration through CELITE™ diatomaceous earth, and the filtrate was lyophilized to yield the title compound. UPLC-MS Method B: m/z=1324.77 (z=1); t_{R}=1.60 min.

### Step 6: Benzyl (6-((2-(((2S,3S,4S,5R,6R)-3,5-dihydroxy-4-(((3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)carbamate

To the suspension of (2S,3S,4S,5S,6R)-2-(((2R,3R,4S,5S,6S)-6-(2-aminoethoxy)-3,5-dihydroxy-4-(((3S,4S,5S,6R)-3,4,5-trihydroxy-6-(hydroxymethyl)tetrahydro-2H-pyran-2-yl)oxy) tetrahydro-2H-pyran-2-yl)methoxy)-6-(hydroxymethyl)tetrahydro-2H-pyran-3,4,5-triol (1.0 g, 1.826 mmol) in DMF (5 mL) at rt was added 2,5-dioxopyrrolidin-1-yl 6-(((benzyloxy)carbonyl) amino)hexanoate (0.827 g, 2.283 mmol), followed by Hünig's base (0.399 mL, 2.283 mmol). The mixture was stirred overnight. The reaction mixture was purified by BIOTAGE SNAP ON 120 g C18 column, elute with 0-30% ACN in water. The fractions were combined and lyophilized to yield the title compound. UPLC-MS Method B: m/z=795.39 (z=1); t_{R}=4.21 min.

### Step 7: 6-Amino-N-(2-(((2S,3S,4S,5R,6R)-3,5-dihydroxy-4-(((3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) hexanamide

To the solution of benzyl (6-((2-(((2S,3S,4S,5R,6R)-3,5-dihydroxy-4-(((3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy) ethyl)amino)-6-oxohexyl)carbamate (1.1 g, 1.384 mmol) in water (20 mL) was added Pd/C (0.221 g, 0.208 mol), and the reaction mixture was placed under H_{2(g)} and stirred at rt overnight. The catalyst was removed by filtration through CELITE™ diatomaceous earth, and the filtrate was lyophilized to yield the title compound. UPLC-MS Method B: m/z=661.36 (z=1); t_{R}=4.30 min.

### Step 8: (3S)-Benzyl 3-(((benzyloxy)carbonyl)amino)-4-((6-((2-(((2S,3S,4S,5R,6R)-3,5-dihydroxy-4-(((3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-4-oxobutanoate

To the solution of Z-ASP(OBZL)-OH (406 mg, 1.135 mmol) in DMF (2 mL) was added EDC (218 mg, 1.135 mmol), followed by HOBT (174 mg, 1.135 mmol), then 6-amino-N-(2-(((2S,3S,4S,5R,6R)-3,5-dihydroxy-4-(((3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy) methyl)tetrahydro-2H-pyran-2-yl)oxy)ethyl)hexanamide (500 mg, 0.757 mmol) was added, and the mixture was stirred at rt for 2d. The mixture was concentrated and purified by BIOTAGE SNAP ON 120 g C18 column and eluted with 0-30% ACN in water. The fractions were combined and lyophilized to yield the title compound. UPLC-MS Method B: m/z=1000.436 (z=1); t_{R}=3.85 min.

### Step 9: (3S)-3-Amino-4-((6-((2-(((2S,3S,4S,5R,6R)-3,5-dihydroxy-4-(((3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-4-oxobutanoic acid

To the solution of (3S)-benzyl 3-(((benzyloxy)carbonyl)amino)-4-((6-((2-(((2S,3S,4S,5R,6R)-3,5-dihydroxy-4-(((3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl) amino)-4-oxobutanoate (500 mg, 0.500 mmol) in water (5 mL) was added Pd/C (133 mg, 0.125 mmol), and the reaction mixture was placed under H_{2(g)} and stirred at rt overnight. The catalyst was removed by filtration through CELITE™ diatomaceous earth, and the filtrate was lyophilized to yield the title compound. UPLC-MS Method B: m/z=776.44 (z=1); t_{R}=0.90 min.

### Step 10: (3S)-3-(6-(Benzyloxy)-6-oxohexanamido)-4-((6-((2-(((2S,3S,4S,5R,6R)-3,5-dihydroxy-4-(((3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-4-oxobutanoic acid

To the solution of (3S)-3-amino-4-((6-((2-(((2S,3S,4S,5R,6R)-3,5-dihydroxy-4-(((3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)amino)-4-oxobutanoic acid (388 mg, 0.500 mmol) in DMF (5 mL) at rt was added benzyl (2,5-dioxopyrrolidin-1-yl) adipate (208 mg, 0.625 mmol), followed by Hünig's base (0.109 mL, 0.625 mmol): the mixture was stirred at rt for 2 h. The mixture was concentrated and purified by BIOTAGE SNAP ON 120 g C18 column, eluted with 0-25% ACN in water 20CV. The fractions were combined and lyophilized to yield the title compound. UPLC-MS Method B: m/z=994.54 (z=1); t_{R}=2.73 min.

### Step 11: (S)-Benzyl 13-(2-((6-(bis(2-(((2S,3S,4S,5S,6R)-3,4,5-trihydroxy-6-(hydroxymethyl) tetrahydro-2H-pyran-2-yl)oxy)ethyl)amino)-6-oxohexyl)amino)-2-oxoethyl)-25-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) carbamoyl)-4,11,15,23,27-pentaoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,13,16,22,26-hexaazadotriacontan-32-oate

To the solution of (S)-3-(6-(benzyloxy)-6-oxohexanamido)-4-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)amino)-4-oxobutanoic acid (125 mg, 0.126 mmol) in DMF (2 mL) and water (0.5 mL) at rt was added 6,6′-((2,2′-((2-((5-aminopentyl)amino)-2-oxoethyl)azanediyl)bis(acetyl))bis(azanediyl))bis(N,N-bis(2-(((2S,3S,4S,5S,6R)-3,4,5-trihydroxy-6-(hydroxymethyl)tetrahydro-2H-pyran-2-yl)oxy)ethyl)hexanamide) (167 mg, 0.126 mmol), followed by EDC (30.1 mg, 0.157 mmol) and HOBT (24.07 mg, 0.157 mmol). The mixture was stirred at rt overnight. The mixture was concentrated and purified by BIOTAGE SNAP ON 120 g C18 column, eluted with 0-15% ACN in water. The fractions were combined and lyophilized to yield the title compound. UPLC-MS Method B: m/z=1151.10 (z=2); t_{R}=2.50 min.

### Step 12: (S)-13-(2-((6-(Bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-25-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)carbamoyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,26-hexaazadotriacontan-32-oic acid

To the solution of (S)-benzyl 13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-25-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)carbamoyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,26-hexaazadotriacontan-32-oate (135 mg, 0.059 mmol) in water (10 mL), was added Pd/C (62.5 mg, 0.059 mmol), and the reaction mixture was placed under H_{2(g)} and stirred at rt overnight. The catalyst was removed by filtration through CELITE™ diatomaceous earth, and the filtrate was lyophilized to yield the title compound. UPLC-MS Method B: m/z=1106.1 (z=2); t_{R}=1.93 min.

### Step 13: (S)-2,5-Dioxopyrrolidin-1-yl 13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-25-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)carbamoyl)-4,11,15,23,27-pentaoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,10,13,16,22,26-hexaazadotriacontan-32-oate

To the solution of (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-25-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)carbamoyl)-4,11,15,23,27-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,26-hexaazadotriacontan-32-oic acid (127 mg, 0.057 mmol) in DMF (2.0 mL) was added TSTU (182 mg, 0.603 mmol) at 0° C., followed by Hünig's Base (10.54 μl, 0.060 mmol). After 30 min, the ice bath was removed, and the mixture was stirred at rt for 3 h. The mixture was concentrated and purified by BIOTAGE SNAP ON 120 g C18 column, eluted with 0-15% ACN in water. The fractions were combined and lyophilized to yield the title compound. UPLC-MS Method B: m/z=1154.56 (z=2); t_{R}=1.98 min.

### Example 44: 2,5-Dioxopyrrolidin-1-yl (25S)-25-(4-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexanamido)butyl)-4,11,15,24,27-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23,26-hexaazadotriacontan-32-oate (ML-44)

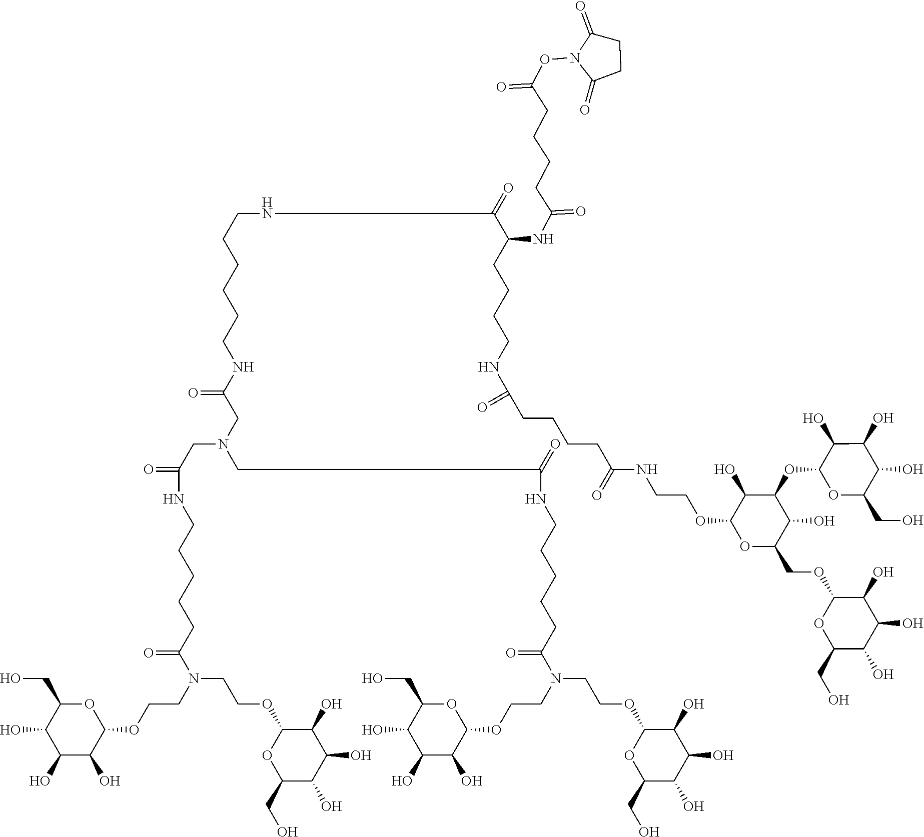 [SMILES: O=C(CCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NCCCC[C@H](NC(=O)CCCCC(=O)ON1C(=O)CCC1=O)C(=O)NCCCCCCNC(=O)CN(CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O]

### Step 1: Benzyl (4,11,15-trioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16-tetraazadocosan-22-yl)carbamate

14-(Carboxymethyl)-3,12-dioxo-1-phenyl-2-oxa-4,11,14-triazahexadecan-16-oic acid (WO2019125878A1) (150 mg, 0.354 mmol) was dissolved in DMF (10 mL). EDC (204 mg, 1.063 mmol) and HOBT (163 mg, 1.063 mol) were added. The mixture was stirred at rt for 30 min. 6-amino-N-(2-(((2S,3S,4S,5S,6R)-3,4,5-trihydroxy-6-(hydroxymethyl)tetrahydro-2H-pyran-2-yl)oxy)ethyl)-N-(2-(((3S,4S,5S,6R)-3,4,5-trihydroxy-6-(hydroxymethyl)tetrahydro-2H-pyran-2-yl)oxy)ethyl)hexanamide (423 mg, 0.779 mmol) was then added. The reaction was stirred at rt for 18h. DMF was removed under reduced pressure. The remainder was purified by C18 Reverse Phase chromatography (eluted with 0-30% ACN). Fractions containing desired product were combined, concentrated and lyophilized to furnish the title compound. UPLC-MS Method B: m/z=1473.79 (z=1); t_{R}=4.07 min.

### Step 2: 6,6′-((2,2′-((2-((6-Aminohexyl)amino)-2-oxoethyl)azanediyl)bis(acetyl))bis (azanediyl)) bis(N-(2-(((2S,3S,4S,5S,6R)-3,4,5-trihydroxy-6-(hydroxymethyl)tetrahydro-2H-pyran-2-yl)oxy)ethyl)-N-(2-(((3S,4S,5S,6R)-3,4,5-trihydroxy-6-(hydroxymethyl)tetrahydro-2H-pyran-2-yl)oxy)ethyl)hexanamide)

Benzyl (4,11,15-trioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16-tetraazadocosan-22-yl)carbamate (250 mg, 0.170 mmol) was dissolved in water (5 L), and hydrogenated under H_{2(g)} for 18 h. The mixture was removed by filtration through CELITE™ diatomaceous earth and washed with MeOH/water (1:1, 10 mL×3). The filtrate was concentrated and lyophilized to furnish the title compound. UPLC-MS Method A: m/z=669.9 (z=2); t_{R}=3.98 min.

### Step 3: N²-((Benzyloxy)carbonyl)-N⁶-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoyl)-L-lysine

To a solution of Z-Lys-OH (363 mg, 1.294 mmol) in DMF (10 mL) at 0° C. was added 2,5-dioxopyrrolidin-1-yl 6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoate (WO2016164288A1) (1000 mg, 1.294 mmol) dropwise, followed by Hunig base (0.226 mL, 1.294 mmol). The reaction was warmed to rt and stirred at rt for 18 h. The solvent was removed by rotary evaporation. The crude product was loaded onto C18 column (ISCO 130 g) eluted with water/ACN (0-30% in 18CV), flow rate 85 mL/min. The fractions containing the desired product were combined and lyophilized overnight. UPLC-MS Method B: m/z=938.34 (z=2); t_{R}=3.83 min.

### Step 4: N⁶-(6-((2-(((2S,3S,4S,5R,6R)-3,5-Dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoyl)-L-lysine

N²-((Benzyloxy)carbonyl)-N6-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoyl)-L-lysine (250 mg, 0.267 mmol) was dissolved in water (5 mL), degassed and filled with N_{2(g)}. To this solution was added Pd/C (28.4 mg, 0.267 mmol). The reaction vessel was degassed and filled with H_{2(g)}, then stirred under H_{2(g)} for 18 h. The mixture was removed by filtration through CELITE™ diatomaceous earth and washed with MeOH/water (1:1, 30 mL). The filtrate was concentrated under reduced pressure and lyophilized to give the title compound. UPLC-MS Method B: m/z=804.4 (z=1); t_{R}=4.27 min.

### Step 5: N²-(6-(Benzyloxy)-6-oxohexanoyl)-N⁶-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoyl)-L-lysine

N⁶-(6-((2-(((2S,3S,4S,5R,6R)-3,5-Dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoyl)-L-lysine (121 mg, 0.151 mmol) was dissolved in DMF (5 mL). The mixture was cooled with ice. To this solution was added benzyl 6-[(2,5-dioxopyrrolidin-1-yl)oxy]-6-oxohexanoate (70.5 mg, 0.211 mmol), followed by Hunig's base (0.037 mL, 0.211 mmol). The reaction was warmed to rt and stirred for 18 h. The DMF was removed under reduced pressure. The crude product was purified by C18 reverse phase chromatography (eluted with ACN/water 0-30%, column, ISCO C18 43 g). The fractions were combined and lyophilized to yield the title compound. UPLC-MS Method B: m/z=1022.58 (z=2); t_{R}=2.72 min.

### Step 6: Benzyl (25S)-25-(4-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)butyl)-4,11,15,24,27-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23,26-hexaazadotriacontan-32-oate

N²-(6-(benzyloxy)-6-oxohexanoyl)-N⁶-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanoyl)-L-lysine (100 mg, 0.098 mmol) was dissolved in DMF (2 mL), to above solution was added TSTU (29.5 mg, 0.098 mmol) followed by addition of Hünig's base (0.017 mL, 0.098 mmol). The reaction was stirred at 25° C. for 2h, followed by dropwise addition of 6,6′-((2,2′-((2-((6-aminohexyl)amino)-2-oxoethyl)azanediyl)bis(acetyl)) bis(azanediyl))bis(N-(2-(((2S,3S,4S,5S,6R)-3,4,5-trihydroxy-6-(hydroxymethyl)tetrahydro-2H-pyran-2-yl)oxy)ethyl)-N-(2-(((3S,4S,5S,6R)-3,4,5-trihydroxy-6-(hydroxymethyl)tetrahydro-2H-pyran-2-yl)oxy)ethyl)hexanamide) (131 mg, 0.098 mmol) in DMF (2 mL), followed by Hunig's base (0.017 mL, 0.098 mmol). The reaction was stirred at 25° C. for 18 h. The DMF was removed under reduced pressure. The crude product was purified by reverse phase C18 chromatography (43 g column, eluted with ACN/water 0-30%). The fractions were combined and lyophilized to yield the title compound. UPLC-MS Method B: m/z=1172.21 (z=2); t_{R}=2.37 min.

### Step 7: (25S)-25-(4-(6-((2-(((2S,3S,4S,5R,6R)-3,5-Dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)butyl)-4,11,15,24,27-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23,26-hexaazadotriacontan-32-oic acid

Benzyl (25S)-25-(4-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy) ethyl)amino)-6-oxohexanamido)butyl)-4,11,15,24,27-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy) ethyl)(2-(((3S,4S,5S,6R)-3,4,5-trihydroxy-6-(hydroxymethyl)tetrahydro-2H-pyran-2-yl)oxy) ethyl)amino)hexyl)amino)ethyl)-1-(((2S,3S,4S,5S,6R)-3,4,5-trihydroxy-6-(hydroxymethyl) tetrahydro-2H-pyran-2-yl)oxy)-3-(2-(((3S,4S,5S,6R)-3,4,5-trihydroxy-6-(hydroxymethyl) tetrahydro-2H-pyran-2-yl)oxy)ethyl)-3,10,13,16,23,26-hexaazadotriacontan-32-oate (30 mg, 0.013 mmol) was dissolved in water (5 mL). To the mixture was added Pd/C (10.41 mg, 9.78 μmol). The reaction was degassed and stirred under H_{2(g)} for 18 h. The mixture was filtered through CELITE™ diatomaceous earth and washed with MeOH (10 mL×3). The filtrate was concentrated and lyophilized to give the title compound (20 mg, 8.88 μmol). UPLC-MS Method B: m/z=1127.10 (z=2); t_{R}=1.66 min.

### Step 8: 2,5-Dioxopyrrolidin-1-yl (25S)-25-(4-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)butyl)-4,11,15,24,27-pentaoxo-13-(2-oxo-2-((6-oxo-6-((2-(((2S,3S,4S,5S,6R)-3,4,5-trihydroxy-6-(hydroxymethyl) tetrahydro-2H-pyran-2-yl)oxy) ethyl)(2-(((3S,4S,5S,6R)-3,4,5-trihydroxy-6-(hydroxymethyl) tetrahydro-2H-pyran-2-yl)oxy) ethyl)amino)hexyl) amino)ethyl)-1-(((2S,3S,4S,5S,6R)-3,4,5-trihydroxy-6-(hydroxymethyl) tetrahydro-2H-pyran-2-yl)oxy)-3-(2-(((3S,4S,5S,6R)-3,4,5-trihydroxy-6-(hydroxymethyl) tetrahydro-2H-pyran-2-yl)oxy) ethyl)-3,10,13,16,23,26-hexaazadotriacontan-32-oate

The title compound was prepared from (25S)-25-(4-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexanamido)butyl)-4,11,15,24,27-pentaoxo-13-(2-oxo-2-((6-oxo-6-((2-(((2S,3S,4S,5S,6R)-3,4,5-trihydroxy-6-(hydroxymethyl) tetrahydro-2H-pyran-2-yl)oxy)ethyl)(2-(((3S,4S,5S,6R)-3,4,5-trihydroxy-6-(hydroxymethyl) tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23,26-hexaazadotriacontan-32-oic acid using TSTU as described in Example 43, Step 13. UPLC-MS Method B: m/z=1175.70 (z=2); t_{R}=1.80 min.

### Example 45: 2,5-Dioxopyrrolidin-1-yl (24S)-24-(4-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexanamido)butyl)-4,11,15,23,26-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,22,25-hexaazahentriacontan-31-oate (ML-45)

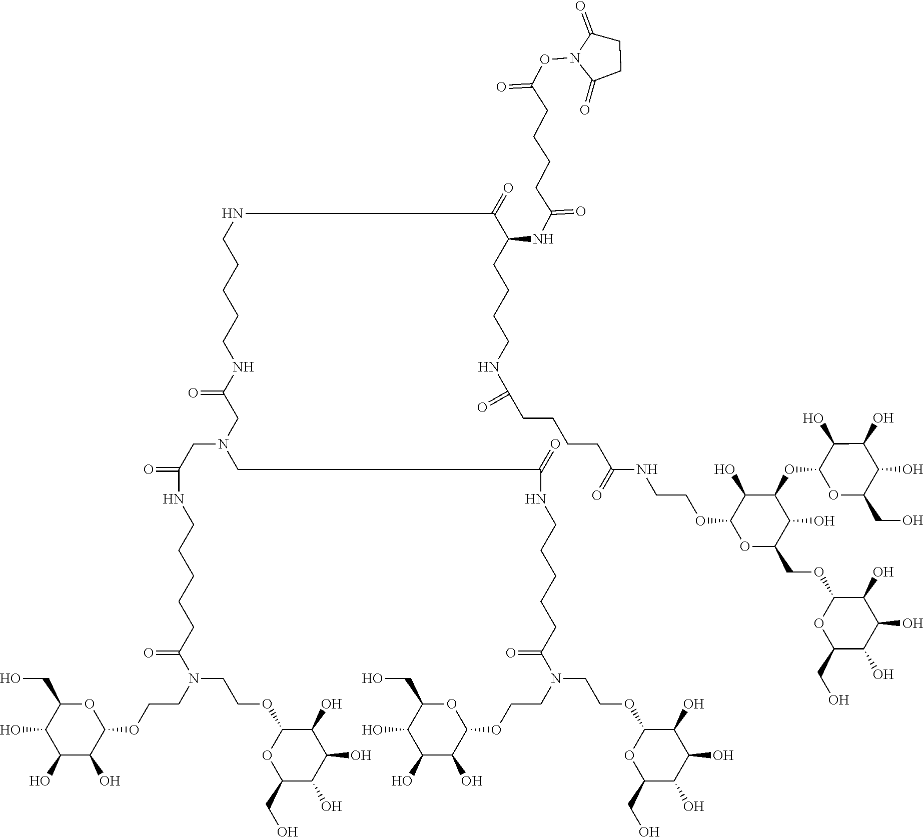 [SMILES: O=C(CCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NCCCC[C@H](NC(=O)CCCCC(=O)ON1C(=O)CCC1=O)C(=O)NCCCCCNC(=O)CN(CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O]

The title compound was prepared using procedures analogous to those described for ML-44 in Example 44, substituting 13-(carboxymethyl)-3,11-dioxo-1-phenyl-2-oxa-4,10,13-triazapentadecan-15-oic acid for 14-(carboxymethyl)-3,12-dioxo-1-phenyl-2-oxa-4,11,14-triazahexadecan-16-oic acid in Step 1. UPLC-MS Method B: m/z=1168.7 (z=2); t_{R}=2.02 min.

### Example 46: 2,5-Dioxopyrrolidin-1-yl (3S,25S)-14-(2-((6-(((2S)-1,4-bis(((3S,4S,5S,6R)-3,4,5-trihydroxy-6-(hydroxymethyl)tetrahydro-2H-pyran-2-yl)oxy)butan-2-yl)amino)-6-oxohexyl) amino)-2-oxoethyl)-25-(4-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)butyl)-5,12,16,24,27-pentaoxo-1-(((3S,4S,5S,6R)-3,4,5-trihydroxy-6-(hydroxymethyl)tetrahydro-2H-pyran-2-yl)oxy)-3-((((3S,4S,5S,6R)-3,4,5-trihydroxy-6-(hydroxymethyl)tetrahydro-2H-pyran-2-yl)oxy)methyl)-4,11,14,17,23,26-hexaazadotriacontan-32-oate (ML-46)

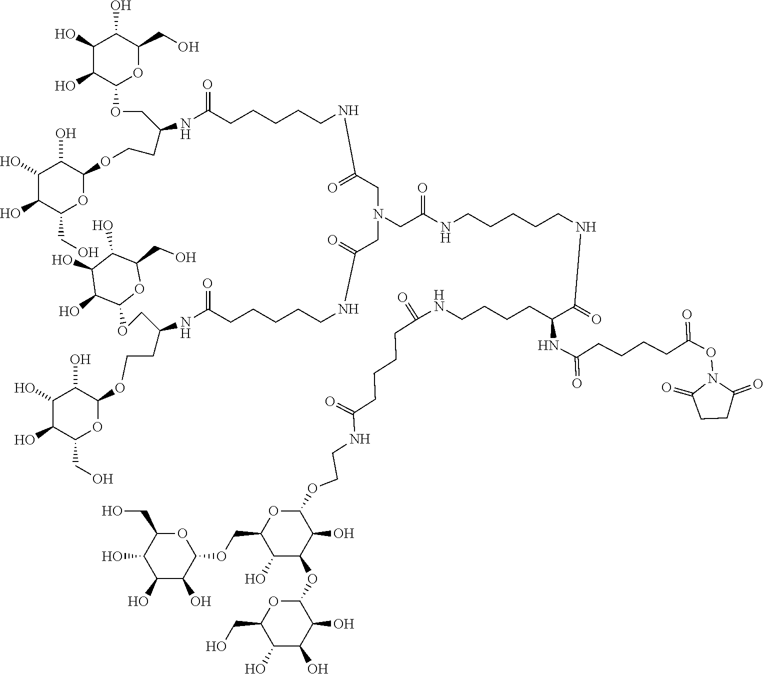 [SMILES: O=C(CCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NCCCC[C@H](NC(=O)CCCCC(=O)ON1C(=O)CCC1=O)C(=O)NCCCCCNC(=O)CN(CC(=O)NCCCCCC(=O)N[C@@H](CCO[C@H]1O[C@H](CO)[C@@H](O)[C@H](O)[C@@H]1O)CO[C@H]1O[C@H](CO)[C@@H](O)[C@H](O)[C@@H]1O)CC(=O)NCCCCCC(=O)N[C@@H](CCO[C@H]1O[C@H](CO)[C@@H](O)[C@H](O)[C@@H]1O)CO[C@H]1O[C@H](CO)[C@@H](O)[C@H](O)[C@@H]1O]

The title compound was prepared using procedures analogous to those described for ML-44 in Example 44, substituting 6-Amino-N—((S)-1,4-bis(((2S,3S,4S,5S,6R)-3,4,5-trihydroxy-6-(hydroxymethyl)tetrahydro-2H-pyran-2-yl)oxy)butan-2-yl)hexanamide for 6-amino-N-(2-(((2S,3S,4S,5S,6R)-3,4,5-trihydroxy-6-(hydroxymethyl)tetrahydro-2H-pyran-2-yl)oxy)ethyl)-N-(2-(((3S,4S,5S,6R)-3,4,5-trihydroxy-6-(hydroxymethyl)tetrahydro-2H-pyran-2-yl)oxy)ethyl) hexanamide in Step 1. UPLC-MS Method B: m/z=2335.4 (z=2); t_{R}=2.04 min.

### Example 47: 2,5-Dioxopyrrolidin 2,5-dioxopyrrolidin-1-yl (28S)-28-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexanamido)-4,11,15,22,29-pentaoxo-13-(2-oxo-2-((6-oxo-6-((2-(((2S,3S,4S,5S,6R)-3,4,5-trihydroxy-6-(hydroxymethyl) tetrahydro-2H-pyran-2-yl)oxy)ethyl)(2-(((3S,4S,5S,6R)-3,4,5-trihydroxy-6-(hydroxymethyl) tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23,30-hexaazahexatriacontan-36-oate (ML-47)

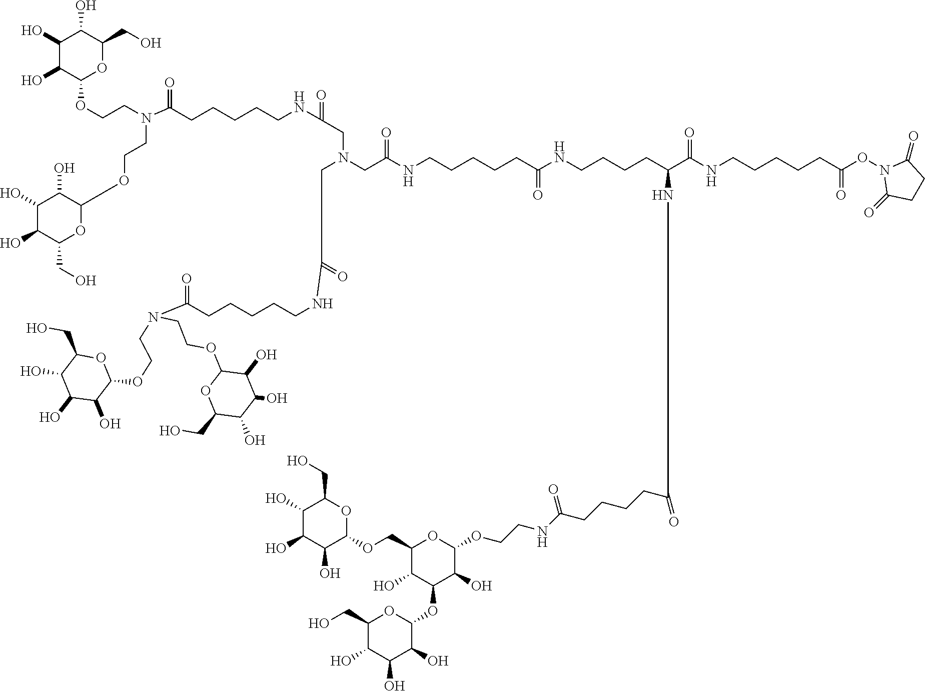 [SMILES: O=C(CCCCCNC(=O)CN(CC(=O)NCCCCCC(=O)N(CCOCC1O[C@H](CO)[C@@H](O)[C@H](O)[C@@H]1O)CCO[C@H]1O[C@H](CO)[C@@H](O)[C@H](O)[C@@H]1O)CC(=O)NCCCCCC(=O)N(CCOC1O[C@H](CO)[C@@H](O)[C@H](O)[C@@H]1O)CCO[C@H]1O[C@H](CO)[C@@H](O)[C@H](O)[C@@H]1O)NCCCC[C@H](NC(=O)CCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)C(=O)NCCCCCC(=O)ON1C(=O)CCC1=O]

### Step 1: Benzyl 4,11,15-trioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16-tetraazadocosan-22-oate

2,2′-((2-((6-(Benzyloxy)-6-oxohexyl)amino)-2-oxoethyl)azanediyl)diacetic acid (150 mg, 0.414 mmol) was dissolved in DMF (10 mL), and EDC (238 mg, 1.242 mmol) and HOBT (190 mg, 1.242 mmol) were added. The mixture was stirred at 25° C. for 30 min. To this solution was added 6-amino-N,N-bis{2-[(a-D-mannopyranosyl)oxy]ethyl}hexanamide (WO2018175272A1) (494 mg, 0.911 mmol). The reaction was stirred at 25° C. for 18 h. The DMF was removed under reduced pressure. The crude product was purified by C18 reverse phase chromatography (86 g column eluted with 0-30% ACN/water). The fractions were combined and lyophilized to yield the title compound.

### Step 2: 4,11,15-Trioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16-tetraazadocosan-22-oic acid

Benzyl 4,11,15-trioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16-tetraazadocosan-22-oate (253 mg, 0.175 mmol) was dissolved in water (5 mL), degassed, and filled with N₂. Pd/C (22.02 mg, 0.021 mmol) was added, and the reaction mixture was placed under H_{2(g)} and stirred for 18 h. The catalyst was removed by filtration through CELITE™ diatomaceous earth and washed with MeOH/water (1:1, 10 mL×3), and the filtrate was lyophilized to yield the title compound.

### Step 3: 2,5-Dioxopyrrolidin-1-yl 4,11,15-trioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16-tetraazadocosan-22-oate

In a 40 mL vial was added 4,11,15-trioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino) ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16-tetraazadocosan-22-oic acid (186 mg, 0.137 mmol) and DMF (3 mL). The reaction vessel was cooled with ice, and TSTU (43.4 mg, 0.144 mmol) was added to the reaction mixture, followed by Hünig's base (0.024 mL, 0.137 mmol). The reaction was warmed to rt and stirred for 30 min and used in the next step without purification.

### Step 4: (28S)-28-(((Benzyloxy)carbonyl)amino)-4,11,15,22-tetraoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oic acid

Z-Lys-OH (120 mg, 0.428 mmol) was dissolved in DMF (5 mL) and cooled to 0° C. 2,5-dioxopyrrolidin-1-yl 4,11,15-trioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl) amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16-tetraazadocosan-22-oate (640 mg, 0.441 mmol) in DMF (5 ml) was added dropwise followed by N-ethyl-N-isopropylpropan-2-amine (0.082 mL, 0.471 mmol). The mixture was warmed to rt and stirred for 18 h. The DMF was removed under reduced pressure, and the crude product was purified by C18 reverse phase chromatography (eluted with 0-40% ACN/water). The product-containing fractions were combined and lyophilized to yield the title compound.

### Step 5: (28S)-28-Amino-4,11,15,22-tetraoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-vy)oxy)-3-(2-(((3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oic acid

(28S)-28-(((benzyloxy)carbonyl)amino)-4,11,15,22-tetraoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oic acid (160 mg, 0.099 mmol) was dissolved in water (5 mL). Pd/C was added (14.6 mg), and the flask was filled with H_{2(g)} and stirred under H_{2(g)} or 18 h. The catalyst was removed by filtration through CELITE™ diatomaceous earth, and the filtrate was concentrated and lyophilized to furnish the title material.

### Step 6: (28S)-28-(6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)-4,11,15,22-tetraoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oic acid

(28S)-28-Amino-4,11,15,22-tetraoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino) ethyl)-1-(((2S,3 S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oic acid (65 mg, 0.044 mmol) was dissolved in DMF (2 mL) and cooled to 0° C. 6-[(2,5-dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-6-oxohexanamide (WO2019125878A1) (33.9 mg, 0.044 mmol) was added, followed by Hünig's base (7.66 μl, 0.044 mmol). The reaction was warmed to rt and stirred for 18 h. The crude product was purified by C18 reverse phase chromatography (26 g column, eluted with 0-30% ACN/water). The fractions were combined and lyophilized to yield the title compound.

### Step 7: Benzyl (28S)-28-(6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexanamido)-4,11,15,22,29-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23,30-hexaazahexatriacontan-36-oate

(28S)-28-(6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)-4,11,15,22-tetraoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23-pentaazanonacosan-29-oic acid (60 mg, 0.028 mmol) was dissolved in DMF (3 mL), cooled with ice, and treated with TSTU (12.67 mg, 0.042 mmol) and TEA (7.82 μl, 0.056 mmol). The reaction mixture was allowed to warm to rt and was treated with benzyl 6-aminohexanoate 4-methylbenzenesulfonate (14.3 mg, 0.036 mmol) and TEA (7.82 μl, 0.056 mmol). The reaction mixture was stirred at rt for 18h, concentrated and purified by C18 reverse phase chromatography (eluted with 0-50% ACN/water). The product-containing fractions were combined and lyophilized to yield the title compound.

### Step 8: (28S)-28-(6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)-4,11,15,22,29-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23,30-hexaazahexatriacontan-36-oic acid

Benzyl (28S)-28-(6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)-4,11,15,22,29-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23,30-hexaazahexatriacontan-36-oate (54 mg, 0.023 mmol) was dissolved in water (5 mL) and stirred over Pd/C (8.95 mg, 8.41 μmol) under H_{2(g)} for 5 h. The catalyst was removed by filtration through CELITE™ diatomaceous earth, and the filtrate was lyophilized to yield the title compound.

### Step 9: 2,5-Dioxopyrrolidin-1-yl (28S)-28-(6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)-4,11,15,22,29-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl) (2-(((3S,4S,5S,6R)-3,4,5-trihydroxy-6-(hydroxymethyl)tetrahydro-2H-pyran-2-yl)oxy)ethyl) amino)hexyl)amino) ethyl)-1-(((2S,3S,4S,5S,6R)-3,4,5-trihydroxy-6-(hydroxymethyl) tetrahydro-2H-pyran-2-yl)oxy)-3-(2-(((3S,4S,5S,6R)-3,4,5-trihydroxy-6-(hydroxymethyl) tetrahydro-2H-pyran-2-yl)oxy)ethyl)-3,10,13,16,23,30-hexaazahexatriacontan-36-oate

(28S)-28-(6-((2-(((2S,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)-4,11,15,22,29-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23,30-hexaazahexatriacontan-36-oic acid (50 mg, 0.022 mmol) was dissolved in DMF (3 mL) and treated with TSTU (8.69 mg, 0.029 mmol) and N-ethyl-N-isopropylpropan-2-amine (5.58 μl, 0.031 mmol). The reaction was stirred at rt for 2h. 20× volumes of acetone were added. The precipitate was collected by centrifugation and dried in vacuum to furnish the title material. UPLC-MS Method B: m/z=1175.7 (z=2); t_{R}=2.39 min.

### Example 48: 2,5-Dioxopyrrolidin-1-yl (24S)-24-(4-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexanamido)butyl)-4,11,15,22,25-pentaoxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16,23,26-hexaazadotriacontan-32-oate (ML-48)

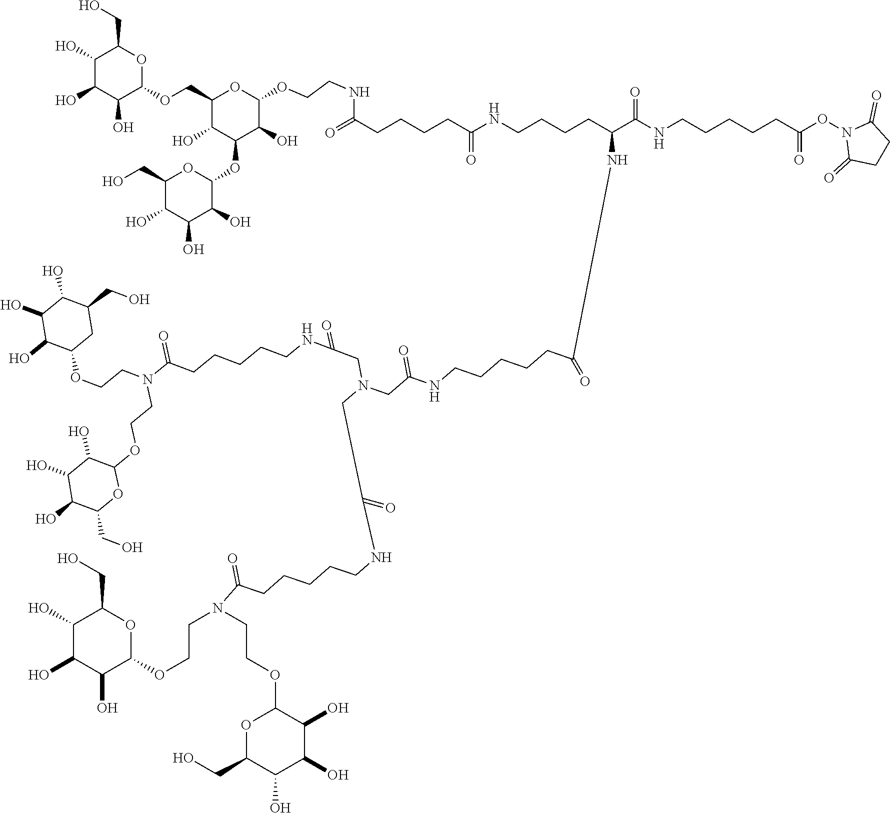 [SMILES: O=C(CCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NCCCC[C@H](NC(=O)CCCCCNC(=O)CN(CC(=O)NCCCCCC(=O)N(CCOC1O[C@H](CO)[C@@H](O)[C@H](O)[C@@H]1O)CCO[C@H]1O[C@H](CO)[C@@H](O)[C@H](O)[C@@H]1O)CC(=O)NCCCCCC(=O)N(CCOC1O[C@H](CO)[C@@H](O)[C@H](O)[C@@H]1O)CCO[C@H]1C[C@H](CO)[C@@H](O)[C@H](O)[C@@H]1O)C(=O)NCCCCCC(=O)ON1C(=O)CCC1=O]

The title compound was prepared using procedures analogous to those described for ML-47 in Example 47, substituting 6-[(2,5-dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-6-oxohexanamide (WO2019125878A1) for 2,5-dioxopyrrolidin-1-yl 4,11,15-trioxo-13-(2-oxo-2-((6-oxo-6-((2-(((2S,3S,4S,5S,6R)-3,4,5-trihydroxy-6-(hydroxymethyl)tetrahydro-2H-pyran-2-yl)oxy)ethyl)(2-(((3S,4S,5S,6R)-3,4,5-trihydroxy-6-(hydroxymethyl)tetrahydro-2H-pyran-2-yl)oxy)ethyl)amino) hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16-tetraazadocosan-22-oate in Step 4 and substituting 2,5-dioxopyrrolidin-1-yl 4,11,15-trioxo-13-(2-oxo-2-((6-oxo-6-((2-(((2S,3S,4S,5S,6R)-3,4,5-trihydroxy-6-(hydroxymethyl) tetrahydro-2H-pyran-2-yl)oxy)ethyl)(2-(((3S,4S,5S,6R)-3,4,5-trihydroxy-6-(hydroxymethyl) tetrahydro-2H-pyran-2-yl)oxy)ethyl)amino)hexyl)amino)ethyl)-1-(((2S,3S,4S,5S,6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-3,10,13,16-tetraazadocosan-22-oate for 6-[(2,5-dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-6-oxohexanamide (WO2019125878A1) in Step 6. UPLC-MS Method B: m/z=1175.19 (z=2); t_{R}=3.63 min.

### Example 49: 5-Dioxopyrrolidin-1-yl (S)-6-(2-(bis(2-(((2S,3S,4S,5S,6R)-3,4,5-trihydroxy-6-(hydroxymethyl)tetrahydro-2H-pyran-2-yl)oxy)ethyl)amino)-2-oxoethyl)-21-((6-((2-(((2S,3S,4S,5R,6R)-3,5-dihydroxy-4-(((2S,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) carbamoyl)-4,8,15,23-tetraoxo-1-(((2S,3S,4S,5S,6R)-3,4,5-trihydroxy-6-(hydroxymethyl) tetrahydro-2H-pyran-2-yl)oxy)-3-(2-(((2S,3S,4S,5S,6R)-3,4,5-trihydroxy-6-(hydroxymethyl) tetrahydro-2H-pyran-2-yl)oxy)ethyl)-3,6,9,16,22-pentaazaoctacosan-28-oate (ML-49)

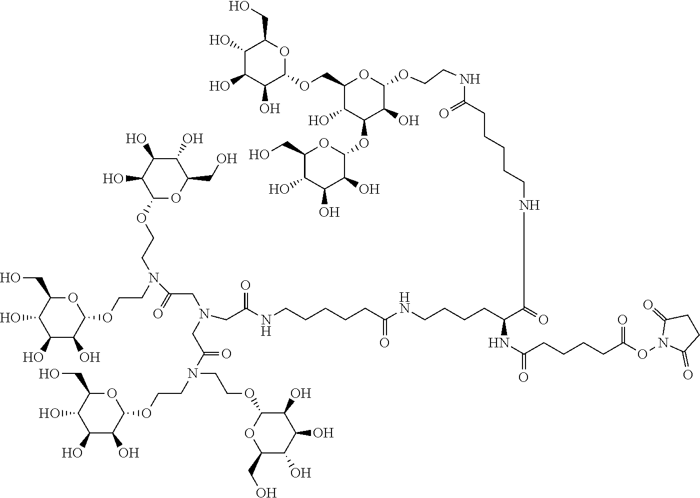 [SMILES: O=C(CCCCCNC(=O)CN(CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)NCCCC[C@H](NC(=O)CCCCC(=O)ON1C(=O)CCC1=O)C(=O)NCCCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

The title compound was prepared using procedures analogous to those described for ML-44 in Example 44, substituting 2,5-dioxopyrrolidin-1-yl 6-(2-(bis(2-(bis(2-(((2S,3S,4S,5S,6R)-3,4,5-trihydroxy-6-(hydroxymethyl)tetrahydro-2H-pyran-2-yl)oxy)ethyl)amino)-2-oxoethyl) amino)acetamido)hexanoate for 2,5-dioxopyrrolidin-1-v 6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl) oxy)methyl)tetrahydro-2H-pyran-2-yl)oxy)ethyl)amino)-6-oxohexanoate in Step 3; substituting 6-amino-N-(2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) hexanamide for 6,6′-((2,2′-((2-((6-aminohexyl) amino)-2-oxoethyl)azanediyl)bis(acetyl)) bis(azanediyl))bis(N-(2-(((2S,3S,4S,5S,6R)-3,4,5-trihydroxy-6-(hydroxymethyl)tetrahydro-2H-pyran-2-yl)oxy)ethyl)-N-(2-(((3S,4S,5S,6R)-3,4,5-trihydroxy-6-(hydroxymethyl)tetrahydro-2H-pyran-2-yl)oxy)ethyl)hexanamide) in Step 6. UPLC-MS Method B: m/z=1062.03 (z=2); t_{R}=3.13 min.

### Example 50: 2,5-Dioxopyrrolidin-1-yl (S)-6-(2-(bis(2-(((2S,3S,4S,5S,6R)-3,4,5-trihydroxy-6-(hydroxymethyl)tetrahydro-2H-pyran-2-yl)oxy)ethyl)amino)-2-oxoethyl)-18-(4-(6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexanamido)butyl)-4,8,17,20-tetraoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,6,9,16,19-pentaazapentacosan-25-oate (ML-50)

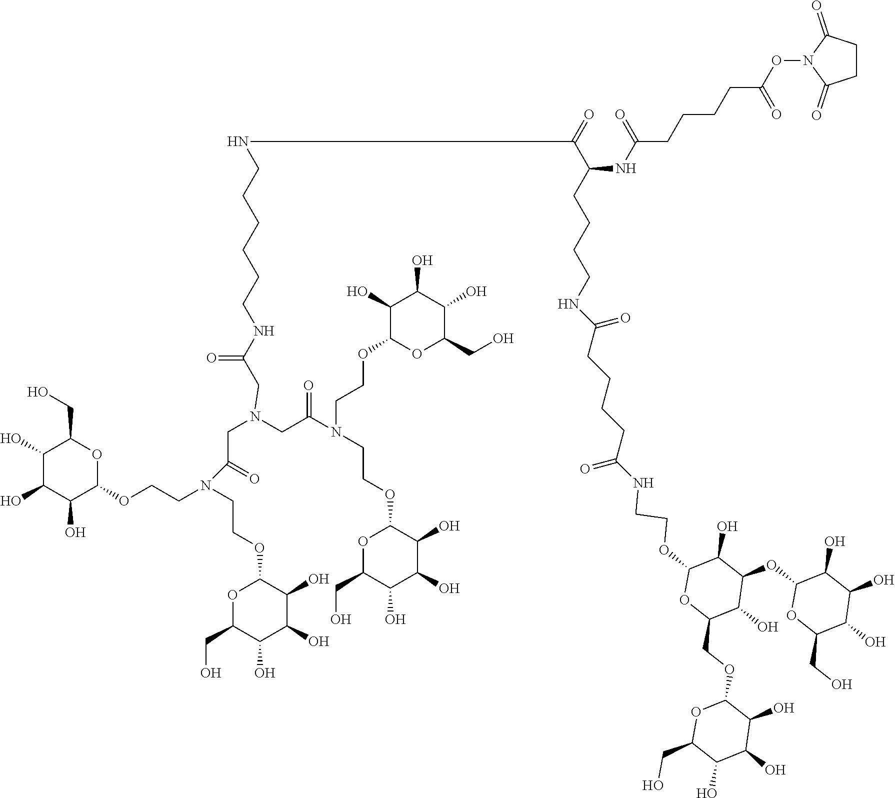 [SMILES: O=C(CCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NCCCC[C@H](NC(=O)CCCCC(=O)ON1C(=O)CCC1=O)C(=O)NCCCCCCNC(=O)CN(CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O]

The title compound was prepared following procedures for Steps 6-8 described for ML-44 in Example 44, where in Step 6 2,2′-((2-((6-aminohexyl)amino)-2-oxoethyl)azanediyl)bis (N,N-bis(2-(((2S,3S,4S,5S,6R)-3,4,5-trihydroxy-6-(hydroxymethyl)tetrahydro-2H-pyran-2-yl) oxy)ethyl) acetamide) was substituted for 6,6′-((2,2′-((2-((6-aminohexyl)amino)-2-oxoethyl) azanediyl)bis(acetyl))bis(azanediyl))bis(N-(2-(((2S,3S,4S,5S,6R)-3,4,5-trihydroxy-6-(hydroxymethyl)tetrahydro-2H-pyran-2-yl)oxy)ethyl)-N-(2-(((3S,4S,5S,6R)-3,4,5-trihydroxy-6-(hydroxymethyl)tetrahydro-2H-pyran-2-yl)oxy)ethyl)hexanamide). UPLC-MS Method B: m/z=1062.07 (z=2); t_{R}=3.81 min.

### Example 51: 2,5-Dioxopyrrolidin-1-yl 16-((3-((6-((2-(((2S,3S,4S,5R,6R)-3,5-dihydroxy-4-(((2S,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-3-oxopropoxy)methyl)-4,11-dioxo-16-((3-oxo-3-((6-oxo-6-((2-(((2S,3S,4S,5S,6R)-3,4,5-trihydroxy-6-(hydroxymethyl) tetrahydro-2H-pyran-2-yl) oxy)ethyl)(2-(((3S,4S,5S,6R)-3,4,5-trihydroxy-6-(hydroxymethyl) tetrahydro-2H-pyran-2-yl)oxy) ethyl)amino)hexyl)amino) propoxy)methyl)-1-(((2S,3S,4S,5S, 6R)-3,4,5-trihydroxy-6-(hydroxymethyl)tetrahydro-2H-pyran-2-yl)oxy)-3-(2-(((3S,4S,5S,6R)-3,4,5-trihydroxy-6-(hydroxymethyl)tetrahydro-2H-pyran-2-yl)oxy)ethyl)-14,18-dioxa-3,10-diazahenicosan-21-oate (ML-51)

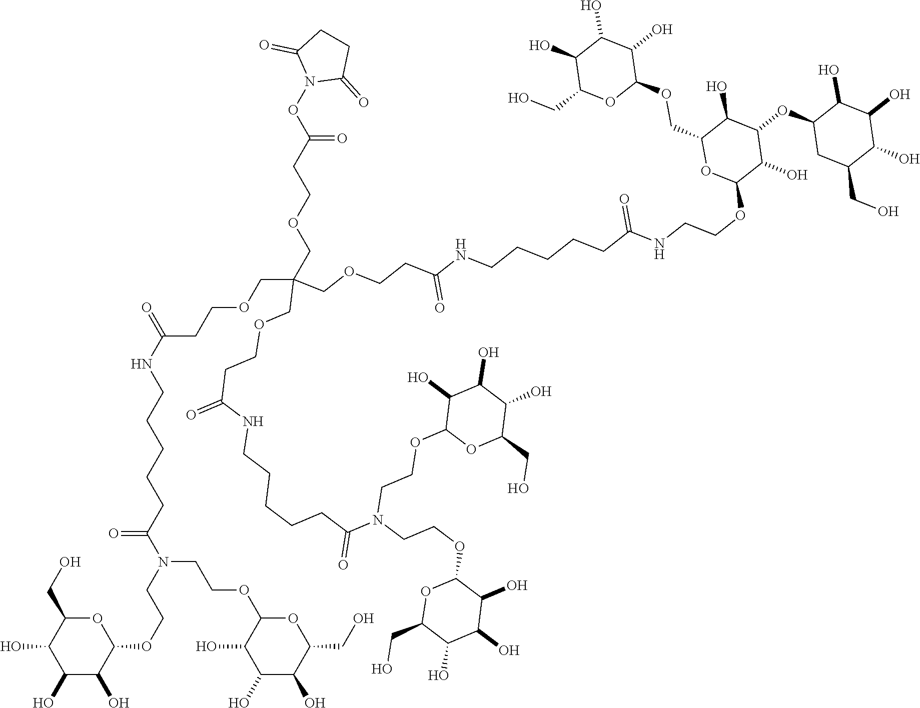 [SMILES: O=C(CCCCCNC(=O)CCOCC(COCCC(=O)NCCCCCC(=O)N(CCOC1O[C@H](CO)[C@@H](O)[C@H](O)[C@@H]1O)CCO[C@H]1O[C@H](CO)[C@@H](O)[C@H](O)[C@@H]1O)(COCCC(=O)NCCCCCC(=O)N(CCOC1O[C@H](CO)[C@@H](O)[C@H](O)[C@@H]1O)CCO[C@H]1O[C@H](CO)[C@@H](O)[C@H](O)[C@@H]1O)COCCC(=O)ON1C(=O)CCC1=O)NCCO[C@H]1O[C@H](CO[C@H]2O[C@H](CO)[C@@H](O)[C@H](O)[C@@H]2O)[C@@H](O)[C@H](O[C@@H]2C[C@H](CO)[C@@H](O)[C@H](O)[C@@H]2O)[C@@H]1O]

The title compound was prepared using procedures analogous to those described for ML-1 in Example 1 of WO2018175272A1, substituting (2S,3S,4S,5S,6R)-2-(((2R,3R,4S,5S,6S)-6-(2-aminoethoxy)-3,5-dihydroxy-4-(((2S,3S,4S,5S,6R)-3,4,5-trihydroxy-6-(hydroxymethyl) tetrahydro-2H-pyran-2-yl)oxy)tetrahydro-2H-pyran-2-yl)methoxy)-6-(hydroxymethyl) tetrahydro-2H-pyran-3,4,5-triol or (2R,3S,4S,5S)-2-(hydroxymethyl)-6-(2-((2-(((2S,3S,4S,5S,6R)-3,4,5-trihydroxy-6-(hydroxymethyl)tetrahydro-2H-pyran-2-yl)oxy)ethyl)amino)ethoxy) tetrahydro-2H-pyran-3,4,5-triol for AEM. UPLC-MS Method B: m/z=1107.6 (z=2); t_{R}=1.74 mm.

### Example 52: 2,5-dioxopyrrolidin-1-yl (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-24-(3-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-3-oxopropyl)-4,11,15,23,26-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25-hexaazahentriacontan-31-oate (ML-52)

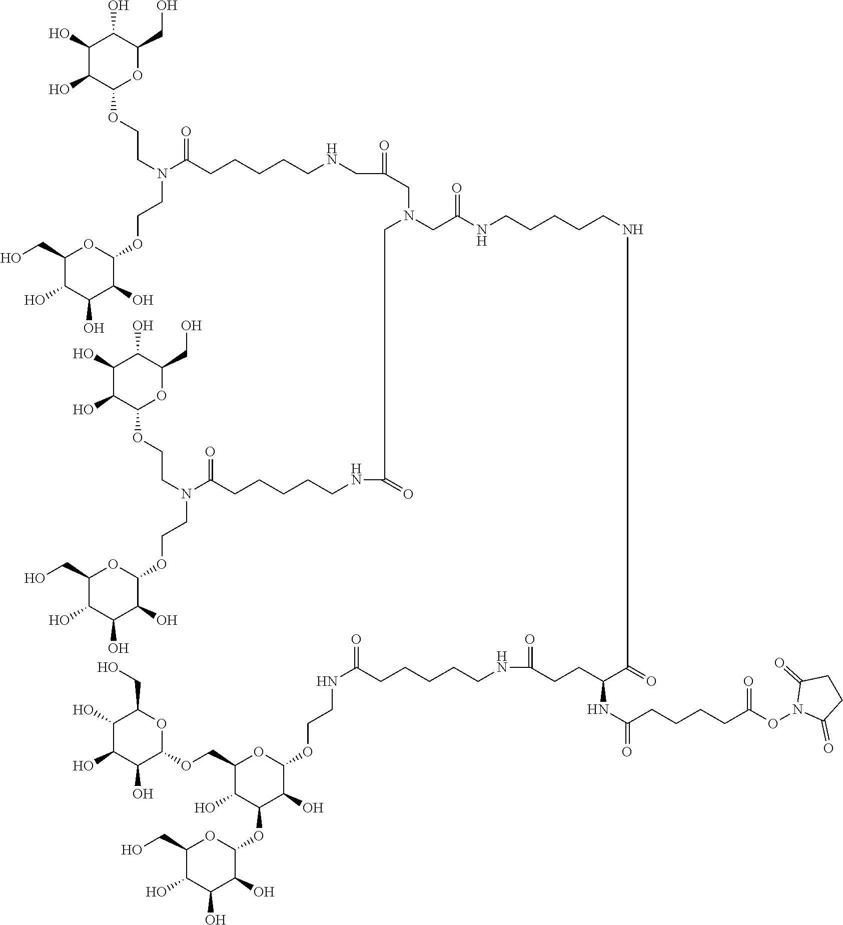 [SMILES: O=C(CNCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O)CN(CC(=O)NCCCCCNC(=O)[C@H](CCC(=O)NCCCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)NC(=O)CCCCC(=O)ON1C(=O)CCC1=O)CC(=O)NCCCCCC(=O)N(CCO[C@H]1O[C@H](CO)[C@@H](O)[C@H](O)[C@@H]1O)CCO[C@H]1O[C@H](CO)[C@@H](O)[C@H](O)[C@@H]1O]

### Step 1: Benzyl N²-((benzyloxy)carbonyl)-N5-(6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)-L-glutaminate

To a solution of 6-amino-N-(2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)hexanamide (284 mg, 0.430 mmol) and Z-GLU-OBZL (176 mg, 0.473 mmol) in DMF (10 mL) was added EDC (124 mg, 0.645 mmol) and HOBT (6.58 mg, 0.043 mmol), and the reaction mixture was stirred overnight. The mixture was concentrated and purified by BIOTAGE SNAP ON 120 g C18 column (0-20% ACN in water). The fractions were combined and lyophilized to yield the title compound. UPLC-MS Method B: m/z=1014.52 (z=1); t_{R}=3.54 min.

### Step 2: N⁵-(6-((2-(((2S,3S,4S,5R,66R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)-L-glutamine

A solution of benzyl N²-((benzyloxy)carbonyl)-N⁵-(6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)-L-glutaminate (309 mg, 0.30 mmol) and PdOH2 (10.70 mg, 0.015 mmol) in water was stirred under H_{2(g)} overnight. The catalyst was removed by filtration through CELITE™ diatomaceous earth, and the filtrate was lyophilized to yield the title compound. UPLC-MS Method B: m/z=790.35 (z=1); t_{R}=1.36 min.

### Step 3: N²-(6-(Benzyloxy)-6-oxohexanoyl)-N⁵-(6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl)-L-glutamine

A mixture of 6-(benzyloxy)-6-oxohexanoic acid (70 mg, 0.296 mmol) in CH₂Cl₂ (10 mL) at 0° C. was added TSTU (89 mg, 0.296 mmol) and TEA (0.052 mL, 0.37 mmol). After stirring at 0° C. for 30 min, the mixture was diluted with ether (20 mL) and washed with brine (1×30 mL). The organic layer was dried over MgSO₄ and concentrated to give an NHS activated ester, which was added in DMF (2.0 mL) to a suspension of N²-(6-(benzyloxy)-6-oxohexanoyl)-N⁵-(6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl)-L-glutamine (195 mg, 0.247 mmol) in DMF (10 mL). The mixture was stirred at rt overnight. The mixture was concentrated and purified by BIOTAGE SNAP ON 120 g C18 column (gradient 0-30% ACN in water). The fractions were combined and lyophilized to yield the title compound. UPLC-MS Method B: m/z=1008.52 (z=2); t_{R}=2.96 min.

### Step 4: Benzyl (S)-13-(2-((6-(bis(2-(((2S,3S,4S,5S,6R)-3,4,5-trihydroxy-6-(hydroxymethyl) tetrahydro-2H-pyran-2-yl)oxy)ethyl)amino)-6-oxohexyl)amino)-2-oxoethyl)-24-(3-((6-((2-(((2S,3S,4S,5R,6R)-3,5-dihydroxy-4-(((2R,3S,4S,5S,6R)-3,4,5-trihydroxy-6-(hydroxymethyl) tetrahydro-2H-pyran-2-yl)oxy)-6-((((2S,3S,4S,5S,6R)-3,4,5-trihydroxy-6-(hydroxymethyl) tetrahydro-2H-pyran-2-yl)oxy)methyl)tetrahydro-2H-pyran-2-yl)oxy)ethyl)amino)-6-oxohexyl) amino)-3-oxopropyl)-4,11,15,23,26-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25-hexaazahentriacontan-31-oate

To a solution of N²-(6-(benzyloxy)-6-oxohexanoyl)-N⁵-(6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl) oxy)-6-((((2S,3S,4S,5S,6R)-3,4,5-trihydroxy-6-(hydroxymethyl)tetrahydro-2H-pyran-2-yl)oxy) methyl)tetrahydro-2H-pyran-2-yl)oxy)ethyl)amino)-6-oxohexyl)-L-glutamine (100 mg, 0.099 mmol) and 6,6′-((2,2′-((2-((5-aminopentyl)amino)-2-oxoethyl)azanediyl)bis(acetyl)) bis(azanediyl))bis(N,N-bis(2-(((2S,3S,4S,5S,6R)-3,4,5-trihydroxy-6-(hydroxymethyl)tetrahydro-2H-pyran-2-yl)oxy)ethyl)hexanamide) (131 mg, 0.099 mmol) in DMF (10 mL) at rt was added EDC (38.0 mg, 0.198 mmol) and HOBT (1.519 mg, 9.92 μmol). The mixture was stirred overnight. The mixture was concentrated and purified by BIOTAGE SNAP ON 120 g C18 column (gradient 0-30% ACN in water). The desired fractions were combined and freeze-dried to give the desired product. UPLC-MS Method B: m/z=1158.1 (z=2); t_{R}=2.41 min.

### Step 5: (S)-13-(2-((6-(Bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-24-(3-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-3-oxopropyl)-4,11,15,23,26-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25-hexaazahentriacontan-31-oic acid

A mixture of benzyl (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-24-(3-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-3-oxopropyl)-4,11,15,23,26-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25-hexaazahentriacontan-31-oate (177.5 mg, 0.077 mmol) and Pd(OH)₂ (2.69 mg, 3.83 μmol) in water (10 mL) was stirred under H_{2(g)} overnight. The catalyst was removed by filtration through CELITE™ diatomaceous earth, and the filtrate was lyophilized to yield the title compound. UPLC-MS m/z=1113.0 (z=2); t_{R}=1.89 min.

### Step 6: 2,5-Dioxopyrrolidin-1-yl (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-24-(3-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-6-oxohexyl) amino)-3-oxopropyl)-4,11,15,23,26-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25-hexaazahentriacontan-31-oate

To a solution of (S)-13-(2-((6-(bis(2-(((2S,3S,4S,5S,6R)-3,4,5-trihydroxy-6-(hydroxymethyl)tetrahydro-2H-pyran-2-yl)oxy)ethyl)amino)-6-oxohexyl)amino)-2-oxoethyl)-24-(3-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl) amino)-6-oxohexyl)amino)-3-oxopropyl)-4,11,15,23,26-pentaoxo-1-(((2S,3S,4S,5S,6R)-3,4,5-trihydroxy-6-(hydroxymethyl)tetrahydro-2H-pyran-2-yl)oxy)-3-(2-(((2S,3S,4S,5S,6R)-3,4,5-trihydroxy-6-(hydroxymethyl)tetrahydro-2H-pyran-2-yl)oxy)ethyl)-3,10,13,16,22,25-hexaazahentriacontan-31-oic acid (137 mg, 0.062 mmol) in DMF (5 mL) at 0° C. was added TSTU (20.40 mg, 0.068 mmol) and Et₃N (9.44 μl, 0.068 mmol). After stirring at 0° C. for 30 min, the mixture was warmed to rt and stirred for 1 h. The reaction mixture was concentrated, and the residue was purified on BIOTAGE SNAP ON 120 g C18 column (gradient 0-30% ACN in water). The desired fractions were combined and lyophilized to furnish the desired product. UPLC-MS m/z=1183.5 (z=2); t_{R}=1.78 min.

### Example 53: Synthesis of IOC-1

Human insulin (1300 mg, 0.224 mmol) was dissolved in aq Na₂CO₃ (9.85 mL, 0.1M) and ACN (10.0 mL). The pH of the resulting solution was adjusted to 10.5, to which 2,5-dioxopyrrolidin-1-yl (S)-27-(2-((6-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl) tetrahydro-2H-pyran-2-yl)oxy)ethyl)amino)-6-oxohexyl)amino)-2-oxoethyl)-4,13,17,25,29-pentaoxo-6-(2-oxo-2-((2-(((2R,3S,4R,5S,6S)-3,4,5-trihydroxy-6-methyltetrahydro-2H-pyran-2-yl)oxy)ethyl)amino)ethyl)-15-(2-oxo-2-((6-oxo-6-((2-(((2R,3S,4R,5S,6S)-3,4,5-trihydroxy-6-methyltetrahydro-2H-pyran-2-yl)oxy)ethyl)amino)hexyl)amino)ethyl)-1-(((2R,3S,4R,5S,6S)-3,4,5-trihydroxy-6-methyltetrahydro-2H-pyran-2-yl)oxy)-7-((2-(((2R,3S,4R,5S,6S)-3,4,5-trihydroxy-6-methyltetrahydro-2H-pyran-2-yl)oxy)ethyl)carbamoyl)-3,6,12,15,18,24,27,30-octaazahexatriacontan-36-oate (ML-1) (597 mg, 0.247 mmol) in DMSO (2.0 mL) was added in 4 portions over 80 min. The reaction mixture was quenched by adding 2-aminoethanol (4.8 μL, 0.079 mmol). After stirring at rt for 15 min, the reaction mixture was diluted with water, and the pH was adjusted to about 2.5 using conc. 1.0N HCl solution. The resulting solution was purified by preparatory scale HPLC using a C8 50×250 mm column, gradient 24-28.5% ACN in H₂O with 0.1% TFA over 25 min, flow rate 85 mL/min. The desired fractions were combined and lyophilized. UPLC-MS Method B: m/z=1622.6 (z=2); t_{R}=4.66 min.

Examples 54 through 105, conjugates IOC-2 to IOC-14, IOC-16 to IOC-43, IOC-46 to IOC-56, as listed in Table 1, were prepared according to procedures analogous to those described with respect to Example 53, Synthesis of IOC-1, substituting the appropriate penta-valent sugar clusters as indicated for ML-1. Insulin Lispro was used in Example 85, IOC-34.

TABLE 1

| Example | Conjugate | Pena-Valent Sugar Cluster |
|---|---|---|
| 54 | IOC-2 | ML-2 |
| 55 | IOC-3 | ML-3 |
| 56 | IOC-4 | ML-4 |
| 57 | IOC-5 | ML-5 |
| 58 | IOC-6 | ML-6 |
| 59 | IOC-7 | ML-7 |
| 60 | IOC-8 | ML-8 |
| 61 | IOC-9 | ML-9 |
| 62 | IOC-10 | ML-10 |
| 63 | IOC-11 | ML-11 |
| 64 | IOC-12 | ML-12 |
| 65 | IOC-13 | ML-13 |
| 66 | IOC-14 | ML-14 |
| 67 | IOC-16 | ML-16 |
| 68 | IOC-17 | ML-17 |
| 69 | IOC-18 | ML-18 |
| 70 | IOC-19 | ML-19 |
| 71 | IOC-20 | ML-20 |
| 72 | IOC-21 | ML-21 |
| 73 | IOC-22 | ML-22 |
| 74 | IOC-23 | ML-23 |
| 75 | IOC-24 | ML-24 |
| 76 | IOC-25 | ML-25 |
| 77 | IOC-26 | ML-26 |
| 78 | IOC-27 | ML-27 |
| 79 | IOC-28 | ML-28 |
| 80 | IOC-29 | ML-29 |
| 81 | IOC-30 | ML-30 |
| 82 | IOC-31 | ML-31 |
| 83 | IOC-32 | ML-32 |
| 84 | IOC-33 | ML-33 |
| 85 | IOC-34 (Lispro) | ML-32 |
| 86 | IOC-35 | ML-35 |
| 87 | IOC-36 | ML-36 |
| 88 | IOC-37 | ML-37 |
| 89 | IOC-38 | ML-38 |
| 90 | IOC-39 | ML-39 |
| 91 | IOC-40 | ML-40 |
| 92 | IOC-41 | ML-41 |
| 93 | IOC-42 | ML-42 |
| 94 | IOC-43 | ML-43 |
| 95 | IOC-46 | ML-46 |
| 96 | IOC-47 | ML-47 |
| 97 | IOC-48 | ML-48 |
| 98 | IOC-49 | ML-49 |
| 99 | IOC-50 | ML-50 |
| 100 | IOC-51 | ML-51 |
| 101 | IOC-52 | ML-52 |
| 102 | IOC-53 | ML-53 |
| 103 | IOC-54 | ML-54 |
| 104 | IOC-55 | ML-55 |
| 105 | IOC-56 | ML-56 |

### Example 106: Synthesis of IOC-15

N^{A1}, N^{ε29}-Bis(trifluoroacetyl) Human Insulin (WO2015051052A1) (100 mg, 0.0083 mmol) was dissolved in DMSO (1 mL). To this solution was added ML-15 (19.6 mg, 0083 mmol) in 1 mL DMSO. Stirring was continued for 90 min, followed by the addition of 2-aminoethanol (0.0028 mL, 0.043 mmol) to the reaction mixture. The mixture was stirred for 15 min to quench the reaction. To the mixture was added aqueous ammonium hydroxide (5 mL) at 4° C. and stored for 18h. UPLC-MS showed fully deprotection of trifluoroacetate groups. The resulting solution was purified by preparatory scale HPLC with a C8 50×250 mm column, using gradient 27-31% ACN in H₂O with 0.1% TFA over 25 min. flow rate 85 mL/min. The combined desired fractions were lyophilized to furnish IOC-15. UPLC-MS Method B: m/z=1705.63 (z=4); t_{R}=3.28 min.

Examples 107 and 108, conjugates IOC-44 and IOC-45, as listed in Table 2, were prepared according to procedures analogous to those described with respect to Examples 106: Synthesis of IOC-15, substituting the appropriate penta-valent sugar clusters as indicated for ML-15.

TABLE 2

| Example | Conjugate | Penta-Valent Sugar Clusters |
|---|---|---|
| 107 | IOC-44 | ML-40 |
| 108 | IOC-45 | ML-41 |

### Example 109: Insulin Receptor Phosphorylation Assays

The insulin receptor phosphorylation assays were performed using the commercially available Meso Scale Discovery (“MSD”) pIR assay (See Meso Scale Discovery, 9238 Gaithers Road, Gaithersburg, Md.). CHO cells stably expressing human IR(B) were in grown in in F12 cell media containing 10% FBS and antibiotics (G418, Penicillin/Strepavidin) for at least 8 h and then serum starved by switching to F12 media containing 0.5% BSA (insulin-free) in place of FBS for overnight growth. Cells were harvested and frozen in aliquots for use in the MSD pIR assay. Briefly, the frozen cells were plated in either 96-well (40,000 cells/well, Method A and Method B) or 384-well (10,000 cells/well. Method C) clear tissue culture plates and allowed to recover. IOC molecules at the appropriate concentrations were added, and the cells were incubated for 8 min at 37° C. The media was aspirated and chilled. MSD cell lysis buffer (cell lysis buffer formulation: 150 mM NaCl; 20 mM Tris, pH 7.5; 1 mM EDTA; 1 mM EGTA and 1% Triton X-100); MSD kit pIR detection plate containing insulin signaling panel) was added as per MSD kit instructions. The cells were lysed on ice for 40 min, and the lysate then mixed for 10 min at rt. The lysate was transferred to the MSD kit pIR detection plates. The remainder of the assay was carried out following the MSD kit recommended protocol.

### Example 110: Insulin Receptor Binding Assays

Insulin Receptor Binding Assays were performed as follows.

Two competition binding assays were utilized to determine IOC affinity for the human insulin receptor type B (IR(B)) against the endogenous ligand, insulin, labeled with ¹²⁵[I].

Method C: IR binding assay was a whole cell binding method using CHO cells overexpressing human IR(B). The cells were grown in F12 media (Ham's F-12 Nutrient Mixture, a nutrient mixture designed to cultivate a wide variety of mammalian and hybridoma cells when used with serum in combination with hormones and transferrin) containing 10% FBS and antibiotics (G418, Penicillin/Strepavidin), plated at 40,000 cells/well in a 96-well tissue culture plate for at least 8 h. The cells were then serum starved by switching to DMEM media containing 1% BSA (insulin-free) overnight. The cells were washed twice with chilled DMEM media containing 1% BSA (insulin-free) followed by the addition of IOC molecules at appropriate concentration in 90 μL of the same media. The cells were incubated on ice for 60 min. The ¹²⁵[I]-insulin (10 L) was added at 0.015 nm final concentration and incubated on ice for 4 h. The cells were gently washed three times with chilled media and lysed with 30 μL of Cell Signaling lysis buffer (Cell Signal Technology, catalog #9803) with shaking for 10 min at rt. The lysate was added to scintillation liquid and counted to determine ¹²⁵[I]-insulin binding to IR and the titration effects of IOC molecules on this interaction.

Method D: IR binding assay was run in a scintillation proximity assay (SPA) in 384-well format using cell membranes prepared from CHO cells overexpressing human IR(B) grown in F12 media containing 10% FBS and antibiotics (G418, Penicillin/Strepavidin). Cell membranes were prepared in 50 mm Tris (tris(hydroxymethyl) aminomethane) buffer, pH 7.8 containing 5 mm MgCl₂. The assay buffer contained 50 mm Tris buffer, pH 7.5, 150 mm NaCl, 1 mm CaCl₂), 5 mm MgCl₂, 0.1% BSA and protease inhibitors (Complete-Mini-Roche). Cell membranes were added to WGA PVT PEI SPA beads (5 mg/ml final concentration) followed by addition of IOC molecules at appropriate concentrations. After 5-15 min incubation at rt, ¹²⁵[I]-insulin was added at 0.015 nm final concentration for a final total volume of 50 μL. The mixture was incubated with shaking at rt for 1 to 12h followed by scintillation counting to determine ¹²⁵[I]-insulin binding to IR and the titration effects of IOC molecules on this interaction.

### Example 111: Human Macrophage Mannose Receptor 1 (MRC1) Binding Assays

Human macrophage mannose receptor 1 (“MRC1”) Binding Assays were performed as follows.

The competition binding assay for MRC1 utilized a ligand, mannosylated-BSA labeled with the DELFIA Eu-N1-ITC reagent (labeling kit for europium labeling of proteins and polypetides for use in dissociation-enhanced time-resolved fluorometric assay), as reported in the literature. Assay was performed either in a 96-well plate with 100 μL well volume (Method E) or in a 384-well plate with 25 μL well volume (Method F). Anti-MRC1 (Mannose Receptor C-Type 1) antibody (2 ng/μl) in PBS containing 1% stabilizer BSA was added to a Protein G plate that had been washed three times with 100 μl of 50 mm Tris buffer, pH 7.5 containing 100 mm NaCl, 5 mm CaCl₂), 1 mm MgCl₂ and 0.1% Tween-20 (wash buffer). The antibody was incubated in the plate for 1 h at rt with shaking. The plate was washed with wash buffer 3-5 times followed by addition of MRC1 (2 ng/μl final concentration) in PBS containing 1% stabilizer BSA. The plate was incubated at rt with gentle shaking for 1 h. The plate was washed three times with wash buffer. The IOC molecules in 12.5 μL (or 50 μL depending on plate format) buffer at appropriate concentrations were added followed by 12.5 μL (or 50 μL) Eu-mannosylated-BSA (0.1 nm final concentration) in 50 mm Tris, pH 7.5 containing 100 mm NaCl, 5 mm CaCl₂), 1 mm MgCl₂ and 0.2% stabilizer BSA. The plate was incubated for 2 h at rt with shaking followed by washing three times with wash buffer. Perkin Elmer Eu-inducer reagent was added and incubated for 30 min at rt prior to detection of the Eu signal (Excitation=340 nm: Emission=615 nm).

### Example 112: Assay Results

The following table lists conjugates that were prepared using appropriate intermediates following one of the General Methods described above. These conjugates were characterized using UPLC Method A or UPLC Method B, exhibiting either four charged, i.e., [(M+4)/4], (or five charged, i.e., [(M+5)/5]) species of parent compound at certain retention time (“t_{R}”). The in vitro biological activities towards insulin receptor (IR) were measured by either ligand competition assays or functional phosphorylation assays, as described above, labeled as following: Method A: IR phosphorylation assay based on 96-well; Method B: IR phosphorylation assay based on 384-well with automated liquid dispense; Method C: cell-based IR binding assay; Method D: SPA IR binding assay method E; Method E: MRC1 assay was performed in a 96-well plate; Method F: MRC1 assay was performed in a 384-well plate. The results are shown in Table 3.

TABLE 3

| | Human pIR | Human IR | Human MRC1 |
|---|---|---|---|
| Conjugate | IC₅₀ (nM) | EC₅₀ (nM) | IC₅₀ (nM) |
| IOC-01 | 1.92 | 2.77 | 1.51 |
| IOC-02 | 1.38 | 3.38 | 1.13 |
| IOC-03 | 1.75 | 2.51 | 0.73 |
| IOC-04 | 1.74 | 8.77 | 1.73 |
| IOC-05 | 1.5 | 5.99 | 1.31 |
| IOC-06 | 3.06 | 5.24 | 10.8 |
| IOC-07 | 2.01 | 6.96 | 0.93 |
| IOC-08 | 1.99 | 8.15 | 3.67 |
| IOC-09 | 1.2 | 11.77 | 9.06 |
| IOC-10 | 1.4 | 7.52 | 21.11 |
| IOC-11 | 1.22 | 7.66 | 5.96 |
| IOC-12 | 3.58 | 1.25 | 2.02 |
| IOC-13 | 1.56 | 0.77 | 3 |
| IOC-14 | 2.68 | 0.87 | 11.76 |
| IOC-15 | 0.92 | 1.02 | 24.38 |
| IOC-16 | | | 16.45 |
| IOC-17 | | | 3.01 |
| IOC-18 | | | 39.8 |
| IOC-19 | | | 9.45 |
| IOC-20 | 4.22 | 1.78 | 52.74 |
| IOC-21 | 2.78 | 1.17 | 6.74 |
| IOC-22 | 2.56 | 2.14 | 54.95 |
| IOC-23 | 2.14 | 2.16 | 65.42 |
| IOC-24 | 5.95 | 3.35 | 24.32 |
| IOC-25 | 1.9 | 3.11 | 8.2 |
| IOC-26 | 4.56 | 2.41 | 18.47 |
| IOC-27 | 2.04 | 0.7 | 15.36 |
| IOC-28 | 2.8 | 1.46 | 5.04 |
| IOC-29 | 2.05 | 1.12 | 1.97 |
| IOC-30 | 2.4 | 1.38 | 3.99 |
| IOC-31 | 2.4 | 1.25 | 8.95 |
| IOC-32 | 2.26 | 1.5 | 7.34 |
| IOC-33 | 1.66 | 2.4 | 16.83 |
| IOC-34 | 6.36 | 1.53 | 82.67 |
| IOC-35 | 1.49 | 9.21 | 19.66 |
| IOC-36 | 1.62 | 10.09 | 14.42 |
| IOC-37 | 1.7 | 9.68 | 34.91 |
| IOC-38 | 2.28 | 12.72 | 3.77 |
| IOC-39 | 1.73 | 9.04 | 1.46 |
| IOC-40 | 2.08 | 2.26 | 13.91 |
| IOC-41 | 1.77 | 1.12 | 7.4 |
| IOC-42 | 1.5 | 0.97 | 10.93 |
| IOC-43 | 2.22 | 1.34 | 11.9 |
| IOC-44 | NA | 4.07 | 51.49 |
| IOC-45 | 3.97 | 3.81 | 24.23 |
| IOC-46 | 3.18 | 2.19 | 16.45 |
| IOC-47 | 2.27 | 2.87 | 17.86 |
| IOC-48 | 7.88 | 1.27 | 13.26 |
| IOC-49 | 3.67 | 2.67 | 17.76 |
| IOC-50 | 3.39 | 2.69 | 37.37 |
| IOC-51 | 4.92 | 1.34 | 9.39 |
| IOC-52 | 3.44 | 1.47 | 15.28 |
| IOC-53 | 2.56 | 1.51 | 20.99 |
| IOC-54 | 0.71 | 0.94 | 15.2 |
| IOC-55 | 3.07 | 2.3 | 40.91 |
| IOC-56 | 3.82 | 1.75 | 7.02 |

### Example 113: Effect of α-Methylmannose (αMM) on PK and PD of Various IOCs in Non-Diabetic Minipigs

Effect of Methyl α-Methylmannose (αMM) on PK and PD of various IOCs in Non-Diabetic Minipigs was evaluated.

Male Yucatan miniature pigs, non-diabetic, instrumented with two Jugular vein vascular access ports (“VAP”), were used in these studies. Animals were fasted overnight prior to the study. On the day of the study, animals were restrained in slings, and VAPs accessed for infusion and sampling. At t=−60 min, a constant infusion of PBS (n=3) or 21.2% αMM (n=3) was started, at a rate of 2.67 mL/kg/h. This infusion was maintained for the duration of the study. At t=0 min, and after collecting a baseline blood sample for plasma glucose measurement, animals were administered IOC as a single bolus IV. Sampling continued for 90 min, with final readouts of plasma glucose and compound levels.

IOCs were formulated at 17-69 nmol/mL in sodium chloride (87 mm), phenol (21 mm), dibasic sodium phosphate (26.5 mm), Osmolality=275 mOsm/kg (milliosmoles per kilogram), pH=7.4; QS (Quantum satis) with Water for Injection.

Time points for sample collection: −60 min, 0 min, 1 min, 2 min, 4 min, 6 min, 8 min, 10 min, 15 min, 20 min, 25 min, 30 min, 35 min, 45 min, 60 min, and 90 min.

Blood was collected in K₃-EDTA (tripotassiuim ethylenediaminetetraacetic acid) tubes, supplemented with 10 μg/ml Aprotinin, and kept on an ice bath until processing, within 30 min of collection. After centrifugation at 3000 rpm, 4° C., for 8 min, plasma was collected and aliquoted for glucose measurement using a Beckman Coulter AU480 Chemistry analyzer and for compound levels measurement by LC-MS.

The IOCs evaluated were IOC-1, IOC-9, IOC-14, IOC-26, IOC-32, IOC-33, IOC-36, IOC-47, IOC-49, IOC-51, IOC-52, and IOC-53.

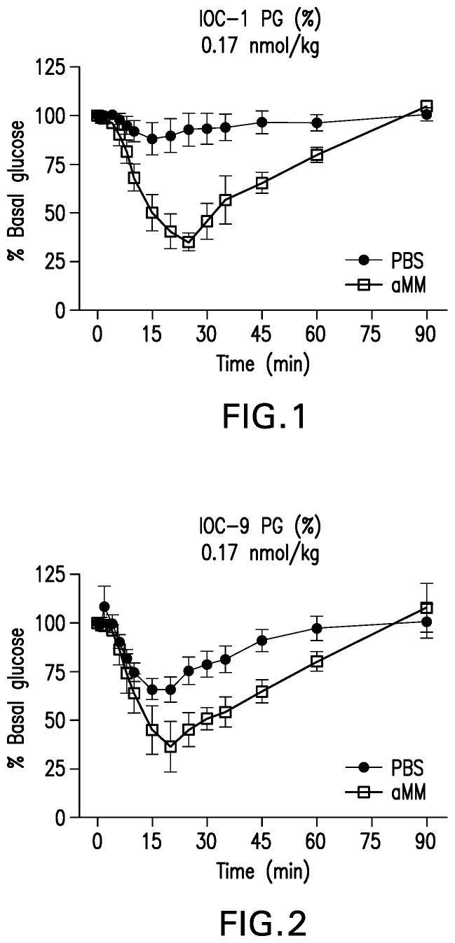

Glucose results are expressed as % changes over baseline values at t=0 min. and the results are shown in FIG. 1 through FIG. 12.

FIG. 1 shows blood glucose depression curves in non-diabetic male Yucatan minipigs equipped with dual vascular access ports (n=3 per study) following i.v. injection of conjugate IOC-1 at 0.69 nmol/kg under conditions of PBS infusion or i.v. αMM infusion.

FIG. 2 shows blood glucose depression curves in non-diabetic male Yucatan minipigs equipped with dual vascular access ports (n=3 per study) following i.v. injection of conjugate IOC-9 at 0.69 nmol/kg under conditions of PBS infusion or i.v. αMM infusion.

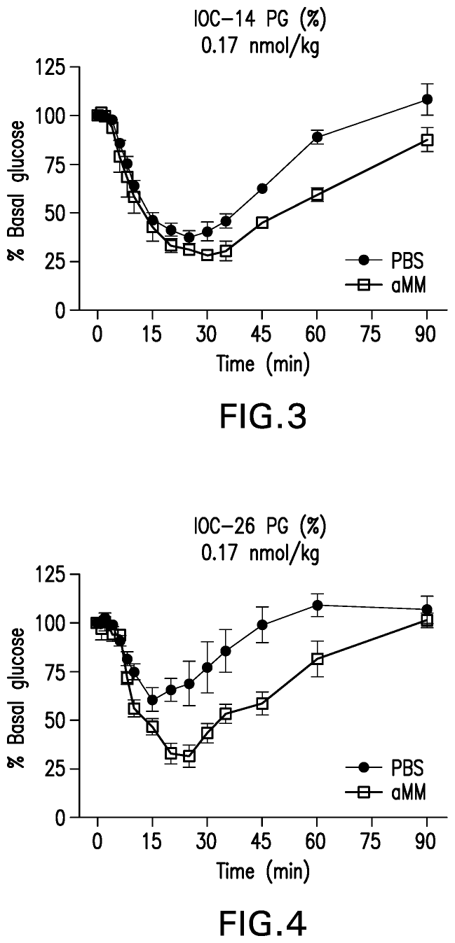

FIG. 3 shows blood glucose depression curves in non-diabetic male Yucatan minipigs equipped with dual vascular access ports (n=3 per study) following i.v. injection of conjugate IOC-14 at 0.69 nmol/kg under conditions of PBS infusion or i.v. αMM infusion.

FIG. 4 shows blood glucose depression curves in non-diabetic male Yucatan minipigs equipped with dual vascular access ports (n=3 per study) following i.v. injection of conjugate IOC-26 at 0.69 nmol/kg under conditions of PBS infusion or i.v. αMM infusion.

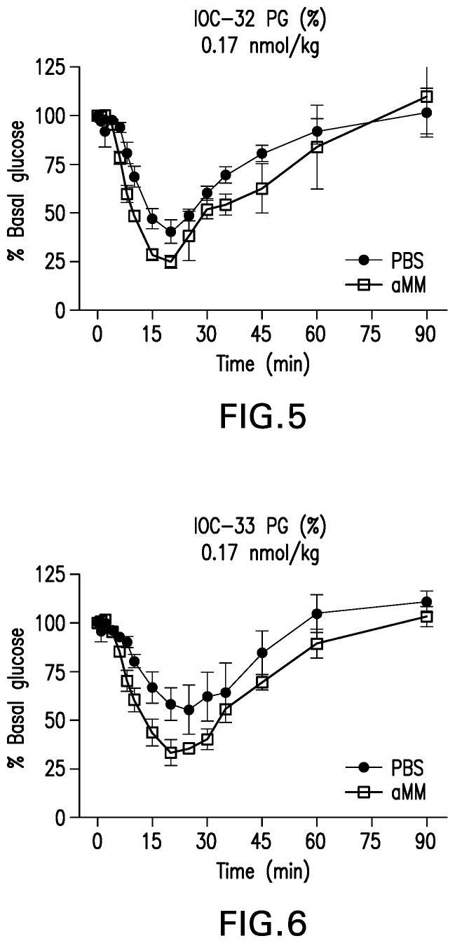

FIG. 5 shows blood glucose depression curves in non-diabetic male Yucatan minipigs equipped with dual vascular access ports (n=3 per study) following i.v. injection of conjugate IOC-32 at 0.69 nmol/kg under conditions of PBS infusion or i.v. αMM infusion.

FIG. 6 shows blood glucose depression curves in non-diabetic male Yucatan minipigs equipped with dual vascular access ports (n=3 per study) following i.v. injection of conjugate IOC-33 at 0.69 nmol/kg under conditions of PBS infusion or i.v. αMM infusion.

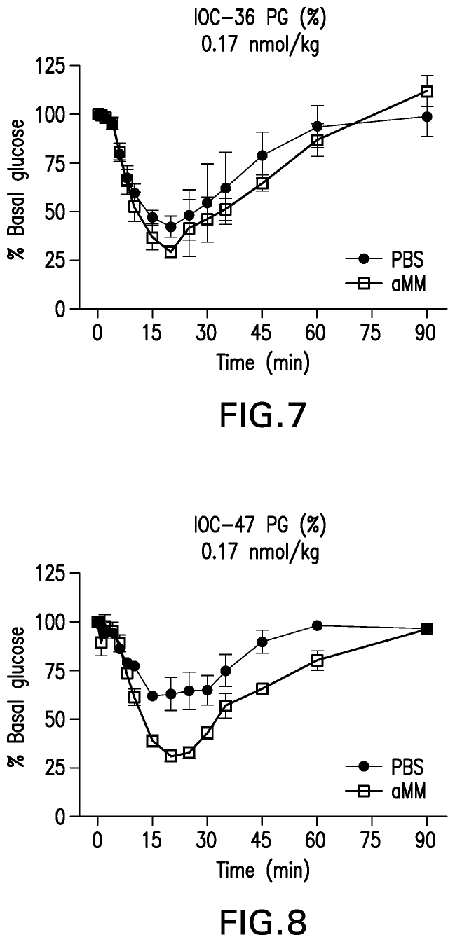

FIG. 7 shows blood glucose depression curves in non-diabetic male Yucatan minipigs equipped with dual vascular access ports (n=3 per study) following i.v. injection of conjugate IOC-36 at 0.69 nmol/kg under conditions of PBS infusion or i.v. αMM infusion.

FIG. 8 shows blood glucose depression curves in non-diabetic male Yucatan minipigs equipped with dual vascular access ports (n=3 per study) following i.v. injection of conjugate IOC-47 at 0.69 nmol/kg under conditions of PBS infusion or i.v. αMM infusion.

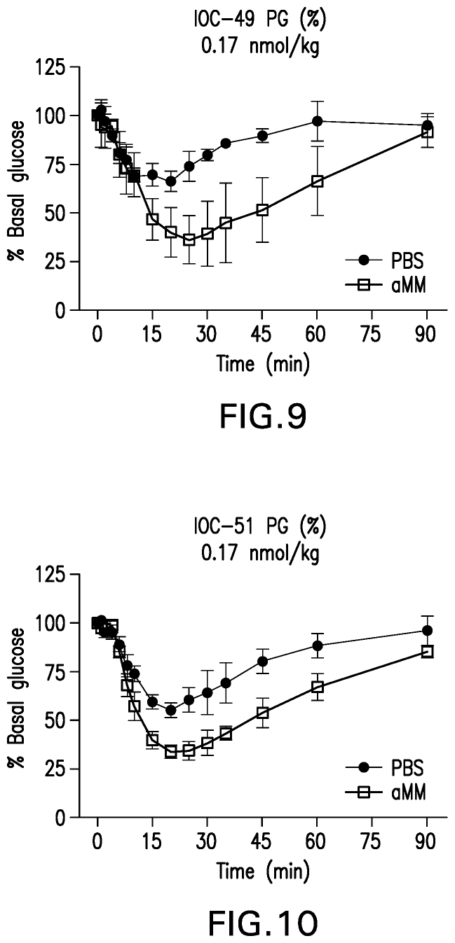

FIG. 9 shows blood glucose depression curves in non-diabetic male Yucatan minipigs equipped with dual vascular access ports (n=3 per study) following i.v. injection of conjugate IOC-49 at 0.69 nmol/kg under conditions of PBS infusion or i.v. αMM infusion.

FIG. 10 shows blood glucose depression curves in non-diabetic male Yucatan minipigs equipped with dual vascular access ports (n=3 per study) following i.v. injection of conjugate IOC-51 at 0.69 nmol/kg under conditions of PBS infusion or i.v. αMM infusion.

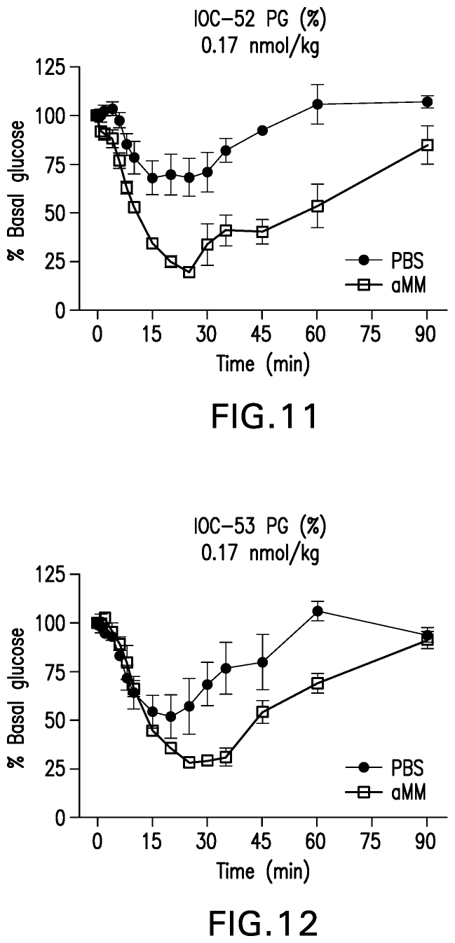

FIG. 11 shows blood glucose depression curves in non-diabetic male Yucatan minipigs equipped with dual vascular access ports (n=3 per study) following i.v. injection of conjugate IOC-52 at 0.69 nmol/kg under conditions of PBS infusion or i.v. αMM infusion.

FIG. 12 shows blood glucose depression curves in non-diabetic male Yucatan minipigs equipped with dual vascular access ports (n=3 per study) following i.v. injection of conjugate IOC-53 at 0.69 nmol/kg under conditions of PBS infusion or i.v. αMM infusion.

It will be appreciated that various of the above-discussed and other features and functions, or alternatives thereof, may be desirably combined into many other different systems or applications. It will also be appreciated that various alternatives, modifications, variations, or improvements therein may be subsequently made by those skilled in the art that are also intended to be encompassed by the following claims.

1. A conjugate comprising an insulin or insulin analog molecule covalently attached to at least one penta-valent sugar cluster, wherein the penta-valent sugar cluster is provided by a penta-dentate linker having five arms, wherein each arm of the penta-dentate linker is independently covalently linked to a ligand comprising a saccharide.

2. The conjugate according to claim 1, wherein the conjugate displays a PD or PK profile that is sensitive to the serum concentration of a serum saccharide when administered to a subject in need thereof in the absence of an exogenous saccharide binding molecule.

3. The conjugate according to claim 1, wherein binding of the conjugate to the endogenous saccharide binding molecule is sensitive to the serum concentration of the serum saccharide.

4. The conjugate according to claim 1, wherein the ligand comprises a saccharide selected from fucose, mannose, glucosamine, glucose, bimannose, trimannose, tetramannose, and branched trimannose.

5. The conjugate according to claim 1, wherein the ligand comprises a saccharide and amine group.

6. The conjugate according to claim 1, wherein the ligand comprises a saccharide selected from aminoethylglucose, aminoethylmannose, aminoethylbimannose, aminoethyltrimannose, β-aminoethyl-N-acetylglucosamine, and aminoethylfucose.

7. The conjugate according to claim 1, wherein the penta-valent sugar cluster is covalently linked to the side chain amino group of B29 lysine of insulin or insulin analog, in the presence or absence of small substitution on A1 and B1 amino groups.

8. The conjugate according to claim 1, wherein the insulin analog is insulin lispro, insulin glargine, insulin aspart, insulin detemir, or insulin glulisine.

9. The conjugate according to claim 1, wherein the conjugate has the general formula I: 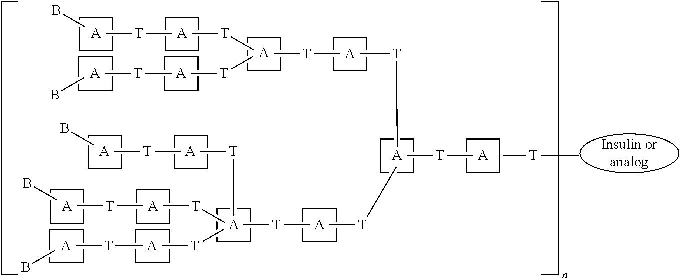
or has the general formula II: 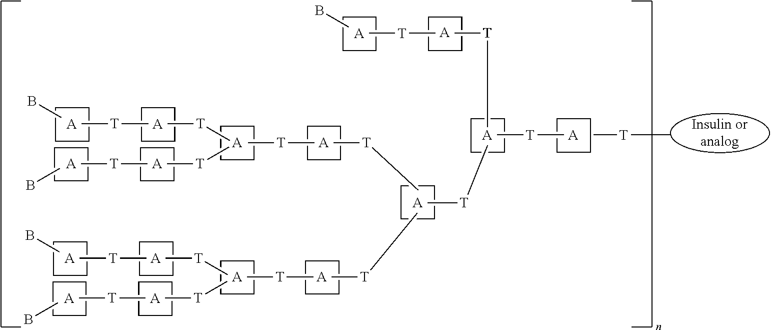
or has the general formula III: 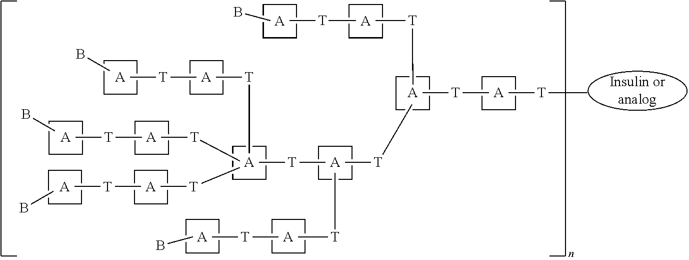
or has the general formula IV: 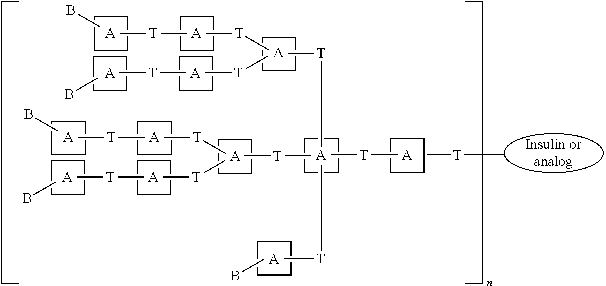
wherein
(i) T is a linker, and each occurrence of T is independently selected from a covalent bond or a bivalent, straight or branched, saturated or unsaturated, optionally substituted C₁₋₃₀ hydrocarbon chain, wherein
(a) one or more methylene units of the hydrocarbon chain of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic moiety, an aryl moiety, or a heteroaryl moiety, and
(b) each occurrence of R is independently hydrogen, a suitable protecting group, an acyl moiety, arylalkyl moiety, aliphatic moiety, aryl moiety, heteroaryl moiety, or heteroaliphatic moiety;
(ii) [symbol] is a branching point, and each occurrence of [symbol] is independently selected from a covalent bond, a carbon atom, a heteroatom, or an optionally substituted group selected from acyl moieties, aliphatic moieties, heteroaliphatic moieties, aryl moieties, heteroaryl moieties, and heterocyclic moieties; and
(iii) B is a sugar-containing moiety, and each occurrence of B is independently -T-L^{B}-X, wherein each occurrence of X is independently a ligand comprising a monosaccharide, disaccharide, trisaccharide, tetrasaccharide, or branched trisaccharide, and each occurrence of L^{B} is independently a covalent bond or a group derived from the covalent conjugation of a T with an X; and,
(iv) n is 1, 2, or 3.

10. The conjugate according to claim 1, wherein the conjugate comprising an insulin or insulin analog is conjugated to a penta-valent sugar cluster that comprises a structure selected from ML-1, ML-2, ML-3, ML-4, ML-5, ML-6, ML-7, ML-8, ML-9, ML-10, ML-11, ML-12, ML-13, ML-14, ML-15, ML-16, ML-17, ML-18, ML-19, ML-20, ML-21, ML-22, ML-23, ML-24, ML-25, ML-26, ML-27, ML-28, ML-29, ML-30, ML-31, ML-32, ML-33, ML-34, ML-35, ML-36, ML-37, ML-38, ML-39, ML-40, ML-41, ML-42, ML-43, ML-44, ML-45, ML-46, ML-47, ML-48, ML-49, ML-50, ML-51, and ML-52.

11. The conjugate according to claim 1, the conjugate is selected from IOC-1, IOC-2, IOC-3, IOC-4, IOC-5, IOC-6, IOC-7, IOC-8, IOC-9, IOC-10, IOC-11, IOC-12, IOC-13, IOC-14, IOC-15, IOC-16, IOC-17, IOC-18, IOC-19, IOC-20, IOC-21, IOC-22, IOC-23, IOC-24, IOC-25, IOC-26, IOC-27, IOC-28, IOC-29, IOC-30, IOC-31, IOC-32, IOC-33, IOC-34, IOC-35, IOC-36, IOC-37, IOC-38, IOC-39, IOC-40, IOC-41, IOC-42, IOC-43, IOC-44, IOC-45, IOC-46, IOC-47, IOC-48, IOC-49, IOC-50, IOC-51, IOC-52, IOC-53, IOC-54, IOC-55, and IOC-56.

12. A composition comprising the conjugate according to claim 1 and a pharmaceutically acceptable carrier.

13. A method for treating diabetes comprising administering to an individual in need thereof a therapeutically effective amount of the conjugate according to claim 1 to treat diabetes.

14. The method according to claim 13, wherein the diabetes is type I diabetes, type II diabetes, or gestational diabetes.

15. A method for treating diabetes comprising administering to an individual in need thereof a therapeutically effective amount of the composition according to claim 12 to treat diabetes.

16. The method according to claim 15, wherein the diabetes is type I diabetes, type II diabetes, or gestational diabetes.

17-20. (canceled)


SEQUENCE LISTING (10 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 30 aa, synthetic construct
FVNQHLCGSHLVEALYLVCGERGFFYTKPT

SEQ ID NO: 4 - protein, 30 aa, synthetic construct
FVNQHLCGSHLVEALYLVCGERGFFYTDKT

SEQ ID NO: 5 - protein, 30 aa, synthetic construct
FVKQHLCGSHLVEALYLVCGERGFFYTPDT

SEQ ID NO: 6 - protein, 21 aa, synthetic construct
GIVEQCCTSICSLYQLENYCG

SEQ ID NO: 7 - protein, 32 aa, synthetic construct
FVNQHLCGSHLVEALYLVCGERGFFYTPKTRR

SEQ ID NO: 8 - protein, 21 aa, synthetic construct
  VARIANT 8: note = T or H
  VARIANT 21: note = N or G
GIVEQCCXSICSLYQLENYCX

SEQ ID NO: 9 - protein, 21 aa, synthetic construct
  VARIANT 1: note = H or T
  VARIANT 5: note = A, G, or S
  VARIANT 6: note = H, D, E, homocysteic acid, or cysteic acid
XLCGXXLVEALYLVCGERGFF

SEQ ID NO: 10 - protein, 32 aa, synthetic construct
  VARIANT 1: note = Phenylalanine or desamino-phenylalanine
  VARIANT 5: note = H or T
  VARIANT 9: note = A, G, or S
  VARIANT 10: note = H, D, E, homocysteic acid, or cysteic acid
  VARIANT 28..29: note = This region may encompass one of the following sequences: DK, KP, or PK
  VARIANT 30..32: note = This region may encompass one of the following sequences: T, A, or TRR
XVNQXLCGXXLVEALYLVCGERGFFYTXXXXX